(19) United States
(12) Patent Application Publication
(10) Pub. No.: US 2024/0229075 A1
(43) Pub. Date: Jul. 11, 2024

(54) IMPROVED NUCLEIC ACID SEQUENCE FOR CELL TYPE SPECIFIC EXPRESSION

(71) Applicant: CureVac SE, Tübingen (DE)

(72) Inventors: Moritz THRAN, Tübingen (DE); Sandra LAZZARO, Tübingen (DE); Mallika RAMAKRISHNAN, Tübingen (DE)

(73) Assignee: CureVac SE, Tübingen (DE)

(21) Appl. No.: 18/558,531

(22) PCT Filed: May 3, 2022
(86) PCT No.: PCT/EP2022/061863

§ 371 Date: Nov. 1, 2023

(30) Foreign Application Priority Data
May 3, 2021 (WO) PCT/EP2021/061553

(51) Int. Cl. C12N 15/88; A61K 38/20; C07K 14/54; C12N 15/113

(52) U.S. Cl. CPC C12N 15/88; A61K 38/208; C07K 14/5434; C12N 15/1136; C12N 2310/141; C12N 2310/317; C12N 2310/531; C12N 2830/50; C12N 2840/007

(57) ABSTRACT

The present invention provides a nucleic acid sequence comprising at least one miRNA binding site sequence containing at least one miRNA binding site. Those miRNA binding site sequences are located within and/or immediately 3 or 5′ of the 5′ UTR of a gene to reduce the off-target side effects and allow a cell type specific expression from the nucleic acid sequence within the target organ or organs. The invention further provides pharmaceutical compositions, as well as a method of promoting cell-type specific expression, comprising the nucleic acid sequence according to the invention for use in therapy.

### INTRODUCTION

Therapeutic nucleic acids including RNA molecules represent an emerging class of drugs. RNA-based therapeutics include mRNA molecules encoding antigens for use as vaccines. In addition, it is envisioned to use RNA molecules for replacement therapies, e.g. providing missing proteins such as growth factors or enzymes to patients. Furthermore, the therapeutic use of noncoding immunostimulatory RNA molecules (e.g. WO2009/095226A2) and other noncoding RNAs such as miRNAs and long noncoding RNAs or RNAs suitable for genome editing (e.g. CRISPR/Cas9 guide RNAs) is considered. Accordingly, RNA-based therapeutics with the use in immunotherapy, gene therapy and (systemic) vaccination belong to the most promising and quickly developing therapeutic fields in modern medicine.

Some approaches also encompass the treatment of cancer diseases, also known as malignant tumors, which are a group of diseases involving abnormal cell growth with the potential to invade or spread to other parts of the body. The standard treatments of cancer include chemotherapy, radiation und surgery, or immunotherapy wherein these treatments are applied individually or in combination. Cancer immunotherapy is focused on stimulating the immune system through vaccination, adoptive cellular immunotherapy, immune checkpoint blockade or other immunostimulants or immunomodulators to elicit an anti-tumor response.

For being effective, RNA is typically delivered by lipid-based carrier systems including liposomes and lipid nanoparticles. These lipid carriers typically encapsulate the RNA and can improve intracellular delivery and effectiveness of the RNA.

Nevertheless, even with localised or targeted administration, it is possible that supplied RNA constructs may encounter or accumulate in organs, tissues, and/or cells for which they were not intended. In particular liver and kidney tissue may accumulate administered compositions, due to the physiological function of these organs. Maintaining on-target activities, tumor- or cell-specificity and reducing side effects is also a major challenge for such therapies. Thus, there is a need to further develop methods and compositions for delivery of RNA-based therapeutics to specific organs and/or tissues, and methods to modulate the expression of the delivered polynucleotide sequences in specific cells.

MiRNAs (miR or microRNA) are small single-stranded, 19-25 nucleotide long, non-coding RNA molecules found in plants, animals and some viruses, that functions in mRNA silencing and post-transcriptional regulation of gene expression. They function via Watson-Crick base-pairing with complementary sequences within the 3′ untranslated regions (3′ UTR) of target mRNA molecules. As result, these mRNA molecules are silenced. Hereby, a “seed sequence” of 2-8 nucleotides, must be perfectly complementary. Functional seeds are generally located in the 3′UTRs of mRNAs. Numerous studies have shown that multiple binding sites for the same miRNA in 3′UTRs can strongly enhance the degree of regulation. MiRNAs are derived from longer, primary transcripts termed “pri-miRNAs”. The pri-miRNAs, which can be more than 1000 nt in length, contain an RNA hairpin in which one of the two strands includes the mature miRNA (Lee et al 2002). When two mature miRNAs originate from opposite arms of the same pri-miRNA and are found in roughly similar amounts, they are denoted with a -3p or -5p suffix.

Expression pattern of miRNAs are highly specific in respect to external stimuli, developmental stage or tissue (Hamzeiy et al 2014, Fang et al 2011). Newly identified miRNAs are increasing in number with every new release of miRBase, which is the main online database providing miRNA sequences and annotation (Kozomara et al 2019). Examples of tissue-specific expression of miRNAs are in liver (miRNA-122, miRNA-125, miRNA-199), heart (miRNA-149), endothelial cells (miRNA-17-92, miRNA-126), adipose tissue (let-7, miRNA-30c), kidney (miRNA-192, miRNA-194, miRNA-204, miRNA-215, miRNA-30b, c), brain (miRNA-124a), myeloid cells (miRNA-142-3p, miRNA-142-5p, miRNA-16, miRNA-21, miRNA-223, miRNA-24, miRNA-27), pancreas (miRNA-375), muscle (miRNA-133, miRNA-206, miRNA-208), colon (miRNA-143, miRNA-145) and lung epithelial cells (let-7, miRNA-133, miRNA-126).

Among others, the miRNA-122, which is highly conserved, was one of the first examples of a tissue-specific miRNA. It is highly expressed in liver, where it constitutes 70% of the total miRNA pool, but is absent in other tissue (Jopling 2012, Bandiera et al 2015, Filipowicz and GroBhans 2011). An integration of the miRNA binding site into the 3′ UTR to improve the off-target expression has been described by the art (Brown and Naldini 2009, EP3434774, WO2019051100, WO2017062513, WO2019158955). Although target sites for endogenous miRNAs can be identified in open reading frames (ORFs) and 5′ UTRs, they are less frequent and appear less effective than those in the 3′ UTR (Lythle et al 2007). Association of miRNAs with 5′ UTR target sites appears to activate rather than repress translation (Fabian 2010, ørom 2008). Ylösmaki (2008) showed the integration of a single miRNA-122 binding site within the 5′UTR and 3′UTR in recombinant adenovirus vectors.

Summarizing the above, there are limited options for targeting expression to particular organs and cell-types and thus reducing off target side effects. This object outlined above is solved by the claimed subject matter of the invention.

### Definitions

For the sake of clarity and readability the following definitions are provided. Any technical feature mentioned for these definitions may be read on each and every embodiment of the invention.

Percentages in the context of numbers should be understood as relative to the total number of the respective items. In other cases, and depending on the context, percentages should be understood as percentages by weight (wt.-%).

About: The term “about” is used when parameters or values do not necessarily need to be identical, i.e. 100% the same. Accordingly, “about” means, that a parameter or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or values may slightly vary based on the method how the parameter was determined. For example, if a certain parameter or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 100 nucleotides; in particular, by 5, 10, 20, 30, 40, 50, 60, 70, 80, 90, 100, 110, 120, 130, 140, 150, 160, 170, 180, 190, 200 nucleotides.

Cationic, cationisable: Unless a different meaning is clear from the specific context, the term “cationic” means that the respective structure bears a positive charge, either permanently or not permanently but in response to certain conditions such as e.g. pH. Thus, the term “cationic” covers both “permanently cationic” and “cationisable”. The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation, which is well known to a person skilled in the art. E.g., if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7 particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be a DNA sequence, preferably an RNA sequence, consisting of a number of nucleotides that may be divided by three, which starts with a start codon and which preferably terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least about 70%, 80, 90%, 91%, 92%, 93%, 94%, 95%, 96%, 97%, 98%, or about 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing U by T throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences, the term “derived from” means that the amino acid sequence, which is derived from (another) amino acid sequence, shares e.g. at least about 70%, 80, 90%, 91%, 92%, 93%, 94%, 95%, 96%, 97%, 98%, or about 99% sequence identity with the amino acid sequence from which it is derived. Thus, it is understood, if e.g. a protein is “derived from” a certain protein, the protein that is “derived from” may represent a variant or fragment of said respective protein, sharing a certain percentage of sequence identity.

Fragment: The term “fragment” as used throughout the present specification in the context of a nucleic acid sequence or an amino acid (aa) sequence may typically be a shorter portion of a full-length sequence of e.g. a nucleic acid sequence or an amino acid sequence. A fragment typically consists of a sequence that is identical to the corresponding stretch within the full-length sequence. The term “fragment” as used throughout the present specification in the context of proteins or peptides may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the aa level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of antigenic proteins or peptides may comprise at least one epitope of those proteins or peptides. Furthermore, also domains of a protein, like the extracellular domain, the intracellular domain or the transmembrane domain and shortened or truncated versions of a protein may be understood to comprise a fragment of a protein.

Heterologous: The terms “heterologous” or “heterologous sequence” as used throughout the present specification in the context of a nucleic acid sequence or an amino acid sequence refers to a sequence (e.g. DNA, RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele, although heterologous sequences may be derivable from the same organism, they naturally (in nature) do not occur in the same nucleic acid molecule, such as e.g. in the same RNA or protein.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or aa sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Messenger RNA (mRNA): The term “messenger RNA” (mRNA) refers to one type of RNA molecule. In vivo, transcription of DNA usually results in the so-called premature RNA, which has to be processed into so-called messenger RNA, usually abbreviated as mRNA. Typically, an mRNA comprises a 5′-cap, a 5′-UTR of a gene, an open reading frame/coding sequence, a 3′-UTR of a gene and a poly(A).

miRNA (miRNA): the term, “miRNA (miRNA or miR)” as used herein, is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22-23 nucleotides long.

miRNA (microRNA) (miR) binding site: The term “miRNA (microRNA) (miR) binding site” as used herein, refers to a miRNA (miR) target site or a miRNA (miR) recognition site, or any nucleotide sequence to which a miRNA (miR) binds or associates. In some embodiments, a miRNA (miR) binding site represents a nucleotide location or region of a polynucleotide (e.g., an mRNA) to which at least the “seed” region of a miRNA (miR) binds. It should be understood that “binding” may follow traditional Watson-Crick hybridization rules or may reflect any stable association of the miRNA with the target sequence at or adjacent to the miRNA site. When referring to a miRNA (miR) binding site, a miRNA (miR) sequence is to be understood as having complementarity (e.g., partial, substantial, or complete (or full) complementarity) with the miRNA that binds to the miRNA binding site. A miRNA (miR) binding site can be partially complementary to a miRNA (miR), e.g., to an endogenous miRNA (miR), as is the case when the miRNA (miR) may exert translational control and/or transcript stability control of its corresponding mRNA. Alternatively, a miRNA (miR) binding site can be fully complementary (complete complementarity) to a miRNA (miR), e.g., to an endogenous miRNA (miR), as is the case when the miRNA (miR) may exert post-transcriptional and/or translational control of its corresponding mRNA. In preferred aspects of the disclosure, a miRNA (miR) binding site is fully complementary to a miRNA (miR), e.g., to an endogenous miRNA (miR), and may cause cleavage of the mRNA comprising said miRNA (miR) in cells and/or tissues in vivo, where the corresponding miR is expressed, e.g., endogenously expressed.

miRNA seed: The term “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, a miRNA seed sequence is to be understood as having complementarity (e.g., partial, substantial, or complete (or full) complementarity) with the seed sequence of the miRNA that binds to the miRNA binding site.

Nucleic acid: The terms “nucleic acid” or “nucleic acid molecule” will be recognized and understood by the person of ordinary skill in the art, and are e.g. intended to refer to a molecule comprising, preferably consisting of nucleic acid components. The term nucleic acid molecule preferably refers to DNA or RNA. It is preferably used synonymous with the term polynucleotide. Preferably, a nucleic acid or a nucleic acid molecule is a polymer comprising or consisting of nucleotide monomers (natural and/or modified), which are covalently linked to each other by phosphodiester-bonds of a sugar/phosphate-backbone. The term “nucleic acid molecule” also encompasses modified nucleic acid molecules, such as base-modified, sugar-modified or backbone-modified DNA or RNA (e.g. coding RNA) molecules as defined herein.

Nucleic acid sequence, RNA sequence, amino acid sequence: The terms “nucleic acid sequence” or “RNA sequence” will be recognized and understood by the person of ordinary skill in the art, and are e.g. intended to refer to particular and individual order of the succession of its nucleotides or amino acids respectively.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. An RNA can be single stranded or double stranded. An RNA can be circular or linear. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA that has to be processed into so-called messenger-RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, a coding sequence, optionally a 3′UTR and a poly(A) sequence. If RNA molecules are of synthetic origin, the RNA molecules are meant not to be produced in vivo, i.e. inside a cell or purified from a cell, but in an in vitro method. An example for a suitable in vitro method is in vitro transcription.

(RNA) in vitro transcription: The terms “RNA in vitro transcription” or “in vitro transcription” relate to a process wherein RNA is synthesized in a cell-free system (in vitro). RNA may be obtained by DNA-dependent in vitro transcription of an appropriate DNA template, which is typically a linear DNA template (e.g. linearized plasmid DNA or PCR product). The promoter for controlling RNA in vitro transcription can be any promoter for any DNA-dependent RNA polymerase (e.g. T7, SP6, T3). Reagents used in RNA in vitro transcription typically include a DNA template, ribonucleotide triphosphates, a cap analog, a DNA-dependent RNA polymerase, a ribonuclease (RNase) inhibitor, MgCl2, a buffer (e.g. TRIS or HEPES) which can also contain antioxidants, and/or polyamines such as spermidine at optimal concentrations.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is preferably a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined herein may comprise conservative amino acid substitution(s) compared to their native, i.e. non-mutated physiological, sequence. A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

### SHORT DESCRIPTION OF THE INVENTION

Maintaining on-target activities, tumor- or cell-specificity and reducing off-target side effects is a major challenge for mRNA-based therapies. Thus, the delivery of such therapeutics to specific organs and/or tissues is of highly interest. The present invention is inter alia based on the inventor's surprising finding that the incorporation of specific miRNA binding sites such as 122, 148a, 101 and 192, or combinations thereof preferably in front of/prior to the 5′ UTR or the coding sequence of an mRNA construct can lead to sufficient reduction of the expression from the mRNA in hepatocytes. The miRNA-122 is described to be highly expressed in liver, where it constitutes 70% of the total miRNA pool, but is absent in other tissue. So far, target sites for endogenous miRNAs were identified in open reading frames (ORFs) and 5′ UTRs, however, they are less frequent and appear to be less effective than those in the 3′ UTR.

In a first aspect, the present invention relates to a nucleic acid sequence comprising at least one coding region encoding at least one therapeutic peptide or protein and at least one miRNA binding site sequence located in 5′ direction or in 3′ direction relative to the coding region.

In particularly preferred embodiments of the first aspect, the nucleic acid sequence comprises
i) at least one 3′ UTR of a gene
ii) at least one coding region encoding at least one peptide or protein of interest
iii) at least one 5′ UTR of a gene
iv) a miRNA binding site sequence wherein the miRNA binding site sequence is located within and/or immediately 3′ or 5′ of the 5′ UTR to allow a cell type specific expression from the nucleic acid sequence within the target organ or organs. In particular, the miRNA binding site sequence comprises at least one, two, three, or four miRNA binding sites, which can be similar, identical or different. In preferred embodiments, the miRNA binding site sequence comprises one or more miRNA-122 binding sites. In some embodiments, the nucleic acid sequence can comprise a second miRNA binding site sequence within and/or immediately 3′ or 5′ of the 3′ UTR.

In a second aspect, the present invention relates to a pharmaceutical composition comprising the nucleic acid sequence as described by the first aspect and optionally comprising one or more pharmaceutically acceptable excipients, carriers, diluents and/or vehicles.

In a further aspect, the invention relates to a vaccine comprising the nucleic acid sequence of the first aspect or the pharmaceutical composition of the second aspect.

In a third aspect, the present invention relates to a kit or kit of parts comprising the nucleic acid sequence as described by the first aspect, the pharmaceutical composition of the second aspect, the vaccine, optionally comprising a liquid vehicle for solubilizing, and, optionally, technical instructions providing information on administration and/or dosage of the components.

In a fourth aspect, the present invention relates to the nucleic acid sequence of the first aspect, the pharmaceutical composition of the second aspect, the vaccine, or the kit or kit of parts of the third aspect for use as a medicament.

Further aspects relate to methods of treating or preventing a disease, disorder, or condition and a method to promote a cell-type specific expression of a peptide or protein within a target organ or organs by using a nucleic acid sequence.

### DETAILED DESCRIPTION OF THE INVENTION

The present application is filed together with a sequence listing in electronic format, which is part of the description of the present application (WIPO standard ST.25). The information contained in the electronic format of the sequence listing filed together with this application is incorporated herein by reference in its entirety. For many sequences, the sequence listing also provides additional detailed information, e.g. regarding certain structural features, sequence modifications, GenBank identifiers, or additional detailed information. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments.

This description should be understood to support and encompass embodiments, which combine the explicitly described embodiments with any number of the disclosed and/or preferred elements. Furthermore, any permutations and combinations of all described elements in this application should be considered as disclosed by the description of the present application, unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present invention is based on the finding that the incorporation of at least one miRNA binding site such as miRNA-122, -148a, -101, 194, or -192 binding site within and/or immediately 3′ or 5′ of the 5′UTR allows a cell type specific expression from the nucleic acid sequence within the target organ or organs and prevents off-target effects by expression in non-target cells. Particularly, the expression of the peptide or protein of interest from the nucleic acid sequence is preferably reduced in the liver. This effect is particularly important if expression e.g. in immune cells such as dendritic cells and muscle cells is preferred, such as in the case of vaccinations. Furthermore liver expression might be unwanted if intratumoral treatment is intended.

In addition, the present invention is based on the findings that the incorporation of at least one miRNA binding site such as miRNA-142 or -223 binding site sequences within and/or immediately 3′ or 5′ of the 5′UTR allows a cell type specific expression from the nucleic acid sequence within the target organ or organs and prevents off-target effects by expression in non-target cells. Particularly, the expression of the peptide or protein of interest from the nucleic acid sequence is preferably reduced in immune cells. This effect is particularly important if expression e.g. in muscle cells or tumor cells is preferred, such as in the case of protein replacement therapy or intratumoral treatment with cytostatic or cytotoxic peptides or proteins.

First Aspect: Nucleic Acid Sequence Comprising a miRNA Binding Site Sequence

According to the first aspect a nucleic acid sequence comprising at least one coding region encoding at least one peptide or protein and at least one first miRNA binding site sequence located in 5′ direction relative to the coding region and/or at least one second miRNA binding site sequence in 3′ direction relative to the coding region.

In the context of the invention, a miRNA binding site sequence that is located in 5′ direction relative to the coding region is herein referred to as “first miRNA binding site sequence”. Notably, the nucleic acid sequence may comprise more than one first miRNA binding site sequence, e.g. 2, 3, 4, 5. These more than one first miRNA binding site sequences may be similar in sequence or different.

A miRNA binding site sequence that is located in 3′ direction relative to the coding region is herein referred to as “second miRNA binding site sequence”. Notably, the nucleic acid sequence may comprise more than one second miRNA binding site sequence, e.g. 2, 3, 4, 5. These more than one first miRNA binding site sequences may be similar in sequence or different.

In particularly preferred embodiments, the nucleic acid sequence comprises at least one coding region encoding at least one therapeutic peptide or protein and at least one first miRNA binding site sequence located in 5′ direction relative to the coding region. In such embodiments, the nucleic acid sequence may additionally comprise at least one second miRNA binding site sequence.

In alternative embodiments, the nucleic acid sequence comprises at least one coding region encoding at least one therapeutic peptide or protein and at least one second miRNA binding site sequence located in 3′ direction relative to the coding region. In such embodiments, the nucleic acid sequence may additionally comprise at least one first miRNA binding site sequence.

In preferred embodiments, the The nucleic acid sequence of the invention comprises at least two, three, or four first miRNA binding site sequences located in 5′ direction relative to the coding region. The at least two, three, or four first miRNA binding site sequences may comprise essentially the same miRNA binding sites (and may therefore be essentially similar in sequence) or may comprise different miRNA binding sites (and may therefore be different in sequence).

The nucleic acid sequence of the invention, additionally comprising at least one 5′ UTR as further defined herein.

Further the nucleic acid sequence of the invention comprising at least one 5′UTR, wherein the at least one 5′ UTR is selected or derived from a gene.

According to preferred embodiments, the nucleic acid sequence comprises at least one first miRNA binding site sequence located in 5′ direction relative to a coding region is
I) located in 5′ direction relative to the 5′ UTR; and/or
II) is located within the 5′ UTR; and/or
III) located between the 5′ UTR and the coding region

In other embodiments, the present invention relates to a nucleic acid sequence comprising
i) at least one 3′ UTR of a gene (as further specified herein);
ii) at least one coding region encoding at least one peptide or protein of interest;
iii) at least one 5′ UTR of a gene
iv) a miRNA binding site sequence wherein the miRNA binding site sequence is located within and/or immediately 3′ or 5′ of the 5′UTR to allow a cell type specific expression from the nucleic acid sequence within the target organ or organs.

In some embodiments, the nucleic acid sequence comprises a 3′ UTR, preferably a 3′ UTR selected or derived from a gene (3′ untranslated region) or a part of a 3′ UTR as described in WO2019077001 and further described below within this invention, particularly at least one 3′ untranslated region (3′ UTR) element derived from a 3′ UTR of a gene selected from the group consisting of PSMB3, CASP1, COX6B1, GNAS, NDUFA1 or and RPS9.

The terms “coding sequence”, “coding region”, or “cds” as used herein will be recognized and understood by the person of ordinary skill in the art, and are e.g. intended to refer to a sequence of several nucleotides which may be translated into a peptide or protein. In the context of the present invention a cds is preferably an RNA sequence, consisting of a number of nucleotide triplets, starting with a start codon and preferably terminating with one stop codon. In embodiments, the cds of the RNA may terminate with one or two or more stop codons. The first stop codon of the two or more stop codons may be TGA or UGA and the second stop codon of the two or more stop codons may be selected from TAA, TGA, TAG, UAA, UGA or UAG.

According to further embodiments at least one coding sequence of the nucleic acid sequence of the invention may encode at least two, three, four, five, six, seven, eight and more, preferably distinct, (poly)peptides or proteins of interest linked with or without an amino acid linker sequence, wherein said linker sequence may comprise rigid linkers, flexible linkers, cleavable linkers (e.g., self-cleaving peptides) or a combination thereof.

In embodiments, the length of the coding sequence may be at least or greater than about 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the coding sequence may be in a range of from about 300 to about 2000 nucleotides.

In preferred embodiments, the nucleic acid sequence is a coding RNA. Most preferably, said coding RNA may be selected from an mRNA, a (coding) self-replicating RNA (replicon RNA), a (coding) circular RNA, or a (coding) viral RNA. A coding RNA can be any type of RNA construct (for example a double stranded RNA, a single stranded RNA, a circular double stranded RNA, or a circular single stranded RNA) characterized in that said coding RNA comprises at least one coding sequence (cds) that is translated into at least one amino-acid sequence (upon administration to e.g. a cell). A viral RNA is defined as the genetic material of an RNA virus. This nucleic acid is usually single-stranded RNA (ssRNA) but may be double-stranded RNA (dsRNA). A retroviral RNA is defined as a ssRNA of retroviruses In some embodiments, the nucleic acid sequence is a circular RNA. As used herein, the terms “circular RNA” or “circRNAs” have to be understood as a circular polynucleotide construct that may encode at least one peptide or protein. Preferably, such a circRNA is a single stranded RNA molecule. In preferred embodiments, said circRNA comprises at least one coding sequence encoding at least one peptide or protein as defined herein, or a fragment or variant thereof.

In other embodiments, the nucleic acid sequence is a replicon RNA. The term “replicon RNA” is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding an antigenic peptide or protein as defined herein). Alternatively, the replicase may be provided on an independent coding RNA construct or a coding DNA construct. Downstream of the replicase may be a sub-genomic promoter that controls replication of the replicon RNA.

In particularly preferred embodiments, the nucleic acid sequence is not a self-replicating RNA or replicon RNA.

In other embodiments the nucleic acid sequence is a DNA, e.g. a plasmid DNA, viral DNA, etc.

In an additional embodiment, the nucleic acid sequence comprises at least one coding sequence encoding at least one peptide or protein as further defined below, and additionally at least one further heterologous peptide or protein element.

Suitably, the at least one further heterologous peptide or protein element may be selected from secretory signal peptides, transmembrane elements, multimerization domains, VLP (virus-like particles) forming sequence, a nuclear localization signal (NLS), peptide linker elements, self-cleaving peptides, immunologic adjuvant sequences or dendritic cell targeting sequences.

In a preferred embodiment the nucleic acid sequence of the invention is selected from DNA or RNA, preferably from plasmid DNA, viral DNA, template DNA, viral RNA, self-replicating RNA, circular RNA, replicon RNA, or an mRNA.

In a particular preferred embodiment, the nucleic acid sequence of the invention is a linear nucleic acid, preferably a single-stranded linear nucleic acid.

In one embodiment the nucleic acid sequence of the invention is not selected or derived from an adenoviral vector or wherein the nucleic acid is not isolated from a cell, tissue, or organism.

In a preferred embodiment the nucleic acid of the invention is an in vitro transcribed RNA, preferably an in vitro transcribed mRNA.

In various embodiments, the nucleic acid sequence comprises a 5′ UTR, preferably a 5′ UTR selected or derived from a gene (5′ untranslated region) or a part of a 5′ UTR as described in WO2019077001 and further described below within this invention, particularly at least one 5′ untranslated region (5′ UTR) element derived from a 5′ UTR of a gene selected from the group consisting of HSD17B4, ASAH1, ATP5A1, MP68, NDUFA4, NOSIP, RPL31, SLC7A3, TUBB4B and UBQLN2.

According to preferred embodiments, the nucleic acid sequence comprises a miRNA binding site sequence wherein the miRNA binding site sequence is located within and/or immediately 3′ or 5′ of the 5′UTR to allow a cell type specific expression from the nucleic acid sequence within the target organ or organs. This miRNA binding site sequence, which is located within, and/or immediately 3′ or 5′ of the 5′UTR is also defined as the first binding site sequence of the nucleic acid sequence.

miRNAs

MiRNAs (microRNAs or miRs) are a class of noncoding RNAs each containing around 20 to 25 nucleotides some of which are believed to be involved in post-transcriptional regulation of gene expression by binding to complementary sequences in the 3′ UTR/and or 5′ UTR of target mRNAs, leading to their silencing. These sequences are also referred to herein as miRNA binding sites. Hereby, one or more miRNA binding sites can be placed within a miRNA binding site sequence within the nucleic acid sequence. Certain miRNAs are highly tissue-specific in their expression; for example, miR-122 and its variants are abundant in the liver and infrequently expressed in other tissues (Lagos-Quintana (2002), Current Biology, Vol. 12, April).

As defined, herein a miRNA binding site sequence is a part or a section of the nucleic acid sequence according to this invention. Hereby, this miRNA binding site sequence comprises at least one miRNA binding site. The term “miRNA binding site” is further described below.

A miRNA comprises a “seed” region or sequence, i.e., a sequence in the region of positions 2-8 of the mature miRNA, which sequence has perfect Watson-Crick complementarity to the miRNA target sequence. The bases of the miRNA seed region or sequence have complete complementarity with the target sequence. MiRNAs derive enzymatically from regions of RNA transcripts that fold back on themselves to form short hairpin structures often termed a pre-miRNA (precursor-miRNA). The pre-miRNA typically has a two-nucleotide overhang at its 3′ end, and has 3′ hydroxyl and 5′ phosphate groups. This precursor-mRNA is processed in the nucleus and subsequently transported to the cytoplasm where it is further processed by DICER (an RNase III enzyme), to form a mature miRNA of approximately 22 nucleotides. The mature miRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives.

A miRNA referred to by number herein can refer to either of the two mature miRNAs originating from opposite arms of the same pre-miRNA (e.g., either the 3p or 5p miRNA). All miRNAs referred to by number herein are intended to include both the 3p and 5p arms/sequences. “5p” means the miRNA is from the 5 prime arm of the pre-miRNA hairpin and “3p” means the miRNA is from the 3 prime end of the pre-miRNA hairpin. A miRNA referred to by number herein can refer to either of the two mature miRNAs originating from opposite arms of the same pre-miRNA (e.g., either the 3p or 5p miRNA). All miRNAs referred to herein are intended to include both the 3p and 5p arms/sequences, unless particularly specified by the 3p or 5p designation.

The miRNA system therefore provides a robust platform by which nucleic acids introduced into cells can be silenced in selected cell types in a target tissue and expressed in others. By including a binding site for a particular given miRNA sequence into an mRNA construct to be introduced into target cells, particularly in or immediately 5′ or 3′ to a UTR, expression from certain introduced genes can be reduced or substantially eliminated in some cell types, while remaining in others (Brown and Naldini, Nature Reviews Genetics volume 10, pages 578-585 (2009)).

The use of the term ‘immediately’ is understood to be synonymous with terms such as ‘highly proximate to’ or ‘very close to’. When referring to 5′ or 3′ positioning relative to a UTR sequence it encompasses variants in which typically up to around twenty, suitably not more than fifty, intervening nucleotide bases may be placed between the miRNA binding sequence and the adjacent UTR. The use of the term ‘immediately’ is understood to be synonymous with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 41, 42, 43, 44, 45, 46, 47, 48, 49 or 50 intervening nucleotide bases placed between the miRNA binding sequence and the adjacent UTR. In a preferred embodiment the term ‘immediately’ is understood to be synonymous with 0, 1, 2, 3, 4, 5, or 6 intervening nucleotide bases placed between the miRNA binding sequence and the adjacent UTR. In particularly preferred embodiment the term ‘immediately’ is understood to be synonymous with 0 intervening nucleotide bases placed between the miRNA binding sequence and the adjacent UTR.

It is contemplated that one, or a plurality, of such miRNA binding sites can be included in the nucleic acid sequence, e.g. an mRNA construct. Where a plurality of miRNA binding sites are present, this plurality may include for example greater than two, greater than three, typically greater than four miRNA binding sites. These miRNA binding sites may be arranged sequentially, in tandem or at predetermined locations within miRNA binding site sequences, 3′ to, or 5′ to a specified UTR within the nucleic acid sequences, e.g. mRNA constructs. Multiple binding sites may be separated with a linker sequence, which may vary, or may comprise a particular sequence, for example, “uuuaaa”. In some embodiments, no linker sequence may be present between binding sites. Other parts of the nucleic acid sequence, e.g. mRNA sequence can incorporate linker sequences, such as between the stop codon of the ORF(cds) and the optional UTR or miRNA binding sites.

As used herein, the term “miRNA (miRNA) binding site” refers to a sequence within a polynucleotide (nucleic acid sequence), e.g., within a DNA or within an RNA or RNA transcript, that has sufficient complementarity to all or a region of a miRNA to interact with, associate with or bind to the miRNA. In exemplary embodiments, miRNA binding sites are included in RNA sequences, e.g. mRNAs, for example, in the 5′ UTR and/or 3′ UTR of an mRNA. A miRNA binding site having sufficient complementarity to the miRNA refers to a degree of complementarity sufficient to facilitate miRNA-mediated regulation of the mRNA, e.g., miRNA-mediated translational repression or degradation of the mRNA. In exemplary aspects of the disclosure, a miRNA binding site having sufficient complementarity to the miRNA refers to a degree of complementarity sufficient to facilitate miRNA-mediated degradation of the mRNA, e.g., miRNA-guided RISC-mediated cleavage of mRNA. The miRNA binding site can have complementarity to, for example, a 19-25 nucleotide long miRNA sequence, to a 19-23 nucleotide long miRNA, most typically to a 22-nucleotide long miRNA sequence. A miRNA binding site may be complementary to only a portion of a miRNA, e.g., to a portion 1, 2, 3 or 4 nucleotides shorter that a naturally occurring miRNA. Full or complete complementarity (e.g., fully complementary or completely complementary over all or a significant portion of a naturally occurring miRNA) is preferred when the desired regulation is RNA or mRNA degradation. In some embodiments, a miRNA binding site includes a sequence that has complementarity (e.g., partial or complete complementarity) with a miRNA seed sequence. In particular embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In particular embodiments, the miRNA binding site includes a sequence that has complete complementarity with a miRNA sequence. In some embodiments, a miRNA binding site has complete complementarity with a miRNA sequence but for 1, 2, 3, 4, 5, 6, 7, 8, 9, 10, 11, 12, 13, 14 or 15 nucleotide substitutions, terminal additions, and/or truncations. In a preferred embodiment, a miRNA binding site has complete complementarity with a miRNA sequence but for 1, 2 or 3 nucleotide substitutions, terminal additions, and/or truncations. In a very preferred embodiment, a miRNA binding site has complete complementarity with a miRNA sequence.

One or more miRNA binding sites can be incorporated in a miRNA binding site sequence (e.g. the first miRNA binding site sequence or the second miRNA binding site sequence) of the nucleic acid sequence of the disclosure for one or more of a variety of different purposes. For example, incorporation of one or more miRNA binding sites into a miRNA binding site sequence of an RNA or mRNA of the disclosure may target the molecule for degradation or reduced translation, provided the miRNA in question is available (e.g., expressed in a target cell or tissue.) In some embodiments, incorporation of one or more miRNA binding sites into a miRNA binding site sequence of an RNA or mRNA of the disclosure may reduce the hazard of off-target effects upon nucleic acid molecule delivery and/or enable tissue-specific regulation of expression of a polypeptide encoded by the RNA or mRNA. In yet other embodiments, incorporation of one or more miRNA binding sites into in a miRNA binding site sequence of an RNA or mRNA of the disclosure can modulate immune responses upon nucleic acid sequence delivery in vivo.

For example, an RNA or mRNA may include one or more miRNA binding sites that are bound by miRNAs that have higher expression in one tissue type as compared to another. In another example, an RNA or mRNA may include one or more miRNA binding sites that are bound by miRNAs that have lower expression in a cancer cell as compared to a non-cancerous cell of the same tissue of origin. When present in a cancer cell that expresses low levels of such a miRNA, the polypeptide encoded by the RNA/mRNA/nucleic acid sequence typically will show increased expression. If the polypeptide is able to induce apoptosis, this may result in preferential cell killing of cancer cells as compared to normal cells. Therefore it is particularly preferred in some embodiments that miRNA binding sites are used which are bound to miRNAs mainly expressed in liver cells to reduce off-targets effects from liver expression. This is particularly of importance in case of vaccination wherein a specific expression in immune cells is preferred and e.g. in case of intratumoral treatment wherein specific expression in cancer cells is intended.

As used herein, the term “miRNA binding site” refers to a miRNA target site or a miRNA recognition site, or any nucleotide sequence to which a miRNA binds or associates. It should be understood that “binding” may follow traditional Watson-Crick hybridization rules or may reflect any stable association of the miRNA with the target sequence at or adjacent to the miRNA site. Accordingly, as defined herein, a miRNA binding site sequence is a sequence, which can contain one or more miRNA binding sites. Those binding sites are complementary to miRNAs, preferably to sequences selected from miRNA-122, miRNA-142, miRNA-148a, miRNA-101, miRNA-192, miRNA-194 or miRNA-223.

In various embodiments, the miRNA binding site sequence (e.g. the first miRNA binding site sequence or the second miRNA binding site sequence) comprises at least one, two, three, or four miRNA binding sites. As used herein, the term ‘miRNA binding sequence’ is synonymous with an ‘miRNA binding pattern’ or ‘miRNA binding element’ or ‘miRNA binding region’.

Accordingly, as defined herein, a miRNA binding site sequence is a sequence, which can contain one or more miRNA binding sites as defined herein (see exemplary FIG. 12).

Further, a nucleic acid sequence may comprise at least one first miRNA binding site sequence (that is, a miRNA binding site sequence located in 5′ direction relative to the cds). Alternatively, a nucleic acid sequence may comprise at least two or more first miRNA binding site sequences.

Alternatively or additionally, a nucleic acid sequence may comprise at least one second miRNA binding site sequence (that is, a miRNA binding site sequence located in 3′ direction relative to the cds). Alternatively, a nucleic acid sequence may comprise at least two or more second miRNA binding site sequences.

The presence of a plurality of miRNA binding sites in the mRNA construct enables improved efficacy of the differential expression of the supplied polypeptide or polypeptides. Without being bound by theory, it is thought that with an increased number of sites for binding, the likelihood of miRNA binding and consequent degradation is increased. Multiple miRNA binding sites can comprise multiple copies of substantially the same binding site, thereby introducing redundancy. Alternatively, or additionally, the multiple binding sites can comprise substantially different sequences, thereby allowing the nucleic acid sequence or mRNA construct to be targeted by more than one species of miRNA. In this way, differential expression from a supplied nucleic acid sequence or mRNA construct can be achieved for more than one cell type, and/or in more than one organ, as is evident from the discussion of organs and their associated miRNA sequences above. Both approaches are considered possible within the same sequence or multiple sequences. An intermediate approach is also envisioned, wherein multiple binding sites are included which are intended to be targets for the same miRNA sequence but have differences in order to bind different variants of the same miRNA sequence.

Some advantages associated with the use of multiple binding sites include an increase in the efficiency of differential expression of polypeptides supplied by the nucleic acid sequence or mRNA sequences of the present invention, within a single organ. Use of different binding sites, which are applicable to more than one tissue or organ type, can enable differential expression to be achieved in different cell types in more than one organ or tissue. This may be desirable when systemic administration of compositions according to the invention is used, and it is necessary to avoid off-target effects in more than one organ, tissue or cell type.

First miRNA Binding Site Sequence

According to preferred embodiments, the nucleic acid sequence comprises at least one first miRNA binding site sequence located in 5′ direction relative to the coding region.

In preferred embodiments, the nucleic acid sequence comprising at least one 5′UTR, preferably selected or derived from a gene.

The nucleic acid sequence of the invention suitably comprises a first miRNA binding site sequence located in 5′ direction relative to a coding region is
I) located in 5′ direction relative to the 5′ UTR; and/or
II) is located within the 5′ UTR; and/or
III) located between the 5′ UTR and the coding region.

In preferred embodiments, the at least one first miRNA binding site sequence is located in 5′ direction relative to the 5′ UTR.

In various embodiments, the nucleic acid sequence of the invention comprises a 5′ terminal cap structure, as described in section “cap” below.

In preferred embodiments, the nucleic acid sequence comprises a 5′ terminal cap structure and the at least one first miRNA binding site sequence that is located between said 5′ terminal cap structure and the 5′ UTR.

In other preferred embodiments, the at least one first miRNA binding site sequence is located in 5′ direction relative to the 5′ UTR and at least one first miRNA binding site sequence is located within the 5′ UTR.

The at least one first miRNA binding site sequence of the invention is located in a distance of less than 20 nucleotides, less than 5 nucleotides, less than 1 nucleotide relative to the 5′ UTR. Preferably, the least one first miRNA binding site sequence is located in sequence with the 5′ UTR (in 5′ direction) without any intermediate nucleotide (that is, the distance is 0 nucleotides).

In preferred embodiments, the first miRNA binding site sequence comprises at least one miRNA binding site for reducing or preventing expression in liver, kidney, immune cells, or endothelial cells, or any combination thereof.

In particularly preferred embodiments, the first miRNA binding site sequence comprises at least one miRNA binding site for reducing or preventing expression in liver cells and/or immune cells.

In preferred embodiments, the at least one first miRNA binding site sequence comprises at least one, two, three, or four miRNA binding sites. In embodiments, the at least one, two, three, or four miRNA binding sites are selected from substantially similar miRNA binding sites. In other embodiments, the at least one, two, three, or four miRNA binding sites are selected from substantially different miRNA binding sites.

Second miRNA Binding Site Sequence

According to preferred embodiments, the nucleic acid sequence comprises at least one second miRNA binding site sequence located in 3′ direction relative to the coding region.

In particularly preferred embodiments, the nucleic acid sequence comprises at least one first miRNA binding site sequence located in 5′ direction relative to the coding region (as defined herein) and additionally at least one second miRNA binding site sequence located in 3′ direction relative to the coding region (as defined herein).

In embodiments, the nucleic acid sequence comprises at least two, three, or four second miRNA binding site sequences located in 3′ direction relative to the coding region.

In preferred embodiments, the nucleic acid sequence of the invention additionally comprises at least one 3′ UTR, preferably at least one 3′ UTR selected or derived from a gene.

In preferred embodiments, the nucleic acid sequence of the invention comprises at least one second miRNA binding site sequence located in 3′ direction relative to the coding region is
I) located in 3′ direction relative to the 3′ UTR; and/or
II) is located within the 3′ UTR; and/or
III) located between the 3′ UTR and the coding region.

In particularly preferred embodiments, the at least one second miRNA binding site sequence is located in 3′ direction relative to the 3′ UTR.

In various embodiments, the nucleic acid of the invention comprises at least one poly(A) sequence, and/or at least one poly(C) sequence, and/or at least one histone stem-loop sequence/structure, as described below in section “PolyA/PolyC/HSL”.

In preferred embodiments, the nucleic acid sequence comprises at least one poly(A) sequence, preferably comprising about 40 to about 200 adenosine nucleotides, most preferably about 100 adenosine nucleotides.

In preferred embodiments, the nucleic acid sequence of the invention comprises at least one poly(A) sequence and the at least one second miRNA binding site sequence is located between the poly(A) sequence and the 3′ UTR.

In other embodiments, the at least one second miRNA binding site sequence is located in 3′ direction relative to the 3′ UTR and at least one second miRNA binding site sequence is located within the 3′ UTR.

In embodiments, the at least one second miRNA binding site sequence is located in a distance of less than 20 nucleotides, less than 5 nucleotides, less than 1 nucleotide relative to the 3′ UTR. Preferably, the least one second miRNA binding site sequence is located in sequence with the 3′ UTR (in 3′ direction) without any intermediate nucleotide (that is, the distance is 0 nucleotides).

In preferred embodiments, the at least one second miRNA binding site sequence comprises at least one miRNA binding site for reducing or preventing expression in liver, kidney, immune cells, or endothelial cells, or any combination thereof. In particularly preferred embodiments, the second miRNA binding site sequence comprises at least one miRNA binding site for reducing or preventing expression in liver cells and/or immune cells.

According to preferred embodiments, the at least one second miRNA binding site sequence comprises at least one, two, three, or four miRNA binding sites. In embodiments, the at least one, two, three, or four miRNA binding sites are selected from substantially similar miRNA binding sites. In another embodiment the at least one, two, three, or four miRNA binding sites are selected from substantially different miRNA binding sites.

miRNA Binding Sites In some embodiments, at least one first or second miRNA binding site sequence comprises at least two substantially similar miRNA binding sites.

As defined herein, “similar” means having a resemblance in appearance or nature; alike though not identical.

In some preferred embodiments, at least one first or second miRNA binding site sequence comprises at least two identical miRNA binding sites.

For example, the first miRNA binding site sequence of the nucleic acid sequence can contain two miRNA-122 binding sites. Thus, those miRNA binding sites can also be identical miRNA binding sites.

In most preferred embodiments, the miRNA binding site sequence of the nucleic acid sequence can contain two miRNA-122-5p binding sites.

In other embodiments, at least one miRNA binding site sequence comprises at least two substantially different miRNA binding sites.

For example, the miRNA binding site sequence of the nucleic acid sequence can contain one miRNA-122 binding site, preferably a miRNA-122-5p binding site, and one miRNA-192 binding site, preferably a miRNA-192-5p binding site. Those different binding sites can be located within one miRNA binding site sequence or within two different miRNA binding site sequences.

Introduction of one or multiple binding sites for different miRNA can be engineered to further decrease the longevity, stability, and protein translation of a nucleic acid sequence.

miRNAs, which are differentially expressed in different tissues and cells, and often associated with different types of diseases (e.g. cancer cells). The decision of removal or insertion of miRNA binding sites, or any combination, is dependent on miRNA expression patterns and their profiling in cells.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), nervous system (mir-124a, miR-9), pluripotent cells (miR-302, miR-367, miR-290, miR-371, miR-373), pancreatic islet cells (miR-375), adipose tissue (let-7, miR-30c), heart (miR-1d, miR-149), kidney (miR-192, miR-194, miR-204), and lung epithelial cells (let-7, miR-133, miR-126).

miRNAs that are known to be expressed in the liver include, but are not limited to, miRNA-101, miR-107, miR-122-3p, miR-122-5p, miRNA-125, miR-148a-5p, miR-148a-3p, miRNA-192, miRNA-194, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, miR-939-5p. miRNA binding sites from any liver specific miRNA can be introduced to or removed from the nucleic acid sequence to regulate the expression from the nucleic acid sequence in the liver. Liver specific miRNAs binding sites can be engineered alone or further in combination with immune cells (e.g. APCs) miRNA binding sites in order to prevent an immune reaction against protein expression in the liver.

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, miR-99b-5p, miR-579-3b, miR-4516. miR-664b-3p, miR-342-3p, miR-1915-3p and miR-4286. Novel miRNAs are discovered in the immune cells in the art through micro-array hybridization and microtome analysis (Jima D D et al, Blood, 2010, 116:e118-e127; Vaz C et al., BMC Genomics, 2010, 11, 288, the content of each of which is incorporated herein by reference in its entirety).

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, miR-381-5p. MiRNA binding sites from any lung specific miRNA can be introduced to or removed from the nucleic acid sequence to regulate the expression from the nucleic acid sequence in the lung. Lung specific miRNAs binding sites can be engineered alone or further in combination with immune cells (e.g. APCs) miRNA binding sites in order to prevent an immune reaction against protein expression in the lung.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p and miR-92b-5p. MiRNA binding sites from any heart specific miRNA can be introduced to or removed from the nucleic acid sequence to regulate the expression from the nucleic acid sequence in the heart. Heart specific miRNAs binding sites can be engineered alone or further in combination with immune cells (e.g. APCs) miRNA binding sites in order to prevent an immune reaction against protein expression in the heart.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p and miR-9-5p. MiRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, miR-657. MiRNA binding sites from any CNS specific miRNA can be introduced to or removed from the nucleic acid sequence to regulate the expression from the nucleic acid sequence in the nervous system. Nervous system specific miRNAs binding sites can be engineered alone or further in combination with immune cells (e.g. APCs) mRNA binding sites in order to prevent an immune reaction against protein expression in the nervous system.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p and miR-944. MiRNA binding sites from any pancreas specific miRNA can be introduced to or removed from the nucleic acid sequence to regulate the expression from the nucleic acid sequence in the pancreas. Pancreas specific microRNAs binding sites can be engineered alone or further in combination with immune cells (e.g. APCs) miRNA binding sites in order to prevent immune reaction against protein expression in the pancreas.

miRNAs that are known to be expressed in the kidney further include, but are not limited to, miR-122-3p, miR-145-5p, miR-17-5p, miR-192-3p, miR-192-5p, miR-194-3p, miR-194-5p, miR-20a-3p, miR-20a-5p, miRNA-20b/c, miR-204-3p, miR-204-5p, miR-210, miRNA-215, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p and miR-562. MiRNA binding sites from any kidney specific miRNA can be introduced to or removed from the nucleic acid sequence to regulate the expression from the nucleic acid sequence in the kidney. Kidney specific miRNAs binding sites can be engineered alone or further in combination with immune cells (e.g. APCs) miRNA binding sites in order to prevent immune reaction against protein expression in the kidney.

miRNAs that are known to be expressed in the muscle further include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p and miR-25-5p. MiRNA binding sites from any muscle specific miRNA can be introduced to or removed from the nucleic acid sequence to regulate the expression from the nucleic acid sequence in the muscle. Muscle specific miRNAs binding sites can be engineered alone or further in combination with immune cells (e.g. APCs) miRNA binding sites in order to prevent an immune reaction against protein expression in the muscle.

miRNAs are differentially expressed in different types of cells, such as endothelial cells, epithelial cells and adipocytes. For example, miRNAs that are expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p and miR-92b-5p. Many novel miRNAs were discovered in endothelial cells from deep-sequencing analysis Voellenkle C et al., RNA, 2012, 18, 472-484, herein incorporated by reference in its entirety).

Many miRNA expression studies have been conducted, and are described in the art, to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma WO2013/011378); colorectal cancer cells (WO2011/0281756, WO2011/076142); cancer positive lymph nodes WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in their entirety).

In preferred embodiments, the nucleic acid sequence comprises at least one miRNA binding site, which is substantially complementary to miRNA sequences selected from at least one or more of the group of Table I consisting of miRNA-122, miRNA-148a, miRNA-101, miRNA-192, miRNA-194, miRNA-142 or miRNA-223.

TABLE I
miRNA binding sites used in embodiments of the present invention

| | Implicated | | |
|---|---|---|---|
| Tissue | miRNA | Expression profile | Reference |
| Liver | miRNA- | Expressed in liver; | Braconi 2011, |
| | 122 | Reduced in cancer cells | Brown 2009 |
| Liver and | miRNA- | Expressed in immune | Ther 2008, |
| immune cells | 148a | cells, liver and various | Yu 2007, |
| | | tissues like cerebral, | Takamizawa |
| | | heart, liver, thymus, | 2004, |
| | | pancreas, renal, placenta, | Yue 2016 |
| | | uterus, testis, and the | |
| | | hematopoietic system | |
| Endothelial cells, | miRNA- | Expressed in endothelial | Xie 2014 |
| epithelial cells | 101 | cells, epithelial cells | |
| and adipocytes, | | and adipocytes and | |
| liver | | liver, liver cancer | |
| Kidney and liver | miRNA- | Kidney-specific | Ren 2021 |
| | 192 | expression, liver and | |
| | | liver disease/cancer | |
| Kidney and liver | miRNA- | Kidney-specific | Sempere 2004, |
| | 194 | expression, liver and | Sun, 2004, |
| | | liver disease/cancer | Wu 2019 |
| Immune cells | miRNA- | Immune cell | miRbase |
| | 142 | | |
| Immune cells | miRNA- | Immune cell | miRbase |
| | 223 | | |

According to preferred embodiments, the nucleic acid sequence of the invention comprises at least one first miRNA binding site sequence that comprises one or more of the group consisting of binding sites for miRNA-122, miRNA-142, miRNA-148a, miRNA-101, miRNA-192, miRNA-194, and miRNA-223.

In preferred embodiments, the nucleic acid sequence of the invention comprises the at least one first miRNA binding site sequence that comprises one or more of the group consisting of the binding sites for miRNA-122-5p, miRNA-142-3p, miRNA-148a-3p, miRNA-101-3p, miRNA-192-5p, miRNA-194-5p, and miRNA-223-3p.

In other preferred embodiments, the nucleic acid sequence of the invention comprises the at least one first miRNA binding site sequence that comprises one or more of miRNA-122, miRNA-148a, and miRNA-223, preferably miRNA-122-5p, miRNA-148a-3p, and miRNA-223-3p.

In embodiments, the nucleic acid sequence of the invention comprises the at least one first miRNA binding site sequence that comprises or consists of at least two or three miRNA-101 binding sites, preferably miRNA-101-3p.

In embodiments, the nucleic acid sequence of the invention comprises the at least one first miRNA binding site sequence that comprises or consists of at least two or three miRNA-192 binding sites, preferably miRNA-192-5p.

In embodiments, the nucleic acid sequence of the invention comprises the at least one first miRNA binding site sequence that comprises or consists of at least two or three miRNA-192 binding sites, preferably miRNA-194-5p.

In embodiments, the nucleic acid sequence of the invention comprises the at least one first miRNA binding site sequence that comprises or consists of at least two or three miRNA-142 binding sites, preferably miRNA-142-3p.

In preferred embodiments, the nucleic acid sequence of the invention comprises the at least one first miRNA binding site sequence that comprises or consists of at least two or three miRNA-122 binding sites, preferably miRNA-122-5p.

In further preferred embodiments, the nucleic acid sequence of the invention comprises the at least one first miRNA binding site sequence that comprises or consists of at least two or three miRNA-148a binding sites, preferably miRNA-148a-3p.

In preferred embodiments, the nucleic acid sequence of the invention comprises the at least one first miRNA binding site sequence that comprises or consists of at least two or three miRNA-223 binding sites, preferably miRNA-223-3p.

According to preferred embodiments, the nucleic acid sequence of the invention comprises at least one second miRNA binding site sequence that comprises one or more of the group consisting of binding sites for miRNA-122, miRNA-142, miRNA-148a, miRNA-101, miRNA-192, miRNA-194, and miRNA-223.

In preferred embodiments, the nucleic acid sequence of the invention comprises at least one second miRNA binding site sequence that comprises one or more of the group consisting of the binding sites for miRNA-122-5p, miRNA-142-3p, miRNA-148a-3p, miRNA-101-3p, miRNA-192-5p, miRNA-194-5p, miRNA-223-3p.

In other preferred embodiments, the nucleic acid of the invention comprises at least one second miRNA binding site sequence that comprises one or more of miRNA-122, miRNA-192 and miRNA-194, preferably miRNA-122-5p and/or miRNA-192-5p and/or miRNA-194-5p.

In one preferred embodiment, the nucleic acid sequence of the invention comprises at least one second miRNA binding site sequence that comprises or consists of at least two or three miRNA-122 binding sites, preferably miRNA-122-5p.

In other preferred embodiments, the nucleic acid sequence of the invention comprises at least one second miRNA binding site sequence that comprises or consists of at least two or three miRNA-192 binding sites, preferably miRNA-192-5p.

In other preferred embodiments, the nucleic acid sequence of the invention comprises at least one second miRNA binding site sequence that comprises or consists of at least two or three miRNA-194 binding sites, preferably miRNA-194-5p.

Preferably, the nucleic acid sequence may comprise a nucleic acid sequence according to SEQ ID NO: 249-258, SEQ ID No: 300-303, SEQ ID NO: 343-382, or a nucleic acid sequence having, in increasing order of preference, at least 50%, 60%, 70%, 80%, 90%, 95%, 96%, 97%, 98%, or 99% sequence identity to any of these nucleic acid sequences, or a variant or fragment of any of these sequences.

In preferred embodiments, the at least one first or second miRNA binding site sequence comprises or consists of miRNA binding site nucleic acid sequences selected or derived from SEQ ID No 249, SEQ ID No 250, SEQ ID No 251, SEQ ID No 252, SEQ ID No 253, SEQ ID No 254, SEQ ID No 255, SEQ ID No 256, SEQ ID No 257 or SEQ ID No 258, SEQ ID No: 300, SEQ ID No: 301, SEQ ID No: 302, SEQ ID No: 303, or a fragment or variant of any of these.

In particularly preferred embodiments, the at least one first miRNA binding site sequence comprises or consists of miRNA binding site nucleic acid sequences selected or derived from SEQ ID NO: 249, SEQ ID NO: 252, SEQ ID NO: 303, or a fragment or variant of any of these.

In particularly preferred embodiments, the at least one second miRNA binding site sequence comprises or consists of miRNA binding site nucleic acid sequences selected or derived from SEQ ID NO: 249, SEQ ID NO: 255, SEQ ID NO 257, or a fragment or variant of any of these.

Suitably, the miRNA binding site sequences of the invention allows a cell type specific expression from the nucleic acid sequence within a target organ or organs.

In particularly preferred embodiments, the protein expression of the nucleic acid sequence is reduced in the liver.

In preferred embodiments in the context of reduced liver expression, the nucleic acid sequence comprises the at least one first miRNA binding site sequence that comprise at least one miRNA binding site for reducing or preventing protein expression in the liver.

Preferably, the at least one first miRNA binding site sequence comprises at least on miRNA binding site for reducing or preventing expression in liver selected or derived from one or more of the group consisting of binding sites for miRNA-122, miRNA-148a, miRNA-101, miRNA-192, miRNA-194.

In embodiments, the at least one first miRNA binding site sequence for reducing or preventing expression in liver comprises or consists of at least one binding site for miRNA-101, preferably miRNA-101-3p.

In embodiments, the at least one first miRNA binding site sequence for reducing or preventing expression in liver comprises or consists of at least one binding site for miRNA-192, preferably miRNA-192-5p.

In embodiments, the at least one first miRNA binding site sequence for reducing or preventing expression in liver comprises or consists of at least one binding site for miRNA-194, preferably miRNA-194-5p.

In preferred embodiments, the at least one first miRNA binding site sequence for reducing or preventing expression in liver comprises or consists of at least one binding site for miRNA-122, preferably miRNA-122-5p.

In other preferred embodiments, the at least one first miRNA binding site sequence for reducing or preventing expression in liver comprises or consists of at least one binding site for miRNA-148a, preferably miRNA-148a-3p.

In preferred embodiments in that context, the nucleic acid sequence comprises the at least one first miRNA binding site sequence that is located in 5′ direction relative to the coding sequence, preferably relative to a 5′ UTR, wherein the miRNA binding site sequence comprises one or more miRNA-122 and/or miRNA-148a binding sites.

In preferred embodiments in that context, the nucleic acid sequence comprises
I) at least one 5′ UTR preferably selected or derived from a gene;
II) at least one first miRNA binding site sequence located in 5′ direction relative to the 5′ UTR, wherein the miRNA binding site sequence comprises or consists of at least two or three miRNA-122 binding sites.

In other preferred embodiments in that context, the nucleic acid sequence comprises
I) at least one 5′ UTR preferably selected or derived from a gene;
II) at least one first miRNA binding site sequence located in 5′ direction relative to the 5′ UTR comprising or consisting of at least one miRNA-122 binding site and at least one first miRNA binding site sequence located within the 5′ UTR comprising or consisting of at least one miRNA-122 binding site.

In preferred embodiments, the nucleic acid of the invention comprises additionally or alternatively at least one second miRNA binding site sequence that comprises at least one miRNA binding site for reducing or preventing expression in liver.

In particularly preferred embodiments, the nucleic acid comprises additionally at least one second miRNA binding site sequence that comprises at least one miRNA binding site for reducing or preventing expression in liver.

Preferably, the at least one second miRNA binding site sequence comprises at least on miRNA binding site for reducing or preventing expression in the liver that is selected or derived from one or more of the group consisting of binding sites for miRNA-122, miRNA-148a, miRNA-101, miRNA-192, miRNA-194.

In embodiments, the at least one second miRNA binding site sequence for reducing or preventing expression in liver comprises or consists of at least one binding site for miRNA-101, preferably miRNA-101-3p.

In embodiments, the at least one second miRNA binding site sequence for reducing or preventing expression in liver comprises or consists of at least one binding site for miRNA-148a, preferably miRNA-148a-3p.

In preferred embodiments, the at least one second miRNA binding site sequence for reducing or preventing expression in liver comprises or consists of at least one binding site for miRNA-122, preferably miRNA-122-5p.

In preferred embodiments, the at least one second miRNA binding site sequence for reducing or preventing expression in liver comprises or consists of at least one binding site for miRNA-192, preferably miRNA-192-5p.

In preferred embodiments, the at least one second miRNA binding site sequence for reducing or preventing expression in liver comprises or consists of at least one binding site for miRNA-194, preferably miRNA-194-5p.

In preferred embodiments in that context, the nucleic acid sequence comprises the at least one second miRNA binding site sequence is located in 3′ direction relative to the coding sequence, preferably relative to a 3′ UTR, wherein the at least one second miRNA binding site sequence comprises one or more miRNA-122 binding sites and/or miRNA-192 binding sites and/or miRNA-194 binding sites.

In other preferred embodiments in that context, the nucleic acid sequence comprises
I) at least one 3′ UTR preferably selected or derived from a gene;
II) at least one second miRNA binding site sequence located in 3′ direction relative to the 3′ UTR, wherein the at least one second miRNA binding site sequence comprises at least one miRNA-192 binding sites and/or at least one miRNA-194 binding sites.

In embodiments, the nucleic acid sequence comprises a first miRNA binding site sequence comprise at least one miRNA binding site for reducing or preventing expression in liver, preferably selected from one or more of the group consisting of binding sites for miRNA-122, miRNA-148a, miRNA-101, miRNA-192, miRNA-194 and at least one second miRNA binding site sequence that comprises at least one miRNA binding site for reducing or preventing expression in liver, preferably selected from one or more of the group consisting of binding sites for miRNA-122, miRNA-148a, miRNA-101, miRNA-192, miRNA-194.

In preferred embodiments, the nucleic acid sequence comprises,
I) optionally, at least one 5′ UTR preferably selected or derived from a gene;
II) at least one first miRNA binding site sequence located in 5′ direction relative to the coding sequence, wherein the at least one first miRNA binding site sequence comprises one or more miRNA-122 and/or miRNA-148a binding sites.
III) at least one 3′ UTR preferably selected or derived from a gene;
IV) at least one second miRNA binding site sequence located in 3′ direction relative to the 3′ UTR, wherein the at least one second miRNA binding site sequence comprises one or more miRNA-122 binding sites and/or miRNA-192 binding sites and/or miRNA-194 binding sites.

In other preferred embodiments, the nucleic acid sequence comprises
I) at least one 5′ UTR preferably selected or derived from a gene;
II) at least one first miRNA binding site sequence located in 5′ direction relative to the 5′ UTR, wherein the at least one first miRNA binding site sequence comprises at least two or three miRNA-122 binding sites;
III) at least one 3′ UTR preferably selected or derived from a gene;
IV) at least one second miRNA binding site sequence located in 3′ direction relative to the 3′ UTR, wherein the at least one second miRNA binding site sequence comprises one or more miRNA-122 binding sites and/or miRNA-192 binding sites and/or miRNA-194 binding sites.

In preferred embodiments, the nucleic acid sequence comprises
I) at least one 5′ UTR preferably selected or derived from a gene;
II) at least one first miRNA binding site sequence located in 5′ direction relative to the 5′ UTR, wherein the at least one miRNA binding site sequence comprises at least two or three miRNA-122 binding sites;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-192 binding site.

In further preferred embodiments, the nucleic acid sequence comprises
I) at least one 5′ UTR preferably selected or derived from a gene;
II) at least one first miRNA binding site sequence located in 5′ direction relative to the 5′ UTR, wherein the at least one first miRNA binding site sequence comprises at least two or three miRNA-122 binding sites;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-194 binding site.

In preferred embodiments, the nucleic acid sequence comprises
I) at least one 5′ UTR preferably selected or derived from a gene;
II) at least one first miRNA binding site sequence located in 5′ direction relative to the 5′ UTR comprising or consisting of at least one first miRNA-122 binding site and at least one first miRNA binding site sequence located within the 5′ UTR comprising or consisting of at least one miRNA-122 binding site;
III) at least one 3′ UTR preferably selected or derived from a gene;
IV) at least one second miRNA binding site sequence located in 3′ direction relative to the 3′ UTR, wherein the at last one second miRNA binding site sequence comprises one or more miRNA-122 binding sites and/or miRNA-192 binding sites and/or miRNA-194 binding sites.

In preferred embodiments, the nucleic acid sequence comprises
I) at least one 5′ UTR preferably selected or derived from a gene;
II) at least one first miRNA binding site sequence located in 5′ direction relative to the 5′ UTR comprising or consisting of at least one first miRNA-122 binding site and at least one first miRNA binding site sequence located within the 5′ UTR comprising or consisting of at least one miRNA-122 binding site;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-192 binding site.

In other preferred embodiments, the nucleic acid sequence comprises
I) at least one 5′ UTR preferably selected or derived from a gene;
II) at least one first miRNA binding site sequence located in 5′ direction relative to the 5′ UTR comprising or consisting of at least one miRNA-122 binding site and at least one first miRNA binding site sequence located within the 5′ UTR comprising or consisting of at least one miRNA-122 binding site;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-194 binding site.

In preferred embodiments, the protein expression of the nucleic acid sequence is reduced in immune cells.

In preferred embodiments in the context of reduced immune cell expression, the nucleic acid sequence comprises the at least one first miRNA binding site sequence comprise at least one miRNA binding site for reducing or preventing protein expression in immune cells.

In embodiments, the at least one first miRNA binding site sequence comprises at least on miRNA binding site for reducing or preventing expression in immune cells selected or derived from miRNA-142 and miRNA-223.

In embodiments, the at least one first miRNA binding site sequence for reducing or preventing expression in immune cells comprises or consists of at least one binding site for miRNA-142, preferably miRNA-142-3p.

In preferred embodiments, the at least one first miRNA binding site sequence for reducing or preventing expression in immune cells comprises or consists of at least one binding site for miRNA-223, preferably miRNA-223-5p.

In preferred embodiments, the nucleic acid sequence comprises the at least one first miRNA binding site sequence is located in 5′ direction relative to the coding sequence, preferably relative to a 5′ UTR, wherein the miRNA binding site sequence comprises miRNA-142 and/or miRNA-223 binding sites.

In preferred embodiments in that context, the nucleic acid sequence comprises
I) at least one 5′ UTR preferably selected or derived from a gene;
II) at least one first miRNA binding site sequence located in 5′ direction relative to the 5′ UTR, wherein the at least one first miRNA binding site sequence comprises at least one miRNA-142 binding sites and/or at least one miRNA-223 binding sites.

According to preferred embodiments, the nucleic acid of the invention comprises additionally or alternatively at least one second miRNA binding site sequence that comprises at least one miRNA binding site for reducing or preventing expression in immune cells.

In preferred embodiments, the nucleic acid additionally comprises at least one second miRNA binding site sequence that comprises at least one miRNA binding site for reducing or preventing expression in immune cells.

In embodiments, the nucleic acid sequence comprises the at least one second miRNA binding site sequence that comprises at least on miRNA binding site for reducing or preventing expression in immune cells selected or derived from one or more from miRNA-142 and miRNA-223.

In embodiments, the at least one second miRNA binding site sequence for reducing or preventing expression in immune cells comprises or consists of at least one binding site for miRNA-142, preferably miRNA-142-3p.

In preferred embodiments, the at least one second miRNA binding site sequence for reducing or preventing expression in immune cells comprises or consists of at least one binding site for miRNA-223, preferably miRNA-223-5p.

In embodiments in that context, the nucleic acid sequence comprises the at least one second miRNA binding site sequence that is located in 5′ direction relative to the coding sequence, preferably relative to a 5′ UTR, wherein the miRNA binding site sequence comprises miRNA-142 and/or miRNA-223 binding sites.

In preferred embodiments in that context, the nucleic acid sequence comprises
I) at least one 5′ UTR preferably selected or derived from a gene;
II) at least one first miRNA binding site sequence located in 5′ direction relative to the 5′ UTR, wherein the at least one first miRNA binding site sequence comprises at least one miRNA-142 binding sites and/or at least one miRNA-223 binding sites.

In preferred embodiments, the protein expression of the nucleic acid sequence is reduced in liver cells and in immune cells.

In embodiment in the context of reduced liver and immune cell expression, the nucleic acid sequence comprises at least one first miRNA binding site sequence that comprises at least one miRNA binding site for reducing or preventing protein expression in liver cells and/or immune cells, preferably wherein the at least one first miRNA binding site sequence is located in 5′ direction relative to the coding sequence.

In a preferred embodiments in that context, the nucleic acid sequence comprises at least one first miRNA binding site sequence comprises at least on miRNA binding site for reducing or preventing protein expression in the liver is selected or derived from one or more of the group consisting of binding sites for miRNA-122, miRNA-148a, miRNA-101, miRNA-192, miRNA-194, preferably miRNA-122 and/or miRNA-148a binding sites.

In particular preferred embodiments, the nucleic acid sequence of the invention comprises at least one first miRNA binding site sequence comprises at least on miRNA binding site for reducing or preventing protein expression in immune cells selected or derived from miRNA-142 and miRNA-223.

In preferred embodiments in that context, the nucleic acid sequence additionally comprises at least one second miRNA binding site sequence that comprise at least one miRNA binding site for reducing or preventing protein expression in liver cells and/or immune cells, preferably wherein the at least one second miRNA binding site sequence is located in 3′ direction relative to the coding sequence.

In preferred embodiments, the nucleic acid sequence comprises at least one second miRNA binding site sequence comprises at least on miRNA binding site for reducing or preventing protein expression in the liver is selected or derived from one or more of the group consisting of binding sites for miRNA-122, miRNA-148a, miRNA-101, miRNA-192, miRNA-194, preferably miRNA-122 binding sites and/or miRNA-192a binding sites and/or miRNA-194 binding sites and at least one second miRNA binding site sequence comprises at least on miRNA binding site for reducing or preventing protein expression in immune cells selected or derived from miRNA-142 and miRNA-223.

In preferred embodiments, the nucleic acid sequence comprises at least one first miRNA binding site sequence for reducing expression in the liver as defined herein and at least one second miRNA binding site sequence for reducing expression in immune cells as defined herein.

In alternative preferred embodiments, the nucleic acid sequence comprises at least one first miRNA binding site sequence for reducing expression in immune cells as defined herein and at least one second miRNA binding site sequence for reducing expression in the liver as defined herein.

In embodiments wherein a preferred expression in immune cells such as for vaccination purposes is intended, the miRNA binding site sequence according to the invention preferably comprises at least one miRNA-122, miRNA-101, miRNA-148a, miRNA-192, and/or a miRNA-194 binding site, preferably at least one miRNA-122 binding site to reduce/avoid liver expression of the target protein.

In a preferred embodiment, the miRNA binding site sequence according preferably comprises at least one miRNA-122-5p, miRNA-101-3p, miRNA-148a-3p, miRNA-192-5p, and/or a miRNA-194-5p binding site, preferably at least one miRNA-122-5p binding site to reduce/avoid liver expression of the target protein.

In embodiments wherein a preferred expression in muscle, tissue or cells and avoid expression in immune cells such as for replacement therapies, gene therapy or intratumoral molecular therapy purposes is intended, the miRNA binding site sequence according to the invention preferably comprises at least one miRNA-142 and/or miRNA-223-3p, preferably at least one miRNA-142-3p binding site.

In embodiments wherein a preferred expression in muscle, tissue or cells such as for replacement therapies, gene therapy or intratumoral molecular therapy purposes is intended, the miRNA binding site sequence according to the invention preferably comprises at least one miRNA-142 and/or miRNA-223-3p, preferably at least one miRNA-223-3p binding site.

In preferred embodiments, the miRNA binding site sequence according to the invention preferably comprises at least one miRNA-142-3p and/or miRNA-223-3p, preferably at least one miRNA-223-3p binding site to reduce/avoid immune cell expression of the target protein.

In embodiments wherein a preferred expression in muscle, tissue or cells and avoid expression in immune cells such as for replacement therapies, gene therapy or intratumoral molecular therapy purposes is intended, the miRNA binding site sequence according to the invention preferably comprises at least one miRNA-122, miRNA-148a, miRNA-101, miRNA-192, and/or a miRNA-194 binding site and at least another miRNA binding site, at least one miRNA-142 and/or miRNA-223 binding site.

In preferred embodiments, the miRNA biding site sequence according to the invention preferably comprises at least one miRNA-122-5p, miRNA-148a-3p, miRNA-192-5p, and/or a miRNA-194-5p binding site, preferably at least one miRNA-122-5p binding site to reduce/avoid liver expression of the target protein and at least one miRNA-142-3p and/or miRNA-223-3p, preferably at least one miRNA-223-3p binding site to reduce/avoid immune cell expression of the target protein.

In other embodiments wherein a preferred expression in tumor cells is intended, the miRNA binding site sequence according to the invention preferably comprises at least one miRNA-122, miRNA-192, and/or a miRNA-194 binding site, preferably at least one miRNA-122 binding site to reduce/avoid liver expression of the target protein.

In further embodiments wherein a preferred expression in immune cells has to be avoided such as for protein replacement therapy the miRNA binding site sequence according to the invention preferably comprises at least one miRNA-148a, miRNA-101, miRNA-194 and/or optionally a miRNA-192 binding site (depending on the target tissue), preferably at least one miRNA-148a binding site.

In preferred embodiments, the miRNA biding site sequence according to the invention preferably comprises at least one miRNA-148a-3p, miRNA-101-3p and/or a miRNA-192-5p binding site, preferably at least one miRNA-148a-3p binding site.

It is known that variants and polymorphisms of miRNA sequences can be found, and that miRNA families exist with similar properties. In the present invention, it is envisioned that all suitable variants and family members of particular miRNA sequences and associated binding sites can be used where appropriate. On the other hand, apparently closely related miRNA sequences can have different expression profiles (Sun et al, World J Gastroenterol. 2017 Nov. 28), so in some situations it will be necessary to determine whether a specific substitution is appropriate, by reference to the literature. For example, Let-7 is part of a wider family with a number of related variants, which can be denoted as Let-7a to Let-7k, and so on.

In some embodiments, the nucleic acid sequence contains a miRNA binding site sequence which comprises one or more miRNA-122 and/or miRNA-148a binding sites.

In preferred embodiments, the nucleic acid sequence contains a miRNA binding site sequence which comprises one or more miRNA-122-5p and/or miRNA-148a-3p binding sites.

In preferred embodiments, the nucleic acid sequence contains a miRNA binding site sequence, which comprises one or more miRNA-122 binding sites. Hereby, in most preferred embodiments, the nucleic acid sequence contains a miRNA binding site sequence, which comprises one miRNA-122 binding site.

The miRNA-122 is an abundant, liver-specific miRNA, which expression is significantly decreased in human primary hepatocarcinoma (HCC). MiRNA-122, despite its abundance in healthy non-diseased liver tissue, is reduced in the majority of liver cancers as well as in diseased cells (Braconi et al. 2011, Semin Oncol; 38(6): 752-763, Brown and Naldini Nature 2009; 10 578). In this invention, by incorporation of miRNA-122 binding site(s) 5′ of the 5′ UTR within the nucleic acid sequence, it has been found that when the target tissue is the liver, translation of the introduced nucleic acid sequences (e.g. mRNA) can be facilitated in other cells and is reduced in liver cells.

In other embodiments, the nucleic acid sequence contains a miRNA binding site sequence, which comprises at least two miRNA-122 binding sites.

In preferred embodiments, the nucleic acid sequence contains a miRNA binding site sequence, which comprises at least two miRNA-122-5p binding sites.

In other embodiments, the nucleic acid sequence contains a miRNA binding site sequence, which comprises at least two miRNA-148a binding sites.

In a preferred embodiment, the nucleic acid sequence contains a miRNA binding site sequence, which comprises at least two miRNA-148a-3p binding sites.

In one preferred embodiment, the miRNA binding site sequence comprises the sequence of SEQ ID NO 252.

In one preferred embodiment, the miRNA binding site sequence comprises the sequence of SEQ ID NO 303.

The nucleic acid sequence of the invention comprises at least one first miRNA binding site sequence comprises or consists of a nucleic acid sequence selected or derived from SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257 or SEQ ID NO: 258, SEQ ID NO: 300, SEQ ID NO: 301, SEQ ID NO: 302, SEQ ID NO: 303, or a fragment or variant of any of these.

In a preferred embodiment the nucleic acid sequence of the invention comprises at least one first miRNA binding site sequence comprises or consists of a nucleic acid sequence selected or derived from SEQ ID NO: 249, SEQ ID NO: 252, SEQ ID NO: 303, or a fragment or variant of any of these.

The nucleic acid sequence of the invention comprises at least one second miRNA binding site sequence comprises or consists of a nucleic acid sequence selected or derived from SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257 or SEQ ID NO: 258, SEQ ID NO: 300, SEQ ID No: 301, SEQ ID NO: 302, SEQ ID NO: 303 or a fragment or variant of any of these.

In a preferred embodiment the nucleic acid sequence of the invention comprises at least one second miRNA binding site sequence comprises or consists of a nucleic acid sequence selected or derived from SEQ ID NO: 249, SEQ ID NO: 255, SEQ ID NO 257, or a fragment or variant of any of these.

In preferred embodiments, the miRNA binding site sequence is located immediately 5′ of the 5′ UTR. Accordingly, 5′ of the 5′ UTR can be also understood as upstream/prior/before/in front of the 5′ UTR on the nucleic acid sequence. In another preferred embodiment first miRNA binding site sequence is located within the 5′UTR. In another embodiment the miRNA biding site sequence is located immediately 3′ of the 5′UTR. Accordingly, 3′ of the 5′UTR can be also understood as downstream of/after/following the 5′UTR on the nucleic acid sequence.

This miRNA binding site sequence is also defined as the first binding site sequence of the nucleic acid sequence (see FIG. 12B).

Hereby, one or more space nucleotides might be placed between the miRNA binding site and the 5′ UTR. The spacer nucleotide might be selected from A, C, U or G. In some embodiments 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 41, 42, 43, 44, 45, 46, 47, 48, 49 or 50 nucleotides might be placed between the miRNA biding sites and the 5′UTR. In some embodiment 0, 1, 2, 3, 4, 5, or 6 nucleotides might be placed between the miRNA biding sites and the 5′UTR. In a preferred embodiment 0 nucleotides might be placed between the miRNA binding sites and the 5′UTR.

In the context of the present invention, a nucleic acid sequence comprising at least one first miRNA binding site sequence located in 5′ direction relative to said coding region. In the context of the present invention a nucleic acid sequence comprising at least two first miRNA binding site sequence located in 5′ direction relative to said coding region. In the context of the present invention a nucleic acid sequence comprising at least three first miRNA binding site sequence located in 5′ direction relative to said coding region.

Preferably, the nucleic acid sequence may comprise a nucleic acid sequence according to SEQ ID NO: 249-SEQ ID NO 258, SEQ ID NO: 300-303, SEQ ID NO: 304-SEQ ID NO: 342, or a nucleic acid sequence having, in increasing order of preference, at least 50%, 60%, 70%, 80%, 90%, 95%, 96%, 97%, 98%, or 99% sequence identity to any of these nucleic acid sequences, or a variant or fragment of any of these sequences.

Preferably, the miRNA-122 binding site, within the first miRNA binding site sequence is located 5′ of the 5′ UTR.

The position 5′ of the 5′ UTR can also be understood as prior/upstream/in front/before of the 5′ UTR.

In some embodiments, upstream/prior/in front of the first miRNA binding site sequence and the first miRNA binding site within the miRNA binding site sequence, six nucleotides are placed after the T7 polymerase start point. Hereby, those six nucleotides are preferably AGGAGA.

In preferred embodiments, the nucleic acid sequence according to this invention comprising
i) at least one 3′ UTR of a gene;
ii) at least one coding region encoding at least one peptide or protein of interest;
iii) at least one 5′ UTR of a gene;
iv) a miRNA binding site sequence comprising one miRNA-122 binding site wherein the miRNA binding site sequence is located immediately 5′ of the 5′UTR to allow a cell type specific expression from the nucleic acid sequence within the target organ or organs.

In another embodiment, the 3′ UTR of the nucleic acid sequence optionally comprises within the 3′UTR and/or immediately 3′ or 5′ of the 3′ UTR a second miRNA binding site sequence comprising at least one miRNA binding site. Accordingly, 3′ of the 3′ UTR can be also understood as downstream of/after/following the ′3 UTR on the nucleic acid sequence. Hereby, one or more space nucleotides might be placed between the miRNA binding site and the 3′ UTR. The spacer nucleotide might be selected from A, C, U or G. This miRNA binding site sequence immediately 3′ (after/downstream/following) or 5′ (prior/before/upstream/in front) of the 3′ UTR is also defined as the second binding site sequence of the nucleic acid sequence. The position 3′ of the 3′ UTR can also be understood as after/downstream/following (of) the 3′ UTR (see FIG. 12C).

In some embodiments, the second miRNA binding site sequence comprises at least one miRNA binding site substantially complementary to a miRNA sequence selected from at least one or more of the group consisting of miRNA-192, miRNA-122, miRNA-148a, miRNA-194 or miR-101.

In some embodiments, the second miRNA binding site sequence comprises at least one miRNA binding site substantially complementary to a miRNA sequence selected from at least one or more of the group consisting of miRNA-192-5p, miRNA-122-3p, miRNA-142-3p, miRNA-148a-3p, miRNA-194-5p, miRNA-223-3p or miR-101-3p.

Hereby, the second miRNA binding site sequence preferably comprises one or more miRNA-192 and/or miRNA-122 binding sites.

In preferred embodiments, the first miRNA binding site sequence of the nucleic acid sequence comprises at least one miRNA-122 binding site, wherein the second miRNA binding side sequence comprises at least one miRNA-192 binding site.

In preferred embodiments, the first miRNA binding site sequence of the nucleic acid sequence comprises at least one miRNA-122-3p binding site, wherein the second miRNA binding side sequence comprises at least one miRNA-192-5p binding site.

In embodiments, the first miRNA binding site sequence of the nucleic acid sequence comprises at least one miRNA-122 binding site, wherein the second binding side sequence comprises at least one miRNA-122 binding site.

In embodiments, the first miRNA binding site sequence of the nucleic acid sequence comprises at least one miRNA-122-3p binding site, wherein the second binding side sequence comprises at least one miRNA-122-3p binding site.

In other preferred embodiment, the first miRNA binding site sequence of the nucleic acid sequence comprises at least one miRNA-122 binding site, wherein the second binding side sequence does not comprise a miRNA binding site.

In other embodiments, the second miRNA binding sequence comprises at least two miR-192 binding sites.

In other embodiments, the second miRNA binding sequence comprises at least two miR-192-5p binding sites. In preferred embodiments, the second miRNA binding site sequence is located immediately 3′ of the 3′ UTR.

Accordingly, 3′ of the 3′ UTR can be also understood as downstream or after of the 3′ UTR on the nucleic acid sequence.

This miRNA binding site sequence is also defined as the second binding site sequence of the nucleic acid sequence.

Hereby, one or more spacer nucleotides might be placed between the 3′ UTR and the miRNA binding site. The spacer nucleotide might be selected from A, C, U or G. Preferably, the miRNA-192 binding site within the miRNA binding site sequence is located 3′ of the 3′ UTR.

### Target Cells/Organs

In one embodiment the cell type specific expression from the nucleic acid sequence of the invention within the target organ or organs is selected from liver cells, tumor cells or immune cells, muscle cells, skin cells, cells in the eye, or lung cells.

In a preferred embodiment the cell type specific expression from the nucleic acid sequence within the target organ or organs is selected from muscle cells and immune cells.

In another preferred embodiment the cell type specific expression from the nucleic acid sequence within the target organ or organs is selected from muscle cells.

In embodiments, the cell type specific expression from the nucleic acid sequence within the target organ or organs is not selected from hepatocytes, hepatic stellate fat storing (ITO) cells, Kupffer cells or liver endothelial cells.

Hepatocytes, hepatic stellate fat storing (ITO) cells, Kupffer cells or liver endothelial cells are commonly described as the four main cell types of the liver. The liver parenchyma is primarily comprised of hepatocytes (80%). Ito cells are also known as stellate cells, fat storing cells, or lipocytes. Ito cells reside in the perisinusoidal region located between endothelial cells and hepatocytes. Kupffer cells are phagocytes (“phago”=eating, “cyto”=cells) derived from monocytes and located within the vascular spaces of hepatic sinusoids lining the endothelial surfaces. Liver endothelial cells form the lining of the smallest blood vessels in the liver.

As used herein, the term ‘organ’ is synonymous with an ‘organ system’ and refers to a combination of tissues and/or cell types that may be compartmentalized within the body of a subject to provide a biological function, such as a physiological, anatomical, homeostatic or endocrine function. Suitably, organs or organ systems may mean a vascularized internal organ, such as a liver or pancreas. Typically, organs comprise at least two tissue types, and/or a plurality of cell types that exhibit a phenotype characteristic of the organ.

Even with localized or targeted administration, it is possible that supplied mRNA constructs may encounter or accumulate in organs, tissues, and/or cells for which they were not intended. In particular, liver and kidney tissue may accumulate administered compositions, due to the physiological function of these organs. In these cases, to avoid off-target effects, it may be advantageous for the supplied constructs to comprise miRNA binding sites, which would enable reduced expression in these tissues. Conversely, it may be desirable for expression to be encouraged in some organs, tissues and/or cell types but not others, which can be achieved by the selection of miRNA binding sites accordingly.

For example, liver cancer cells (e.g., hepatocellular carcinoma cells) typically express low levels of miR-122 as compared to normal liver cells. Therefore, an mRNA encoding a polypeptide that includes at least one miR-122 binding site (e.g., in front of the 5′-UTR of a gene of the mRNA) will typically express comparatively low levels of the polypeptide in normal liver cells and comparatively high levels of the polypeptide in liver cancer cells. If the polypeptide is able to induce apoptosis, this can cause preferential cell killing of liver cancer cells (e.g., hepatocellular carcinoma cells) as compared to normal liver cells.

In preferred embodiments, the cell type specific expression from the nucleic acid sequence within the target organ or organs is not in hepatocytes.

The nucleic acid sequence is preferably not expressed within hepatocytes. Accordingly, the nucleic acid sequence of this invention comprises a miRNA-122 binding site within its first binding site sequence to reduce the expression from the nucleic acid sequence within the target organ (liver).

In another preferred embodiment the cell type specific expression from the nucleic acid sequence within the target organ or cells are not immune cells.

The nucleic acid sequence is preferably not expressed within immune cells. Accordingly, the nucleic acid sequence of this invention comprises a miRNA-223 binding site within its first binding site sequence to reduce the expression from the nucleic acid sequence within the target cells, immune cells.

In further preferred embodiments, the cell type specific expression from the nucleic acid sequence within the target organ or organs may be selected from tumor cells, immune cells or other cells of interest.

By using different combinations of miRNA binding sites in the first and second miRNA binding site sequences of the nucleic acid sequence (e.g. mRNA), different cell types or target organs can express, or be protected from the expression of certain peptides or proteins, according to the desired objective. For example, at least one liver-specific miRNA-122 binding site can be placed prior the 5′ UTR in combination with at least one kidney-specific miRNA-192, and/or a miRNA-194 binding site to reduce/avoid expression of the target protein in both tissues. In preferred embodiments, wherein the expression in the liver is not intended to unwanted side effects of the therapeutic target protein, a miRNA-122 binding site is preferably placed prior to the 5′ UTR. In preferred embodiments, wherein the expression in the liver is not intended to unwanted side effects of the therapeutic target protein, a miRNA-122 binding site is preferably placed prior and within the 5′ UTR. Thus, the therapeutic effect of the target protein is limited to the target organ or organs which might be selected from tumor cells, immune cells or other cells of interest.

### Modified Nucleotides

In some embodiments, the nucleic acid sequence comprises at least one modified nucleotide and/or at least one nucleotide analogue or nucleotide derivative.

In this context, the modified nucleotide as defined herein are nucleotide analogs/modifications, e.g. backbone modifications, sugar modifications or base modifications. A backbone modification in connection with the present invention is a modification, in which phosphates of the backbone of the nucleotides are chemically modified. A sugar modification in connection with the present invention is a chemical modification of the sugar of the nucleotides. Furthermore, a base modification in connection with the present invention is a chemical modification of the base moiety of the nucleotides. In this context, nucleotide analogs or modifications are preferably selected from nucleotide analogs which are applicable for transcription and/or translation.

In preferred embodiments, the nucleic acid sequence comprises least one modified nucleotide and/or at least one nucleotide analogues which is selected from a backbone modified nucleotide, a sugar modified nucleotide and/or a base modified nucleotide, or any combination thereof.

### Sugar Modification:

The modified nucleosides and nucleotides, which may be incorporated into the nucleic acid sequence as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH2CH20)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

### Backbone Modifications:

The phosphate backbone may further be modified in the modified nucleosides and nucleotides, which may be incorporated into the nucleic acid sequence as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

### Base Modifications:

The modified nucleosides and nucleotides, which may be incorporated into the nucleic acid sequence as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group. In particularly preferred embodiments, the nucleotide analogues/modifications which may be incorporated into a nucleic acid sequence as described herein, are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine. Preferably, at least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, 5-(isopentenylaminomethyl)-2′-O-methyluridine, N-1-methylpseudouridine, or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or O(6)-methylguanine.

In another preferred embodiment the nucleic acid sequence of the invention comprises the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, N-1-methylpseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, 2-aminoadenosine or 5-(isopentenylaminomethyl)-2′-O-methyluridine or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or O(6)-methylguanine.

In some embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyluridine.

In preferred embodiments, 100% of the uracil in the coding sequence as defined herein have a chemical modification, preferably a chemical modification is in the 5′-position of the uracil.

In embodiments, 100% of the uracil in the cds of the nucleic acid sequence have a chemical modification, preferably a chemical modification that is in the 5-position of the uracil. In other embodiments, at least 10%, 20%, 30%, 40%, 50%, 60%, 70%, 80%, or 90% of the uracil nucleotides in the cds have a chemical modification, preferably a chemical modification that is in the 5-position of said uracil nucleotides. Such modifications are suitable in the context of the invention, as a reduction of natural uracil may reduce the stimulation of the innate immune system (after in vivo administration of the RNA comprising such a modified nucleotide) potentially caused by the first component upon administration to a cell.

Preferably, all uridine bases of the nucleic acid sequence mRNA are fully chemically modified, even more preferably wherein all uridine bases of the mRNA are pseudouridine or N1-methylpseudouridine (N1MPU) bases.

The terms “cds” or “coding sequence” or “coding region” as used herein will be recognized and understood by the person of ordinary skill in the art, and are e.g. intended to refer to a sequence of several nucleotide triplets, which may be translated into a peptide or protein

In preferred embodiments, at least one modified nucleotide is selected from pseudouridine (ψ), N1-methylpseudouridine (m1ψ), 5-methylcytosine, and/or 5-methoxyuridine Suitably, the nucleic acid sequence, in particular, the cds of said nucleic acid sequence, may comprise at least one modified nucleotide, wherein said at least one modified nucleotide may be selected from pseudouridine (ψ), N1-methylpseudouridine (m1ψ), 5-methylcytosine, and 5-methoxyuridine, wherein pseudouridine (ψ) is preferred.

In alternative embodiments, the nucleic acid sequence is composed of (chemically) non-modified ribonucleoside triphosphates (NTPs) GTP, ATP, CTP and UTP.

In the context of the invention, the terms “modified nucleotides” or “chemically modified nucleotides” do not encompass 5′ cap structures (e.g. cap0, cap1 as defined herein). Additionally, the term “modified nucleotides” does not relate to modifications of the codon usage of e.g. a respective coding sequence. The terms “modified nucleotides” or “chemically modified nucleotides” do encompass all potential natural and non-natural chemical modifications of the building blocks of an RNA, namely the ribonucleotides A, G, C, U.

Accordingly, the nucleic acid sequence is not a (chemically) modified RNA, wherein the modification may refer to chemical modifications comprising backbone modifications as well as sugar modifications or base modifications.

### Cap

In one embodiment the nucleic acid sequence of the invention consists of non-modified nucleotides and optionally comprises a 5′ terminal cap structure.

In various embodiments, the nucleic acid sequence comprises a cap.

The term “cap” or “5′-cap structure” as used herein is intended to refer to the 5′ structure of the nucleic acid sequence, particularly a guanine nucleotide, positioned at the 5′-end of an nucleic acid sequence; an RNA, e.g. an mRNA.

Preferably, the 5′-cap structure is connected via a 5-5-triphosphate linkage to the RNA. Notably, a “5-cap structure” or a “cap analogue” is not considered to be a “modified nucleotide” or “chemically modified nucleotides” in the context of the invention. 5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g. phosphothioate modARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Accordingly, in preferred embodiments the cap is a cap0, cap1, cap2, a modified cap0 or a modified cap1, preferably a cap.

A 5′-cap (cap0 or cap1) structure may be formed in chemical RNA synthesis, using capping enzymes, or in RNA in vitro transcription (co-transcriptional capping) using cap analogs.

The term “cap analog” as used herein is intended to refer to a non-polymerizable di-nucleotide or tri-nucleotide that has cap functionality in that it facilitates translation or localization, and/or prevents degradation of the nucleic acid sequence, e.g. RNA, when incorporated at the 5′-end of the RNA. Non-polymerizable means that the cap analogue will be incorporated only at the 5-terminus because it does not have a 5′ triphosphate and therefore cannot be extended in the 3′-direction by a template-dependent polymerase, (e.g. a DNA-dependent RNA polymerase). Examples of cap analogues include m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures relating to cap analogues are incorporated herewith by reference.

In particularly preferred embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap analog derivable from the structure disclosed in claim 1-5 of WO2017/053297 may be suitably used to co-transcriptionally generate a cap1 structure. Further, any cap analog derivable from the structure defined in claim 1 or claim 21 of WO2018/075827 may be suitably used to co-transcriptionally generate a cap1 structure.

In preferred embodiments, the cap1 structure of the nucleic acid sequence is formed using co-transcriptional capping using tri-nucleotide cap analog m7G(5′)ppp(5′)(2′OMeA)pG or m7G(5′)ppp(5′)(2′OMeG)pG. A preferred cap1 analog in that context is m7G(5′)ppp(5′)(2′OMeA)pG.

In principle, 5′ cap structures can be introduced into the nucleic acid sequence by using one of two protocols. In the first protocol, capping occurs concurrently with the initiation of transcription (co-transcriptional capping). In this approach, a dinucleotide cap analog such as m7G(5′)ppp(5′)G (m7G) is added to the reaction mixture. The DNA template is usually designed in such a way that the first nucleotide transcribed is a guanosine. The cap analog directly competes with GTP for incorporation as initial nucleotide and is incorporated as readily as any other nucleotide (WO2006/004648). A molar excess of the cap analog relative to GTP facilitates the incorporation of the cap dinucleotide at the first position of the transcript. However, this approach always yields a mixture of capped and uncapped RNAs. Uncapped mRNAs can usually not be translated after transfection into eukaryotic cells, thus reducing the efficacy of the RNA therapeutic. The effective concentration of co-transcriptionally capped mRNAs with the standard cap analog (m7GpppG) is further reduced because the analog can be incorporated in the reverse orientation (Gpppm7G), which is less competent for translation (Stepinski et al., 2001. RNA 7(10):1 486-95). The issue of cap analog orientation can be solved by using anti-reverse cap analogs (ARCA) such as (3′-O-methyl)GpppG which cannot be incorporated in the reverse orientation (Grudzien et al., 2004. RNA 10(9): 1479-87).

In the second protocol, capping is done in a separate enzymatic reaction after in vitro transcription (post-transcriptional or enzymatic capping). Vaccinia Virus Capping Enzyme (VCE) possesses all three enzymatic activities necessary to synthesize a m7G cap structure (RNA 5′-triphosphatase, ganylyltransferase, and guanine-7-methyltransferase). Using GTP as substrate the VCE reaction yields RNA caps in the correct orientation. In addition, a type 1 cap can be created by adding a second Vaccinia enzyme, 2′-O-methyltransferase, to the capping reaction (Tcherepanova et al., 2008. BMC Mol. Biol. 9:90).

In some embodiments about 70%, 75%, 80%, 85%, 90%, 95% of the nucleic acid sequence comprises a cap1 structure as determined by using a capping detection assay. In most preferred embodiments, less than about 20%, 15%, 10%, 5%, 4%, 3%, 2%, 1% of the nucleic acid sequence does not comprises a cap structure as determined using a capping assay.

In preferred embodiments at least 70%, 80%, or 90% of the nucleic acid sequence comprise a cap1 structure. For determining the presence/absence of a cap0 or a cap1 structure, a capping assays as described in published PCT application WO2015/101416, in particular, as described in claims 27 to 46 of published PCT application WO2015/101416 may be used. Other capping assays that may be used to determine the presence/absence of a cap0 or a cap1 structure of an RNA are described in PCT/EP2018/08667, or published PCT applications WO2014/152673 and WO2014/152659.

In preferred embodiments, the nucleic acid sequence comprises an m7G(5′)ppp(5′)(2′OMeA) cap structure. In such embodiments, the RNA comprises a 5-terminal m7G cap, and an additional methylation of the ribose of the adjacent nucleotide of m7GpppN, in that case, a 2′O methylated adenosine. Preferably, about 70%, 75%, 80%, 85%, 90%, 95% of the nucleic acid sequence comprises such a cap1 structure as determined using a capping assay. Preferably, about 95% of the nucleic acid sequence comprises a cap1 structure in the correct orientation (and less that about 5% in reverse orientation) as determined using a capping assay.

In other preferred embodiments, the nucleic acid sequence comprises an m7G(5′)ppp(5′)(2′OMeG) cap structure. In such embodiments, the, the nucleic acid sequence comprises a 5-terminal m7G cap, and an additional methylation of the ribose of the adjacent nucleotide, in that case, a 2′O methylated guanosine. Preferably, about 70%, 75%, 80%, 85%, 90%, 95% of the, the nucleic acid sequence comprises such a cap1 structure as determined using a capping assay.

Accordingly, the first nucleotide of said, the nucleic acid sequence, that is, the nucleotide downstream of the m7G(5′)ppp structure, may be a 2′O methylated guanosine or a 2′O methylated adenosine.

### Coding Region Encodes a Therapeutic Peptide or Protein

In preferred embodiments, the nucleic acid sequence comprises at least one coding region encoding at least one peptide or protein of interest wherein the at least one peptide or protein is a therapeutic peptide or protein or is derived from a therapeutic peptide or protein.

The term “therapeutic” in that context has to be understood as “providing a therapeutic function” or as “being suitable for therapy or administration”. However, “therapeutic” in that context should not at all to be understood as being limited to a certain therapeutic modality. Examples for therapeutic modalities may be the provision of a coding sequence (via said nucleic acid sequence) that encodes for a peptide or protein (wherein said peptide or protein has a certain therapeutic function, e.g. an antigen for a vaccine, or an enzyme for protein replacement therapies). A further therapeutic modality may be genetic engineering, wherein the RNA provides or orchestrates factors to e.g. manipulate DNA and/or RNA in a cell or a subject.

Another therapeutic modality may be cytotoxic or cytoprotective therapeutics to specific cells such as, but not limited to, normal and/or cancerous cells.

In the context of the invention, the nucleic acid sequence may provide at least one coding sequence encoding a peptide or protein that is translated into a (functional) peptide or protein after administration (e.g. after administration to a subject, e.g. a human subject).

In some embodiments the nucleic acid sequence according to the invention may comprise at least one coding region encoding a therapeutic protein replacing an absent, deficient or mutated protein; a therapeutic protein beneficial for treating or preventing inherited or acquired diseases; infectious diseases, or neoplasms e.g. cancer or tumor diseases); an adjuvant or immuno-stimulating therapeutic protein; a therapeutic antibody or an antibody fragment, variant or derivative; a peptide hormone; a gene editing agent; an immune checkpoint inhibitor; a T cell receptor, or a fragment, variant or derivative T cell receptor; cytostatic or cytotoxic polypeptides and/or an enzyme.

In preferred embodiments, the nucleic acid sequence comprises at least one coding sequence encoding at least one peptide or protein suitable for use in treatment or prevention of a disease, disorder or condition.

In preferred embodiments, the length of the cds may be at least or greater than about 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the cds may be in a range of from about 300 to about 2000 nucleotides.

According to further preferred embodiments, the nucleic acid sequence comprises at least one coding sequence which encodes at least one (therapeutic) peptide or protein as defined below, and additionally at least one further heterologous peptide or protein element.

In various embodiments, the length of the encoded peptide or protein, e.g. the therapeutic peptide or protein, may be at least or greater than about 20, 50, 100, 150, 200, 300, 400, 500, 600, 700, 800, 900, 1000, or 1500 amino acids.

According to certain embodiments, the nucleic acid sequence is mono-, bi-, or multicistronic, as defined herein. The coding sequences is preferably bi- or multicistronic. The nucleic acid sequence preferably encodes a distinct peptide or protein as defined herein or a fragment or variant thereof.

The term “monocistronic” will be recognized and understood by the person of ordinary skill in the art, and is e.g. intended to refer to an obtained in vitro transcribed RNA that comprises only one coding sequences. The terms “bicistronic”, or “multicistronic” as used herein will be recognized and understood by the person of ordinary skill in the art, and are e.g. intended to refer to a nucleic acid sequence that may have two (bicistronic) or more (multicistronic) coding sequences.

In other embodiments, the nucleic acid sequence is monocistronic and the cds of said nucleic acid sequence encodes at least two different peptides or proteins as defined herein. Accordingly, said coding regions may e.g. encode at least two, three, four, five, six, seven, eight and more therapeutic peptides or proteins, linked with or without an peptide linker sequence, wherein said linker sequence can comprise rigid linkers, flexible linkers, cleavable linkers, or a combination thereof. Such constructs are herein referred to as “multi-protein-constructs”.

In further embodiments, the nucleic acid sequence may be bicistronic or multicistronic and comprises at least two coding sequences, wherein the at least two coding sequences encode two or more peptides or proteins as defined herein. Accordingly, the coding sequences in a bicistronic or multicistronic RNA suitably encode distinct peptides or proteins as defined herein. Preferably, the coding sequences in said bicistronic or multicistronic constructs may be separated by at least one IRES (internal ribosomal entry site) sequence. In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

In preferred embodiments, the A/U (A/T) content in the environment of the ribosome binding site of the nucleic acid sequence may be increased compared to the A/U (A/T) content in the environment of the ribosome binding site of its respective wild or reference type nucleic acid. This modification (an increased A/U (A/T) content around the ribosome binding site) increases the efficiency of ribosome binding to the RNA. An effective binding of the ribosomes to the ribosome binding site in turn has the effect of an efficient translation the RNA. Accordingly, in a particularly preferred embodiment, the nucleic acid sequence comprises a ribosome binding site, also referred to as “Kozak sequence” identical to or at least 80%, 85%, 90%, 95% identical to any one of the sequences SEQ ID NOs: 180 or 181 of PCT/EP2020/052775, or fragments or variants thereof.

In particularly preferred embodiments the nucleic acid sequence contains a ribosome binding site, also referred to as “Kozak sequence” identical to or at least 80%, 85%, 90%, 95% identical to any one of the sequences SEQ ID NOs: 202 to 209, or fragments or variants thereof.

In a preferred embodiment, the therapeutic peptide or protein is or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a cytokine, a structural protein, a cytoplasmic protein, a cytoskeletal protein, a viral antigen, a bacterial antigen, a protozoan antigen, an allergen, a tumor antigen, an autoimmune antigen, cytostatic or cytotoxic polypeptides or fragments, variants, or combinations of any of these.

In preferred embodiments, the nucleic acid sequence comprises at least one codon modified coding sequence, wherein the amino acid sequence encoded by the at least one codon modified coding sequence is preferably not being modified compared to the amino acid sequence encoded by the corresponding reference coding sequence. The term “codon modified coding sequence” relates to coding sequences or region that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (Table II) to optimize/modify the coding sequence for in vivo applications as outlined above.

In preferred embodiments, the at least one cds of the nucleic acid sequence is a codon modified cds, wherein the amino acid sequence encoded by the at least one codon modified cds is preferably not being modified compared to the amino acid sequence encoded by the corresponding wild type or reference cds.

TABLE II
Human codon usage with respective codon
frequencies indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

In other preferred embodiments, the nucleic acid sequence comprises at least one codon modified coding sequence wherein the cds is selected from a C increased coding sequence, a CAI increased coding sequence, a human codon usage adapted coding sequence, a G/C content modified coding sequence, or a G/C optimized coding sequence, or any combination thereof.

In preferred embodiments, the nucleic acid sequence may be codon modified, wherein the C content of the at least one coding sequence may be increased, preferably maximized, compared to the C content of the corresponding wild type or reference coding sequence (herein referred to as “C maximized coding sequence”). The amino acid sequence encoded by the C maximized coding sequence of the nucleic acid is preferably not modified compared to the amino acid sequence encoded by the respective wild type or reference coding sequence. The generation of a C maximized RNA sequences be carried out using a modification method according to WO2015/062738. In this context, the disclosure of WO2015/062738 is included herewith by reference.

In other preferred embodiments, the nucleic acid sequence may be codon modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the nucleic acid sequence is preferably modified such that the frequency of the codons encoding the same amino acid corresponds to the naturally occurring frequency of that codon according to the human codon usage. Such a procedure may be applied for each amino acid encoded by the coding sequence of the nucleic acid sequence to obtain sequences adapted to human codon usage.

In further preferred embodiments, the nucleic acid sequence may be codon modified, wherein the codon adaptation index (CAI) may be increased or preferably maximised in the at least one coding sequence (herein referred to as “CAI maximized coding sequence”). It is preferred that all codons of the wild type or reference sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table II), most frequent human codons are marked with asterisks). Suitably, the RNA may comprise at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). Such a procedure (as exemplified for Ala) may be applied for each amino acid encoded by the coding sequence of the nucleic acid to obtain CAI maximized coding sequences.

In preferred embodiments, the nucleic acid sequence may be codon modified, wherein the G/C content of the at least one coding sequence may be optimized compared to the G/C content of the corresponding wild type or reference coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the nucleic acid sequence is preferably not modified as compared to the amino acid sequence encoded by the respective wild type or reference coding sequence. The generation of a G/C content optimized RNA sequences may be carried out using a method according to WO2002/098443. In this context, the disclosure of WO2002/098443 is included in its full scope in the present invention.

In preferred embodiments, the nucleic acid sequence may be codon modified, wherein the G/C content of the at least one coding sequence may be modified compared to the G/C content of the corresponding wild type or reference coding sequence (herein referred to as “G/C content modified coding sequence”). In this context, the terms “G/C optimization” or “G/C content modification” relate to an nucleic acid sequence that comprises a modified, preferably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Advantageously, nucleic acid sequences having an increased G/C content may be more stable or may show a better expression than sequences having an increased A/U. The amino acid sequence encoded by the G/C content modified coding sequence of the nucleic acid sequence is preferably not modified as compared to the amino acid sequence encoded by the respective wild type or reference sequence. Suitably, the G/C content of the coding sequence of the nucleic acid sequence is increased by at least 10%, 20%, 30%, preferably by at least 40% compared to the G/C content of the corresponding wild type or reference coding sequence.

In various embodiments, the nucleic acid sequence has a GC content of about 50% to about 80%. In preferred embodiments, the nucleic acid sequence has a GC content of at least about 50%, preferably at least about 55%, more preferably of at least about 60%. In specific embodiments, the nucleic acid sequence has a GC content of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%.

In various embodiments, the coding sequence of the nucleic acid sequence has a GC content of about 60% to about 90%. In preferred embodiments, the coding sequence of the nucleic acid sequence has a GC content of at least about 60%, preferably at least about 65%, more preferably of at least about 70%. In specific embodiments, the nucleic acid sequence of the composition has a GC content of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%.

### PolyA/PolyC/HSL

In various embodiments, the nucleic acid sequence comprises at least one poly(A) sequence, and/or at least one poly(C) sequence, and/or at least one histone stem-loop sequence/structure.

In preferred embodiments, the nucleic acid sequence comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of an RNA of up to about 1000 adenosine nucleotides. Preferably, said poly(A) sequence is essentially homopolymeric, e.g. a poly(A) sequence of e.g. 100 adenosine nucleotides has essentially the length of 100 nucleotides.

In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide). For example, the poly(A) sequence may comprise about 100 A nucleotides being interrupted by at least one nucleotide different from A (e.g. a linker (L), typically about 2 to 20 nucleotides in length), e.g. A30-L-A70 or A70-L-A30.

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides. In preferred embodiments, the at least one nucleic acid comprises at least one poly(A) sequence comprising about 30 to about 200 adenosine nucleotides. In particularly preferred embodiments, the poly(A) sequence comprises about 64 adenosine nucleotides (A64). In other particularly preferred embodiments, the poly(A) sequence comprises about 100 adenosine nucleotides (A100). In other embodiments, the poly(A) sequence comprises about 150 adenosine nucleotides.

The poly(A) sequence as defined herein may be located directly at the 3′ terminus of the at least one nucleic acid sequence, preferably directly located at the 3′ terminus of a nucleic acid sequence In preferred embodiments, the nucleic acid sequence may comprise a poly(A) sequence obtained by enzymatic polyadenylation, wherein the majority of nucleic acid molecules comprise about 100 (+/−20) to about 500 (+/−50), preferably about 250 (+/−20) adenosine nucleotides.

In embodiments, the nucleic acid sequence comprises a poly(A) sequence derived from a template DNA and additionally comprises at least one poly(A) sequence generated by enzymatic polyadenylation, e.g. as described in WO2016/091391.

In embodiments, the nucleic acid sequence comprises at least one polyadenylation signal.

In further embodiments, the nucleic acid sequence comprises at least one poly(C) sequence.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

In embodiments, the nucleic acid sequence comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence may preferably be derived from formulae (I) or (II) of WO2012/019780. According to a further preferred embodiment, the nucleic acid sequence comprises at least one histone stem-loop sequence derived from at least one of the specific formulae (Ia) or (IIa) of the patent application WO2012/019780.

In preferred embodiments, the nucleic acid sequence comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 178 or 179 of PCT/EP2020/052775, or fragments or variants thereof.

In other preferred embodiments, the nucleic acid sequence comprises a 3′-terminal sequence element. Said 3′-terminal sequence element comprises a poly(A) sequence and a histone-stem-loop sequence. Accordingly, the nucleic acid sequence comprises at least one 3-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182 to 230 of PCT/EP2020/052775, or a fragment or variant thereof.

In preferred embodiments, the nucleic acid sequence comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 210 or 211, or fragments or variants thereof.

### UTR

In one embodiment the nucleic acid sequence comprises at least one 5′-UTR and/or the at least one 3′-UTR are heterologous UTRs.

In preferred embodiments, the nucleic acid sequence comprises at least one 5′-UTR and/or 3′-UTR are heterologous UTRs of a gene.

Notably, UTRs may harbor regulatory sequence elements that determine nucleic acid, e.g. RNA turnover, stability, and localization. Moreover, UTRs may harbor sequence elements that enhance translation. In medical application of nucleic acid sequences (including DNA and RNA), translation of the RNA into at least one peptide or protein is of paramount importance to therapeutic efficacy. Certain combinations of 3′-UTRs and/or 5′-UTRs may enhance the expression from operably linked coding sequences encoding peptides or proteins of the invention. Nucleic acid molecules harboring said UTR combinations advantageously enable rapid and transient expression of antigenic peptides or proteins after administration to a subject, preferably after intramuscular administration. Suitably, the nucleic acid sequence of the invention comprises at least one heterologous 5′-UTR and/or at least one heterologous 3′-UTR. Said heterologous 5′-UTRs or 3′-UTRs may be derived from naturally occurring genes or may be synthetically engineered. In preferred embodiments, the nucleic acid, preferably the RNA comprises at least one coding sequence operably linked to at least one (heterologous) 3′-UTR and/or at least one (heterologous) 5′-UTR. Those UTR sequences can also be only a part of a UTR sequence of a gene. In some embodiments the 5′ and/or the 3′ UTRs used in this invention can also be designed synthetically using a predictive model based on polysome profiling as described by Sample et al 2019 (Sampel et al, Nat Biotechnol. 2019 July; 37(7):803-809. doi: 10.1038/s41587-019-0164-5. Epub 2019 Jul. 1).

In preferred embodiments, at least one heterologous 3′-UTR comprises a nucleic acid sequence derived from a 3′-UTR of a gene selected from PSMB3, ALB7, alpha-globin, CASP1, COX6B1, GNAS, NDUFA1 or RPS9 from a homolog, a fragment or a variant of any one of these genes

The term “3-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of a nucleic acid, e.g. a DNA or an RNA, located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

Preferably, the nucleic acid sequence comprises a 3′-UTR, which may be derivable from a gene that relates to an RNA with enhanced half-life (i.e. that provides a stable RNA).

In some embodiments, a 3′-UTR comprises one or more polyadenylation signals, a binding site for proteins that affect nucleic acid stability or location in a cell, or one or more miRNA or binding sites for miRNAs. The 3′ UTR may comprise one or more miRNA binding sites, miRNA target sequences, miRNA sequences, or miRNA seeds.

Such sequences may e.g. correspond to any known miRNA such as those taught in US2005/0261218 and US2005/0059005.

Accordingly, miRNA, or binding sites miRNAs as defined above may be removed from the 3′-UTR or introduced into the 3′-UTR in order to tailor the expression from the nucleic acid, e.g. the RNA to desired cell types or tissues (e.g. muscle cells).

In preferred embodiments, the nucleic acid sequence comprises at least one heterologous 3′-UTR, wherein the at least one heterologous 3′-UTR comprises a nucleic acid sequence derived from a 3′-UTR of a gene selected from PSMB3, ALB7, alpha-globin (referred to as “muag”), CASP1, COX6B1, GNAS, NDUFA1 and RPS9, RSP10 or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 87-194 or a fragment or a variant of any of these. Particularly preferred nucleic acid sequences in that context can be derived from published PCT application WO2019/077001, in particular, claim 9 of WO2019/077001. The corresponding 3′-UTR sequences of claim 9 of WO2019/077001 are herewith incorporated by reference (e.g., SEQ ID NOs: 23-34 of WO2019/077001, or fragments or variants thereof).

In some embodiments, the nucleic acid sequence may comprise a 3-UTR derived from an alpha-globin gene. Said 3′-UTR derived from a alpha-globin gene (“muag”) may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 89 or 90 or a fragment or a variant thereof.

In preferred embodiments, the nucleic acid sequence may comprise a 3-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 87 or 88 or a fragment or a variant thereof.

The nucleic acid sequence of the invention comprises a PSMB3 3-UTR and at least one miRNA binding site sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 343-347, SEQ ID NO: 352-377, SEQ ID NO: 379-381, or fragments or variants of any of these.

In other embodiments, the nucleic acid sequence may comprise a 3′-UTR as described in WO2016/107877, the disclosure of WO2016/107877 relating to 3-UTR sequences herewith incorporated by reference. Suitable 3′-UTRs are SEQ ID NOs: 1-24 and SEQ ID NOs: 49-318 of WO2016/107877, or fragments or variants of these sequences. In other embodiments, the nucleic acid comprises a 3′-UTR as described in WO2017/036580, the disclosure of WO2017/036580 relating to 3-UTR sequences herewith incorporated by reference. Suitable 3′-UTRs are SEQ ID NOs: 152-204 of WO2017/036580, or fragments or variants of these sequences. In other embodiments, the nucleic acid comprises a 3′-UTR as described in WO2016/022914, the disclosure of WO2016/022914 relating to 3′-UTR sequences herewith incorporated by reference. Particularly preferred 3-UTRs are nucleic acid sequences according to SEQ ID NOs: 20-36 of WO2016/022914, or fragments or variants of these sequences.

In preferred embodiments, at least one heterologous 5′-UTR of a gene comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

The terms “5′-untranslated region” or “5′-UTR” or “5′-UTR element” will be recognized and understood by the person of ordinary skill in the art, and are e.g. intended to refer to a part of the nucleic acid sequence located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, or according to this invention miRNA binding sites etc. The 5′-UTR may be post-transcriptionally modified, e.g. by enzymatic or post-transcriptional addition of a 5′ cap structure (e.g. for mRNA as defined above).

Preferably, the nucleic acid sequence comprises a 5′-UTR, which may be derivable from a gene that relates to an RNA with enhanced half-life (i.e. that provides a stable RNA).

In preferred embodiments, a 5′-UTR comprises one or more of a binding site for proteins that affect an RNA stability of location in a cell, or one or more miRNA or binding sites for miRNAs (as defined above).

Accordingly, miRNA or binding sites for miRNAs as defined above may be removed from the 5′-UTR or introduced into the 5′-UTR in order to tailor the expression or activity of the therapeutic RNA to desired cell types or tissues.

In other preferred embodiments, the nucleic acid sequence comprises at least one heterologous 5′-UTR, wherein the at least one heterologous 5′-UTR comprises a nucleic acid sequence derived from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 1-86 or a fragment or a variant of any of these. Particularly preferred nucleic acid sequences in that context can be selected from published PCT application WO2019/077001, in particular, claim 9 of WO2019/077001. The corresponding 5′-UTR sequences of claim 9 of WO2019/077001 are herewith incorporated by reference (e.g. SEQ ID NOs: 1-20 of WO2019/077001, or fragments or variants thereof).

In preferred embodiments, the nucleic acid sequence may comprise a 5′-UTR derived from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1 or 2 or a fragment or a variant thereof.

The nucleic acid of the invention comprises a HSD17B4 5′-UTR and at least one miRNA binding site sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 304-342, or fragments or variants of any of these.

In other embodiments, the nucleic acid sequence comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the coding RNA comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016/107877, or fragments or variants of these sequences. In other embodiments, the nucleic acid sequence comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1-151 of WO2017/036580, or fragments or variants of these sequences. In other embodiments, the nucleic acid sequence comprises a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3-19 of WO2016/022914, or fragments or variants of these sequences.

In some embodiments the 5′ UTR used in this invention can also be designed synthetically using a predictive model based on polysome profiling as described by Sample et al 2019 (Sampel et al, Nat Biotechnol. 2019 July; 37(7):803-809. doi: 10.1038/s41587-019-0164-5. Epub 2019 Jul. 1).

According to the invention the nucleic acid sequence comprising the 5′ UTR is selected or derived from HSD17B4 and wherein the 3′ UTR is selected or derived from PSMB3 and wherein the nucleic acid additionally comprises at least one 5′ Cap structure, preferably a Cap1, and at least one 3′ terminal Poly(A) sequence.

In various embodiments, the nucleic acid sequence may comprise a 5-terminal sequence element according to SEQ ID NOs: 176 or 177 of PCT/EP2020/052775, or a fragment or variant thereof. Such a 5-terminal sequence element comprises e.g. a binding site for T7 RNA polymerase. Further, the first nucleotide of said 5-terminal start sequence may preferably comprise a 2′O methylation, e.g. 2′O methylated guanosine or a 2′O methylated adenosine (which is an element of a Cap1 structure).

In particularly preferred embodiments, the nucleic acid sequence comprises at least one coding sequence as defined wherein said coding sequence is operably linked to a HSD17B4 5′-UTR and a PSMB3 3′-UTR (HSD17B4/PSMB3).

In particularly preferred embodiments, the nucleic acid sequence comprises at least one coding sequence as defined herein, wherein said coding sequence is operably linked to an alpha-globin (“muag”) 3′-UTR.

The nucleic acid sequence according to any of the preceding claims, wherein the nucleic acid is selected from DNA or RNA, preferably from plasmid DNA, viral DNA, template DNA, viral RNA, self-replicating RNA or replicon RNA, and most preferably from an mRNA.

In particularly preferred embodiments, the nucleic acid sequence is an mRNA.

The terms “RNA” and “mRNA” are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

In vivo, transcription of DNA usually results in the so-called premature RNA, which has to be processed into so-called messenger RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of mRNA. The mature messenger RNA usually provides the nucleotide sequence that may be translated into an amino acid sequence of a particular peptide or protein. Typically, a mature mRNA comprises a 5′-cap, a 5′-UTR, an open reading frame, a 3′-UTR and a poly(A) and/or a poly(C) sequence. In the context of the present invention, an mRNA may also be an artificial molecule, i.e. a molecule not occurring in nature. This means that the mRNA in the context of the present invention may, e.g., comprise a combination of a 5′-UTR, open reading frame, 3′-UTR and poly(A) sequence, which does not occur in this combination in nature. A typical mRNA (messenger RNA) in the context of the invention provides the coding sequence that is translated into an amino-acid sequence of a peptide or protein after e.g. in vivo administration to a cell.

### Target Expression

In embodiments, the expression of the encoded peptide or protein by the nucleic acid sequence of this invention is reduced or prevented in the liver and/or liver associated cells, e.g. hepatocytes, hepatic stellate fat storing (ITO) cells, Kupffer cells or liver endothelial cells.

In another embodiment, the expression of the encoded peptide or protein is reduced or prevented in immune cells.

Accordingly, the expression of the encoded peptide or protein by the nucleic acid sequence may be elevated in other tissue/organs. Those might be selected from the group consisting of: brain; lung; breast; pancreas; colon, cancer cells, immune cells, or kidney.

For example, if nucleic acid sequence is not intended to be delivered to the liver but ends up there, then miRNA-122, a miRNA abundant in liver, can inhibit the expression from the gene of interest if one or multiple target sites of miRNA-122 are engineered prior (5′) to the 5′ UTR of nucleic acid sequence. Introduction of one or multiple binding sites for different miRNA can be engineered to further decrease the longevity, stability, and protein translation of a nucleic acid sequence. As used herein, the term “miRNA target site” refers to a miRNA binding site or a miRNA recognition site, or any nucleotide sequence to which a miRNA binds or associates. It should be understood that “binding” may follow traditional Watson-Crick hybridization rules or may reflect any stable association of the miRNA with the target sequence at or adjacent to the miRNA site.

Conversely, for the purposes of the nucleic acid sequence of the present invention, miRNA binding sites can be engineered out of (i.e. removed from) sequences in which they naturally occur in order to increase protein expression in specific tissues. For example, miRNA-122 binding sites may be removed to improve protein expression in the liver.

Therefore, in such a preferred embodiments the nucleic acid sequence according to the invention preferably comprises at least one miRNA-122 binding site, preferably 5′ to the 5′-UTR most preferably 5′ to the 5′UTR and within the 5′UTR.

In preferred embodiments, the expression of the encoded peptide or protein by the nucleic acid sequence is reduced in the cells, organ or tissue by at least 10%, 20%, 30%, 40%, 50%, 60%, 70%, 80% or 90% compared to a reference nucleic acid sequence without miRNA binding sites. In most preferred embodiments, the expression of the encoded peptide or protein by the nucleic acid sequence is reduced the cells, organ or tissue by at least 70%, 80% 90% or up to 95% compared to a reference nucleic acid sequence without miRNA binding sites. Hereby, the expression can be determined by various well-established expression assays, for example, protein expression can be determined using antibody-based detection methods (western blots, FACS) or quantitative mass spectrometry. The same conditions (e.g. the same cell lines, same organism, same application route, the same detection method, the same amount of nucleic acid sequence) have to be used (if feasible) to allow a valid comparison. The person of skill in the art understands how to perform a comparison of the inventive combination and a respective reference or control nucleic acid sequence (e.g. a nucleic acid sequence without miRNA binding sites).

In preferred embodiments, the expression of the encoded peptide or protein by the nucleic acid sequence is reduced in the liver and/or liver associated cells, eg, hepatocytes, hepatic stellate fat storing (ITO) cells, Kupffer cells or liver endothelial cells, by at least 10%, 20%, 30%, 40%, 50%, 60%, 70%, 80% or 90% compared to a reference nucleic acid sequence without miRNA binding sites. In most preferred embodiments, the expression of the encoded peptide or protein by the nucleic acid sequence is reduced in the liver and/or liver associated cells, eg, hepatocytes, hepatic stellate fat storing (ITO) cells, Kupffer cells or liver endothelial cells, by at least 70%, 80% 90% or up to 95% compared to a reference nucleic acid sequence without miRNA binding sites. Hereby, the expression can be determined by various well-established expression assays, for example, protein expression can be determined using antibody-based detection methods (western blots, FACS) or quantitative mass spectrometry. The same conditions (e.g. the same cell lines, same organism, same application route, the same detection method, the same amount of nucleic acid sequence) have to be used (if feasible) to allow a valid comparison. The person of skill in the art understands how to perform a comparison of the inventive combination and a respective reference or control nucleic acid sequence (e.g. a nucleic acid sequence without miRNA binding sites).

In preferred embodiments, upon administration of the nucleic acid of the invention to a cell or subject, the encoded peptide or protein is expressed in non-liver cells, preferably in immune cells or muscle cells.

In other preferred embodiments, upon administration of the nucleic acid to a cell or subject, 80%, 85%, 90%, 95%, 96%, 97%, 98%, 99% of the expressed peptide or protein is produced in non-liver cells, preferably in immune cells or muscle cells.

In various embodiments, the nucleic acid is administered intramuscular.

In preferred embodiments, the encoded peptide or protein is selected or derived from an antigen or epitope of an antigen.

In preferred embodiments, the antigen or epitope of an antigen is selected from a pathogen antigen, preferably a viral antigen, a bacterial antigen.

In preferred embodiments, the antigen or epitope of an antigen is selected from a tumor antigen.

In preferred embodiments, the expression of the encoded peptide or protein by the nucleic acid sequence is reduced in immune cells by at least 10%, 20%, 30%, 40%, 50%, 60%, 70%, 80% or 90% compared to a reference nucleic acid sequence without miRNA binding sites. In most preferred embodiments, the expression of the encoded peptide or protein by the nucleic acid sequence is reduced in immune cells by at least 70%, 80% 90% or up to 95% compared to a reference nucleic acid sequence without miRNA binding sites. Hereby, the expression can be determined by various well-established expression assays, for example, protein expression can be determined using antibody-based detection methods (western blots, FACS) or quantitative mass spectrometry. The same conditions (e.g. the same cell lines, same organism, same application route, the same detection method, the same amount of nucleic acid sequence) have to be used (if feasible) to allow a valid comparison. The person of skill in the art understands how to perform a comparison of the inventive combination and a respective reference or control nucleic acid sequence (e.g. a nucleic acid sequence without miRNA binding sites).

In preferred embodiments, upon administration of the nucleic acid to a cell or subject, the encoded peptide or protein is expressed in non-immune cells, preferably in liver cells.

In other preferred embodiments, upon administration of the nucleic acid to a cell or subject, 80%, 85%, 90%, 95%, 96%, 97%, 98%, 99% of the expressed peptide or protein is produced in non-immune cells, preferably in the liver.

In one embodiment the nucleic acid sequence is administered intravenously.

The nucleic acid sequence of the invention, wherein the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, cytostatic or cytotoxic peptide or protein, or fragments, variants, or combinations of any of these. In preferred embodiments, the expression of the encoded peptide or protein by the nucleic acid sequence is reduced in immune cells and liver cells by at least 10%, 20%, 30%, 40%, 50%, 60%, 70%, 80% or 90% compared to a reference nucleic acid sequence without miRNA binding sites. In most preferred embodiments, the expression of the encoded peptide or protein by the nucleic acid sequence is reduced in immune cells and liver cells by at least 70%, 80% 90% or up to 95% compared to a reference nucleic acid sequence without miRNA binding sites. Hereby, the expression can be determined by various well-established expression assays, for example, protein expression can be determined using antibody-based detection methods (western blots, FACS) or quantitative mass spectrometry. The same conditions (e.g. the same cell lines, same organism, same application route, the same detection method, the same amount of nucleic acid sequence) have to be used (if feasible) to allow a valid comparison. The person of skill in the art understands how to perform a comparison of the inventive combination and a respective reference or control nucleic acid sequence (e.g. a nucleic acid sequence without miRNA binding sites).

In preferred embodiments, upon administration of the nucleic acid to a cell or subject, the encoded peptide or protein is expressed in non-immune cells and in non-liver cells.

In other preferred embodiments, upon administration of the nucleic acid to a cell or subject, 80%, 85%, 90%, 95%, 96%, 97%, 98%, 99% of the expressed peptide or protein is produced in non-immune cells and non liver cells.

In embodiments, the nucleic acid is administered intravenously, intrapulmonal, intratumoral, or intraocular administration.

In various embodiments, the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, cytostatic or cytotoxic peptide or protein, or fragments, variants, or combinations of any of these.

In some embodiments, the expression of the encoded peptide or protein by the nucleic acid sequence can be detected within tumor cells.

Without being limited thereto, tumor cells in this context, for example, may be selected from affected tissues of cancer or tumor diseases chosen from melanomas, malignant melanomas, colon carcinomas, lymphomas, sarcomas, blastomas, kidney carcinomas, gastrointestinal tumors, gliomas, prostate tumors, bladder cancer, rectal tumors, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, diverse virus-induced tumors, such as e.g. papilloma virus-induced carcinomas (e.g. cervix carcinoma=cervical cancer), adenocarcinomas, herpes virus-induced tumors (e.g. Burkitt's lymphoma, EBV-induced B cell lymphoma), hepatitis B-induced tumors (hepatocell carcinomas), HTLV-1- and HTLV-2-induced lymphomas, acusticus neurinoma, lung carcinomas (=lung cancer=bronchial carcinoma), small cell lung carcinomas, throat cancer, anal carcinoma, glioblastoma, rectum carcinoma, astrocytoma, brain tumors, retinoblastoma, basalioma, brain metastases, medulloblastomas, vaginal cancer, testicular cancer, thyroid carcinoma, Hodgkin's syndrome, meningeomas, Schneeberger's disease, pituitary tumor, mycosis fungoides, carcinoids, neurinoma, spinalioma, Burkitt's lymphoma, laryngeal cancer, kidney cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal carcinoma (=oesophageal cancer), wart conditions, small intestine tumors, craniopharyngeomas, ovarian carcinoma, soft tissue tumors, ovarian cancer (=ovarian carcinoma), pancreatic carcinoma (=pancreatic cancer), endometrium carcinoma, liver metastases, penis cancer, tongue cancer, gallbladder cancer, leukaemia, plasmocytoma, lid tumor, prostate cancer (=prostate tumors) etc.

The term ‘cancer’ as used herein refers to neoplasms in tissue, including malignant tumors that may be primary cancer starting in a particular tissue, or secondary cancer having spread by metastasis from elsewhere. The terms cancer, neoplasm and malignant tumors are used interchangeably herein. Cancer may denote a tissue or a cell located within a neoplasm or with properties associated with a neoplasm. Neoplasms typically possess characteristics that differentiate them from normal tissue and normal cells. Among such characteristics are included, but not limited to: a degree of anaplasia, changes in morphology, irregularity of shape, reduced cell adhesiveness, the ability to metastasize, and increased cell proliferation. Terms pertaining to and often synonymous with ‘cancer’ include sarcoma, carcinoma, malignant tumor, epithelioma, leukaemia, lymphoma, transformation, neoplasm and the like. As used herein, the term ‘cancer’ includes premalignant, and/or precancerous tumors, as well as malignant cancers.

In preferred embodiments, the coding sequence encoding at least one therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, a viral antigen, a bacterial antigen, a protozoan antigen, an allergen, an autoimmune antigen, a tumor antigen, cytostatic or cytotoxic peptide or protein, or fragments, variants, or combinations of any of these.

Accordingly, the nucleic acid sequence of this invention preferably comprises a miRNA-122 binding site within the miRNA binding site sequence located immediately 5′ (prior) of the 5′ UTR and least one coding region which encodes a peptide or protein that can be detected within tumor cells.

In other embodiments, the peptide or protein encoded by the nucleic acid sequence is a cytokine. “Cytokine” quite generally is to be understood as a protein, which influences the behavior of cells. The action of cytokines takes place via specific receptors on their target cells. Cytokines include, for example, monokines, lymphokines or also interleukins, interferons, immunoglobulins and chemokines.

Preferably, the peptide or protein encoded by the nucleic acid sequence is a cytokine, for example, cytokines of class I of the cytokine family that contain 4 position-specific conserved cysteine residues (CCCC) and a conserved sequence motif Trp-Ser-X-Trp-Ser (WSXWS), wherein X represents an unconserved amino acid. Cytokines of class I of the cytokine family include the GM-CSF sub-family, for example IL-3, IL-5, GM-CSF, the IL-6 sub-family, for example IL-6, IL-11, IL-12, or the IL-2 sub-family, for example IL-2, IL-4, IL-7, IL-9, IL-15, etc., or the cytokines IL-1a, IL-1β, IL-10 etc. By analogy, such the peptide or protein can also include cytokines of class II of the cytokine family (interferon receptor family), which likewise contain 4 position-specific conserved cysteine residues (CCCC) but no conserved sequence motif Trp-Ser-X-Trp-Ser (WSXWS). Cytokines of class II of the cytokine family include, for example, IFN-α, IFN-p, IFN-γ, etc. The peptide or protein encoded by the nucleic acid sequence according to the invention can further include also cytokines of the tumor necrosis family, for example TNF-α, TNF-β, TNF-RI, TNF-RII, CD40, Fas, etc., or cytokines of the chemokine family, which contain 7 transmembrane helices and interact with G-protein, for example IL-8, MIP-1, RANTES, CCR5, CXR4, etc. Such proteins can also be selected from apoptosis factors or apoptosis-related or -linked proteins, including AIF, Apaf, for example Apaf-1, Apaf-2, Apaf-3, or APO-2 (L), APO-3 (L), apopain, Bad, Bak, Bax, Bcl-2, BcI-xL, BcI-xS, bik, CAD, calpain, caspases, for example caspase-1, caspase-2, caspase-3, caspase-4, caspase-5, caspase-6, caspase-7, caspase-8, caspase-9, caspase-10, caspase-11, ced-3, ced-9, c-Jun, c-Myc, crm A, cytochrome C, CdR1, DcR1, DD, DED, DISC, DNA-PKcs, DR3, DR4, DR5, FADD/MORT-1, FAK, Fas (Fas ligand CD95/fas (receptor)), FLICE/MACH, FLIP, fodrin, fos, G-actin, Gas-2, gelsolin, granzymes A/B, ICAD, ICE, JNK, lamin A/B, MAP, MCL-1, Mdm-2, MEKK-1, MORT-1, NEDD, NF-κB, NuMa, p53, PAK-2, PARP, perforin, PITSLRE, PKC6, pRb, presenilin, prICE, RAIDD, Ras, RIP, sphingomyelinase, thymidine kinase from Herpes simplex, TRADD, TRAF2, TRAIL, TRAIL-R1, TRAIL-R2, TRAIL-R3, transglutaminase, etc.

In preferred embodiments, the peptide or protein encoded by the nucleic acid sequence is selected from interleukins, chemokines, interferons or lymphokines.

As defined herein, Interleukins (ILs) are a group of cytokines (secreted proteins and signal molecules) that were first seen to be expressed by white blood cells (leukocytes). The majority of interleukins are synthesized by helper CD4 T lymphocytes, as well as through monocytes, macrophages, and endothelial cells and promote the development and differentiation of T and B lymphocytes, and hematopoietic cells. They are particularly important in stimulating immune responses, such as inflammation. Chemokines are a group of small hormone-like molecules that are secreted by cells and that stimulate the movement of cells of the immune system toward specific sites in the body. The major role of chemokines is to act as a chemoattractant to guide the migration of cells. Interferons (IFN) IFNs belong to the large class of proteins molecules used for communication between cells to trigger the protective defenses of the immune system that help eradicate pathogens. More than twenty distinct IFN genes and proteins have been identified in animals, including humans. They are typically divided among three classes: Type I IFN, Type II IFN, and Type III IFN. IFNs belonging to all three classes are important for fighting viral infections and for the regulation of the immune system. Lymphokines are a subset of cytokines that are produced by lymphocytes. They are protein mediators to direct the immune system response by signaling between its cells. Lymphokines have many roles, including the attraction of other immune cells, including macrophages and other lymphocytes, to an infected site and their subsequent activation to prepare them to mount an immune response. Important lymphokines include Interleukin 2, Interleukin 3, Interleukin 4, Interleukin 5, Interleukin 6, interleukin 12, Granulocyte-macrophage colony-stimulating factor or Interferon-gamma.

In most preferred embodiments, the peptide or protein encoded by the nucleic acid sequence is selected from interleukins, preferably the interleukin-12 (IL-12).

“Interleukin-12” (IL-12) herein, includes interleukin-12 subunit alpha (IL-12A), or variants or fragments thereof, and/or interleukin-12 subunit beta (IL-12B), or variants or fragments thereof, or heterodimers or fusion products or analogs thereof. Naturally occurring IL-12 is typically a heterodimeric cytokine encoded by two separate genes, IL-12A (p35) and IL-12B (p40). The naturally occurring heterodimer is also referred to as p70. In the context of the present invention, the term “IL-12” refers to a protein consisting of or comprising a naturally occurring form of heterodimeric IL-12, a monomeric IL-12A, a monomeric IL-12B, as well as fragments or variants thereof, and fusion proteins of IL-12A (or a fragment or variant thereof) and IL-12B (or a fragment or variant thereof) wherein said fusion protein comprises IL-12A and IL-12B, which are covalently coupled to each other either directly, e.g. via a peptide bond, or via a suitable linker, e.g. a peptide linker. Fragments, variants, monomers, heterodimers, or analogs of the IL-12 (poly-)peptide or protein are preferably functional, i.e. capable of specifically binding to the IL-12 receptor and preferably inducing the JAK-STAT signaling pathway.

Accordingly, the peptide or protein encoded by the nucleic acid sequence is selected from at least one interleukin-12 (IL-12) subunit. Preferably, the peptide or protein encoded by the nucleic acid sequence is selected from at least one IL-12 alpha (IL-12A) subunit, or a variant or fragment thereof and/or at least one IL-12 beta (IL-12B) subunit, or a variant or fragment thereof, or combinations thereof, preferably connected via a suitable linker.

Accordingly, the nucleic acid sequence preferably comprises at least one miRNA-122 binding site within the miRNA binding site sequence located immediately 5′ (prior) of the 5′ UTR and least one coding region which encodes a interleukin, preferably the interleukin 12 (IL-12).

Most preferably, the nucleic acid sequence according to the invention comprises the sequence according to SEQ ID No. 249, SEQ ID No. 294 or SEQ ID NO. 295.

In specific preferred embodiments, the nucleic acid sequence according to the invention is suitable for use in intratumoral applications.

Preferably, the nucleic acid sequence according to the invention is suitable for an intratumoral administration/application, preferably by injection into tumor tissue.

The term “intratumoral administration/application” refers to the direct delivery into or adjacent to a tumor or cancer and/or immediate vicinity of a tumor or cancer. In the context of the present invention, the term “intratumoral administration/application” thus typically also refers to locoregional or peritumoral application/administration. Multiple injections into separate regions of the tumor or cancer are also included. Furthermore, intratumoral administration/application includes delivery into one or more metastases of the primary tumor, e.g. to lymph nodes, skin, soft tissues, bone, visceral organs or other organs of the body. Intratumoral administration/application can be accomplished by conventional needle injection, needle-free jet injection or electroporation or combinations thereof into tumor or cancer (tissue). Intratumoral administration/application may involve direct injection into tumor or cancer (tissue) with great precision by imaging-guided injection, preferably using an imaging technique, such as computer tomography, ultrasound, gamma camera imaging, positron emission tomography, or magnetic resonance tumor imaging. Further procedures are selected from the group including, but not limited to, direct intratumoral injection by endoscopy, bronchoscopy, cystoscopy, colonoscopy, laparoscope and catheterization. Methods for intratumoral delivery of drugs are known in the art (Brincker, 1993. Crit. Rev. Oncol. Hematol. 15(2):91-8; Celikoglu et al., 2008. Cancer Therapy 6, 545-552).

Preferred examples of tumors or cancers that are suitable for intratumoral, including peritumoral or locoregional administration, preferably imaging guided loco-regional administration, are prostate cancer, lung cancer, breast cancer, brain cancer, head and neck cancer including cancer of the lips, mouth, or tongue, nasopharyngal cancers or lymphoma, thyroid cancer, thymic cancer, colon cancer, stomach cancer, esophageal cancer, liver cancer, biliary cancer, pancreas cancer, ovary cancer, skin cancer, (melanoma and non-melanoma skin cancer), urinary bladder and urothel, uterus and cervix, anal cancer, bone cancers, kidney cancer, adrenal cancer, testicular cancer, cutaneous T cell lymphoma, cutaneous B cell lymphoma, plasmocytoma, other Hodgkin and non-hodgkin lymphomas with injectable, solitary lesions, adenocystic carcinoma, other salivary gland cancers, neuroendocrine tumors, vulvar cancer, sarcoma (incl. pediatric sarcoma), hepatocellular carcinoma or penile cancer lymphomas. In such embodiments it is particularly preferred that the nucleic acid sequence comprises at least one miRNA-122 binding site within the miRNA binding site sequence located immediately 5′ (prior) of the 5′ UTR and least one coding region which encodes the target protein.

In other embodiments, the expression of the encoded peptide or protein by the nucleic acid sequence can be detected within non-liver cells preferably selected from immune cells, muscle cells or lung cells.

Preferably, the encoded peptide or protein by the nucleic acid sequence can be detected within immune cells. Typically, immune cells are defined as a part of the immune system. All the cells of the immune system are white blood cells. These white blood cells are of five types (Lymphocytes, Monocytes and Macrophages, Basophils, Neutrophils, Eosinophils) and all of them have a role in the immune system.

The term “immune system” will be recognized and understood by the person of ordinary skill in the art, and is e.g. intended to refer to a system of the organism that protects the organisms from infection. If a pathogen succeeds in passing a physical barrier of an organism and enters this organism, the innate immune system provides an immediate non-specific response. If pathogens evade this innate response, vertebrates possess a second layer of protection, the adaptive immune system. The immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Accordingly, the nucleic acid sequence of this invention preferably comprises at least one miRNA-122 binding site within the miRNA binding site sequence located immediately 5′ (prior) of the 5′ UTR and least one coding region which encodes a peptide or protein that can be detected within immune cells. Such an embodiment is particularly preferred in vaccination approaches. Therefore, the encoded protein is preferably an antigen or an epitope from an antigen in this context.

In preferred embodiments, the peptide or protein encoded by the nucleic acid sequence according to this invention is selected or derived from an antigen or epitope of an antigen.

The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein, which may be presented by the MHC to T-cells. In addition, fragments, variants and derivatives of peptides or proteins comprising at least one epitope are understood as antigens in the context of the invention. In the context of the present invention, an antigen may be the product of translation of a provided nucleic acid sequence as specified herein.

The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface.

Preferably, the nucleic acid sequence according to the invention may comprise at least one coding region encoding a tumor antigen, a pathogenic antigen, an autoantigen, an alloantigen, or an allergenic antigen.

The term “tumor antigen” refers to antigenic (poly-)peptides or proteins derived from or associated with a (preferably malignant) tumor or a cancer disease. As used herein, the terms “cancer” and “tumor” are used interchangeably to refer to a neoplasm characterized by the uncontrolled and usually rapid proliferation of cells that tend to invade surrounding tissue and to metastasize to distant body sites. The term encompasses benign and malignant neoplasms. Malignancy in cancers is typically characterized by anaplasia, invasiveness, and metastasis; whereas benign malignancies typically have none of those properties. The terms “cancer” and “tumor” in particular refer to neoplasms characterized by tumor growth, but also to cancers of blood and lymphatic system. A “tumor antigen” is typically derived from a tumor/cancer cell, preferably a mammalian tumor/cancer cell, and may be located in or on the surface of a tumor cell derived from a mammalian, preferably from a human, tumor, such as a systemic or a solid tumor. “Tumor antigens” generally include tumor-specific antigens (TSAs) and tumor-associated-antigens (TAAs). TSAs typically result from a tumor specific mutation and are specifically expressed by tumor cells. TAAs, which are more common, are usually presented by both tumor and “normal” (healthy, non-tumor) cells.

The protein or polypeptide may comprise or consist of a tumour antigen, a fragment, variant or derivative of a tumour antigen. Such nucleic acid molecules are particularly useful for therapeutic purposes, particularly genetic vaccination. Preferably, the tumour antigen may be selected from the group comprising a melanocyte-specific antigen, a cancer-testis antigen or a tumour-specific antigen, preferably a CT-X antigen, a non-X CT-antigen, a binding partner for a CT-X antigen or a binding partner for a non-X CT-antigen or a tumour-specific antigen, more preferably a CT-X antigen, a binding partner for a non-X CT-antigen or a tumour-specific antigen or a fragment, variant or derivative of said tumour antigen; and wherein each of the nucleic acid sequences encodes a different peptide or protein; and wherein at least one of the nucleic acid sequences encodes for 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, A T-4, ARTC1/m, B7H4, BAGE-1, BCL-2, bcr/abl, beta-catenin/m, BING-4, BRCAI/m, BRCA2/m, CA 1 5-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R1 71, HLA-A1 1/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1 R, IL-13Ra2, IL-2R, IL-5, immature laminin receptor, kallikrein-2, kallikrein-4, i67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B1 6, MAGE-B1 7, MAGE-C1, MAGE-C2, MAGE-C3, MAGE-D1, MAGE-D2, MAGE-D4, MAGE-E1, MAGE-E2, MAGE-F1, MAGE-H I, MAGEL2, mammaglobin A, MART-1/melan-A, MART-2, MART-2/m, matrix protein 22, MC1 R, M-CSF, ME 1/m, mesothelin, MG50/PXDN, MMP1 1, MN/CA IX-antigen, MRP-3, MUC-1, MUC-2, MUM-1/m, MUM-2/m, MUM-3/m, myosin class I/m, NA88-A, N-acetylgl ucosaminy transferase-V, Neo-PAP, Neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, osteocalcin, osteopontin, pi 5, p190 minor bcr-abl, p53, p53/m, PAGE-4, PAl-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRll, TGM-4, TPI/m, TRAG-3, TRG, TRP-1, TRP-2/6b, TRP/INT2, TRP-p8, tyrosinase, UPA, VEGFR1, VEGFR-2/FLK-1, WT1 and a immunoglobulin idiotype of a lymphoid blood cell or a T cell receptor idiotype of a lymphoid blood cell, or a homolog, fragment, variant or derivative of any of these tumor antigens; preferably survivin or a homologue thereof, an antigen from the MAGE-family or a binding partner thereof or a fragment, variant or derivative of said tumour antigen.

In embodiments, the peptide or protein is selected from an antigen or epitope of an antigen for example a pathogen selected or derived from List 1 provided below.

The term “pathogenic antigen” refers to antigenic (poly-)peptides or proteins derived from or associated with pathogens, i.e. viruses, microorganisms, or other substances causing infection and typically disease, including, besides viruses, bacteria, protozoa or fungi. In particular, such “pathogenic antigens” may be capable of eliciting an immune response in a subject, preferably a mammalian subject, more preferably a human. Typically, pathogenic antigens may be surface antigens, e.g. (poly-)peptides or proteins (or fragments of proteins, e.g. the exterior portion of a surface antigen) located at the surface of the pathogen (e.g. its capsid, plasma membrane or cell wall).

Accordingly, in some preferred embodiments, the nucleic acid sequence may encode in its at least one coding region at least one pathogenic antigen selected from a bacterial, viral, fungal or protozoal antigen. The encoded (poly-)peptide or protein may consist or comprise of a pathogenic antigen or a fragment, variant or derivative thereof.

### List 1: Suitable Pathogens of the Invention

Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 0157:H7, 0111 and 0104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, SARS-CoV-2 coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Westem equine encephalitis virus, Wuchereria bancrofti, Yellow fever virus, Yersinia enterocolitica, Yersinia pestis, and Yersinia pseudotuberculosis.

Particularly preferred in this context are the following antigens:
HIV p24 antigen, HIV envelope proteins (Gp120, Gp41, Gp160), polyprotein GAG, negative factor protein Nef, trans-activator of transcription Tat if the pathogen is Human immunodeficiency virus,
pp65 antigen, membrane protein pp15, capsid-proximal tegument protein pp150, protein M45, DNA polymerase UL54, helicase UL105, glycoprotein gM, glycoprotein gN, glcoprotein H, glycoprotein B gB, protein UL83, protein UL94, protein UL99 if the pathogen is Cytomegalovirus (CMV);
capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 if the pathogen is a Dengue virus (DEN-1, DEN-2, DEN-3 and DEN-4);
hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 if the pathogen is Hepatitis B Virus (HBV);
replication protein E1, regulatory protein E2, protein E3, protein E4, protein E5, protein E6, protein E7, protein E8, major capsid protein L1, minor capsid protein L2 if the pathogen is Human papillomavirus (HPV);
fusion protein F, hemagglutinin-neuramidase HN, glycoprotein G, matrix protein M, phosphoprotein P, nucleoprotein N, polymerase L if the infectious disease is Human parainfluenza virus infection, preferably an infection with Human parainfluenza viruses (HPIV);
Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein, if the pathogen is an Influenza virus;
nucleoprotein N, large structural protein L, phophoprotein P, matrix protein M, glycoprotein G if the pathogen is Rabies virus;
fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 if the pathogen is Respiratory syncytial virus (RSV);
genome polyprotein, protein E, protein M, capsid protein C, protease NS3, protein NS1, protein NS2A, protein AS2B, protein NS4A, protein NS4B, protein NS5 if the pathogen is Yellow fever virus.
spike protein (S), envelope protein (E), membrane protein (M) or nucleocapsid protein (N) from a Coronavirus particularly from SARS-CoV-2.

In some embodiments, the nucleic acid sequence according to the invention is suitable for use in systemic vaccination.

Administration route for systemic vaccination in general include the intravenous administration route.

Hereby in preferred embodiment, the vaccination is suitable for use as therapeutic or prophylactic vaccination.

Within a therapeutic vaccination as described herein, a vaccine is administered after a disease or infection, which has already occurred. A prophylactic vaccination refers to the artificial establishment of specific immunity before a disease or infection.

In most preferred embodiments, the nucleic acid sequence according to the invention is suitable for use in systemic vaccination.

Hereby, “systemic vaccination” as used herein will be understand as a vaccination which relates to a whole system, e.g. an vaccination which affects the whole body, especially as opposed to a particular part.

Administration route for systemic vaccination in general include the intramuscular administration route.

Accordingly, the nucleic acid sequence of this invention comprises a miRNA-122 binding site within the miRNA binding site sequence located immediately 5′ (prior) of the 5′ UTR and least one coding region which encodes a peptide or protein which is derived from an antigen or epitope; the nucleic acid sequence is suitable for use in systemic vaccination. In this context, the nucleic acid sequence may be suitable for use in genetic vaccination.

Genetic vaccination may typically be understood to be a vaccination by administration of a nucleic acid sequence or molecule encoding an antigen or an immunogen or fragments thereof. The nucleic acid sequence may be administered to a subject's body or to isolated cells of a subject. Upon transfection of certain cells of the body or upon transfection of the isolated cells, the antigen or immunogen may be expressed by those cells and subsequently presented to the immune system, eliciting an adaptive, i.e. antigen-specific immune response.

In other embodiments, the therapeutic peptide or protein acid sequence according to the invention wherein the encoded peptide or protein is not selected or derived from an antigen or epitope of an antigen.

In preferred embodiments, the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, cytostatic or cytotoxic peptide or protein, or fragments, variants, or combinations of any of these.

Some advantages associated with the use of multiple binding sites include an increase in the efficiency of differential expression of polypeptides supplied by the nucleic acid sequence of the present invention, within a single organ. Use of different binding site sequences, or sequences, which are applicable to more than one tissue or organ type, can enable differential expression to be achieved in different cell types in more than one organ or tissue. This may be desirable when systemic administration of compositions according to the invention is used, and it is necessary to avoid off-target effects in more than one organ. Even with localized or targeted administration, it is possible that supplied nucleic acid sequences (e.g. mRNA) may encounter or accumulate in organs, tissues, and/or cells for which they were not intended. In particular, liver and kidney tissue may accumulate administered compositions, due to the physiological function of these organs. In these cases, to avoid off-target effects, it may be advantageous for the supplied constructs to comprise miRNA binding site sequences, which would enable reduced expression in these tissues. Conversely, it may be desirable for expression to be encouraged in some organs, tissues and/or cell types but not others, which can be achieved by the selection of miRNA binding site sequences accordingly.

### Second Aspect: Pharmaceutical Composition

In the following, advantageous embodiments and features regarding the formulation/complexation of the nucleic acid sequence according to this invention are described. All described embodiments and features regarding formulation in the context of the inventive method of producing a nucleic acid sequence comprising a miRNA binding site sequence wherein the miRNA binding site sequence is located within and/or immediately 3′ or 5′ of the 5′UTR to allow a cell type specific expression from the nucleic acid sequence within the target organ or organs (first aspect) are likewise be applicable to the pharmaceutical composition (second aspect). Additionally, they are likewise applicable to the kit or kit of parts (third aspect), or the use as a medicament (fourth aspect), and to further aspects of this invention (methods of treating or preventing a disease, disorder, or condition and a method to promote a cell-type specific expression of a peptide or protein within a target organ or organs by using a nucleic acid sequence).

In a second aspect, the present invention provides a pharmaceutical composition comprising the nucleic acid sequence as defined herein or a composition obtained by the method according to this invention optionally comprising one or more pharmaceutically acceptable excipients, carriers, diluents and/or vehicles.

In the context of the invention, a “composition” refers to any type of composition in which the specified ingredients (e.g. nucleic acid sequence, e.g. in association with a polymeric carrier or LNP), may be incorporated, optionally along with any further constituents, usually with at least one pharmaceutically acceptable carrier or excipient. The composition may be a dry composition such as a powder or granules, or a solid unit such as a lyophilized form. Alternatively, the composition may be in liquid form, and each constituent may be independently incorporated in dissolved or dispersed (e.g. suspended or emulsified) form.

The term “pharmaceutically acceptable carrier” or “pharmaceutically acceptable excipient” as used herein preferably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂), Cal₂, CaBr₂, CaCO₃, CaSO₄, Ca(OH)₂.

Furthermore, organic anions of the mentioned cations may be in the buffer. Accordingly, in embodiments, the nucleic acid composition may comprise pharmaceutically acceptable carriers or excipients using one or more pharmaceutically acceptable carriers or excipients to e.g. increase stability, increase cell transfection, permit the sustained or delayed, increase the translation of encoded protein in vivo, and/or alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid and, optionally, a plurality of nucleic acids of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The pharmaceutical composition suitably comprises a safe and effective amount of the nucleic acid sequence as specified herein. As used herein, “safe and effective amount” means an amount of the therapeutic RNA, preferably the mRNA, sufficient to result in expression and/or activity of the encoded protein after administration.

At the same time, a “safe and effective amount” is small enough to avoid serious side-effects caused by administration of said nucleic acid sequence.

Further advantageous embodiments and features of the pharmaceutical composition of the invention are described below. Notably, embodiments and features described in the context of the pharmaceutical composition may likewise be applicable to the kit or kit of parts of the fourth aspect.

Pharmaceutical compositions of the present invention may suitably be sterile and/or pyrogen-free.

The choice of a pharmaceutically acceptable carrier as described above is determined in particular by the mode in which the pharmaceutical composition according to the invention is administered.

### Cationic or Polycationic Peptides and Polymeric Carrier

In preferred embodiments, the nucleic acid sequence is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof

Accordingly, the nucleic acid sequence as defined herein is attached to one or more cationic or polycationic compounds, preferably cationic or polycationic polymers, cationic or polycationic polysaccharide, cationic or polycationic lipid, cationic or polycationic protein, or cationic or polycationic peptide, or any combinations thereof. The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, or histones. More preferably, the coding RNA, preferably the mRNA, is complexed with one or more polycations, preferably with protamine or oligofectamine, most preferably with protamine.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In this context it is particularly preferred that the nucleic acid sequence is complexed or at least partially complexed with a cationic or polycationic compound and/or a polymeric carrier, preferably cationic proteins or peptides. In this context, the disclosure of WO2010/037539 and WO2012/113513 is incorporated herewith by reference. Partially means that only a part of the nucleic acid is complexed with a cationic compound and that the rest of the nucleic acid is in uncomplexed form (“free”).

Further preferred cationic or polycationic proteins or peptides that may be used for complexation can be derived from formula (Arg)l;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In one embodiment the N/P ratio of the nucleic acid sequence to the one or more cationic or polycationic compound is in the range of about 0.1 to 10, including a range of about 0.3 to 4, of about 0.5 to 2, of about 0.7 to 2 and of about 0.7 to 1.5.

In some embodiments, the nucleic acid sequence is complexed, or at least partially complexed, with at least one cationic or polycationic proteins or peptides preferably selected from SEQ ID NOs: 244-248, or any combinations thereof.

In various embodiments, the one or more cationic or polycationic peptides are selected from SEQ ID NOs: 244-248, or any combinations thereof.

Accordingly, in preferred embodiments, the nucleic acid sequence, is complexed or associated with or at least partially complexed or partially associated with one or more cationic or polycationic peptides selected from SEQ ID NOs: 244-246, or any combinations thereof.

Accordingly, in preferred embodiments, the nucleic acid sequence is complexed or associated with or at least partially complexed or partially associated with one or more cationic or polycationic peptides selected from SEQ ID NOs: 247 or 248, or any combinations thereof.

In embodiments, the nucleic acid sequence as defined herein is complexed or associated with or at least partially complexed or partially associated with one or more cationic or polycationic polymer.

In embodiments, the nucleic acid sequence is complexed or associated with or at least partially complexed or partially associated with one or more cationic or polycationic polymer.

Accordingly, in embodiments, the nucleic acid sequence comprises at least one polymeric carrier.

The term “polymeric carrier” as used herein will be recognized and understood by the person of ordinary skill in the art, and are e.g. intended to refer to a compound that facilitates transport and/or complexation of another compound (e.g. cargo nucleic acid). A polymeric carrier is typically a carrier that is formed of a polymer. A polymeric carrier may be associated to its cargo (e.g. DNA, or RNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(p-aminoester), poly(4-hydroxy-L-proine ester) (PHP), poly(allylamine), poly(a-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(p-aminoester), PHP, PAGA, PLGA, PPZ, PPE, PPA and PPE-EA.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via —SH groups).

In this context, polymeric carriers according to formula {(Arg)l;(Lys)m;(His)n;(Orn)o;(Xaa′)x(Cys)y} and formula Cys,{(Arg)l;(Lys)m;(His)n;(Orn)o;(Xaa)x}Cys₂ of the patent application WO2012/013326 are preferred, the disclosure of WO2012/013326 relating thereto incorporated herewith by reference.

In embodiments, the polymeric carrier used to complex the at least one nucleic acid, preferably the at least one nucleic acid sequence may be derived from a polymeric carrier molecule according formula (L-P¹—S—[S—P²—S]ₙ—S—P³-L) of the patent application WO2011/026641, the disclosure of WO2011/026641 relating thereto incorporated herewith by reference.

In some embodiments, the polymeric carrier compound is formed by, or comprises or consists of the peptide elements CysArg12Cys (SEQ ID NO: 244) or CysArg12 (SEQ ID NO: 245) or TrpArg12Cys (SEQ ID NO: 246). In particularly preferred embodiments, the polymeric carrier compound consists of a (R₁₂C)—(R₁₂C) dimer, a (WR₁₂C)—(WR₁₂C) dimer, or a (CR₁₂)—(CR₁₂C)—(CR₁₂) trimer, wherein the individual peptide elements in the dimer (e.g. (WR12C)), or the trimer (e.g. (CR12)), are connected via —SH groups.

In a preferred embodiment of the second aspect, at least one nucleic acid sequence of the second aspect is complexed or associated with a polyethylene glycol/peptide polymer comprising HO-PEG5000-S—(S—CHHHHHHRRRRHHHHHHC—S-)7-S-PEG5000-OH (SEQ ID NO: 247 as peptide monomer), HO-PEG5000-S—(S—CHHHHHHRRRRHHHHHHC—S-)4-S-PEG5000-OH (SEQ ID NO: 247 as peptide monomer), HO-PEG5000-S—(S-CGHHHHHRRRRHHHHHGC-S-)7-S-PEG5000-OH (SEQ ID NO: 248 as peptide monomer) and/or a polyethylene glycol/peptide polymer comprising HO-PEG5000-S—(S-CGHHHHHRRRRHHHHHGC-S-)4-S-PEG5000-OH (SEQ ID NO: 248 of the peptide monomer).

In other embodiments, the composition comprises at least one nucleic acid sequence which is complexed or associated with polymeric carriers and, optionally, with at least one lipid component as described in WO2017/212008, WO2017/212006, WO2017/212007, and WO2017/212009. In this context, the disclosures of WO2017/212008, WO2017/212006, WO2017/212007, and WO2017/212009 are herewith incorporated by reference.

### Formulation in Lipid-Based Carriers:

In particularly preferred embodiments, the at least one artificial nucleic acid, preferably the at least one RNA of the pharmaceutical composition is formulated in lipid-based carriers.

In the context of the invention, the term “lipid-based carriers” encompass lipid-based delivery systems for nucleic acid (e.g. RNA) that comprise a lipid component. A lipid-based carrier may additionally comprise other components suitable for encapsulating/incorporating/complexing a nucleic acid (e.g. RNA) including a cationic or polycationic polymer, a cationic or polycationic polysaccharide, a cationic or polycationic protein, a cationic or polycationic peptide, or any combinations thereof.

In the context of the invention, a typical “lipid-based carrier” is selected from liposomes, lipid nanoparticles (LNPs), lipoplexes, solid lipid nanoparticles, and/or nanoliposomes. The nucleic acid, preferably the RNA of the pharmaceutical composition may completely or partially incorporated or encapsulated in a lipid-based carrier, wherein the nucleic acid (e.g. RNA) may be located in the interior space of the lipid-based carrier, within the lipid layer/membrane of the lipid-based carrier, or associated with the exterior surface of the lipid-based carrier. The incorporation of nucleic acid, preferably the RNA into lipid-based carriers may be referred to as “encapsulation”. A “lipid-based carrier” is not restricted to any particular morphology, and include any morphology generated when e.g. an aggregation reducing lipid and at least one further lipid are combined, e.g. in an aqueous environment in the presence of nucleic acid (e.g. RNA). For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the at least one nucleic acid (e.g. RNA) is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanoparticles (LNPs), a specific type of lipid-based carrier, are characterized as microscopic lipid particles having a solid core or partially solid core. Typically, an LNP does not comprise an interior aqua space sequestered from an outer medium by a bilayer. In an LNP, the at least one nucleic acid (e.g. RNA) may be encapsulated or incorporated in the lipid portion of the LNP enveloped by some or the entire lipid portion of the LNP. An LNP may comprise any lipid capable of forming a particle to which the nucleic acid (e.g. RNA) may be attached, or in which the nucleic acid may be encapsulated. Preferably, said lipid-based carriers are particularly suitable for ocular administration.

In preferred embodiments, the lipid-based carriers of the pharmaceutical composition are selected from liposomes, lipid nanoparticles, lipoplexes, solid lipid nanoparticles, lipo-polylexes, and/or nanoliposomes. In preferred embodiments, the lipid-based carriers of the pharmaceutical composition are lipid nanoparticles (LNPs). In particularly preferred embodiments, the lipid nanoparticles of the pharmaceutical composition encapsulate the at least one nucleic acid, preferably the at least one RNA of the invention.

The term “encapsulated”, e.g. incorporated, complexed, encapsulated, partially encapsulated, associated, partially associated, refers to the essentially stable combination of nucleic acid, preferably RNA with one or more lipids into lipid-based carriers (e.g. larger complexes or assemblies) preferably without covalent binding of the nucleic acid. The lipid-based carriers—encapsulated nucleic acid (e.g. RNA) may be completely or partially located in the interior of the lipid-based carrier (e.g. the lipid portion and/or an interior space) and/or within the lipid layer/membrane of the lipid-based carriers. The encapsulation of an nucleic acid (e.g. RNA) into lipid-based carriers is also referred to herein as “incorporation” as the nucleic acid (e.g. RNA) is preferably contained within the interior of the lipid-based carriers. Without wishing to be bound to theory, the purpose of incorporating or encapsulating nucleic acid into lipid-based carriers may be to protect the nucleic acid from an environment which may contain enzymes, chemicals, or conditions that degrade the nucleic acid (e.g. RNA). Moreover, incorporating nucleic acid into lipid-based carriers may promote the uptake of the nucleic acid and their release from the endosomal compartment, and hence, may enhance the therapeutic effect of the nucleic acid (e.g. RNA) when administered to a cell or a subject.

In preferred embodiments, the lipid-based carriers of the pharmaceutical composition comprise at least one or more lipids selected from at least one aggregation-reducing lipid, at least one cationic lipid, at least one neutral lipid or phospholipid, or at least one steroid or steroid analog, or any combinations thereof.

In preferred embodiments, the lipid-based carriers of the pharmaceutical composition comprise (i) an aggregation-reducing lipid, (ii) a cationic lipid or ionizable lipid, and (iii) a neutral lipid/phospholipid or a steroid/steroid analog.

In particularly preferred embodiments, the lipid-based carriers of the pharmaceutical composition comprise an (i) aggregation-reducing lipid, (ii) a cationic lipid or ionizable lipid, (iii) a neutral lipid or phospholipid, (iv) and a steroid or steroid analog.

### LNP

In preferred embodiments, the nucleic acid sequence is complexed or associated with one or more lipids, thereby forming liposomes, lipid nanoparticles (LNP), lipoplexes, and/or nanoliposomes.

In most preferred embodiments, the pharmaceutical composition comprising the nucleic acid sequence which is complexed with one or more lipids thereby forming lipid nanoparticles (LNP).

The liposomes, lipid nanoparticles (LNPs), lipoplexes, and/or nanoliposomes-incorporated nucleic acid (e.g. RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the nucleic acid sequence is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA from an environment which may contain enzymes or chemicals or conditions that degrade nucleic acid and/or systems or receptors that cause the rapid excretion of the nucleic acid. Moreover, incorporating nucleic acid, preferably RNA into liposomes, lipid nanoparticles (LNPs), lipoplexes, and/or nanoliposomes may promote the uptake of the nucleic acid, and hence, may enhance the therapeutic effect of the nucleic acid.

In this context, the terms “complexed” or “associated” refer to the essentially stable combination of nucleic acid with one or more lipids into larger complexes or assemblies without covalent binding.

In a specifically preferred embodiment, the nucleic acid sequence is complexed with one or more lipids thereby forming lipid nanoparticles (LNP).

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphiphilic polymers and surfactants (e.g., polymerosomes, niosomes, etc.). In the context of the present invention, an LNP typically serves to transport the at least one nucleic acid, preferably the at least one nucleic acid sequence to a target tissue.

LNPs typically comprise a cationic lipid and one or more excipients selected from neutral lipids, charged lipids, steroids and polymer conjugated lipids (e.g. PEGylated lipid). The coding nucleic acid sequence may be encapsulated in the lipid portion of the LNP or an aqueous space enveloped by some or the entire lipid portion of the LNP. The coding RNA or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5,1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7,13-tris(3-oxo-3-(undecylamino)propyl)-NI,N 16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US2015/0140070).

In embodiments, the cationic lipid may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US2010/0324120).

In embodiments, the cationic lipid may an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531, hereby incorporated by reference.

In another embodiment, suitable lipids can also be the compounds as disclosed in WO2015/074085 (i.e. ATX-001 to ATX-032 or the compounds as specified in claims 1-26), U.S. Appl. Nos. 61/905,724 and 15/614,499 or U.S. Pat. Nos. 9,593,077 and 9,567,296 hereby incorporated by reference in their entirety.

In other embodiments, suitable cationic lipids can also be the compounds as disclosed in WO2017/117530 (i.e. lipids 13, 14, 15, 16, 17, 18, 19, 20, or the compounds as specified in the claims), hereby incorporated by reference in its entirety.

In other embodiments, suitable cationic lipids may be selected from published PCT patent application WO2017/117530 (i.e. lipids 13, 14, 15, 16, 17, 18, 19, 20, or the compounds as specified in the claims), the specific disclosure hereby incorporated by reference.

In preferred embodiments, ionizable or cationic lipids may also be selected from the lipids disclosed in WO2018/078053 (i.e. lipids derived from formula I, II, and III of WO2018/078053, or lipids as specified in claims 1 to 12 of WO2018/078053), the disclosure of WO2018/078053 hereby incorporated by reference in its entirety.

In that context, lipids disclosed in Table 7 of WO2018/078053 (e.g. lipids derived from formula I-1 to I-41) and lipids disclosed in Table 8 of WO2018/078053 (e.g. lipids derived from formula II-1 to II-36) may be suitably used in the context of the invention. Accordingly, formula I-1 to formula I-41 and formula II-1 to formula II-36 of WO2018/078053, and the specific disclosure relating thereto, are herewith incorporated by reference.

In preferred embodiments, cationic lipids may be derived from formula III of published PCT patent application WO2018/078053. Accordingly, formula III of WO2018/078053, and the specific disclosure relating thereto, are herewith incorporated by reference.

Accordingly, the lipid-based carriers (e.g. LNPs) of the pharmaceutical composition may comprise at least one cationic lipid according to formula (III) or derived from formula (III):

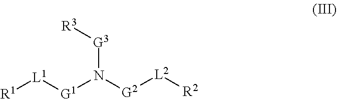

Formula (III) is further defined in that:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa—, —NRaC(═O)NRa—, —OC(═O)NRa—or —NRaC(═O)O— or a direct bond;
G1 and G2 are each independently unsubstituted C1-C12 alkylene or C1-C12 alkenylene;
G3 is C1-C24 alkylene, C1-C24 alkenylene, C3-C8 cycloalkylene, C3-C8 cycloalkenylene;
Ra is H or C1-C12 alkyl;
R1 and R2 are each independently C6-C24 alkyl or C6-C24 alkenyl;
R3 is H, OR5, CN, C(═O)OR4, OC(═O)R4 or —NR5C(═O)R4;
R4 is C1-C12 alkyl;
R5 is H or C1-C6 alkyl; and
x is 0, 1 or 2.

In particularly preferred embodiments, the nucleic acid sequence as defined herein is complexed with one or more lipids thereby forming LNPs, wherein the cationic lipid of the LNP is selected from structures III-1 to III-36 of Table 9 of published PCT patent application WO2018/078053. Accordingly, formula III-1 to III-36 of WO2018/078053, and the specific disclosure relating thereto, are herewith incorporated by reference.

In particularly preferred embodiment, the nucleic acid sequence as defined herein is complexed with one or more lipids thereby forming LNPs, wherein the LNP comprises the following cationic lipid:

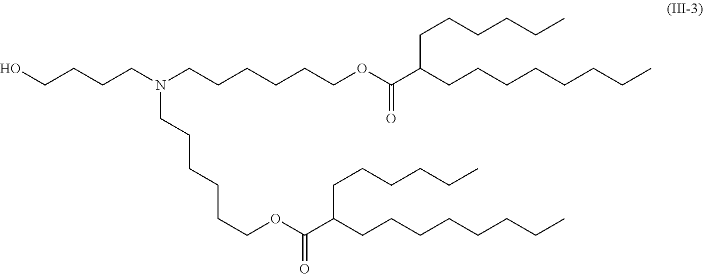 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, more preferably cationic lipid compound III-3, is present in the LNP in an amount from about 30 to about 95 mol %, relative to the total lipid content of the LNP. If more than one cationic lipid is incorporated within the LNP, such percentages apply to the combined cationic lipids.

In a preferred embodiment the at least one cationic lipid is a lipid selected or derived from ALC-0315 (lipid of formula III), SM-102, SS-33/4PE-15, HEXA-CSDE-PipSS, or compound C26, preferably wherein the at least one cationic lipid is ALC-0315.

In embodiments, the cationic lipid is present in the LNP in an amount from about 30 to about 70 mol %. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mol %, respectively. In embodiments, the cationic lipid is present in the LNP in an amount from about 47 to about 48 mol %, such as about 47.0, 47.1, 47.2, 47.3, 47.4, 47.5, 47.6, 47.7, 47.8, 47.9, 50.0 mol %, respectively, wherein 47.7 mol % are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to coding nucleic acid sequence is from about 3 to about 15, such as from about 5 to about 13 or from about 7 to about 11.

Other suitable (cationic or ionizable) lipids are disclosed in published patent applications WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, and U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US20130178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US20130178541, US2013/0225836 and US2014/0039032 and WO2017/112865 specifically relating to (cationic) lipids suitable for LNPs are incorporated herewith by reference.

In some embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the nucleic acid, which is used as cargo. The N/P ratio may be calculated on the basis that, for example, 1 μg RNA typically contains about 3nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

### PEG

In a preferred embodiment, the lipid nanoparticles (LNP) comprise a PEGylated lipid.

In vivo characteristics and behavior of LNPs can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(w-methoxy(polyethoxy)ethyl)carbamate.

In preferred embodiments, the PEGylated lipid is preferably derived from formula (IV) of published PCT patent application WO2018/078053. Accordingly, PEGylated lipids derived from formula (IV) of published PCT patent application WO2018/078053, and the respective disclosure relating thereto, are herewith incorporated by reference.

In a particularly preferred embodiments, the at least one coding nucleic acid sequence of the composition is complexed with one or more lipids thereby forming LNPs, wherein the LNP comprises a PEGylated lipid, wherein the PEG lipid is preferably derived from formula (IVa) of published PCT patent application WO2018/078053. Accordingly, PEGylated lipid derived from formula (IVa) of published PCT patent application WO2018/078053, and the respective disclosure relating thereto, is herewith incorporated by reference.

In a particularly preferred embodiment, the at least one nucleic acid, preferably the at least one RNA is complexed with one or more lipids thereby forming lipid nanoparticles (LNP), wherein the LNP comprises a PEGylated lipid/PEG lipid. Preferably, said PEG lipid is of formula (IVa):

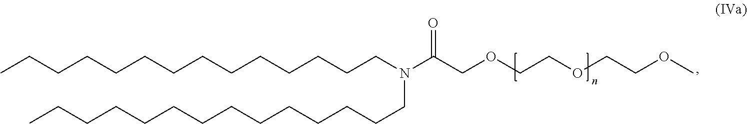

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49.

Further examples of PEG-lipids suitable in that context are provided in US2015/0376115 and WO2015/199952, each of which is incorporated by reference in its entirety.

In some embodiments, LNPs include less than about 3, 2, or 1 mol % of PEG or PEG-modified lipid, based on the total moles of lipid in the LNP. In further embodiments, LNPs comprise from about 0.1% to about 20% of the PEG-modified lipid on a molar basis, e.g. about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In one embodiment, the LNP comprises
(i) at least one cationic lipid;
(ii) at least one neutral lipid;
(iii) at least one steroid or steroid analogue; and
(iv) at least one a PEG-lipid.

In another embodiment, the LNP comprises
(i) at least one cationic lipid;
(ii) at least one neutral lipid;
(iii) at least one steroid or steroid analogue; and
(iv) at least one polymer-conjugated lipid, preferably a PEG-lipid, wherein (i) to (iv) are in a molar ratio of about 20-60% cationic lipid, 5-25% neutral lipid, 25-55% sterol, and 0.5-15% polymer-conjugated lipid, preferably a PEG-lipid.

In preferred embodiments, the LNP comprises one or more additional lipids, which stabilize the formation of particles during their formation or during the manufacturing process (e.g. neutral lipid and/or one or more steroid or steroid analogue).

Suitable stabilizing lipids include neutral lipids and anionic lipids. The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

In embodiments, the LNP comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In a preferred embodiment the at least one neutral lipid is selected or derived from DSPC, DHPC, or DphyPE, preferably wherein the at least one neutral lipid is DSPC.

In other embodiments of the second aspect, the LNP comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments, the neutral lipid is 1,2-distearoyl-sn-glycero-3-phosphocholine (DSPC). The molar ratio of the cationic lipid to DSPC may be in the range from about 2:1 to about 8:1.

In a preferred embodiment the at least one steroid or steroid analog selected or derived from cholesterol, cholesteryl hemisuccinate (CHEMS), preferably cholesterol.

In preferred embodiments, the steroid is cholesterol. The molar ratio of the cationic lipid to cholesterol may be in the range from about 2:1 to about 1:1. In some embodiments, the cholesterol may be PEGylated.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

Preferably, lipid nanoparticles (LNPs) comprise: (a) the at least one nucleic acid, preferably the at least one RNA of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20: 10,40:30:25:5 or 40:32:20:8, 40:32:25:3 or 40:33:25:2, respectively.

In some embodiments, the LNPs comprise a lipid of formula (III), the at least one nucleic acid, preferably the at least one RNA as defined herein, a neutral lipid, a steroid and a PEGylated lipid. In preferred embodiments, the lipid of formula (III) is lipid compound 111-3, the neutral lipid is DSPC, the steroid is cholesterol, and the PEGylated lipid is the compound of formula (IVa).

In a preferred embodiment the polymer conjugated lipid is a PEG-conjugated lipid preferably selected or derived from DMG-PEG 2000, C10-PEG2K, Cer8-PEG2K, or ALC-0159 (lipid of formula IVa), preferably wherein the polymer conjugated lipid is ALC-0159.

In a preferred embodiment of the second aspect, the LNP consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid: 5-25% neutral lipid: 25-55% sterol; 0.5-15% PEG-lipid.

In one embodiment the polymer conjugated lipid is not a PEG-conjugated lipid.

In some embodiments, the LNP of the pharmaceutical composition comprises (i) at least one cationic lipid; (ii) at least one neutral lipid; (iii) at least one steroid or steroid analogue; and (iv) at least one a PEG-lipid wherein (i) to (iv) are in a molar ratio of about 20-60% cationic lipid, 5-25% neutral lipid, 25-55% sterol, and 0.5-15% PEG-lipid.

Most preferably, the LNP comprises
(i) at least one cationic lipid, preferably a lipid of formula (III), more preferably lipid III-3;
(ii) at least one neutral lipid, preferably 1,2-distearoyl-sn-glycero-3-phosphocholine (DSPC);
(iii) at least one steroid or steroid analog, preferably cholesterol; and
(iv) at least one aggregation reducing lipid, preferably a PEG-conjugated lipid ALC-0159 derived from formula (IVa); and
   wherein (i) to (iv) are in a molar ratio of about 47.4% cationic lipid, 10% neutral lipid, 40.9% steroid or steroid analog, and 1.7% aggregation reducing lipid.

In particularly preferred embodiments, the LNP comprises (i) to (iv) in a molar ratio of about 20-60% cationic lipid: 5-25% neutral lipid: 25-55% steroid or steroid analogue I; 0.5-15% aggregation reducing lipid.

In one preferred embodiment, the lipid nanoparticle comprises: a cationic lipid with formula (III) and/or PEG lipid with formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the LNPs have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol and PEG-lipid (preferably PEG-lipid of formula (IVa) with n=49); solubilized in ethanol).

The total amount of nucleic acid in the lipid nanoparticles may vary and is defined depending on the e.g. nucleic acid to total lipid w/w ratio. In one embodiment of the invention the nucleic acid, in particular the RNA to total lipid ratio is less than 0.06 w/w, preferably between 0.03 w/w and 0.04 w/w.

In some embodiments, the lipid nanoparticles (LNPs), which are composed of only three lipid components, namely imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K).

In one embodiment, the lipid nanoparticle of the composition comprises a cationic lipid, a steroid; a neutral lipid; and a polymer conjugated lipid, preferably a pegylated lipid. Preferably, the polymer conjugated lipid is a pegylated lipid or PEG-lipid. In a specific embodiment, lipid nanoparticles comprise a cationic lipid resembled by the cationic lipid COATSOME© SS-EC (former name: SS-33/4PE-15; NOF Corporation, Tokyo, Japan), in accordance with the following structure:

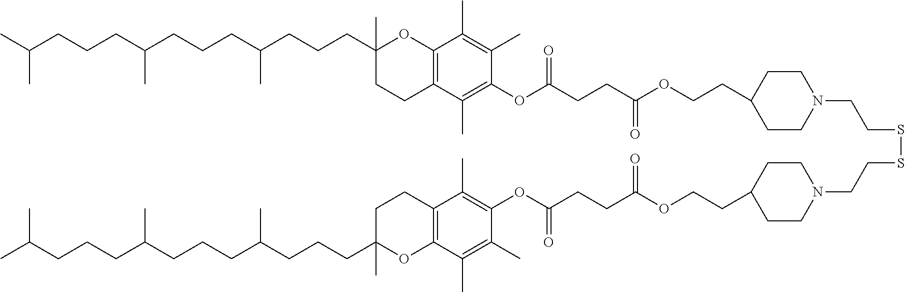 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

Furthermore, in a specific embodiment, the GN01 lipid nanoparticles comprise a neutral lipid being resembled by the structure 1,2-diphytanoyl-sn-glycero-3-phosphoethanolamine (DPhyPE):

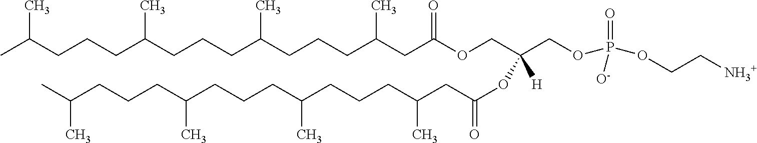 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

Furthermore, in a specific embodiment, the GN01 lipid nanoparticles comprise a polymer conjugated lipid, preferably a pegylated lipid, being 1,2-dimyristoyl-rac-glycero-3-methoxypolyethylene glycol 2000 (DMG-PEG 2000) having the following structure:

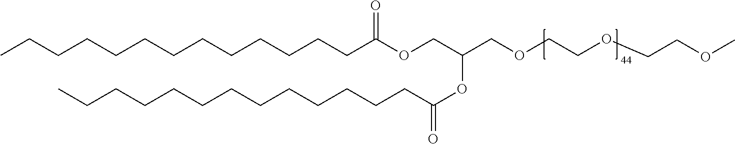

As used in the art, “DMG-PEG 2000” is considered a mixture of 1,2-DMG PEG2000 and 1,3-DMG PEG2000 in ˜97:3 ratio.

Accordingly, GN01 lipid nanoparticles (GN01-LNPs) according to one of the preferred embodiments comprise a SS-EC cationic lipid, neutral lipid DPhyPE, cholesterol, and the polymer conjugated lipid (pegylated lipid) 1,2-dimyristoyl-rac-glycero-3-methoxypolyethylene glycol (PEG-DMG).

In a preferred embodiment, the GN01 LNPs comprise:
(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;
each amount being relative to the total molar amount of all lipidic excipients of the GN01 lipid nanoparticles.

In a further preferred embodiment, the GN01 lipid nanoparticles as described herein comprises 59 mol % cationic lipid, 10 mol % neutral lipid, 29.3 mol % steroid and 1.7 mol % polymer conjugated lipid, preferably pegylated lipid. In a most preferred embodiment, the GN01 lipid nanoparticles as described herein comprise 59 mol % cationic lipid SS-EC, 10 mol % DPhyPE, 29.3 mol % cholesterol and 1.7 mol % DMG-PEG 2000.

The amount of the cationic lipid relative to that of the nucleic acid in the GN01 lipid nanoparticle may also be expressed as a weight ratio (abbreviated f.e. “m/m”). For example, the GN01 lipid nanoparticles comprise the at least one nucleic acid, preferably the at least one RNA at an amount such as to achieve a lipid to RNA weight ratio in the range of about 20 to about 60, or about 10 to about 50. In other embodiments, the ratio of cationic lipid to nucleic acid or RNA is from about 3 to about 15, such as from about 5 to about 13, from about 4 to about 8 or from about 7 to about 11. In a very preferred embodiment of the present invention, the total lipid/RNA mass ratio is about 40 or 40, i.e. about 40 or 40 times mass excess to ensure RNA encapsulation. Another preferred RNA/lipid ratio is between about 1 and about 10, about 2 and about 5, about 2 and about 4, or preferably about 3. Further, the amount of the cationic lipid may be selected taking the amount of the nucleic acid cargo such as the nucleic acid sequence into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the GN01 lipid nanoparticles or of the composition in the range from about 10 to about 20. In a further very preferred embodiment, the N/P is 14 (i.e. 14 times mol excess of positive charge to ensure nucleic acid encapsulation). In a preferred embodiment, GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. A further inventive advantage connected with the use of DPhyPE is the high capacity for fusogenicity due to its bulky tails, whereby it is able to fuse at a high level with endosomal lipids. For “GN01”, N/P (lipid to nucleic acid, e.g. RNA mol ratio) preferably is 14 and total lipid/RNA mass ratio preferably is 40 (m/m).

In other embodiments, the at least one nucleic acid sequence, is complexed with one or more lipids thereby forming lipid nanoparticles (LNP), wherein the LNP comprises
I at least one cationic lipid;
Ii at least one neutral lipid;
Iii at least one steroid or steroid analogue; and
liii at least one PEG-lipid as defined herein,
   wherein the cationic lipid is DLin-KC2-DMA (50 mol %) or DLin-MC3-DMA (50 mol %), the neutral lipid is DSPC (10 mol %), the PEG lipid is PEG-DOMG (1.5 mol %) and the structural lipid is cholesterol (38.5 mol %).

In other embodiments, the at least one nucleic acid (e.g. DNA or RNA), preferably the at least one nucleic acid sequence is complexed with one or more lipids thereby forming lipid nanoparticles (LNP), wherein the LNP comprises SS15/Chol/DOPE (or DOPC)/DSG-5000 at mol % 50/38.5/10/1.5.

In other embodiments, the nucleic acid of the invention may be formulated in liposomes, e.g. in liposomes as described in WO2019/222424, WO2019/226925, WO2019/232095, WO2019/232097, or WO2019/232208, the disclosure of WO2019/222424, WO2019/226925, WO2019/232095, WO2019/232097, or WO2019/232208 relating to liposomes or lipid-based carrier molecules herewith incorporated by reference.

In most preferred embodiment the lipid nanoparticles (LNP) additionally comprise a PEGylated lipid.

In one embodiment the LNP comprises of
(i) at least one cationic lipid;
(ii) at least one neutral lipid;
(iii) at least one steroid or steroid analogue; and
(iv) at least one a PEG-lipid, wherein (i) to (iv) are in a molar ratio of about 20-60% cationic lipid, 5-25% neutral lipid, 25-55% sterol, and 0.5-15% PEG-lipid.

In various embodiments, LNPs that suitably encapsulates the at least one nucleic acid of the invention have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

The polydispersity index (PDI) of the nanoparticles is typically in the range of 0.1 to 0.5. In a particular embodiment, a PDI is below 0.2. Typically, the PDI is determined by dynamic light scattering.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In embodiments where more than one or a plurality, e.g. 2, 3, 4, 5, 6, 7, 8, 9, 10, 11, 12, 13, 14, 15 of nucleic acid species of the invention are comprised in the composition, said more than one or said plurality e.g. 2, 3, 4, 5, 6, 7, 8, 9, 10, 11, 12, 13, 14, 15 of nucleic acid species of the invention may be complexed within one or more lipids thereby forming LNPs comprising more than one or a plurality, e.g. 2, 3, 4, 5, 6, 7, 8, 9, 10, 11, 12, 13, 14, 15 of different nucleic acid species.

In embodiments, the LNPs described herein may be lyophilized in order to improve storage stability of the formulation and/or the nucleic acid sequence. In embodiments, the LNPs described herein may be spray dried in order to improve storage stability of the formulation and/or the nucleic acid. Lyoprotectants for lyophilization and or spray drying may be selected from trehalose, sucrose, mannose, dextran and inulin. A preferred lyoprotectant is sucrose, optionally comprising a further lyoprotectant. A further preferred lyoprotectant is trehalose, optionally comprising a further lyoprotectant.

Accordingly, the composition, e.g. the composition comprising LNPs is lyophilized (e.g. according to WO2016/165831 or WO2011/069586) to yield a temperature stable dried nucleic acid (powder) composition as defined herein (e.g. RNA or DNA). The composition, e.g. the composition comprising LNPs may also be dried using spray-drying or spray-freeze drying (e.g. according to WO2016/184575 or WO2016/184576) to yield a temperature stable composition (powder) as defined herein.

Accordingly, in preferred embodiments, the composition is a dried composition.

The term “dried composition” as used herein has to be understood as composition that has been lyophilized, or spray-dried, or spray-freeze dried as defined above to obtain a temperature stable dried composition (powder) e.g. comprising LNP complexed nucleic acid sequence (as defined above).

In some aspects, the nucleic acid sequence species of the pharmaceutical composition may encode a different therapeutic peptide or protein as defined.

The term “nucleic acid sequence species” as used herein is not intended to refer to only one single molecule. The term “nucleic acid sequence species” has to be understood as an ensemble of essentially identical RNA molecules, wherein each of the RNA molecules of the RNA ensemble, in other words each of the molecules of the RNA species, encodes the same therapeutic protein (in embodiments, where the nucleic acid sequence is a coding RNA, having essentially the same nucleic acid sequence. However, the RNA molecules of the nucleic acid sequence ensemble may differ in length or quality, which may be caused by the enzymatic or chemical manufacturing process.

In some embodiments, the pharmaceutical composition comprises more than one or a plurality of different nucleic acid sequence species wherein the more than one or a plurality of different nucleic acid sequence species is selected from coding RNA species each encoding a different protein.

In other embodiments, the pharmaceutical composition comprises more than one or a plurality of different nucleic acid sequence species of the first component, wherein at least one of the more than one or a plurality of different nucleic acid sequence species is selected from a coding RNA species (e.g., an mRNA encoding a CRISPR associated endonuclease), and at least one is selected from a non-coding RNA species (e.g., a guide RNA).

In preferred embodiments, the pharmaceutical composition comprises the nucleic acid sequence, preferably an mRNA, wherein said nucleic acid sequence is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof. Complexation/association (“formulation”) to carriers as defined herein facilitates the uptake of the nucleic acid sequence into cells.

### Lipidoid

In some embodiments the pharmaceutical composition may comprise least one lipid or lipidoid as described in published PCT applications WO2017/212008, WO2017/212006, WO2017/212007, and WO2017/212009, the disclosures of WO2017/212008, WO2017/212006, WO2017/212007, and WO2017/212009 herewith incorporated by reference.

In particularly preferred embodiments, the polymeric carrier (of the nucleic acid sequence) is a peptide polymer, preferably a polyethylene glycol/peptide polymer as defined above, and a lipid, preferably a lipidoid.

A lipidoid (or lipidoit) is a lipid-like compound, i.e. an amphiphilic compound with lipid-like physical properties. The lipidoid is preferably a compound that comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention, the term lipid is considered to also encompass lipidoids. In some embodiments of the inventions, the lipidoid may comprise a PEG moiety.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In a preferred embodiment, the lipidoid component may be any one selected from the lipidoids of the lipidoids provided in the table of page 50-54 of published PCT patent application WO2017/212009, the specific lipidoids provided in said table, and the specific disclosure relating thereto herewith incorporated by reference.

In preferred embodiments, the lipidoid component may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat., 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017/212009 (pages 50-54)). Particularly preferred are 3-C12-OH or 3-C12-OH-cat.

In preferred embodiments, the polyethylene glycol/peptide polymer comprising a lipidoid as specified above (e.g. 3-C12-OH or 3-C12-OH-cat), is used to complex the at least one nucleic acid to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017/212009, in particular claims 1 to 10 of WO2017/212009, and the specific disclosure relating thereto is herewith incorporated by reference.

Further suitable lipidoids may be derived from published PCT patent application WO2010/053572. In particular, lipidoids derivable from claims 1 to 297 of published PCT patent application WO2010/053572 may be used in the context of the invention, e.g. incorporated into the peptide polymer as described herein, or e.g. incorporated into the lipid nanoparticle (as described below). Accordingly, claims 1 to 297 of published PCT patent application WO2010/053572, and the specific disclosure relating thereto, is herewith incorporated by reference.

In particularly preferred embodiments, the at least one nucleic acid, preferably the nucleic acid sequence is complexed with one or more lipids thereby forming lipid nanoparticles (LNP), wherein the LNP comprises
(i) at least one cationic lipid as defined herein, preferably a lipid of formula (III), more preferably lipid III-3;
(ii) at least one neutral lipid as defined herein, preferably 1,2-distearoyl-sn-glycero-3-phosphocholine (DSPC);
(iii) at least one steroid or steroid analogue as defined herein, preferably cholesterol; and
   at least one PEG-lipid as defined herein, e.g. PEG-DMG or PEG-cDMA, preferably a PEGylated lipid that is or is derived from formula (IVa).

In various embodiments, the pharmaceutical composition comprises Ringer or Ringer-Lactate solution.

Accordingly, the pharmaceutical composition may comprise and/or is administered in Ringer or Ringer-Lactate solution as described in WO2006/122828.

In embodiments, pharmaceutical composition may be provided in lyophilized or dried form (using e.g. lyophilisation or drying methods as described in WO2016/165831, WO2011/069586, WO2016/184575 or WO2016/184576).

Preferably, the lyophilized or dried pharmaceutical composition is reconstituted in a suitable buffer, advantageously based on an aqueous carrier, prior to administration, e.g. Ringer- or Ringer-Lactate solution or a phosphate buffer solution.

### Administration

In preferred embodiments, the pharmaceutical composition is administered to a cell or subject.

The term “subject” or “cell” as used herein generally includes humans and non-human animals or cells and preferably mammals, including chimeric and transgenic animals and disease models. Subjects to which administration of the compositions, preferably the pharmaceutical composition, is contemplated include, but are not limited to, humans and/or other primates; mammals, including commercially relevant mammals such as cattle, pigs, horses, sheep, cats, dogs; and/or birds, including commercially relevant birds such as poultry, chickens, ducks, geese, and/or turkeys. Preferably, the term “subject” refers to a non-human primate or a human, most preferably a human.

In most preferred embodiments, the subject is a human subject.

In the context of this invention the administration of the pharmaceutical composition to a cell or subject results in translation of the nucleic acid sequence into a (functional) peptide or protein.

Suitably, the administration of the nucleic acid sequence may be advantageous for various medical applications of the pharmaceutical composition. In particular, the nucleic acid sequence may be used for chronic administration or may e.g. enhance or improve the therapeutic effect of a in the nucleic acid sequence encoding an antigen (e.g. viral antigen, tumor antigen). Accordingly, the nucleic acid sequence or pharmaceutical composition of the nucleic acid sequence of the invention leads to an increased efficiency of a therapeutic RNA (e.g. upon administration to a cell or a subject).

In some embodiments, detectable levels of the therapeutic protein are produced in the serum of the subject at about 1 to about 72 hours post administration.

Moreover, in that context, the method of this invention allows the reduction of reactogenicity of a coding therapeutic nucleic acid sequence (comprising a cds encoding e.g. an antigen). The term reactogenicity refers to the property of e.g. a vaccine of being able to produce adverse reactions, especially excessive immunological responses and associated signs and symptoms-fever, sore arm at injection site, etc. Other manifestations of reactogenicity typically comprise bruising, redness, induration, and swelling.

In various embodiments, the administration of the pharmaceutical composition is systemically or locally.

In preferred embodiments, the administration of the pharmaceutical composition is transdermally, intradermally, intravenously, intramuscularly, intraaterially, intranasally, intraocularily, intrapulmonally, intracranially, intralesionally, intratumorally, intravitreally, subcutaneously or via sublingual, preferably intramuscularly, intranodally, intradermally, intratumorally or intravenously, preferably intramuscularly, intradermally, intravenously or intratumorally.

In preferred embodiments, the pharmaceutical composition is intramuscular administered. In other preferred embodiments the pharmaceutical composition an intravenously administered.

In some embodiments, the administration of the pharmaceutical composition is orally, parenterally, by inhalation spray, topically, rectally, nasally, buccally, vaginally or via an implanted reservoir.

The term parenteral, as used herein, includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intratumoral.

In other preferred embodiments, the administration of the pharmaceutical composition more than once, for example once or once more than once a day, once or more than once a week, once or more than once a month.

Advantageously, the pharmaceutical composition is suitable for repetitive administration, e.g. for chronic administration.

In particularly preferred embodiments, administration of the pharmaceutical composition is performed intravenously or intratumorally.

In other particular embodiments, the pharmaceutical composition is administered intravenously as a chronic treatment (e.g. more than once, for example once or more than once a day, once or more than once a week, once or more than once a month.

In preferred embodiments, the pharmaceutical composition comprises at least one nucleic acid sequence comprising at least one miRNA binding site sequence for reducing or preventing protein expression in the liver, preferably wherein the nucleic acid sequence is characterized by any one of the features as defined in the disclosure.

Suitably in that context, the nucleic acid sequence of the composition comprises at least one miRNA binding site sequence (e.g. a first and/or a second miRNA binding site sequence) for reducing liver expression as defined in the context of the first aspect and lacks miRNA binding site sequences for reducing expression in immune cells.

Preferably in that context, the nucleic acid sequence of the composition is formulated in lipid-based carriers, preferably in LNPs as defined herein. Preferably in that context, the nucleic acid sequence is an RNA. Preferably in that context, the therapeutic peptide or protein is and antigen (tumor antigen or antigen of a pathogen).

In embodiments in that context, upon administration of the composition to a cell or subject, the expression of the encoded peptide or protein is reduced in the liver by at least 10%, 20%, 30%, 40%, 50%, 60%, 70%, 80% or 90% compared to a reference composition comprising a nucleic acid sequence lacking the respective miRNA binding site sequence.

In other embodiments in that context, upon administration of the composition to a cell or subject, the encoded peptide or protein is expressed in non-liver cells, preferably in immune cells or muscle cells.

In preferred embodiments in that context, upon administration of the composition to a cell or subject, 80%, 85%, 90%, 95%, 96%, 97%, 98%, 99% of the expressed peptide or protein is produced in non-liver cells, preferably in immune cells or muscle cells.

In other preferred embodiments, the pharmaceutical composition, wherein the pharmaceutical composition comprises at least one nucleic acid sequence comprising at least one miRNA binding site sequence for reducing or preventing protein expression in immune cells, preferably wherein the nucleic acid sequence is characterized by any one of the features as defined in the disclosure.

Suitably in that context, the nucleic acid sequence of the composition comprises at least one miRNA binding site sequence (e.g. a first and/or a second miRNA binding site sequence) for reducing expression in immune cells as defined in the context of the first aspect and lacks miRNA binding site sequences for reducing expression in liver cells.

Preferably in that context, the nucleic acid sequence of the composition is formulated in lipid-based carriers, preferably in LNPs as defined herein. Preferably in that context, the nucleic acid sequence is an RNA. Preferably in that context, the therapeutic peptide or protein is a peptide or protein where liver expression is desired.

In embodiments in that context, upon administration of the composition to a cell or subject the expression of the encoded peptide or protein is reduced in immune cells by at least 10%, 20%, 30%, 40%, 50%, 60%, 70%, 80% or 90% compared to a reference composition comprising a nucleic acid sequence lacking the respective miRNA binding site sequence.

In other embodiments in that context, upon administration of the composition to a cell or subject, the encoded peptide or protein is expressed in non-immune cells, preferably in liver cells.

In preferred embodiments in that context, upon administration of the composition to a cell or subject, 80%, 85%, 90%, 95%, 96%, 97%, 98%, 99% of the expressed peptide or protein is produced in non-immune cells, preferably in liver cells.

In other preferred embodiments, the pharmaceutical composition, wherein the pharmaceutical composition comprises at least one nucleic acid sequence comprising at least one miRNA binding site sequence for reducing or preventing protein expression in immune cells and in liver cells, preferably wherein the nucleic acid sequence is characterized by any one of the features as defined in the disclosure.

Suitably in that context, the nucleic acid sequence of the composition comprises at least one miRNA binding site sequence (e.g. a first and/or a second miRNA binding site sequence) for reducing expression in immune cells as defined in the context of the first aspect and at least one miRNA binding site sequence (e.g. a first and/or a second miRNA binding site sequence) for reducing expression in liver cells as defined in the context of the first aspect.

Preferably in that context, the nucleic acid sequence of the composition is formulated in lipid-based carriers, preferably in LNPs as defined herein. Preferably in that context, the nucleic acid sequence is an RNA. Preferably in that context, the therapeutic peptide or protein is a peptide or protein where liver expression and expression in immune cells is not desired.

In embodiments in that context, upon administration of the composition to a cell or subject the expression of the encoded peptide or protein is reduced in immune cells and liver cells by at least 10%, 20%, 30%, 40%, 50%, 60%, 70%, 80% or 90% compared to a reference composition comprising a nucleic acid sequence lacking the respective miRNA binding site sequence.

In preferred embodiments in that context, upon administration of the composition to a cell or subject, the encoded peptide or protein is expressed in non-immune cells and non-liver cells.

In other preferred embodiments in that context, upon administration of the composition to a cell or subject, 80%, 85%, 90%, 95%, 96%, 97%, 98%, 99% of the expressed peptide or protein is produced in non-immune cells and non-liver cells.

In various embodiments, the pharmaceutical composition is formulated as a vaccine.

Accordingly, in those embodiments, the vaccine comprises the nucleic acid sequence according to this invention.

In a further aspect, the present invention relates to a vaccine comprising the nucleic acid of the invention, preferably the RNA of the vaccine, is formulated in lipid-based carriers, preferably LNPs as defined in the disclosure.

Such a vaccine suitably comprises at leason one miRNA binding site sequence for reducing liver expression as defined herein, but does not comprise a miRNA binding site sequence for reducing expression in immune cells.

Suitable nucleic acid sequences comprising miRNA binding site sequences for preventing liver expression are provided in the context of the first aspect.

In preferred embodiments, the vaccine is against a pathogen, preferably against a virus.

In preferred embodiments, the vaccine comprises at least one nucleic acid sequence, preferably an RNA sequence, having the following features:
I) at least one 5′ UTR preferably selected or derived from a gene;
II) at least one first miRNA binding site sequence located in 5′ direction relative to the coding sequence, wherein the at least one first miRNA binding site sequence comprises one or more miRNA-122 and/or miRNA-148a binding sites.
III) optionally, at least one 3′ UTR preferably selected or derived from a gene;
IV) optionally, at least one second miRNA binding site sequence located in 3′ direction relative to the 3′ UTR, wherein the at least one second miRNA binding site sequence comprises one or more miRNA-122 binding sites and/or miRNA-192 binding sites and/or miRNA-194 binding sites.

In preferred embodiments, the nucleic acid sequence, preferably the RNA of the vaccine, is formulated in lipid-based carriers, preferably LNP as defined in the context of the second aspect.

In preferred embodiments, wherein upon administration of the vaccine to a cell or subject, 80%, 85%, 90%, 95%, 96%, 97%, 98%, 99% of the expressed peptide or protein is produced in muscle cells or immune cells and expression of the encoded peptide or protein is reduced in liver cells by at least 10%, 20%, 30%, 40%, 50%, 60%, 70%, 80% or 90% compared to a reference composition comprising a nucleic acid sequence lacking the respective miRNA binding site sequence.

In Other Preferred Embodiments the Vaccine is a Tumor Vaccine. Third Aspect: Kit of Parts

In a third aspect, the present invention provides a kit or kit of parts, preferably comprising the individual components of the nucleic acid sequence (e.g. as defined in the context of the first aspect) and/or comprising the pharmaceutical composition (e.g. as defined in the context of the second aspect), or the vaccine Notably, embodiments relating to the first and the second aspect of the invention or to the vaccine are likewise applicable to embodiments of the third aspect of the invention, and embodiments relating to the third aspect of the invention are likewise applicable to embodiments of the first and second aspect of the invention. In addition, the kit or kit of parts may comprise a liquid vehicle for solubilising, and/or technical instructions providing information on administration and dosage of the components.

In preferred embodiments, the kit or kit of parts comprising the nucleic acid sequence or pharmaceutical composition or vaccine, optionally comprising a liquid vehicle for solubilizing, and, optionally, technical instructions providing information on administration and/or dosage of the components.

In preferred embodiments, the kit or the kit of parts comprises:
(a) at least one composition or vaccine as defined in the context of the second aspect;
(b) optionally, a liquid vehicle for solubilizing, and optionally technical instructions providing information on administration and dosage of the components.

In most preferred embodiments, the kit or the kit of parts comprises:
(a) the nucleic acid sequence as defined herein, preferably an mRNA encoding a therapeutic peptide or protein, e.g. an antibody, an enzyme, an antigen, preferably
   wherein said mRNA does not comprise modified nucleotides, preferably wherein said mRNA does comprise a cap1 structure, preferably wherein said first component is formulated in a lipid nanoparticle or in a polyethylene glycol/peptide polymer.
(b) optionally, a liquid vehicle for solubilising (a) and/or (b), and optionally technical instructions providing information on administration and dosage of the components.

The technical instructions of said kit or kit of parts may comprise information about administration and dosage and patient groups. Such kits, preferably kits of parts, may be applied e.g. for any of the applications or medical uses mentioned herein.

Preferably, the individual components of the kit or kit of parts may be provided in lyophilised form. The kit may further contain as a part a vehicle (e.g. pharmaceutically acceptable buffer solution) for solubilising the nucleic acid sequence, and/or the pharmaceutical composition of the second aspect.

In preferred embodiments, the kit or kit of parts comprises Ringer- or Ringer lactate solution.

In preferred embodiments, the kit or kit of parts comprise an injection needle, a microneedle, an injection device, a catheter, an implant delivery device, or a micro cannula.

Any of the above kits may be used in applications or medical uses as defined in the context of the invention.

### Fourth Aspect: Medical Use

In a fourth aspect the present invention relates to the medical use of the nucleic acid sequence of the first aspect obtainable by the method of further aspects, the pharmaceutical composition of the second aspect, the vaccine, or the kit or kit of parts of the third aspect.

Notably, embodiments relating to the nucleic acid sequence of the first aspect by the method of further aspects, the pharmaceutical composition of the second aspect, the vaccine, or the kit or kit of parts of the third aspect may likewise be read on and be understood as suitable embodiments of medical uses of the invention.

Accordingly, the invention provides the nucleic acid sequence of the first aspect obtainable by the method of further aspects, the pharmaceutical composition of the second aspect, the vaccine, or the kit or kit of parts of the third aspect for use as a medicament or the kit or kit of parts as defined in the third aspect for use as a medicament.

In embodiments, the nucleic acid sequence of the first aspect obtainable by the method of further aspects, the pharmaceutical composition of the second aspect, the vaccine, or the kit or kit of parts of the third aspect may be used for human medical purposes and also for veterinary medical purposes, preferably for human medical purposes.

In embodiments, the nucleic acid sequence of the first aspect obtainable by the method of further aspects, the pharmaceutical composition of the second aspect, the vaccine, or the kit or kit of parts of the third aspect may be in particular used and suitable for human medical purposes, in particular for young infants, newborns, immunocompromised recipients, pregnant and breast-feeding women, and elderly people.

In various aspects the invention relates to the medical use of the nucleic acid sequence of the first aspect obtainable by the method of further aspects, the pharmaceutical composition of the second aspect, the vaccine, or the kit or kit of parts of the third aspect for use in the prevention or treatment of cancer, autoimmune diseases, infectious diseases, allergies or protein deficiency disorders. Hereby, the nucleic acid according to the invention can also be used a vaccine.

In a further aspect, the invention relates to the nucleic acid sequence, or a pharmaceutical composition, the vaccine, or the kit or kit of parts as defined herein, for use in treating or preventing a non-liver disease and/or a disease where a production of the target peptide or protein in the liver causes side effects.

In a further aspect, the invention relates to
the nucleic acid sequence, or a pharmaceutical composition, or the kit or kit of parts as defined herein, for use in treating or preventing a non-immune cell disease and/or a disease where a production of the target peptide or protein in immune cells causes side effects. In a not limiting example this pharmaceutical composition of the invention, of the kit or kit of parts as defined in the disclosure, could be used for the long term treatment with therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, or fragments, variants, or combinations of any of these.

In a further aspect, the invention relates to the nucleic acid sequence, or a pharmaceutical composition, or the kit or kit of parts as defined in the disclosure, for use in treating or preventing a non-immune cell and non-liver disease and/or a disease where a production of the target peptide or protein in immune cells and the liver causes side effects. In a not limiting example this pharmaceutical composition of the invention, of the kit or kit of parts as defined in the disclosure, could be used for the treatment of cancer with encoded cytokines or cytostatic/cytotoxic peptides.

In yet another aspect, the invention relates to the medical use of the nucleic acid sequence of the first aspect obtainable by the method of further aspects, the pharmaceutical composition of the second aspect, the vaccine, or the kit or kit of parts of the third aspect for use in the treatment or prophylaxis of a tumor disease, or of a disorder related to such tumor disease.

Accordingly, in said embodiments, the nucleic acid sequence may encode at least one tumor or cancer antigen and/or at least one therapeutic antibody (e.g. checkpoint inhibitor). In further embodiments the nucleic acid sequence may encode at least one cytostatic or cytotoxic polypeptide.

In yet another aspect, the invention relates to the medical use of the nucleic acid sequence of the first aspect obtainable by the method of further aspects, the pharmaceutical composition of the second aspect or the kit or kit of parts of the third aspect for use in the treatment or prophylaxis of a genetic disorder or condition.

Such a genetic disorder or condition may be a monogenetic disease, i.e. (hereditary) disease, or a genetic disease in general, diseases which have a genetic inherited background and which are typically caused by a defined gene defect and are inherited according to Mendel's laws.

Accordingly, in said embodiments, the nucleic acid sequence may encode a CRISPR-associated endonuclease or another protein or enzyme suitable for genetic engineering.

In yet another aspect, the invention relates to the medical use of the nucleic acid sequence of the first aspect obtainable by the method of further aspects, the pharmaceutical composition of the second aspect or the kit or kit of parts of the third aspect for use in the treatment or prophylaxis of a protein or enzyme deficiency or protein replacement.

Accordingly, in said embodiments, the nucleic acid sequence may encode at least one protein or enzyme. “Protein or enzyme deficiency” in that context has to be understood as a disease or deficiency where at least one protein is deficient, e.g. A1AT deficiency.

In yet another aspect, the invention relates to the medical use of the nucleic acid sequence of the first aspect obtainable by the method of further aspects, the pharmaceutical composition of the second aspect or the kit or kit of parts of the third aspect for use in the treatment or prophylaxis of autoimmune diseases, allergies or allergic diseases, cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, and ear diseases.

In yet another aspect, the invention relates to the medical use of the nucleic acid sequence of the first aspect obtainable by the method of further aspects, the pharmaceutical composition of the second aspect, the vaccine, or the kit or kit of parts of the third aspect for use in the treatment or prophylaxis of an infection, or of a disorder related to such an infection.

In the context of a use in the treatment or prophylaxis of an infection, the nucleic acid sequence of the first aspect obtainable by the method of further aspects, the pharmaceutical composition of the second aspect, the vaccine, or the kit or kit of parts of the third aspect may preferably be administered locally or systemically. In that context, administration may be by an intradermal, subcutaneous, intranasal, or intramuscular route. In embodiments, administration may be by conventional needle injection or needle-free jet injection. In preferred embodiments in that context, administration may be by an intramuscular needle injection.

### Method of Treatment

In another aspect, the present invention relates to a method of treating or preventing a disorder.

Notably, embodiments relating to the nucleic acid sequence of the first aspect obtainable by the method of further aspects, the pharmaceutical composition of the second aspect, the vaccine, or the kit or kit of parts of the third aspect may likewise be read on and be understood as suitable embodiments of methods of treatment and use as provided herein. Furthermore, specific features and embodiments relating to method of treatments as provided herein may also apply for medical uses of the invention.

Accordingly, the nucleic acid sequence of the first aspect obtainable by the method of further aspects, the pharmaceutical composition of the second aspect, the vaccine, or the kit or kit of parts of the third aspect may be used in the prevention or treatment of cancer, autoimmune diseases, infectious diseases, allergies or protein deficiency disorders.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the present invention relates to a method of treating or preventing a disorder, wherein the method comprises applying or administering to a subject in need thereof the nucleic acid sequence of the first aspect obtainable by the method of further aspects, the pharmaceutical composition of the second aspect, the vaccine, or the kit or kit of parts of the third aspect.

In other embodiments, the disorder is a tumor disease or a disorder related to such tumor disease, a protein or enzyme deficiency, or a genetic disorder or condition.

In preferred embodiments, the present invention relates to a method of treating or preventing a disorder as defined above, wherein the method comprises applying or administering to a subject in need thereof the nucleic acid sequence of the first aspect obtainable by the method of further aspects, the pharmaceutical composition of the second aspect, the vaccine, or the kit or kit of parts of the third aspect.

In particularly preferred embodiments, the subject in need is a mammalian subject, preferably a human subject, e.g. new-born human subject, pregnant human subject, immunocompromised human subject, and/or elderly human subject.

In particular, the method of treating or preventing a disorder may comprise the steps of:
a) providing the the nucleic acid sequence of the first aspect obtainable by the method of further aspects, the pharmaceutical composition of the second aspect, the vaccine, or the kit or kit of parts of the third aspect;
b) applying or administering said pharmaceutical composition, vaccine, or kit or kit of parts to a subject as a first dose;
c) optionally, applying or administering said pharmaceutical composition, vaccine, or kit or kit of parts to a subject as a second dose or a further dose, preferably at least 1, 2, 3, 4, 5, 6, 7, 8, 9, 10, 11, 12, months after the first dose.

In other embodiments the present invention relates to a chronic medical treatment of a disorder, wherein the method comprises applying or administering to a subject in need thereof the nucleic acid sequence of the first aspect obtainable by the method of further aspects, the pharmaceutical composition of the second aspect or the kit or kit of parts of the third aspect. The term “chronic medical treatment” relates to treatments that require the administration of the nucleic acid sequence, the pharmaceutical composition, or the kit or kit of parts more than once, for example once or more than once a day, once or more than once a week, once or more than once a month.

The method of treating or preventing a disorder comprises applying or administering to a subject in need thereof the nucleic acid sequence of the first aspect obtainable by the method of further aspects, the pharmaceutical composition of the second aspect or the kit or kit of parts of the third aspect, preferably wherein applying or administering is performed more than once, for example once or more than once a day, once or more than once a week, once or more than once a month.

Accordingly, the administration is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, intranodal, or intratumoral, preferably intramuscular, intradermal, intravenous, or intratumoral, most preferably intramuscular.

In preferred embodiments, the subject in need treated to prevent a disorder is a mammalian subject, preferably a human subject.

Preferably, a human subject is selected from e.g. newborn human subject, pregnant human subject, immunocompromised human subject, and/or elderly human subject.

In another aspect, the present invention relates to a method of promoting cell-type-specific expression induced by a nucleic acid sequence upon administration of said nucleic acid sequence to a cell or a subject.

### Method to Promote a Cell-Type Specific Expression

In a further aspect, the present invention relates to a method to promote a cell-type specific expression of a peptide or protein within a target organ or organs by using a nucleic acid sequence of the first aspect, the pharmaceutical composition of the second aspect, the vaccine, or the kit or kit of parts of the third aspect. In a preferred embodiment, the method to promote a cell-type specific expression of a peptide or protein within a target organ or organs uses the nucleic acid sequence of the invention, which is formulated with a cationic compound. Accordingly, the nucleic acid sequence is formulated with a cationic compound, which comprises one or more lipids suitable to form liposomes, lipid nanoparticles (LNP), lipoplexes, and/or nanoliposomes.

### Items

In the following, preferred embodiments of the present invention are provided as a numbered item list (item 1 to item 68).
1. A nucleic acid sequence comprising
   i) at least one 3′ UTR of a gene
   ii) at least one coding region encoding at least one peptide or protein of interest
   iii) at least one 5′ UTR of a gene
   iv) a miRNA binding site sequence wherein the miRNA binding site sequence is located within and/or immediately 3′ or 5′ of the 5′ UTR to allow a cell type specific expression from the nucleic acid sequence within the target organ or organs.
2. The nucleic acid sequence according to item 1, wherein the miRNA binding site sequence comprises at least one, two, three, or four miRNA binding sites.
3. The nucleic acid sequence according to item 1 or 2, wherein the at least one miRNA binding site sequence comprises at least two substantially similar miRNA binding sites.
4. The nucleic acid sequence according to item 1 or 2, wherein the at least one miRNA binding site sequence comprises at least two substantially different miRNA binding sites.
5. The nucleic acid sequence according to any of items 1 to 4, wherein the at least one miRNA binding site is substantially complementary to miRNA sequences selected from at least one or more of the group consisting of miRNA-122, miRNA-148a, miRNA-101, miRNA-192, miRNA-194.
6. The nucleic acid sequence according to item 5, wherein the miRNA binding site sequence preferably comprises one or more miRNA-122 and/or miRNA-148a binding sites.
7. The nucleic acid sequence according to item 5 or 6, wherein the miRNA binding site sequence comprises one or more miRNA-122 binding sites.
8. The nucleic acid sequence according to item 6 and 7, wherein the miRNA binding site sequence comprises at least two miRNA-122 binding sites.
9. The nucleic acid sequence according to any of the preceding items wherein the miRNA binding site sequence comprises a sequence selected from SEQ ID No 249, SEQ ID No 250, SEQ ID No 251, SEQ ID No 252, SEQ ID No 253, SEQ ID No 254, SEQ ID No 255, SEQ ID No 256, SEQ ID No 257 or SEQ ID No 258.
10. The nucleic acid sequence according to item 5 or 6, wherein the miRNA binding site sequence comprises at least two miRNA-148a binding sites.
11. The nucleic acid sequence according to any of the preceding items, wherein the miRNA binding site sequence is located immediately 5′ of the 5′ UTR.
12. The nucleic acid sequence according to any of the preceding items, wherein the at least one 3′ UTR optionally comprises within and/or immediately 3′ or 5′ of the 3′ UTR a second miRNA binding site sequence comprising at least one miRNA binding site.
13. The nucleic acid sequence according to item 12, wherein the second miRNA binding site sequence comprises at least one miRNA binding site substantially complementary to a miRNA sequence selected from at least one or more of the group consisting of miRNA-192, miRNA-122, miRNA-148a, miRNA-194 or miR-101.
14. The nucleic acid sequence according to item 12 or 13, wherein the second miRNA binding site sequence preferably comprises one or more miRNA-192 and/or miRNA-122 binding sites.
15. The nucleic acid sequence according to item 13 or 14, wherein the second miRNA binding sequence comprises at least two miR-192 binding sites.
16. The nucleic acid sequence according to item 12, wherein the second miRNA binding site sequence is located immediately 3′ of the 3′ UTR.
17. The nucleic acid sequence according any of the preceding items, wherein the cell type specific expression from the nucleic acid sequence within the target organ or organs is not selected from hepatocytes, hepatic stellate fat storing (ITO) cells, Kupffer cells or liver endothelial cells.
18. The nucleic acid sequence according to item 17, wherein the cell type specific expression from the nucleic acid sequence within the target organ or organs is not in hepatocytes.
19. The nucleic acid sequence according to any of the preceding items, wherein the target organ is not the liver.
20. The nucleic acid sequence according to any of the preceding items, wherein the cell type specific expression from the nucleic acid sequence within the target organ or organs is selected from tumor cells or immune cells.
21. The nucleic acid sequence according to any of the preceding items, wherein the nucleic acid comprises at least one modified nucleotide and/or at least one nucleotide analogue or nucleotide derivative.
22. The nucleic acid sequence according to item 21, wherein the at least one modified nucleotide and/or at least one nucleotide analogues is selected from a backbone modified nucleotide, a sugar modified nucleotide and/or a base modified nucleotide, or any combination thereof.
23. The nucleic acid sequence according to item 21 or 22, wherein the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, N-1-methylpseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, 2-aminoadenosine or 5-(isopentenylaminomethyl)-2′-O-methyluridine or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or O(6)-methylguanine.
24. The nucleic acid sequence according to items 21 to 23, wherein the nucleic acid comprises a cap.
25. The nucleic acid sequence according to item 24, wherein the cap is a cap0, cap1, cap2, a modified cap0 or a modified cap1, preferably a cap1.
26. The nucleic acid sequence according to any of the preceding items which comprises at least one coding region encoding at least one peptide or protein of interest wherein the at least one peptide or protein is a therapeutic peptide or protein or is derived from a therapeutic peptide or protein.
27. The nucleic acid sequence according to item 24, wherein the therapeutic peptide or protein is or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a cytokine, a structural protein, a cytoplasmic protein, a cytoskeletal protein, a viral antigen, a bacterial antigen, a protozoan antigen, an allergen, an autoimmune antigen, a tumor antigen, or fragments, variants, or combinations of any of these.
28. The nucleic acid sequence according to any of the preceding items, wherein the at least one coding region is a codon modified coding sequence, wherein the amino acid sequence encoded by the at least one codon modified coding sequence is preferably not being modified compared to the amino acid sequence encoded by the corresponding reference or original coding sequence.
29. The nucleic acid sequence according to item 28, wherein the at least one codon modified coding sequence is selected from a C increased coding sequence, a CAI increased coding sequence, a human codon usage adapted coding sequence, a G/C content modified coding sequence, or a G/C optimized coding sequence, or any combination thereof.
30. The nucleic acid sequence according to any of the preceding items, wherein the nucleic acid sequence comprises at least one poly(A) sequence, and/or at least one poly(C) sequence, and/or at least one histone stem-loop sequence/structure.
31. The nucleic acid sequence according to any of the preceding items, wherein the at least one 5′-UTR and/or 3′-UTR are heterologous UTRs.
32. The nucleic acid sequence according to item 31, wherein the at least one heterologous 3′-UTR comprises a nucleic acid sequence derived from a 3′-UTR of a gene selected from PSMB3, ALB7, alpha-globin, CASP1, COX6B1, GNAS, NDUFA1 and RPS9 or from a homolog, a fragment or a variant of any one of these genes.
33. The nucleic acid sequence according to item 31, wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.
34. The nucleic acid sequence according to any of the preceding items, wherein the nucleic acid is selected from DNA or RNA, preferably from plasmid DNA, viral DNA, template DNA, viral RNA, self-replicating RNA or replicon RNA, and most preferably from an mRNA.
35. The nucleic acid sequence according to any of the preceding items, wherein the expression of the encoded peptide or protein is reduced in the liver.
36. The nucleic acid sequence according to item 35, wherein the expression of the encoded peptide or protein in the liver is reduced by at least 10%, 20%, 30%, 40%, 50%, 60%, 70%, 80% or 90%.
37. The nucleic acid sequence according to any of the preceding items, wherein the expression of the encoded peptide or protein can be detected within tumor cells.
38. The nucleic acid sequence according to item 27, wherein the encoded peptide or protein is a cytokine.
39. The nucleic acid sequence according to item 38, wherein the cytokine is selected from interleukins, chemokines, interferons or lymphokines.
40. The nucleic acid sequence according to item 39, wherein the cytokine is selected from interleukins, preferably the interleukin-12 (IL-12).
41. The nucleic acid sequence according to any of the preceding items, wherein the nucleic acid is suitable for use in intratumoural applications.
42. The nucleic acid sequence according to any of the preceding items, wherein the expression of the encoded peptide or protein can be detected within non-liver cells preferably selected from immune cells, muscle cells or lung cells.
43. The nucleic acid sequence according to item 27, wherein the encoded peptide or protein is selected or derived from an antigen or epitope of an antigen.
44. The nucleic acid sequence according to item 43, wherein the antigen or epitope of an antigen is selected from a pathogen antigen.
45. The nucleic acid sequence according to any of the preceding items, wherein the nucleic acid is suitable for use in vaccination.
46. The nucleic acid sequence according to item 45, wherein the vaccination is suitable for use as therapeutic or prophylactic vaccination.
47. The nucleic acid sequence according to any of the preceding items, wherein the nucleic acid is suitable for use in systemic vaccination.
48. A pharmaceutical composition comprising a nucleic acid sequence as defined in items 1 to 47, optionally comprising one or more pharmaceutically acceptable excipients, carriers, diluents and/or vehicles.
49. Pharmaceutical composition according to item 48, wherein the nucleic acid sequence is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.
50. Pharmaceutical composition according to items 48 or 49, wherein the nucleic acid sequence is complexed or associated with one or more lipids, thereby forming liposomes, lipid nanoparticles (LNP), lipoplexes, and/or nanoliposomes.
51. Pharmaceutical composition according to item 50, wherein the nucleic acid sequence is complexed with one or more lipids thereby forming lipid nanoparticles (LNP).
52. Pharmaceutical composition according to item 50 and 51, wherein the lipid nanoparticles (LNP) comprise a PEGylated lipid.
53. Pharmaceutical composition according to items 50 to 52, wherein the LNP comprises
   (i) at least one cationic lipid;
   (ii) at least one neutral lipid;
   (iii) at least one steroid or steroid analogue; and
   (iv) at least one polymer-conjugated lipid, preferably a PEG-lipid,
   wherein (i) to (iv) are in a molar ratio of about 20-60% cationic lipid, 5-25% neutral lipid, 25-55% sterol, and 0.5-15% polymer-conjugated lipid, preferably a PEG-lipid.
54. Pharmaceutical composition according to items 48 to 53, wherein the pharmaceutical composition comprises Ringer or Ringer-Lactate solution.
55. Pharmaceutical composition according to items 48 to 54, wherein the pharmaceutical composition is administered to a cell or subject.
56. Pharmaceutical composition according to items 55, wherein the subject is a human subject.
57. Pharmaceutical composition according to item 55 or 56, wherein the administration is systemically or locally.
58. Pharmaceutical composition according to item 55 to 57, wherein the administration is transdermally, intradermally, intravenously, intramuscularly, intraaterially, intranasally, intrapulmonally, intracranially, intralesionally, intratumorally, intravitreally, subcutaneously or via sublingual, preferably intramuscularly, intranodally, intradermally, intratumorally or intravenously,
59. Pharmaceutical composition according to items 55 to 56, wherein the administration is more than once, for example once or once more than once a day, once or more than once a week, once or more than once a month.
60. Pharmaceutical composition according to items 48 to 59, wherein the pharmaceutical composition is formulated as a vaccine.
61. Pharmaceutical composition according to item 60, wherein the vaccine comprises the nucleic acid sequence as defined in items 1 to 48.
62. Kit or kit of parts comprising the nucleic acid sequence as defined in items 1 to 47, or the pharmaceutical composition as defined in items 48 to 61, optionally comprising a liquid vehicle for solubilizing, and, optionally, technical instructions providing information on administration and/or dosage of the components.
63. A nucleic acid sequence as defined in items 1 to 47, or a pharmaceutical composition as defined in items 48 to 61, or a nucleic acid sequence used as a vaccine as defined in items 42 to 47 and 60 to 61 or a kit or kit of parts as defined in item 52, for use as medicament.
64. A nucleic acid sequence as defined in items 1 to 47, or a pharmaceutical composition as defined in items 48 to 61, or a nucleic acid sequence used as a vaccine as defined in items 42 to 47 and 60 to 61 or a kit or kit of parts as defined in item 62, for use in the prevention or treatment of cancer, autoimmune diseases, infectious diseases, allergies or protein deficiency disorders.
65. A method of treatment or preventing a disorder, wherein the method comprises applying or administering to a subject in need thereof the nucleic acid sequence as defined in items 1 to 47, or the pharmaceutical composition as defined in items 48 to 61, or the kit or kit of parts as defined in item 62, preferably wherein applying or administering is performed more than once, for example once or more than once a day, once or more than once a week, once or more than once a month.
66. Method of treatment or preventing a disorder according to item 65, wherein the administration or applying is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, intranodal, or intratumoral, preferably intramuscular, intradermal, intravenous, or intratumoral.
67. Method of treatment according to item 65 or 66, wherein the subject in need is a mammalian subject, preferably a human subject.
68. A method to promote a cell-type specific expression of a peptide or protein within a target organ or organs by using a nucleic acid sequence as defined in items 1 to 47, the pharmaceutical composition as defined in items 48 to 61, the nucleic acid sequence used as a vaccine as defined in items 42 to 47 and 60 to 61 or the kit according to items as defined in item 62.

### BRIEF DESCRIPTION OF LIST AND TABLES

### List 1: Suitable Pathogens of the Invention

Table I: miRNA binding sites used in embodiments of the present invention
Table II: Human codon usage with respective codon frequencies indicated for each amino acid
Table III: Single miRNA binding sites in mRNA encoding PpLuc
Table IV: Repeats and tandem repeats of two identical miRNA binding sites in mRNA encoding PpLuc
Table V: Repeats and tandem repeats of two different miRNA binding sites in mRNA encoding PpLuc
Table VI: Repeats and tandem repeats of three identical miRNA binding sites in mRNA encoding PpLuc
Table VII: Comparison of % expression of mRNA encoding PpLuc (see Table III) in PHH cells comprising single miRNA binding sites (FIG. 1A)
Table VIII: Comparison of % expression of mRNA encoding PpLuc (see Table IV) in PHH cells comprising repeats and tandem repeats of two identical miRNA binding sites (FIG. 2A)
Table IX: Comparison of % expression of mRNA encoding PpLuc (see Table V) in PHH cells comprising repeats and tandem repeats of two different miRNA binding sites (FIG. 3A)
Table X: Comparison of % expression of mRNA encoding PpLuc (see Table Ill, Table IV and Table VI) in PHH cells comprising repeats and tandem repeats of three identical miRNA binding sites (FIG. 4A)
Table XI: Comparison of % expression of mRNA encoding PpLuc (see Table III, Table IV and Table VI) in PHH cells comprising single, repeat and tandem repeats of two or three miRNA-122-5p binding sites (FIG. 5A)
Table XII: Single miRNA binding sites in mRNA encoding PpLuc transfected in various cell types
Table XIII: Comparison of % expression of mRNA encoding PpLuc comprising single miRNA binding site transfected in PHH cells (FIG. 6A)
Table XIV: Comparison of % expression of mRNA encoding PpLuc comprising single miRNA-122-5p binding site transfected in PHH cells (FIG. 7A)
Table XV: Single miRNA binding sites in mRNA encoding IL-12 transfected in PHH and HeLa
Table XVI: Comparison of % expression of mRNA encoding IL-12 comprising miRNA-122-5p binding site transfected in PHH cells (FIG. 8A)
Table XVII: Single miRNA binding sites in mRNA encoding PpLuc transfected in PHH cells
Table XVIII: Comparison of expression of mRNA encoding PpLuc transfected in different doses comprising miRNA-122-5p binding site transfected in PHH (FIG. 9)
Table XIX: Different miRNA binding sites in mRNA encoding PpLuc used for in vivo experiments
Table XX: Lipid composition and lipid ratios of lipid nanoparticles used in experiment
Table XXI: miRNA binding sites in LNP formulated mRNA encoding PpLuc administered i.m.
Table XXII: miRNA binding sites in LNP formulated mRNA encoding PpLuc administered i.v.

### BRIEF DESCRIPTION OF DRAWINGS

The figures shown in the following are merely illustrative and shall describe the present invention in a further way. These figures shall not be construed to limit the present invention thereto.

FIG. 1 shows the expression from PpLuc encoding mRNA constructs comprising single miRNA binding sites from miRNA-122-5p ((C) upstream of 5′UTR; (G) downstream of 3′UTR), miRNA-148a-3p ((B) upstream of 5′UTR; (F) downstream of 3′UTR), miRNA-101-3p ((D) upstream of 5′UTR, (H) downstream of 3′UTR) and miRNA-192-5p ((E) upstream of 5′UTR; (1) downstream of 3′UTR). miRNA binding sites were cloned into the sequence in front of the 5′UTR or following the 3′UTR. PpLuc expression is shown in Figure A: PHH (primary human hepatocytes), see Table VII. Figure B: THP-1 cells (a human monocytic cell line derived from an acute monocytic leukemia patient) and Figure C: JAWSII cells (immortalized immature dendritic cell line established from the bone marrow of C57Bl/6 mice). (A) Control=reference sequence without miRNA binding sites. Further details are provided in example 2.

FIG. 2 shows the expression from PpLuc encoding mRNA constructs comprising two identical miRNA binding sites selected from miRNA-122-5p ((C) 5p upstream and within 5′ UTR; (F) downstream of 3′ UTR), miRNA-148a-3p ((B) upstream of 5′ UTR; (E) downstream of 3′ UTR) and miRNA-101-3p ((D) upstream and within 5′ UTR; (G) downstream of 3′ UTR) PpLuc expression is shown in Figure A: PHH (primary human hepatocytes), see Table VIII; Figure B: THP-1 cells (a human monocytic cell line derived from an acute monocytic leukemia patient) and Figure C: JAWSII cells (immortalized immature dendritic cell line established from the bone marrow of C57Bl/6 mice). (A) Control=reference sequence without miRNA binding sites. Further details are provided in example 2.

FIG. 3 shows the expression from PpLuc encoding mRNA constructs comprising two different miRNA binding sites selected from miRNA-122-5p and 192-5p ((B) 122-5p upstream of 5′ UTR/192-5p downstream of 3′ UTR), miRNA-148a-3p and 194-5p ((D) 148a-3p upstream of 5′ UTR/194-5p downstream of 3′ UTR), miRNA-101-3p and 148a-3p ((C) 101-3p upstream of 5′ UTR/148a-3p downstream of 3′ UTR), miRNA-101-3p and 194-5p ((E) 101-3p within 5′ UTR/194-5p downstream of 3′ UTR), 148a-3p and 122-5p ((F) 148a-3p upstream of 5′ UTR and 122-5p within the 5′ UTR) and miRNA-192-5p and 194-5p ((G) 192-5p upstream of 5′ UTR/194-5p within 3′ UTR). PpLuc expression is shown in Figure A: PHH (primary human hepatocytes), see Table IX; Figure B: THP-1 cells (a human monocytic cell line derived from an acute monocytic leukemia patient) and Figure C: JAWSII cells (immortalized immature dendritic cell line established from the bone marrow of C57Bl/6 mice). (A) Control=reference sequence without miRNA binding sites. Further details are provided in example 2.

FIG. 4: shows the expression of PpLuc encoding mRNA constructs comprising tandem repeats of three identical miRNA binding sites selected from miRNA-122-5p ((C) 3×122-5p upstream of 5′ UTR; (F) 3×122-5p downstream of 3′ UTR), miRNA-148a-3p ((B) 3×148a-3p upstream of 5′ UTR; (E) 3×148a-3p downstream of 3′ UTR), miRNA-101-3p ((D) 3×101-3p upstream of 5′ UTR; (G) 101-3p downstream of 3′ UTR). PpLuc expression is shown in Figure A: PHH (primary human hepatocytes), see Table X; Figure B: THP-1 cells (a human monocytic cell line derived from an acute monocytic leukemia patient) and Figure C: JAWSII cells (immortalized immature dendritic cell line established from the bone marrow of C57B11/6 mice). (A) Control=reference sequence without miRNA binding sites. Further details are provided in example 2.

FIG. 5 shows the expression from PpLuc encoding mRNA constructs comprising single ((B) 1×122-5p upstream of 5′ UTR; (C) 1×122-5p downstream of 3′ UTR), two identical ((D) 2×122-5p upstream and within 5′ UTR; (E) 2×122-5p downstream of 3′ UTR) and tandem repeats of three identical ((F) 3×122-5p upstream of 5′ UTR and (G) 3×122-5p downstream of 3′ UTR) miRNA binding sites of miRNA-122-5p binding sites. PpLuc expression is shown in Figure A: PHH (primary human hepatocytes), see Table XI; Figure B: THP-1 cells (a human monocytic cell line derived from an acute monocytic leukemia patient) and Figure C: JAWSII cells (immortalized immature dendritic cell line established from the bone marrow of C57B11/6 mice). (A) Control=reference sequence without miRNA binding sites. Further details are provided in example 2.

FIG. 6 shows the expression from PpLuc encoding mRNA constructs comprising single miRNA binding sites from miRNA-122-5p ((C) Upstream of 5′ UTR; (F) Downstream of 3′ UTR), miRNA-148a-3p ((A) Upstream of 5′ UTR; (D) Downstream of 3′ UTR) and miRNA-192-5p ((B) Upstream of 5′ UTR; (E) Downstream of 3′ UTR). miRNA binding sites were cloned into the sequence in front of the 5′UTR or following the 3′UTR. PpLuc expression is shown in Figure A: PHH (primary human hepatocytes), see Table XIII; Figure B: HeLa (immortal cell line of cervical cancer), Figure C: LLC1 (murine lewis lung carcinoma cell line), Figure D: A375 (human epithelial malignant melanoma), Figure E: B16F10 (murine skin cell melanoma cell line), Figure F: MC38 (murine colon adenocarcinoma) and Figure G: CT26 (murine colon carcinoma line). (G) Control=reference sequence without miRNA binding sites, (H) mock transfection. Further details are provided in example 3.

FIG. 7 shows the expression from PpLuc encoding mRNA constructs comprising single miRNA binding sites from miRNA-122-5p ((C) upstream of 5′ UTR; (F) downstream of 3′ UTR). miRNA binding sites were cloned into the sequence in front of the 5′UTR or following the 3′UTR. PpLuc expression is shown in Figure A: PHH (primary human hepatocytes), see Table XIV and Figure B: HeLa (immortal cell line of cervical cancer). (G) Control=reference sequence without miRNA binding sites. Further details are provided in example 3.

FIG. 8 shows the expression from IL-12 construct (IL12B-Linker-IL12A) encoding mRNA constructs comprising single miRNA binding sites from miRNA-122-5p ((B) Upstream of 5′ UTR; (C) Downstream of 3′ UTR). miRNA binding sites were cloned into the sequence in front of the 5′UTR or following the 3′UTR. IL-12 expression is shown in Figure A: PHH (primary human hepatocytes), see Table XVI and Figure B: HeLa (immortal cell line of cervical cancer). (A) Control=reference sequence without miRNA binding sites. Further details are provided in example 4.

FIG. 9 shows the expression from PpLuc encoding mRNA constructs comprising a single miRNA binding site miRNA-122-5p upstream of the 5′UTR (Group B, E, H) and downstream of the 3′UTR (Group C, F, I) or a control/reference sequence without miRNA binding site (Group A, D and G) in 3 different application doses (10 ng: Group A, B and C; 50ng: Group D, E and F; 100ng: G, H and I). Further details are provided in example 5, table XVIII.

FIG. 10 shows the expression from PpLuc encoding formulated mRNA constructs comprising a single miRNA binding site from miRNA-122-5p ((B) upstream of the 5′UTR; (C) downstream of the 3′UTR), miRNA-142-3p ((D) upstream of the 5′UTR; (E) downstream of the 3′UTR), miRNA-223-3p ((F) upstream of the 5′UTR; (G) downstream of the 3′UTR) or a control/reference construct without a miRNA binding site (A) or mock (H) after i.v. injection in the liver (FIG. 10A) and the spleen (FIG. 10B). Timepoints of measurement were 4 hours or 24 hours after injection. Further details are provided in example 6, table XX.

FIG. 11 shows the expression from PpLuc encoding formulated mRNA constructs comprising a single miRNA binding site from miRNA-122-5p upstream of the 5′UTR (Group B and E), downstream of the 3′UTR (Group C and F) or a control/reference sequence without miRNA binding sites (Group A and D) after i.m. injection in the muscle (FIG. 11A), the liver (FIG. 11B), spleen (FIG. 11C) and poplietal lymph nodes (FIG. 11D). Further details are provided in example 6.

FIG. 12 displays a schematic example of miRNA binding sites within a target mRNA. Figure A: mRNA comprising a miRNA binding site (miRNA BS) within the first miRNA binding site sequence prior to the 5′UTR sequence. Figure B: mRNA comprising two miRNA binding sites (miRNA BS) within the first miRNA binding site sequence (miRNA BS sequence) prior to the 5′UTR sequence. Figure C: mRNA comprising a miRNA binding site (miRNA BS) within the first miRNA binding site sequence prior to the 5′UTR sequence and another miRNA BS within the second miRNA binding site sequence after the 3′UTR. Figure D: miRNA binding to the target mRNA which leads to no expression of the target mRNA. The mature miRNA binds to the target mRNA at the miRNA binding site (miRNA BS). MiRNAs base-pair with miRNA BS located on their mRNA targets, prior the 5′ UTR, through a critical region called the ‘seed region’ which includes nucleotides 2-8 from the 5-end of the miRNA or miRNA base-pair with the complete sequence of the miRNA binding site. Figure E: mRNA comprising two miRNA binding sites (miRNA BS) prior and after the 5′ UTR sequence. Figure F: mRNA comprising two miRNA binding sites (miRNA BS) prior and within the 5′UTR sequence. Figure G: mRNA comprising three miRNA binding sites (miRNA BS) prior, within and after the 5′UTR sequence.

### EXAMPLES

The following examples are given to enable those skilled in the art to more clearly understand and to practice the present invention. The present invention, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below.

### Example 1: Generation of RNA Constructs

### 1.1 Preparation of DNA Templates

A DNA sequence encoding Photinus pyralis luciferase (PpLuc luciferase) or Interleukin IL-12 construct (IL-12B-Linker-IL12A) was prepared and used for subsequent RNA in vitro transcription. Said DNA sequences were prepared by modifying the wild type cds sequences by introducing a GC optimized cds. Sequences were introduced into a plasmid vector comprising UTR sequences, a stretch of adenosines, a histone-stem-loop structure, and, optionally, a stretch of 30 cytosines. Obtained plasmid DNA was transformed and propagated in bacteria using common protocols and plasmid DNA was extracted, purified, and used for subsequent RNA in vitro transcription as outlined below.

1.2. RNA In Vitro Transcription from Plasmid DNA Templates:
Preparation of mRNA Encoding PpLuc or IL-12 Construct:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) and cap- or cap analogue (e.g., m7GpppG or m7G(5′)ppp(5′)(2′OMeA)pG or m7G(5′)ppp(5′)(2′OMeG)pG)) under suitable buffer conditions. The obtained RNA was purified using RP-HPLC (PureMessenger®; WO2008/077592) and used for in vitro experiments.

### Example 2: Regulation of PpLuc Liver Expression with miRNA Binding Sites

### 2.1 Experimental Design

mRNA constructs encoding PpLuc were generated according to Example 1. Different miRNA binding sites were cloned in front of (upstream) the 5′UTR, within the 5′ UTR, or following (downstream) the 3′UTR and the degree of silencing of the resulting constructs has been determined in several cell lines. Respective UTR elements used are indicated therein within the sequence protocol (mRNA design (HSD17B4 (5′ UTR)/PSMB3 (3′ UTR))). Every mRNA construct was transfected in PHH (primary human hepatocytes), JAWSII cells (immortalized immature dendritic cell line established from the bone marrow of C57Bl/6 mice) and THP-1 cells (a human monocytic cell line derived from an acute monocytic leukemia patient). The miRNA binding sites were selected from miRNA-122-5p, miRNA-148a-3p, miRNA-101-3p, miRNA-194-5p and miRNA-192-5p and used as monomers/single miRNA binding site (1× miRNA binding site), tandem repeats of two identical or two different miRNA binding sites (2× miRNA binding sites) and tandem repeats of three identical miRNA binding sites (3× miRNA binding sites). The expression levels of the constructs encoding PpLuc and containing the different miRNA binding sites were analyzed using HTS (High Throughput Screening) assay plates in a luciferase assay.

2.2 Formulation and Delivery of PpLuc Constructs into Human Cells

PHH were seeded in a collagen-coated 96-well flat bottom plate in triplicates. The cells were transfected with 50 ng of mRNA constructs using Lipofectamine® MessengerMAX in triplicates. The RNA (ug):Lipofectamine® MessengerMAX (ul) ratio of 1:4 was used. Mock transfected cells served as negative control. After 24 hours cell lysates were prepared and frozen at −80° C. until the expression of luciferase was analyzed by luciferase assay as described below.

THP1 cells were seeded in a 96-well flat bottom plate in triplicates. The cells were transfected with 500 ng of mRNA constructs using Lipofectamine® 2000 in triplicates, including positive control (mRNA construct not containing miRNA binding sites) and mock transfected cells served as negative control. The RNA (ug):Lipofectamine (ul) ratio of 1:1.5 to 1:2 was used. After 24 hours cell lysates were prepared and frozen at −80° C. until the expression of luciferase was analyzed by luciferase assay as described below.

JAWSII were seeded in a 96-well flat bottom plate in triplicates. The cells were transfected with 500 ng of mRNA constructs using Lipofectamine® 2000 in triplicates, including positive control (mRNA construct not containing miRNA binding sites) and mock transfected cells served as negative control. The RNA (ug):Lipofectamine (ul) ratio of 1:1.5 to 1:2 was used. After 24 hours cell lysates were prepared and frozen at −80° C. until the expression of luciferase was analyzed by luciferase assay as described below.

TABLE III
Single miRNA binding sites in mRNA encoding PpLuc

| miRNA | 5′cap | | | | Position of miRNA | SEQ ID |
|---|---|---|---|---|---|---|
| binding site | structure | 5′ elements | 3′ elements | 3′ end | binding site | NO: |
| 1x 148a-3p | Cap1 | miR148a-3p_5′UTR HSD17B4 | 3′UTR PSMB3 | hsl-A100 (Sapl) | Upstream of 5′ UTR | 259 |
| 1x 148a-3p | Cap1 | 5′UTR HSD17B4 | 3′UTR PSMB3_miR148a-3p | hsl-A100 (Sapl) | Downstream of 3′ UTR | 260 |
| 1x 122-5p | Cap1 | miR122-5p_5′UTR HSD17B4 | 3′UTR PSMB3 | hsl-A100 (Sapl) | Upstream of 5′ UTR | 261 |
| 1x 122-5p | Cap1 | 5′UTR HSD17B4 | 3′UTR PSMB3_miR122-5p | hsl-A100 (Sapl) | Downstream of 3′ UTR | 262 |
| 1x 101-3p | Cap1 | miR101-3p_5′UTR HSD17B4 | 3′UTR PSMB3 | hsl-A100 (Sapl) | Upstream of 5′ UTR | 263 |
| 1x 101-3p | Cap1 | 5′UTR HSD17B4 | 3′UTR PSMB3_miR101-3p | hsl-A100 (Sapl) | Downstream of 3′ UTR | 264 |
| 1x 192-5p | Cap1 | miR192-5p_5′UTR HSD17B4 | 3′UTR PSMB3 | hsl-A100 (Sapl) | Upstream of 5′ UTR | 265 |
| 1x 192-5p | Cap1 | 5′UTR HSD17B4 | 3′UTR PSMB3_miR192-5p | hsl-A100 (Sapl) | Downstream of 3′ UTR | 266 |
| / | Cap1 | 5′UTR HSD17B4 | 3′UTR PSMB3 | hsl-A100 (Sapl) | / | 267 |

TABLE IV
Repeats and tandem repeats of two identical miRNA binding sites in mRNA encoding PpLuc

| miRNA | 5′cap | | | | Position of miRNA | SEQ ID |
|---|---|---|---|---|---|---|
| binding site | structure | 5′ elements | 3′ elements | 3′ end | binding site | NO: |
| 2x 148a-3p | Cap1 | miR148a-3p_miR148a-3p_{—} | 3′UTR PSMB3 | hsl-A100 (Sapl) | Upstream of 5′ UTR | 268 |
| | | 5′UTR HSD17B4 | | | | |
| 2x 148a-3p | Cap1 | 5′UTR HSD17B4 | 3′UTR PSMB3_miR148a- | hsl-A100 (Sapl) | Downstream of 3′ UTR | 269 |
| | | | 3p_miR148a-3p | | | |
| 2x 122-5p | Cap1 | miR122-5p_5′UTR HSD17B4(1- | 3′UTR PSMB3 | hsl-A100 (Sapl) | Upstream and within 5′ | 270 |
| | | 20)_miR122-5p_5′UTR | | | UTR | |
| | | HSD17B4(21-62) | | | | |
| 2x 122-5p | Cap1 | 5′UTR HSD17B4 | 3′UTR PSMB3_miR122- | hsl-A100 (Sapl) | Downstream of 3′ UTR | 271 |
| | | | 5p miR122a-5p | | | |
| 2x 101-3p | Cap1 | miR101-3p_5′UTR HSD17B4(1- | 3′UTR PSMB3 | hsl-A100 (Sapl) | Upstream and within 5′ | 272 |
| | | 20)_miR101-3p_5′UTR | | | UTR | |
| | | HSD17B4(21-62) | | | | |
| 2x 101-3p | Cap1 | 5′UTR HSD17B4 | 3′UTR PSMB3_miR101- | hsl-A100 (Sapl) | Downstream of 3′ UTR | 273 |
| | | | 3p_miR101-3p | | | |
| / | Cap1 | 5′UTR HSD17B4 | 3′UTR PSMB3 | hsl-A100 (Sapl) | / | 267 |

TABLE V
Repeats and tandem repeats of two different miRNA binding sites in mRNA encoding PpLuc

| miRNA | 5′cap | | | | Position of miRNA | SEQ ID |
|---|---|---|---|---|---|---|
| binding site | structure | 5′ elements | 3′ elements | 3′ end | binding site | NO: |
| 1x 122-5p | Cap1 | miR122-5p_5′UTR HSD17B4 | 3′UTR PSMB3_miR192-5p | hsl-A100 (Sapl) | Upstream of 5′ UTR and | 274 |
| 1x 192-5p | | | | | Downstream of 3′ UTR | |
| 1x 101-3p | Cap1 | miR101-3p_5′UTR HSD17B4 | 3′UTR PSMB3_miR148a-3p | hsl-A100 (Sapl) | Upstream of 5′ UTR and | 275 |
| 1x 148a-3p | | | | | Downstream of 3′ UTR | |
| 1x 148a-3p | Cap1 | miR148a-3p_5′UTR HSD17B4 | 3′UTR PSMB3_miR 194-5p | hsl-A100 (Sapl) | Upstream of 5′ UTR and | 276 |
| 1x 194-5p | | | | | Downstream of 3′ UTR | |
| 1x 101-3p | Cap1 | 5′UTR HSD17B4(1-20)_miR101- | 3′UTR PSMB3_miR194-5p | hsl-A100 (Sapl) | Within the 5′ UTR and | 277 |
| 1x 194-5p | | 3p_5′UTR HSD17B4(21-62) | | | Downstream of 3′ UTR | |
| 1x 148a-3p | Cap1 | miR148a-3p_5′UTR | Hs_3UTR_PSMB3.1 | hsl-A100 (Sapl) | Upstream and within the | 278 |
| 1x 122-5p | | HSD17B4(1-20)_miR122- | | | 5′ UTR | |
| | | 5p_5′UTR HSD17B4(21-62) | | | | |
| 1x 192-5p | Cap1 | miR192-5p_5′UTR HSD17B4 | 3′UTR PSMB3(1-40)_miR194- | hsl-A100 (Sapl) | Upstream of 5′ UTR and | 279 |
| 1x 194-5p | | | 5p_3′UTR PSMB3(41-57) | | within the 3′ UTR | |
| / | Cap1 | 5′UTR HSD17B4 | 3′UTR PSMB3 | hsl-A100 (Sapl) | Upstream of 5′ UTR vs. | 267 |
| | | | | | Downstream of 3′ UTR | |

TABLE VI
Repeats and tandem repeats of three identical miRNA binding sites in mRNA encoding PpLuc

| miRNA | 5′cap | | | | Position of | SEQ ID |
|---|---|---|---|---|---|---|
| binding site | structure | 5′ elements | 3′ elements | 3′ end | miRNA BS | NO: |
| 3x 148a-3p | Cap1 | miR-148a-3p_miR48a-3p_{—} | 3′UTR PSMB3 | hsl-A100 | Upstream of | 280 |
| | | miR148a-3p_5′UTR HSD17B4 | | (Sapl) | 5′ UTR | |
| 3x 148a-3p | Cap1 | 5′UTR HSD17B4 | 3′UTR PSMB3_miR148a- | hsl-A100 | Downstream | 281 |
| | | | 3p_miR148a-3p_miR148a-3p | (Sapl) | of 3′ UTR | |
| 3x 122-5p | Cap1 | miR-122-5p_miR-122-5p_miR- | 3′UTR PSMB3 | hsl-A100 | Upstream of | 282 |
| | | 122-5p_5′UTR HSD17B4 | | (Sapl) | 5′ UTR | |
| 3x 122-5p | Cap1 | 5′UTR HSD17B4 | 3′UTR PSMB3_miR122- | hsl-A100 | Downstream | 283 |
| | | | 5p_miR122-5p_miR122-5p | (Sapl) | of 3′ UTR | |
| 3x 101-3p | Cap1 | Hs_hsa-miR-101-3p_binding_{—} | 3′UTR PSMB3 | hsl-A100 | Upstream and | 284 |
| | | site.Hs_5UTR_HSD17B4_var_1- | | (Sapl) | within 5′ UTR | |
| | | 20.Hs_hsa-miR-101-3p_binding_{—} | | | | |
| | | site.Hs_5UTR_HSD17B4_var_21- | | | | |
| | | 52.Hs_hsa-miR-101-3p_binding_{—} | | | | |
| | | site.Hs_5UTR_HSD17B4_var_53-62 | | | | |
| 3x 101-3p | Cap1 | 5′UTR HSD17B4 | 3′UTR PSMB3_miR101- | hsl-A100 | Downstream | 285 |
| | | | 3p_miR101-3p_miR101-3p | (Sapl) | of 3′ UTR | |
| / | Cap1 | / | / | hsl-A100 | | 267 |
| | | | | (Sapl) | | |

### 2.3 Luciferase Assay

After thawing, 20 μl of lysates were used to detect and measure luciferase activity via chemi-luminescence using ATP and D-Luziferin in a Beetlejuice buffer system (p.j.k.). To this end, plates were introduced into a plate reader (Tristar 2S Berthold) with injection device for Beetle-juice containing substrate for firefly luciferase. Per well, 50 μl of beetle-juice were added.

Raw data containing relative light units were used to plot differences between mRNAs.

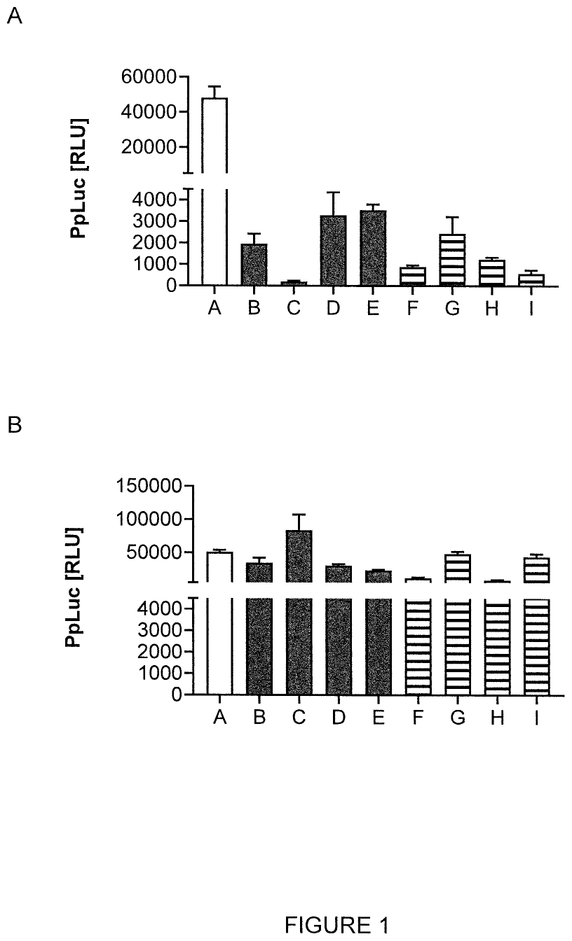

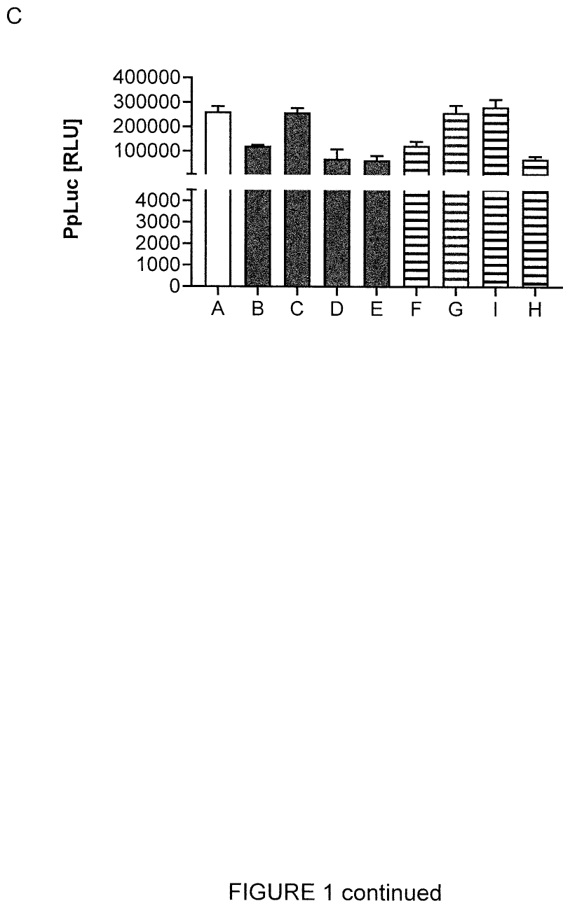

TABLE VII
Comparison of % expression of mRNA encoding PpLuc (see Table III)
in PHH cells comprising single miRNA binding sites (FIG. 1A)

| | | | | | Average | PpLuc |
|---|---|---|---|---|---|---|
| Group | miRNA binding site | Value 1 | Value 2 | Value 3 | PpLuc [RLU] | expression |
| A | Control (no miRNA binding site) | 41569 | 48932 | 54157 | 48219.33333 | 100% |
| B | 148a-3p upstream of 5′ UTR | 2431 | 1939 | 1513 | 1961 | 4.07% |
| C | 122-5p upstream of 5′ UTR | 232 | 186 | 171 | 196.3333 | 0.41% |
| D | 101-3p upstream of 5′ UTR | 3517 | 4242 | 2139 | 3299.333 | 6.84% |
| E | 192-5p upstream of 5′ UTR | 3486 | 3821 | 3308 | 3538.333 | 7.34% |
| F | 148a-3p downstream of 3′ UTR | 924 | 804 | 934 | 887.3333 | 1.84% |
| G | 122-5p downstream of 3′ UTR | 1691 | 2385 | 3248 | 2441.333 | 5.06% |
| H | 101-3p downstream of 3′ UTR | 1333 | 1130 | 1241 | 1234.667 | 2.56% |
| I | 192-5p downstream of 3′ UTR | 448 | 550 | 747 | 581.6667 | 1.21% |

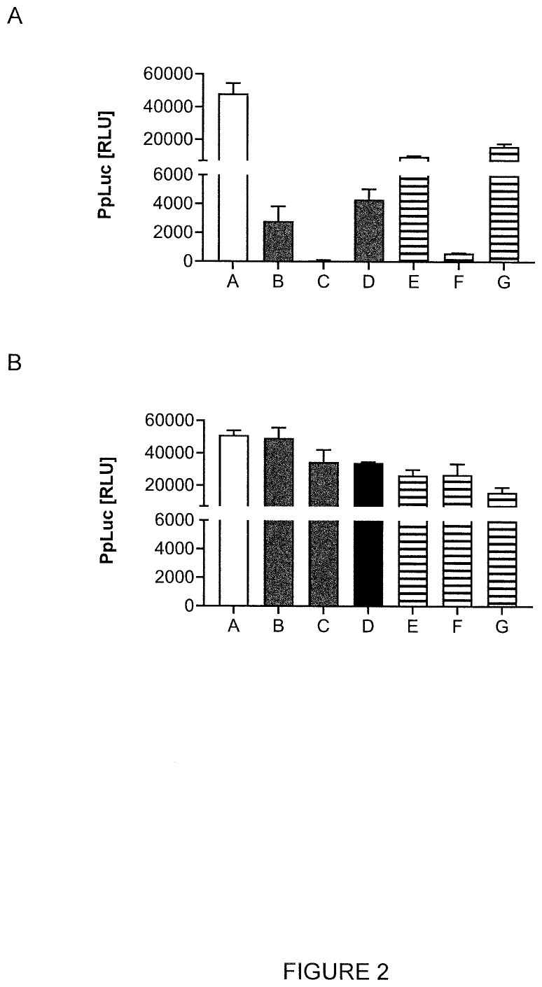

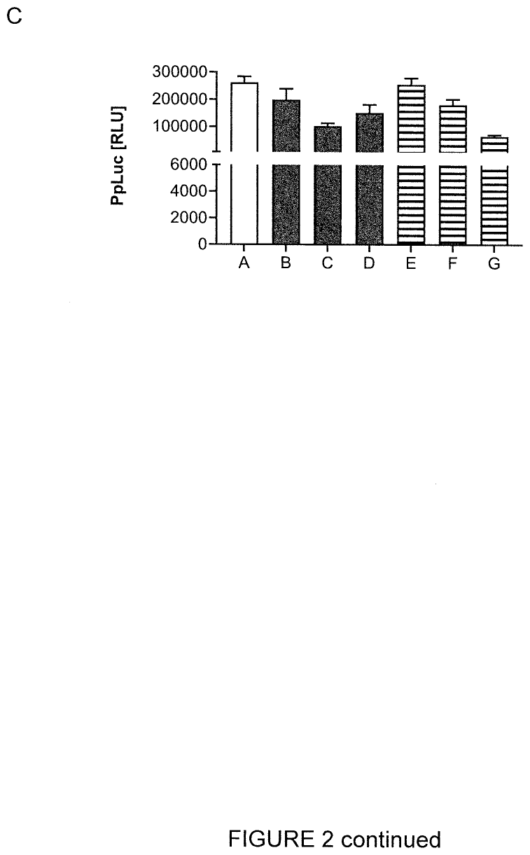

TABLE VIII
Comparison of % expression of mRNA encoding PpLuc (see Table IV) in PHH cells comprising
repeats and tandem repeats of two identical miRNA binding sites (FIG. 2A)

| | | | | | Average | PpLuc |
|---|---|---|---|---|---|---|
| Group | miRNA binding site | Value 1 | Value 2 | Value 3 | PpLuc [RLU] | expression |
| A | No miRNA binding site | 41569 | 48932 | 54157 | 48219.33333 | 100% |
| B | 2x 148a-3p upstream of 5′ UTR | 3050 | 3681 | 1715 | 2815.333333 | 5.84% |
| C | 2x 122-5p upstream and within 5′ UTR | 122 | 98 | 55 | 91.66666667 | 0.19% |
| D | 2x 101-3p upstream and within 5′ UTR | 4550 | 4888 | 3538 | 4325.333333 | 8.97% |
| E | 2x 148a-3p downstream of 3′ UTR | 10385 | 9818 | 9573 | 9925.333333 | 20.58% |
| F | 2x 122-5p downstream of 3′ UTR | 634 | 607 | 616 | 619 | 1.28% |
| G | 2x 101-3p downstream of 3′ UTR | 17801 | 14544 | 16355 | 16233.33333 | 33.67% |

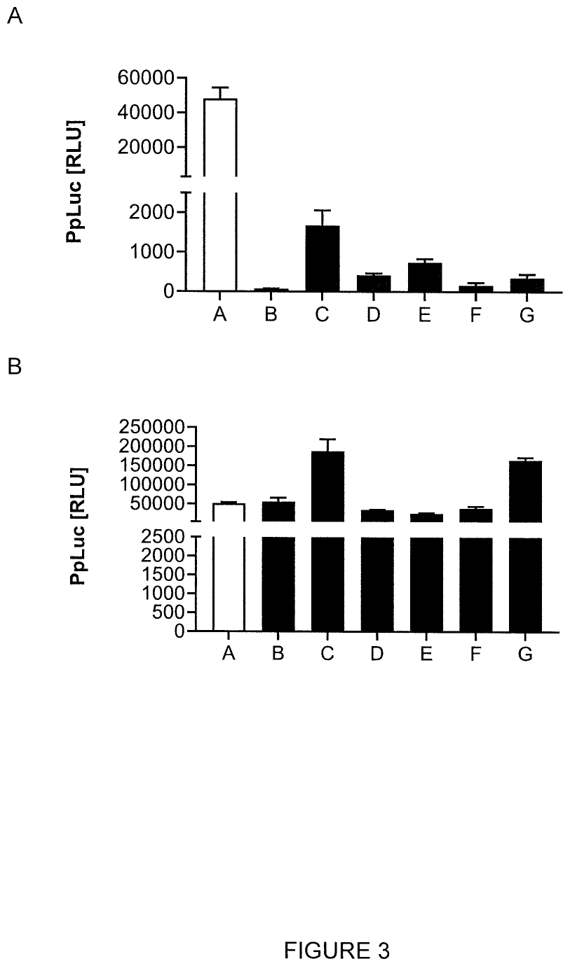

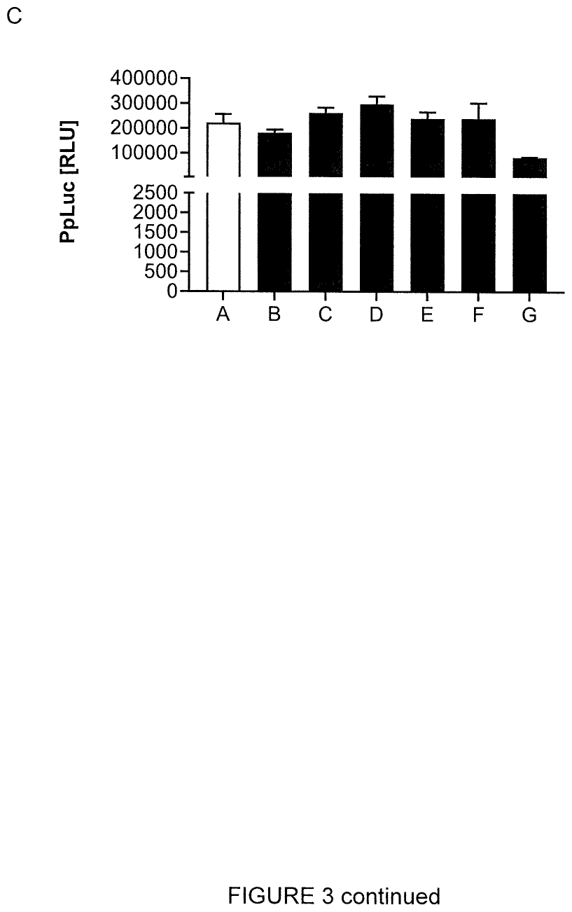

TABLE IX
Comparison of % expression of mRNA encoding PpLuc (see Table V) in PHH cells comprising
repeats andtandem repeats of two different miRNA binding sites (FIG. 3A)

| | | | | | Average | PpLuc |
|---|---|---|---|---|---|---|
| Group | miRNA binding site | Value 1 | Value 2 | Value 3 | PpLuc [RLU] | expression |
| A | No miRNA binding site | 41569 | 48932 | 54157 | 48219.33333 | 100% |
| B | 122-5p upstream of 5′ UTR/ | 77 | 77 | 79 | 77.66666667 | 0.16% |
| | 192-5p downstream of 3′ UTR | | | | | |
| C | 101-3p upstream of 5′ UTR/ | 1290 | 1718 | 2041 | 1683 | 3.49% |
| | 148a-3p downstream of 3′ UTR | | | | | |
| D | 148a-3p upstream of 5′ UTR/ | 447 | 377 | 451 | 425 | 0.88% |
| | 194-5p downstream of 3′ UTR | | | | | |
| E | 101-3p within 5′ UTR/ | 748 | 654 | 836 | 746 | 1.55% |
| | 194-5p downstream of 3′ UTR | | | | | |
| F | 148a-3p upstream of 5′ UTR and | 186 | 219 | 99 | 168 | 0.35% |
| | 122-5p within the 5′ UTR | | | | | |
| G | 192-5p upstream of 5′ UTR/ | 397 | 420 | 252 | 356.3333333 | 0.74% |
| | 194-5p within 3′ UTR | | | | | |

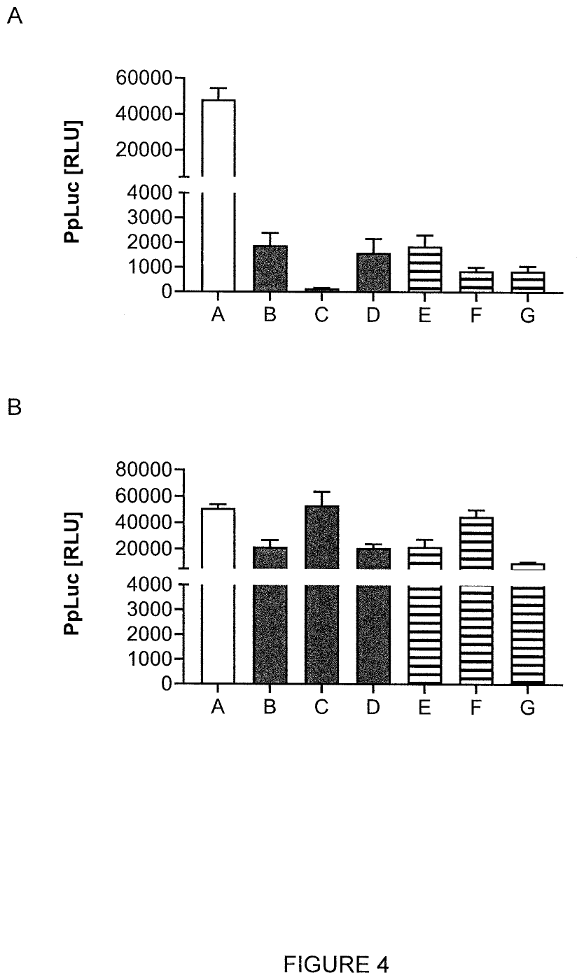

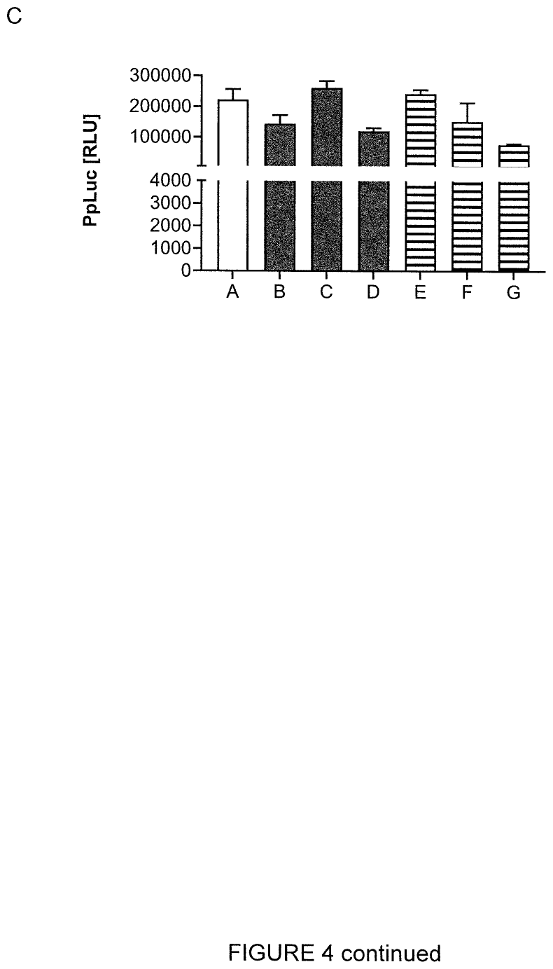

TABLE X
Comparison of % expression of mRNA encoding PpLuc (see Table VI) in PHH cells comprising
repeats and tandem repeats of three identical miRNA binding sites (FIG. 4A)

| | | | | | Average | PpLuc |
|---|---|---|---|---|---|---|
| Group | miRNA binding site | Value 1 | Value 2 | Value 3 | PpLuc [RLU] | expression |
| A | No miRNA binding site | 41569 | 48932 | 54157 | 48219.33333 | 100% |
| B | 3x 148a-3p upstream of 5′ UTR | 2462 | 1686 | 1547 | 1898.333333 | 3.94% |
| C | 3x 122-5p upstream of 5′ UTR | 171 | 147 | 143 | 153.6666667 | 0.32% |
| D | 3x 101-3p upstream of 5′ UTR | 2074 | 1747 | 988 | 1603 | 3.32% |
| E | 3x 148a-3p downstream of 3′ UTR | 2370 | 1704 | 1533 | 1869 | 3.88% |
| F | 3x 122-5p downstream of 3′ UTR | 1032 | 767 | 830 | 876.3333333 | 1.82% |
| G | 3x 101-3p downstream of 3′ UTR | 1061 | 690 | 860 | 870.3333333 | 1.80% |

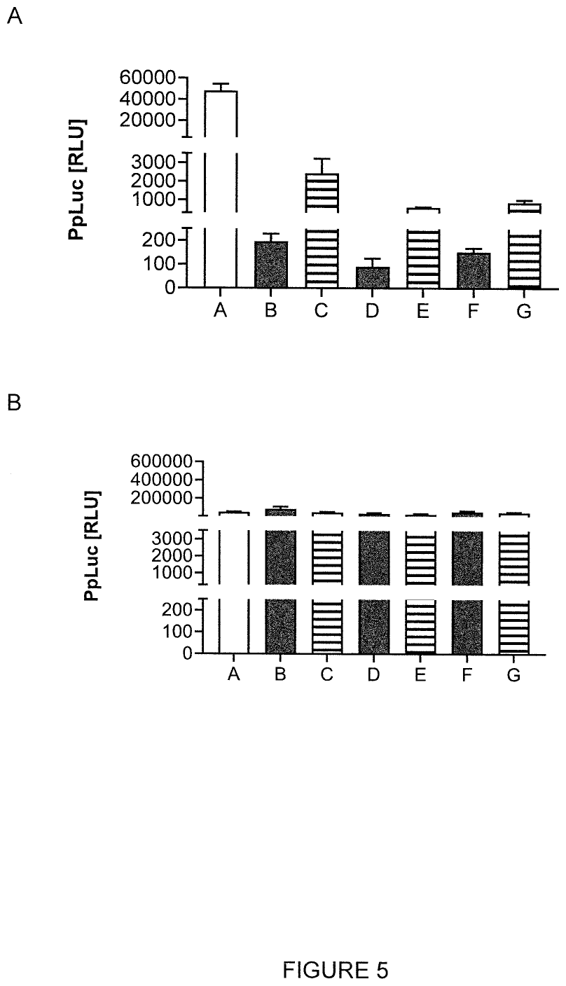

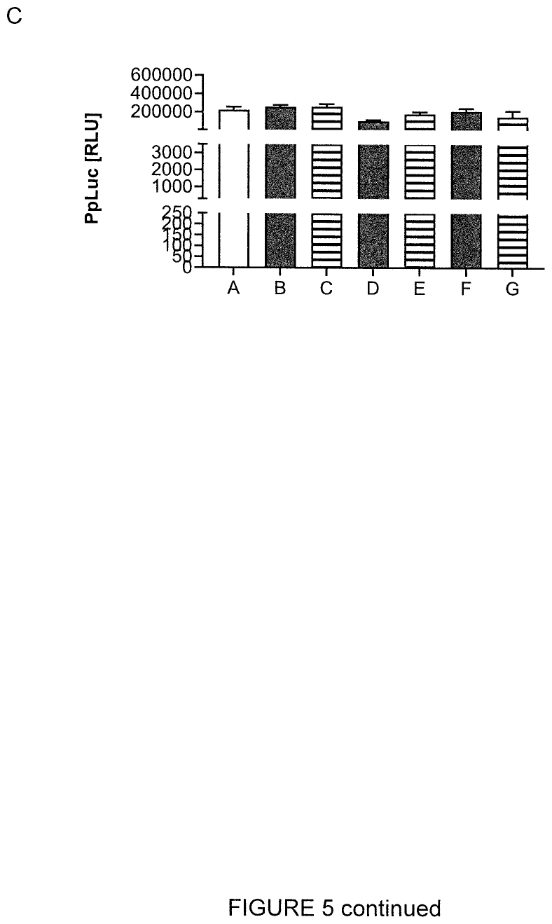

TABLE XI
Comparison of % expression of mRNA encoding PpLuc (see Table III, Table IV and Table VI) in PHH cells
comprising single, repeat and tandem repeats of two or three miRNA-122-5p binding sites (FIG. 5A)

| | | | | | Average | PpLuc |
|---|---|---|---|---|---|---|
| Group | miRNA binding site | Value 1 | Value 2 | Value 3 | PpLuc [RLU] | expression |
| A | No miRNA binding site | 41569 | 48932 | 54157 | 48219.33333 | 100% |
| B | 1x 122-5p upstream of 5′ UTR | 232 | 186 | 171 | 196.3333333 | 0.41% |
| C | 1x 122-5p downstream of 3′ UTR | 1691 | 2385 | 3248 | 2441.333333 | 5.06% |
| D | 2x 122-5p upstream and within 5′ UTR | 122 | 98 | 55 | 91.66666667 | 0.19% |
| E | 2x 122-5p downstream of 3′ UTR | 634 | 607 | 616 | 619 | 1.28% |
| F | 3x 122-5p upstream of 5′ UTR | 171 | 147 | 143 | 153.6666667 | 0.32% |
| G | 3x 122-5p downstream of 3′ UTR | 1032 | 767 | 830 | 876.3333333 | 1.82% |

2.4 Summary of the Findings Silencing capacities of different miRNA binding sites are shown in FIGS. 1 to 5. The comparison of the % expression of PpLuc are shown in Tables VII-XI. The transfection of PHH (FIG. 1A, Table VII) with single miRNA binding sites (monomers) before the 5′UTR or after the 3′UTR leads to a reduction of the PpLuc expression in liver cells compared to the control reference sequence not containing miRNA binding sites. Surprisingly, the construct containing a miRNA-122-5p binding site cloned before the 5′ UTR showed a much lower expression of PpLuc in PHH compared to the construct in which the miRNA-122-5p binding site was cloned after the 3′ UTR. There are no distinct differences in expression of PpLuc in immune cells, THP-1 (FIG. 1B, Table III) and JAWSII cells (FIG. 1C). Same results could also be shown for the constructs containing homo-dimers of the miRNA-122-5p binding site cloned before and within the 5′ UTR (FIG. 2, Table VIII). The combination of the miRNA-122-5p binding site within the 5′ UTR and miRNA-192-5p binding site within the 3′ UTR also led to a reduced expression of PpLuc in PHH (FIG. 3A, Table IX), compared to the other constructs containing hetero-dimers of miRNA binding sites and stable expression from all constructs in THP1 and JAWSII (FIGS. 3C and D). The same results for miRNA-122-5p monomers (FIG. 1) and dimers (FIG. 2) could also be shown for trimers of the miRNA-122-5p binding site cloned before the 5′ UTR of the construct (FIG. 4, Table X). FIG. 5 shows the direct comparison of mRNA constructs containing monomers, dimers- and trimers of miRNA-122-5p binding sites before the 5′UTR or after the 3′UTR in PHH, THP1 and JAWIIS, wherein a stronger reduction of the PpLuc expression in liver cells (PHH, FIG. 5A) is shown for the placement of the miRNA-122-5p binding site before and within the 5′UTR.

### Example 3: Regulation of Tumor-Specific Gene Expression by mRNA Encoding PpLuc Comprising Different miRNA Binding Sites

### 3.1 Experimental Design

mRNA constructs encoding PpLuc were generated according to Example 1. Different miRNA binding sites were cloned in front of (upstream) the 5′UTR or following (downstream) the 3′UTR and the degree of silencing of the resulting constructs has been determined in several cell lines (see Table XII). Respective UTR elements used are indicated therein within the sequence protocol (mRNA design (HSD17B4 (5′ UTR)/PSMB3 (3′ UTR))). Every mRNA construct was transfected into primary human hepatocytes (PHH), an immortal cell line of cervical cancer (HeLa), murine lewis lung carcinoma cell line (LLC1), murine skin cell melanoma cell line (B16F10), murine colon adenocarcinoma (MC38), human epithelial malignant melanoma (A375) and murine colon carcinoma line (CT26). The miRNA binding sites were selected from miRNA-122-5p, miRNA-148a-3p and miRNA-192-5p and used as monomers/singles miRNA binding site (1× miRNA binding site).

### 3.2 Formulation and Delivery of PpLuc Constructs

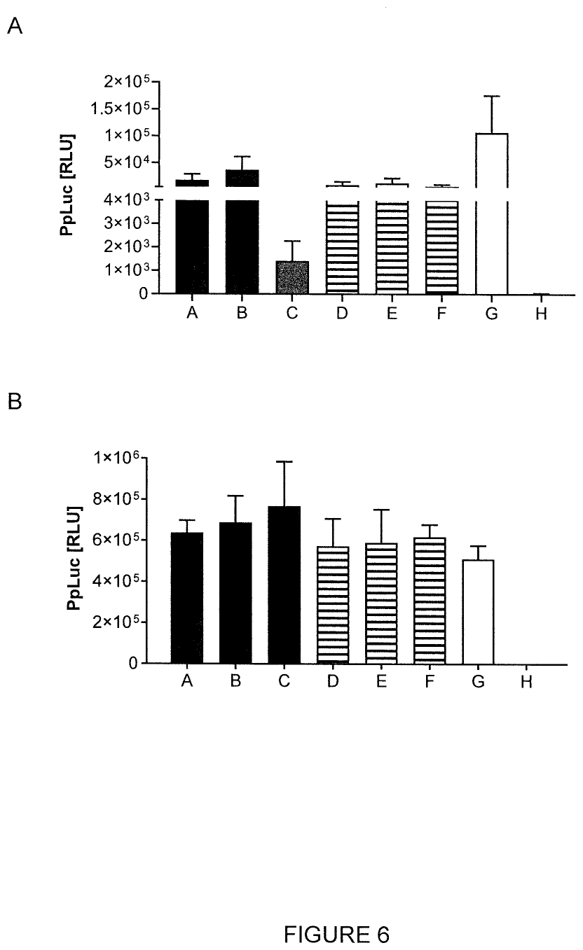

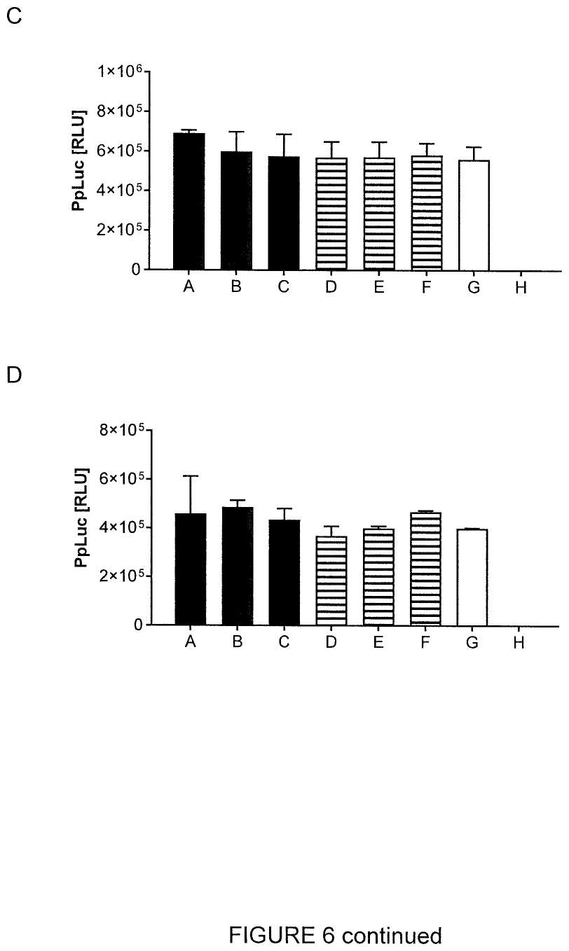

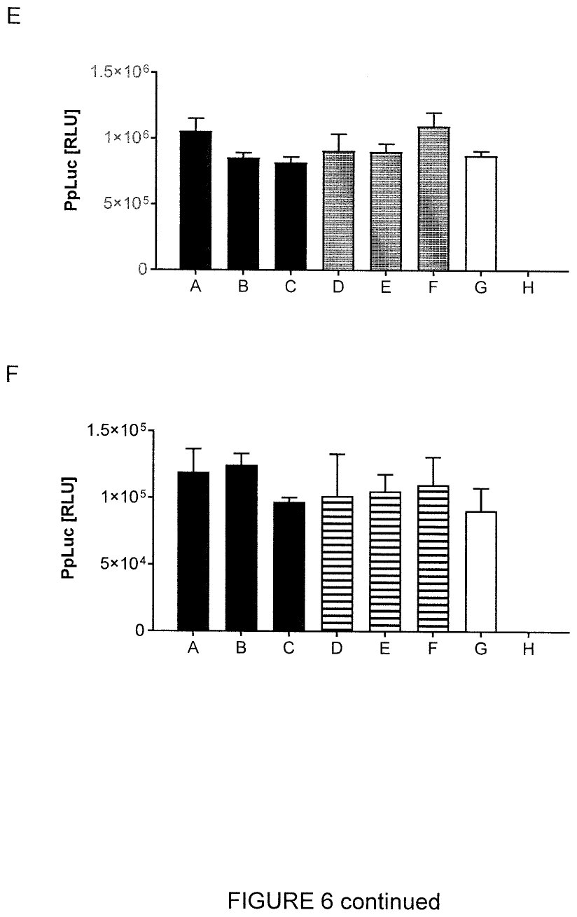

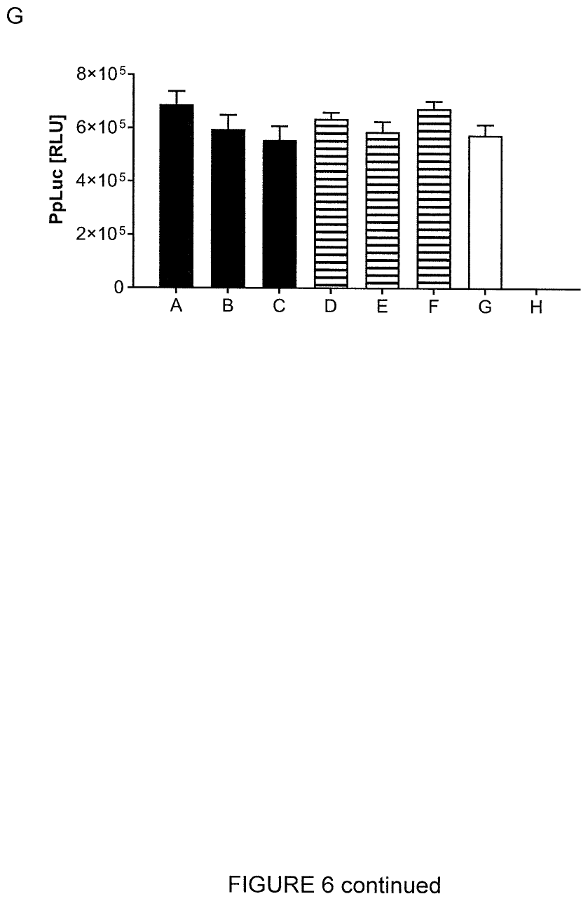

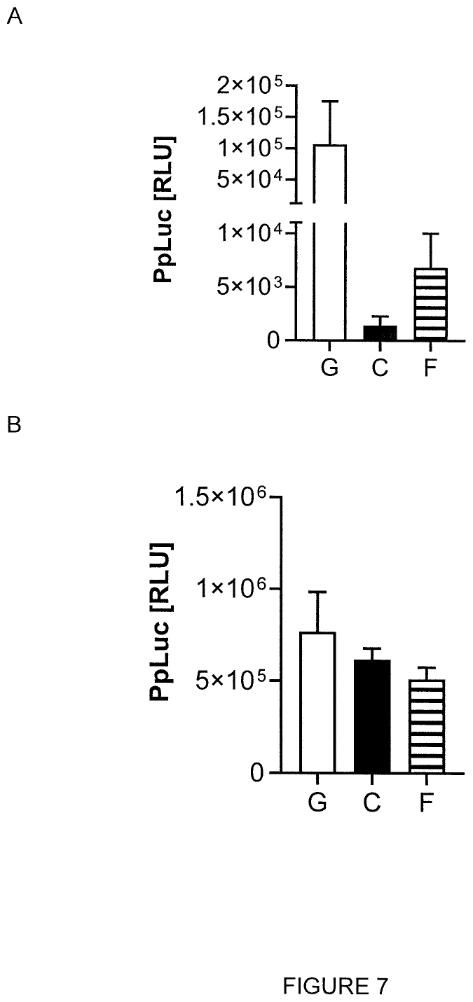

40.000 PHH were seeded in a collagen-coated 96-well flat bottom plate in triplicates. The cells were transfected with 100 ng (FIG. 6) or 50 ng (FIG. 7) of mRNA constructs using Lipofectamine® MessengerMAX. The RNA (ug):Lipofectamine® MessengerMAX (ul) ratio of 1:4 was used. Mock transfected cells served as negative control. After 24 hours cell lysates were prepared and frozen at −80° C. until the expression of luciferase was analyzed by luciferase assay as described in Example 2.3.

10.000 HeLa cells were seeded in a 96-well flat bottom plate in triplicates one day before transfection. The cells were transfected with 100 ng (FIG. 6) or 50 ng (FIG. 7) of mRNA constructs using Lipofectamine® 2000. The RNA (ug):Lipofectamine® 2000 (ul) ratio of 1:1.5 was used. After 24 hours cell lysates were prepared and frozen at −80° C. until the expression of luciferase was analyzed by luciferase assay as described in Example 2.3.

The five tumor cell lines (B16F10, MC38, LLC1, CT26 and A375) were seeded in a 96-well flat bottom plate in triplicates. The cells were transfected with 100 ng (FIG. 6) of different mRNA constructs using Lipofectamine® 2000 in triplicates. RNA (ug):Lipofectamine® 2000 (ul) ratio, 1:1.5 to 1:2 was used. Mock transfected cells serves as negative control. After 24 hours cell lysates were prepared and frozen at −80° C. until the expression of luciferase was analyzed by luciferase assay as described in Example 2.3.

TABLE XII
Single miRNA binding sites in mRNA encoding PpLuc transfected in various cell types

| | mRNA | miRNA | 5′cap | | Position of miRNA | Timepoint | SEQ ID |
|---|---|---|---|---|---|---|---|
| Group | ID | binding site | structure | 3′ end | binding site | of analysis | NO: |
| A | R9523 | 148a-3p | Cap1 | hsl-A100 (Sapl) | Upstream of 5′ UTR | 24 h | 286 |
| B | R9525 | 192-5p | Cap1 | hsl-A100 (Sapl) | Upstream of 5′ UTR | 24 h | 287 |
| C | R9526 | 122-5p | Cap1 | hsl-A100 (Sapl) | Upstream of 5′ UTR | 24 h | 288 |
| D | R9528 | 148a-3p | Cap1 | hsl-A100 (Sapl) | Downstream of 3′ UTR | 24 h | 289 |
| E | R9530 | 192-5p | Cap1 | hsl-A100 (Sapl) | Downstream of 3′ UTR | 24 h | 290 |
| F | R9531 | 122-5p | Cap1 | hsl-A100 (Sapl) | Downstream of 3′ UTR | 24 h | 291 |
| G | R8730 | No binding site | Cap1 | hsl-A100 (Sapl) | / | 24 h | 292 |

### 3.3 Luciferase Assay

Luciferase assay to measure the expression activity was performed according to Example 2.3.

TABLE XIII
Comparison of % expression of mRNA encoding PpLuc comprising
single miRNA binding site transfected in PHH cells (FIG. 6A)

| | | | | | Average | PpLuc |
|---|---|---|---|---|---|---|
| Group | miRNA binding site | Value 1 | Value 2 | Value 3 | PpLuc [RLU] | expression |
| A | 148a-3p upstream of 5′ UTR | 30819 | 9224 | 14058 | 18033.7 | 16.79% |
| B | 192-5p upstream of 5′ UTR | 64492 | 17046 | 30011 | 37183 | 34.61% |
| C | 122-5p upstream of 5′ UTR | 2250 | 564 | 1461 | 1425 | 1.33% |
| D | 148a-3p downstream of 3′ UTR | 1589 | 4048 | 7639 | 4425.33 | 4.12% |
| E | 192-5p downstream of 3′ UTR | 22321 | 5018 | 12264 | 13201 | 12.29% |
| F | 122-5p downstream of 3′ UTR | 9611 | 3343 | 7511 | 6821.67 | 6.35% |
| G | no miRNA binding site | 174740 | 38207 | 109330 | 107426 | 100% |
| H | Mock | 0 | 0 | 0 | 0 | 0 |

TABLE XIV
Comparison of % expression of mRNA encoding PpLuc
comprising single miRNA-122-5p binding site
transfected in PHH cells (FIG. 7A)

| | | | | Average | PpLuc |
|---|---|---|---|---|---|
| miRNA | Value | Value | Value | PpLuc | expres- |
| binding site | 1 | 2 | 3 | [RLU] | sion |
| G no miRNA | 174740 | 38207 | 109330 | 107425.6667 | 100% |
| binding site | | | | | |
| C 122-5p up- | 2250 | 564 | 1461 | 1425 | 1.33% |
| stream of 5′ UTR | | | | | |
| F 122-5p down- | 9611 | 3343 | 7511 | 6821.666667 | 6.35% |
| stream of 3′ UTR | | | | | |

### 3.4 Summary of the Findings

Strong silencing capacities of the construct containing miRNA-122 binding site cloned before the 5′UTR was shown in PHH (FIG. 6A, Table XII). The other constructs comprising miRNA binding sites miRNA-148a-3p or miRNA 192-5p cloned before the 5′UTR showed reduced expression levels compared to the reference construct as well (FIG. 6A). The comparison of the % expression of PpLuc are shown in Tables XIII-XIV. A comparable expression from constructs containing miRNA binding sites to the reference construct not containing miRNA binding sites (G, Table XII) could be seen for the tumor cell lines HeLa, LLC1, A375, B16F10, MC38 and CT26 (FIG. 6B-F, Table XII).

The expression of the encoded target protein is not affected in different tumor cell lines but can be reduced in hepatocytes (PHH cells).

A stronger reduction in the expression of PpLuc could be shown also in PHH with the incorporation of miRNA-122-5p binding site cloned before the 5′ UTR compared to the incorporation after the 3′ UTR (FIG. 7A, Table XIV). No significant reduction of the PpLuc expression compared to the reference construct was shown in HeLa cells (FIG. 7B).

### Example 4: Regulation of Tumor-Specific Gene Expression by mRNA Encoding IL-12 Construct Comprising miRNA-122 Binding Site

### 4.1 Experimental Design

mRNA constructs encoding subunit beta and alpha of interleukin 12 (IL-12B-Linker-IL12A) were generated according to Example 1. miRNA-122 binding site was cloned in front of (upstream) the 5′UTR or following (downstream) the 3′UTR and the degree of silencing of the resulting constructs has been determined in primary human hepatocytes (PHH) and in an immortal cell line of cervical cancer (HeLa). Respective UTR elements used are indicated therein within the sequence protocol (mRNA design (HSD17B4 (5′ UTR)/PSMB3 (3′ UTR))).

4.2 Formulation and Delivery of mRNA Encoding IL-12 Construct

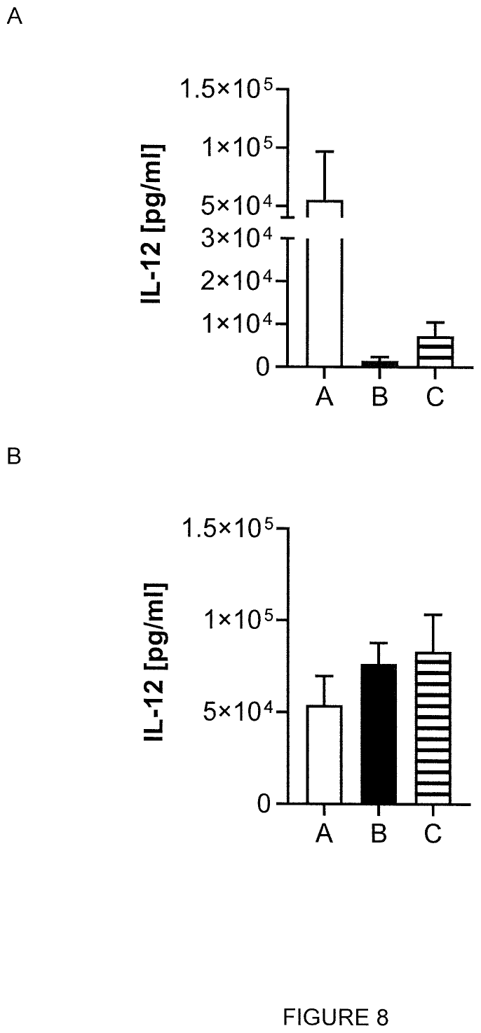

40.000 PHH were seeded in a collagen-coated 96-well flat bottom plate in triplicates on the day of transfection. The cells were transfected with 20 ng (FIG. 8A) of mRNA constructs using Lipofectamine® MessengerMAX. The RNA (ug):Lipofectamine® MessengerMAX (ul) ratio of 1:4 was used. After 24 hours cell supernatant was collected and frozen at −80° C.

10.000 HeLa were seeded in a 96-well flat bottom plate in triplicates on the day of transfection. The cell line was transfected with 50 ng (FIG. 8B) of mRNA constructs using Lipofectamine® 2000. The RNA (ug):Lipofectamine® 2000 (ul) ratio of 1:1.5 was used. After 24 hours supernatant was collected and frozen at −80° C.

### 4.3 ELISA

Active heterodimer II-12 was measured in supernatant by ELISA (Human II-12 p70 DuoSet ELISA). The capture antibody was diluted to the working concentration in PBS without carrier protein and used to coat a Nunc MaxiSorp® flat bottom 96-well plates (Thermo Fischer) with 100 μl per well overnight at room temperature. After coating, wells were washed three times (PBS pH 7.4 and 0.05% Tween-20) and blocked overnight in 300 μl blocking buffer (Reagent Diluent) at room temperature for 1 hour. All further incubations were carried out at room temperature. Afterwards, wells were washed three times and 100 μl of sample or standards in Reagent Diluent was added and incubated for 2 hours. Afterwards, wells were washed three times and 100 μl of the working dilution of Streptavidin-HRP (BD Pharmingen™, Cat. 554066, diluted 1:1000 in blocking buffer) was added into each well. The plate was covered and incubated for 20 minutes. After further washing steps, 100 μl of Substrate Solution was added to each well and incubated for 20 minutes. Then, 50 μl of Stop Solution was added. The absorbance was measured using a microplate reader at a wavelength of 450 nm.

TABLE XV
Single miRNA binding sites in mRNA encoding IL-12 transfected in PHH and HeLa

| | | | | | | | Time point of | |
|---|---|---|---|---|---|---|---|---|
| | | miRNA | 5′cap | | Position of miRNA | | analysis (PHH/ | SEQ ID |
| Group | mRNA ID | binding sites | structure | 3′ end | binding site | cell type | HeLa cells) | NO: |
| A | R7551 | / | Cap1 | A64 (Sapl) | / | PHH/HeLa | 24 h/20 h | 293 |
| B | R7555 | 1x 122-5p | Cap1 | A64 (Sapl) | Upstream of 5′ UTR | PHH/HeLa | 24 h/20 h | 295 |
| C | R7556 | 1x 122-5p | Cap1 | A64 (Sapl) | Downstream of 3′ UTR | PHH/HeLa | 24 h/20 h | 294 |

### 4.4 Summary of the Findings

Silencing capacities of the mRNA construct encoding IL-12 containing miRNA-122 binding site cloned before the 5′UTR was shown in PHH (FIG. 8A, Table XV). The comparison of the % expression of IL12 in PHH cells is shown in Table XVI. A stronger reduction in the expression of IL-12 could be shown with the incorporation of miRNA-122-5p binding site cloned before the 5′ UTR of the RNA constructs compared to the incorporation after the 3′ UTR. No reduction of the IL-12 expression from constructs containing miRNA-122 binding sites compared to the reference construct was seen in HeLa cells (FIG. 8B, Table XV).

TABLE XVI
Comparison of % expression of mRNA encoding
IL-12 comprising miRNA-122-5p binding site
transfected in PHH cells (FIG. 8A)

| | | | | Average | PpLuc |
|---|---|---|---|---|---|
| miRNA | Value | Value | Value | IL12B | expres- |
| binding site | 1 | 2 | 3 | [pg/ml] | sion |
| A no miRNA | 57609 | 12799 | 95614 | 55340.66667 | 100% |
| binding site | | | | | |
| B 1 × miRNA- | 2516 | 700 | 1293 | 1503 | 2.72% |
| 122-5p upstream | | | | | |
| of 5′ UTR | | | | | |
| C 1 × miRNA- | 7359 | 4098 | 10476 | 7311 | 13.21% |
| 122-5p down- | | | | | |
| stream of 3′ UTR | | | | | |

### Example 5: Regulation of PpLuc Expression with miRNA Binding Sites in Different Doses

### 5.1 Experimental Design

mRNA constructs encoding PpLuc were generated according to Example 1. miRNA binding site 122-5p was cloned in front of (upstream) the 5′UTR or following (downstream) the 3′UTR and the degree of silencing in correlation to the transfected dose of mRNA has been determined in PHH (see Table XVII). Respective UTR elements used are indicated therein within the sequence protocol (mRNA design (HSD17B4 (5′ UTR)/PSMB3 (3′ UTR))).

Every mRNA construct was transfected into primary human hepatocytes (PHH).

### 5.2 Formulation and Delivered of PpLuc Constructs

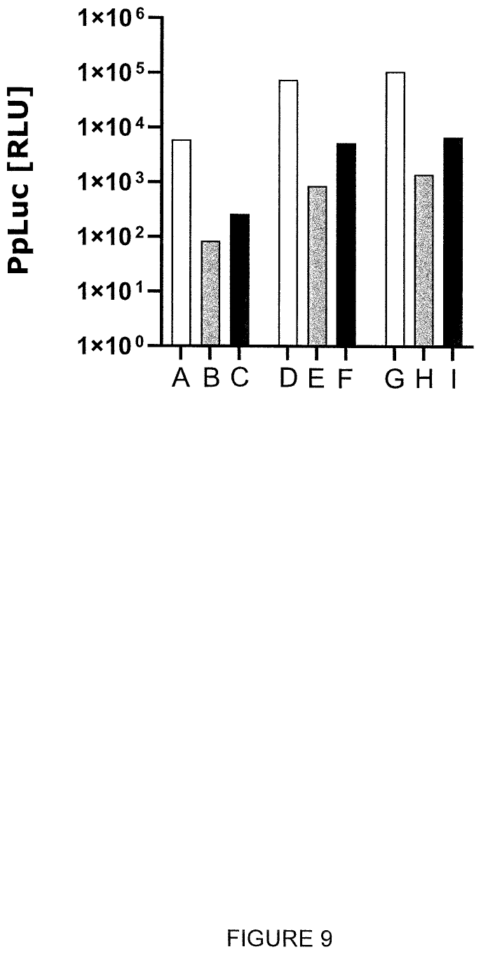

40.000 PHH were seeded in a collagen-coated 96-well flat bottom plate in triplicates. The cells were transfected with 10 ng, 50 ng or 100 ng (FIG. 9) of mRNA constructs using Lipofectamine® MessengerMAX. The RNA (ug):Lipofectamine® MessengerMAX (ul) ratio of 1:4 was used. Mock transfected cells served as negative control. After 24 hours cell lysates were prepared and frozen at −80° C. until the expression of luciferase was analyzed by luciferase assay as described in Example 2.3.

TABLE XVII
Single miRNA binding sites in mRNA encoding PpLuc transfected in PHH cells

| | miRNA | 5′cap | | Position of miRNA | Time point | SEQ ID |
|---|---|---|---|---|---|---|
| mRNA ID | binding site | structure | 3′ end | binding site | of analysis | NO: |
| R8730 | No binding site | Cap1 | hsl-A100 (Sapl) | / | 24 h | 292 |
| R9526 | 122-5p | Cap1 | hsl-A100 (Sapl) | Upstream of 5′ UTR | 24 h | 288 |
| R9531 | 122-5p | Cap1 | hsl-A100 (Sapl) | Downstream of 3′ UTR | 24 h | 291 |

### 5.3 Luciferase Assay

Luciferase assay to measure the expression activity was performed according to Example 2.3.

TABLE XVIII
Comparison of expression of mRNA encoding PpLuc transfected in different
doses comprising miRNA-122-5p binding site transfected in PHH (FIG. 9)

| | | | | | | | | Average |
|---|---|---|---|---|---|---|---|---|
| Group | mRNA ID | Dose | | miRNA binding site | Value 1 | Value 2 | Value 3 | [pg/ml] |
| A | R8730 | 10 | ng | no miRNA binding site | 8062 | 5618 | 4196 | 5959 |
| B | R9526 | 10 | ng | 1 x miRNA-122-5p | 131 | 68 | 55 | 85 |
| | | | | upstream of 5′ UTR | | | | |
| C | R9531 | 10 | ng | 1 x miRNA-122-5p | 275 | 304 | 211 | 263 |
| | | | | downstream of 3′ UTR | | | | |
| D | R8730 | 50 | ng | no miRNA binding site | 70143 | 62330 | 94094 | 75522 |
| E | R9526 | 50 | ng | 1 x miRNA-122-5p | 717 | 946 | 921 | 861 |
| | | | | upstream of 5′ UTR | | | | |
| F | R9531 | 50 | ng | 1 x miRNA-122-5p | 3857 | 4601 | 7438 | 5299 |
| | | | | downstream of 3′ UTR | | | | |
| G | R8730 | 100 | ng | no miRNA binding site | 174740 | 38207 | 109330 | 107426 |
| H | R9526 | 100 | ng | 1 x miRNA-122-5p | 2250 | 564 | 1461 | 1425 |
| | | | | upstream of 5′ UTR | | | | |
| I | R9531 | 100 | ng | 1 x miRNA-122-5p | 9611 | 3343 | 7511 | 6822 |
| | | | | downstream of 3′ UTR | | | | |

### 5.4 Summary of the Findings

Silencing capacities of the mRNA constructs encoding PpLuc containing miRNA-122 binding site cloned before (upstream) the 5′UTR and after (downstream) the 3TUTR was shown in all doses in PHH (FIG. 9, Table XVIII). A stronger reduction in the expression of PpLuc in liver cells could be shown with the incorporation of miRNA-122-5p binding site cloned before the 5′ UTR (FIG. 9, Group B, E, H) of the RNA constructs compared to the incorporation after the 3′ UTR (FIG. 9, Group C, F, 1). The mRNA constructs with miRNA binding sites before the 5′UTR showed more reduction in the PpLuc expression compared to the construct with the miRNA biding site after the 3′UTR or the control group, even in very high transfection doses (see FIG. 9, Group H). This finding indicates that miRNA binding sites incorporated before the 5′UTR can reduce offside target translation of mRNA in specific cell types (e.g. liver cells), independent of the administered dose.

### Example 6: Regulation of mRNA Expression with miRNA Binding Sites In Vivo

### 5.1 Experimental Design

mRNA constructs encoding PpLuc were generated according to Example 1, see Table XIX. MiRNA binding sites miR-122-5p, miR-142-3p and miR-223-3p were cloned in front of (upstream) the 5′UTR or following (downstream) the 3′UTR. Respective UTR elements used are indicated therein within the sequence protocol (mRNA design (HSD17B4 (5′ UTR)/PSMB3 (3′ UTR))). Additionally, the mRNA constructs were formulated in different lipid nanoparticles (see chapter 5.2 “Formulation into lipid nanoparticles” below).

TABLE XIX
Different miRNA binding sites in mRNA encoding PpLuc used for in vivo experiments

| | miRNA | | 5′cap | | Position of miRNA | SEQ ID |
|---|---|---|---|---|---|---|
| mRNA ID | binding site | Silencing | structure | 3′ end | binding site | NO: |
| R8730 | No binding site | / | Cap1 | hsl-A100 (Sapl) | / | 292 |
| R9526 | 122-5p | Liver | Cap1 | hsl-A100 (Sapl) | Upstream of 5′ UTR | 288 |
| R9531 | 122-5p | Liver | Cap1 | hsl-A100 (Sapl) | Downstream of 3′ UTR | 291 |
| R10121 | 142-3p | Immune cells | Cap1 | hsl-A100 (Sapl) | Upstream of 5′ UTR | 297 |
| R10118 | 142-3p | Immune cells | Cap1 | hsl-A100 (Sapl) | Downstream of 3′ UTR | 296 |
| R10120 | 223-3p | Immune cells | Cap1 | hsl-A100 (Sapl) | Upstream of 5′ UTR | 298 |
| R10119 | 223-3p | Immune cells | Cap1 | hsl-A100 (Sapl) | Downstream of 3′ UTR | 299 |

5.2 Formulation into Lipid Nanoparticles:

The individual mRNA-LNPs (see Table XX below) were prepared by mixing appropriate volumes of lipid stock solutions in ethanol buffer with an aqueous phase (50 mM sodium acetate, pH 4.0) containing appropriate amounts of mRNA as indicated herein; cholesterol, phospholipid and polymer conjugated lipid: 20 mg/ml in EtOH, cationic lipids and added to the ethanol premix of lipids. Briefly, mRNAs were diluted to 0.16 mg/ml to 50 mM acetate buffer, pH 4. Syringe pumps were installed into inlet parts of the Ignite™ (Precision NanoSystems Inc., Vancouver, BC) and used to mix the ethanolic lipid solution with the mRNA aqueous solution at a ratio of 1:3 (vol/vol) with total flow rates from about 20 mL/min. The ethanol was then removed and the external buffer replaced with PBS/sucrose buffer (pH 7.4, 75 mM NaCl, 10 mM phosphate, 150 mM sucrose) by dialysis (Slide-A-Lyzer™ Dialysis Cassettes, ThermoFisher). Finally, the lipid nanoparticles were up-concentrated (Vivaspin Turbo) and filtered through a 0.2 μm pore sterile filter.

TABLE XX
Lipid composition and lipid ratios of
lipid nanoparticles used in experiment

| | | | Admin- |
|---|---|---|---|
| | | | istration |
| LNP ID | Lipid composition | Lipid ratios | route |
| LNP-01 | SM-102:Chol:DSPC: | 48.5:38.9:11.1:1.5 | i.m. |
| | DMG-PEG 2000 | | |
| LNP-02 | HEXA-C5DE-PipSS:Chol: | 59:29.3:10:1.7 | i.v. |
| | DPhyPE:DMG-PEG 2000 | | |

5.3 In Vivo Delivery of PpLuc Constructs Comprising miRNA Binding Sites

### Intramuscular Administration:

mRNA formulated in LNPs (see Table XXI) were injected intramuscularly into both M. tibialis muscle of female Balb/c mice at a dose of 5 pg in a volume of 25 p1 per injection giving rise to a total dose of 10 pg per mouse. Mice were organized in two individual cohorts of four mice per cohort. Cohort 1 were sacrificed after six hours, and organs (liver, spleen, muscle and popliteal lymph nodes) were prepared. Cohort 2 were sacrificed after 24 hours, and organs were prepared. Prepared organs were frozen at −80° C.

TABLE XXI
miRNA binding sites in LNP formulated mRNA encoding PpLuc administered i.m.

| | | Timepoint of | | miRNA | 5′cap | | Position of miRNA | SEQ ID |
|---|---|---|---|---|---|---|---|---|
| Group | mRNA ID | measurement | LNP ID | binding site | structure | 3′ end | binding site | No: |
| A | R8730 | 6 h/24 h | LNP-01 | No binding site | Cap1 | hsl-A100 (Sapl) | / | 292 |
| B | R9526 | 6 h/24 h | LNP-01 | 122-5p | Cap1 | hsl-A100 (Sapl) | Upstream of 5′ UTR | 288 |
| C | R9531 | 6 h/24 h | LNP-01 | 122-5p | Cap1 | hsl-A100 (Sapl) | Downstream of 3′ UTR | 291 |

### Intravenous Administration:

mRNA formulated in LNPs (see Table XX) were injected intravenously into the tail vain of female Balb/c mice at a dose of 5 μg in a volume of 100 μl. Mice were organized in two individual cohorts of four mice per cohort. Cohort 1 were sacrificed after 4 hours, and liver and spleen were prepared. Cohort 2 were sacrificed after 24 hours, and liver and spleen were prepared. Prepared organs were frozen at −80° C.

TABLE XXII
miRNA binding sites in LNP formulated mRNA encoding PpLuc administered i.v.

| | | Time point | | | | | | | |
|---|---|---|---|---|---|---|---|---|---|
| | | of | LNP | miRNA | | 5′cap | | Position of miRNA | SEQ ID |
| Group | mRNA ID | measurement | formulation | binding site | Silencing | structure | 3′ end | binding site | No: |
| A | R8730 | 4/24 h | LNP-02 | No binding site | / | Cap1 | hsl-A100 (Sapl) | / | 292 |
| B | R9526 | 4/24 h | LNP-02 | 122-5p | Liver | Cap1 | hsl-A100 (Sapl) | Upstream of 5′ UTR | 288 |
| C | R9531 | 4/24 h | LNP-02 | 122-5p | Liver | Cap1 | hsl-A100 (Sapl) | Downstream of 3′ UTR | 291 |
| D | R10120 | 4/24 h | LNP-02 | 142-3p | Immune cells | Cap1 | hsl-A100 (Sapl) | Upstream of 5′ UTR | 297 |
| E | R10118 | 4/24 h | LNP-02 | 142-3p | Immune cells | Cap1 | hsl-A100 (Sapl) | Downstream of 3′ UTR | 296 |
| F | R10121 | 4/24 h | LNP-02 | 223-3p | Immune cells | Cap1 | hsl-A100 (Sapl) | Upstream of 5′ UTR | 298 |
| G | R10119 | 4/24 h | LNP-02 | 223-3p | Immune cells | Cap1 | hsl-A100 (Sapl) | Downstream of 3′ UTR | 299 |
| H | Mock | 4/24 h | — | — | — | — | — | — | |

5.4 Measuring Firefly Luciferase Activity from Organs

For measuring firefly luciferase activity frozen organs were treated in a bead mill (Tissue Lyzer II, Qiagen) followed by adding 1× lysis buffer (Passive lysis buffer, Cat El194A, Promega) in a ratio of 100 ml buffer per 10 mg of organ weight followed by a second treatment in a bead mill. Lysates were centrifuged and supernatants were used to measure firefly luciferase activity in a microplate reader (TriStar2 S LB 942, Berthold). The expression levels of Ppluc were obtained for individual organs, such as liver and spleens, or pooled such as for muscle or popliteal lymph nodes.

### 5.5 Summary of Findings

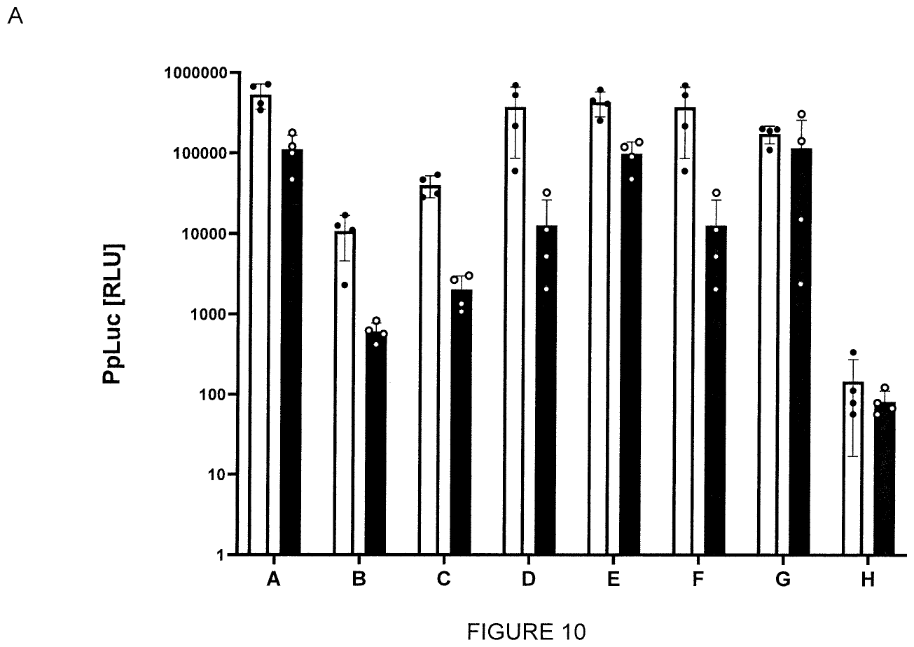

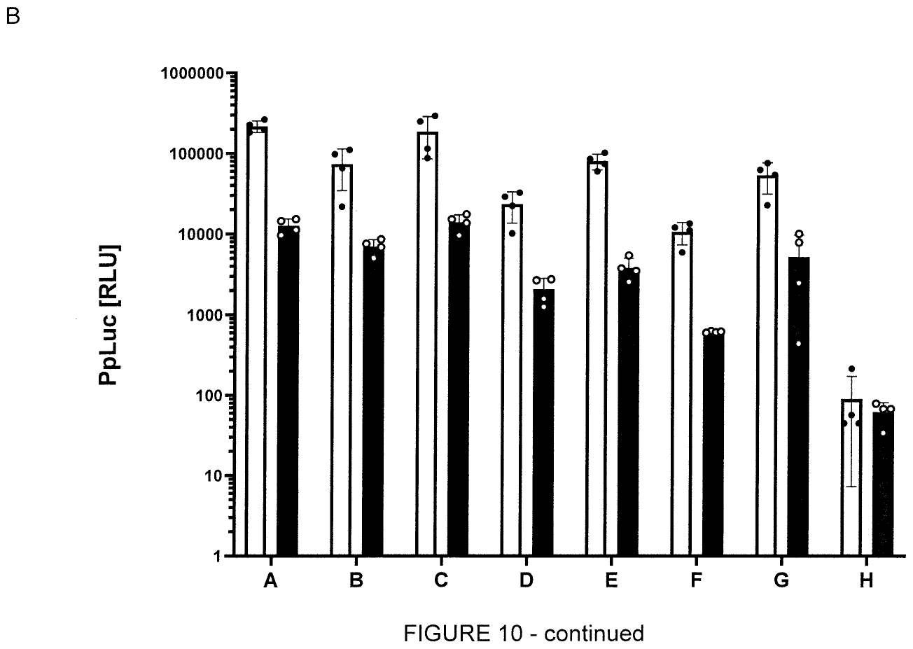

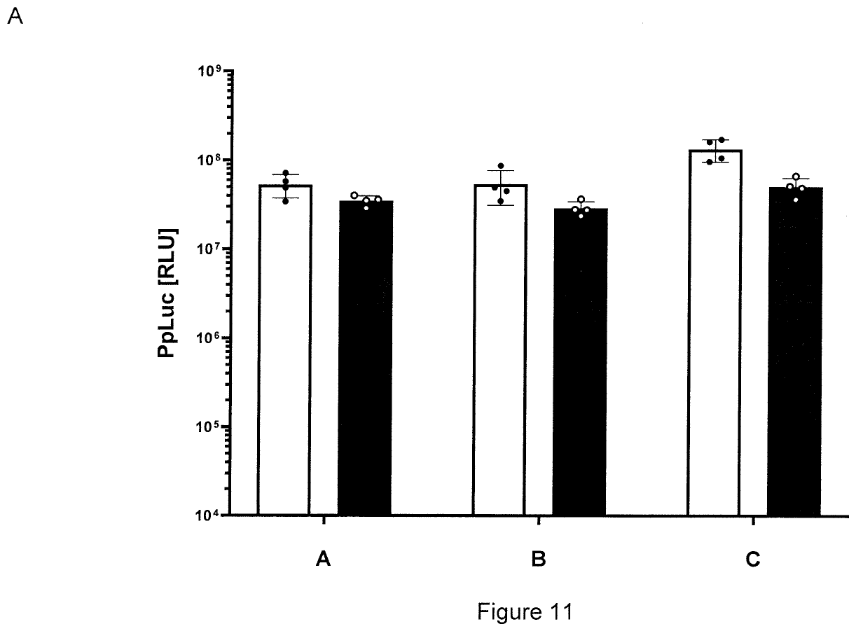

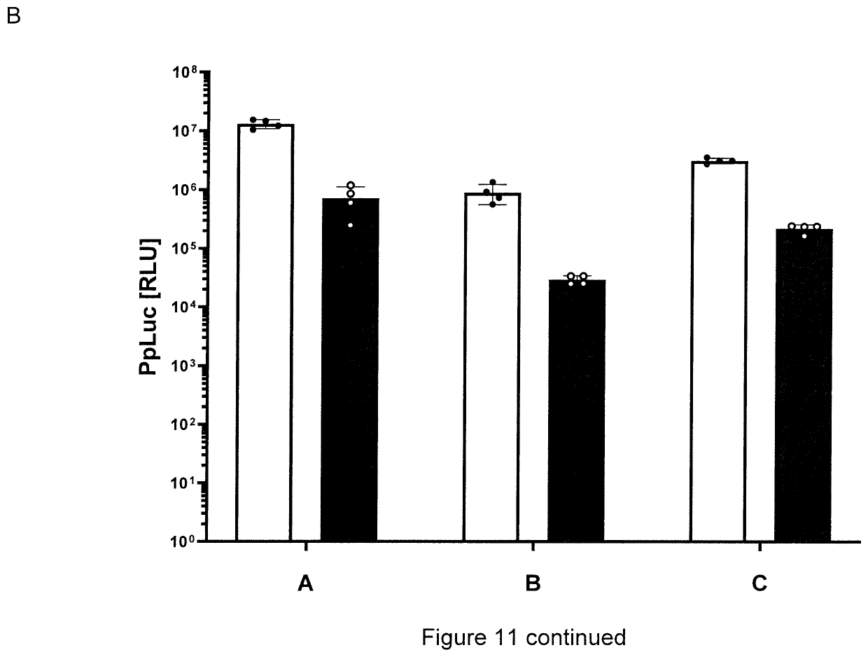

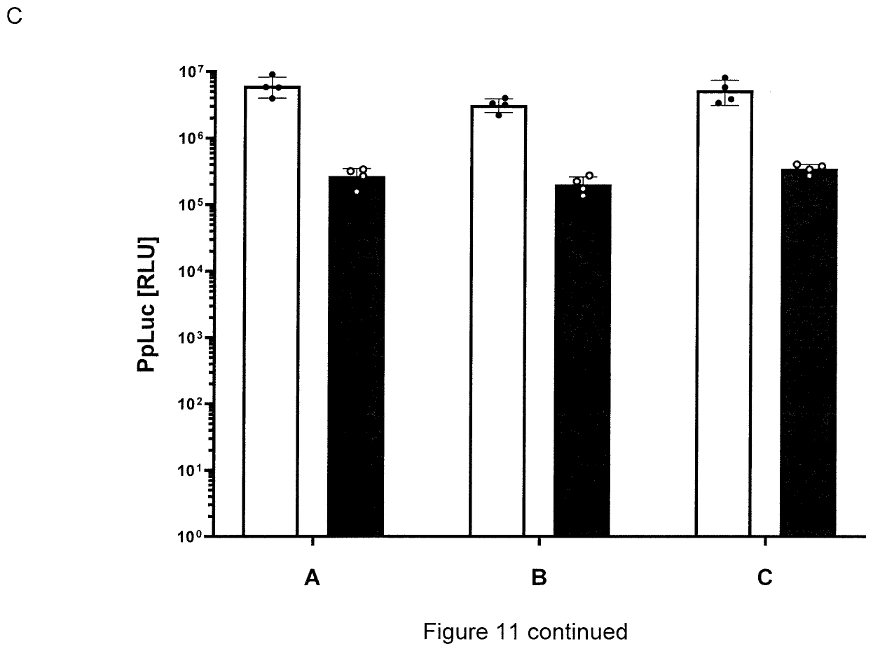

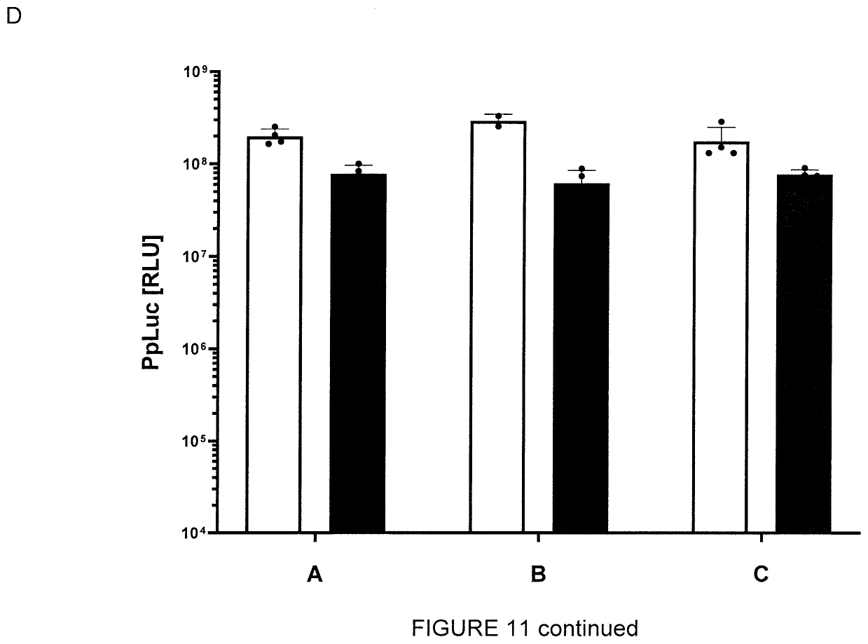

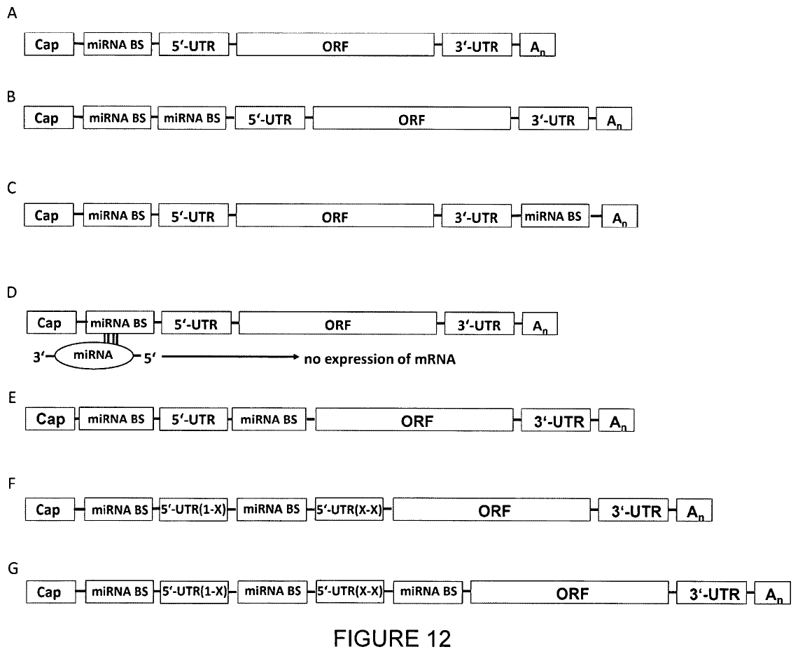

Stronger silencing capacities of the formulated mRNA construct encoding PpLuc comprising miRNA-122-5p binding site cloned before the 5′UTR (FIG. 10 and FIG. 11, Group B) compared to the formulated mRNA construct encoding PpLuc comprising miRNA-122 binding site cloned after the 3′UTR (FIG. 10 and FIG. 11, Group C) was shown in the liver after i.v. and i.m. injection (FIG. 10A and FIG. 11B). Both constructs containing miRNA-122-5p binding sites showed high expression levels in the muscle after i.m. administration (FIG. 11A, Group A and B).

The silencing effect of miRNA binding sites cloned before the 5′UTR was shown for the miRNA-142-3p (FIG. 10, Group D) and miRNA-223-3p (FIG. 10, Group F) as well.

No silencing effect of the miRNA-122-5p binding site in immune cells, independent of the position, was shown in the spleen after i.v. injection (FIG. 10B, Group B and C) and spleen and lymph nodes after i.m. injection (FIG. 11C and D, Group B and C). The immune cell silencing of the miRNA-binding sites miRNA-142-3p (FIG. 10, Group D and E) and miRNA-223-3p (FIG. 10, Group F and G) was shown in the spleen after i.v. injection, with a stronger silencing effect of the formulated mRNA constructs comprising the miRNA binding site cloned before the 5′UTR (FIG. 10, Group D and F) compared to the constructs with the miRNA biding site after the 3′UTR (FIG. 10, Group E and F). The immune cell silencing capacity of the miRNA-223-3p (FIG. 10, Group F) was stronger compared to the miRNA-142-3p (FIG. 10, Group D).

These findings can lead to mRNA constructs comprising different miRNA binding sites dependent to the administration route and/or indication/therapy and/or target protein encoded by the mRNA construct.

For (formulated) mRNA constructs encoding an antigen for vaccination, expression in the liver should avoided/reduced but expression in the immune cells might be useful to induce an immune response to the encoded antigen. These constructs should comprise at least one miRNA binding site with silencing capacity in the hepatocytes (liver), e.g. miRNA-122-5p.

For (formulated) mRNA constructs encoding a target protein which should not expressed in immune cells (e.g. protein replacement therapies, molecular therapy, therapy with cytotoxic or cytostatic proteins) the mRNA construct should comprise at least one miRNA binding site with silencing capacity in the hepatocytes (liver), e.g. miRNA-122-5p.

For (formulated) mRNA constructs encoding a target protein which should not expressed in the liver nor immune cells (e.g. protein replacement therapies, molecular therapy etc.) the mRNA construct should comprise at least one miRNA binding site with silencing capacity in the hepatocytes (liver), e.g. miRNA-122-5p and at least one miRNA biding site with silencing capacity in immune cells, e.g. miRNA-142-3p and/or miRNA-223-3p, preferably miRNA-223-3p.

1. A nucleic acid sequence comprising at least one coding region encoding at least one therapeutic peptide or protein and at least one first miRNA binding site sequence located in 5′ direction relative to the coding region.

2. The nucleic acid sequence of claim 1, wherein the nucleic acid sequence comprises at least two, three, or four first miRNA binding site sequences located in 5′ direction relative to the coding region.

3. The nucleic acid sequence of claim 1 or 2, additionally comprising at least one 5′ UTR

4. The nucleic acid sequence of claim 3, wherein the at least one 5′ UTR is selected or derived from a gene.

5. The nucleic acid sequence of claim 3 or 4, wherein the at least one first miRNA binding site sequence located in 5′ direction relative to the coding region is
I) located in 5′ direction relative to the 5′ UTR; and/or
II) is located within the 5′ UTR; and/or
III) located between the 5′ UTR and the coding region.

6. The nucleic acid sequence of claims 3 to 5, wherein the at least one first miRNA binding site sequence is located in 5′ direction relative to the 5′ UTR.

7. The nucleic acid sequence of claims 1 to 6, wherein the nucleic acid sequence comprises a 5′ terminal cap structure, preferably a cap1.

8. The nucleic acid sequence of claims 3 to 7, wherein the nucleic acid sequence comprises a 5′ terminal cap structure and the at least one first miRNA binding site sequence is located between said 5′ terminal cap structure and the 5′ UTR.

9. The nucleic acid sequence of claims 3 to 8, wherein at least one first miRNA binding site sequence is located in 5′ direction relative to the 5′ UTR and at least one first miRNA binding site sequence is located within the 5′ UTR.

10. The nucleic acid sequence of claims 3 to 9, wherein the at least one first miRNA binding site sequence is located in a distance of less than 20 nucleotides, less than 5 nucleotides, less than 1 nucleotide relative to the 5′ UTR.

11. The nucleic acid sequence of claims 1 to 10, wherein the at least one first miRNA binding site sequence comprises at least one miRNA binding site for reducing or preventing expression in liver, kidney, immune cells, or endothelial cells, or any combination thereof, preferably in liver cells and/or immune cells.

12. The nucleic acid sequence of any of the preceding claims, wherein the at least one first miRNA binding site sequence comprises one or more of the group consisting of binding sites for miRNA-122, miRNA-142, miRNA-148a, miRNA-101, miRNA-192, miRNA-194, and miRNA-223.

13. The nucleic acid sequence of claim 12, wherein the at least one first miRNA binding site sequence comprises one or more of miRNA-122, miRNA-148a, and miRNA-223.

14. The nucleic acid sequence of any of the preceding claims, wherein the at least one first miRNA binding site sequence comprises one or more of the group consisting of the binding sites for miRNA-122-5p, miRNA-142-3p, miRNA-148a-3p, miRNA-101-3p, miRNA-192-5p, miRNA-194-5p, and miRNA-223-3p.

15. The nucleic acid sequence of claim 14, wherein the at least one first miRNA binding site sequence comprises one or more of miRNA-122-5p, miRNA-148a-3p, and miRNA-223-3p.

16. The nucleic acid sequence of any of the preceding claims, wherein the at least one first miRNA binding site sequence comprises at least one, two, three, or four miRNA binding sites.

17. The nucleic acid sequence of claim 16, wherein the at least one, two, three, or four miRNA binding sites are selected from substantially similar miRNA binding sites.

18. The nucleic acid sequence of claim 16 or 17, wherein the at least one, two, three, or four miRNA binding sites are selected from substantially different miRNA binding sites.

19. The nucleic acid sequence of any of the preceding claims, wherein the at least one first miRNA binding site sequence comprises or consists of at least two or three miRNA-122 binding sites, preferably miRNA-122-5p.

20. The nucleic acid sequence of any of the preceding claims, wherein the at least one first miRNA binding site sequence comprises or consists of at least two or three miRNA-148a binding sites, preferably miRNA-148a-3p.

21. The nucleic acid sequence of any of the preceding claims, wherein the at least one first miRNA binding site sequence comprises or consists of at least two or three miRNA-223 binding sites, preferably miRNA-223-3p.

22. The nucleic acid sequence of any of the preceding claims, wherein the at least one first miRNA binding site sequence comprises or consists of a nucleic acid sequence selected or derived from SEQ ID No 249, SEQ ID No 250, SEQ ID No 251, SEQ ID No 252, SEQ ID No 253, SEQ ID No 254, SEQ ID No 255, SEQ ID No 256, SEQ ID No 257 or SEQ ID No 258, SEQ ID No: 300, SEQ ID No: 301, SEQ ID No: 302, SEQ ID No: 303, or a fragment or variant of any of these.

23. The nucleic acid sequence of claim 22, wherein the at least one first miRNA binding site sequence comprises or consists of a nucleic acid sequence selected or derived from SEQ ID NO: 249, SEQ ID NO: 252, SEQ ID NO: 303, or a fragment or variant of any of these.

24. The nucleic acid sequence of any of the preceding claims, additionally comprising at least one second miRNA binding site sequence located in 3′ direction relative to the coding region.

25. The nucleic acid sequence of claim 24, wherein the nucleic acid sequence comprises at least two, three, or four second miRNA binding site sequences located in 3′ direction relative to the coding region.

26. The nucleic acid sequence of claim 24 or 25, additionally comprising at least one 3′ UTR

27. The nucleic acid sequence of claim 26, wherein the at least one 3′ UTR is selected or derived from a gene.

28. The nucleic acid sequence of claim 26 or 27, wherein the at least one second miRNA binding site sequence located in 3′ direction relative to the coding region is
I) located in 3′ direction relative to the 3′ UTR; and/or
II) is located within the 3′ UTR; and/or
III) located between the 3′ UTR and the coding region.

29. The nucleic acid sequence of claims 26 to 28, wherein the at least one second miRNA binding site sequence is located in 3′ direction relative to the 3′ UTR.

30. The nucleic acid sequence of to any of the preceding claims, wherein the nucleic acid sequence comprises at least one poly(A) sequence, and/or at least one poly(C) sequence, and/or at least one histone stem-loop sequence/structure.

31. The nucleic acid sequence of any of the preceding claims, wherein the nucleic acid sequence comprises at least one poly(A) sequence, preferably comprising about 40 to about 200 adenosine nucleotides, most preferably about 100 adenosine nucleotides.

32. The nucleic acid sequence of claim 26 to 31, wherein the nucleic acid sequence comprises at least one poly(A) sequence and the at least one second miRNA binding site sequence is located between the poly(A) sequence and the 3′ UTR.

33. The nucleic acid sequence of claims 26 to 32, wherein at least one second miRNA binding site sequence is located in 3′ direction relative to the 3′ UTR and at least one second miRNA binding site sequence is located within the 3′ UTR.

34. The nucleic acid sequence of claims 26 to 33, wherein the at least one second miRNA binding site sequence is located in a distance of less than 20 nucleotides, less than 5 nucleotides, less than 1 nucleotide relative to the 3′ UTR.

35. The nucleic acid sequence of claims 24 to 34, wherein the at least one second miRNA binding site sequence comprises at least one miRNA binding site for reducing or preventing expression in liver, kidney, immune cells, or endothelial cells, or any combination thereof, preferably in liver cells and/or immune cells.

36. The nucleic acid sequence of claims 24 to 35, wherein the at least one second miRNA binding site sequence comprises one or more of the group consisting of binding sites for miRNA-122, miRNA-142, miRNA-148a, miRNA-101, miRNA-192, miRNA-194, and miRNA-223.

37. The nucleic acid sequence of claim 36, wherein the at least one second miRNA binding site sequence comprises one or more of miRNA-122, miRNA-192 and miRNA-194.

38. The nucleic acid sequence according to claims 24 to 37, wherein the at least one second miRNA binding site sequence comprises one or more of the group consisting of the binding sites for miRNA-122-5p, miRNA-142-3p, miRNA-148a-3p, miRNA-101-3p, miRNA-192-5p, miRNA-194-5p, miRNA-223-3p.

39. The nucleic acid sequence of claim 38, wherein the at least one second miRNA binding site sequence comprises one or more of miRNA-122-5p and/or miRNA-192-5p and/or miRNA-194-5p.

40. The nucleic acid sequence of claims 24 to 39, wherein the at least one second miRNA binding site sequence comprises at least one, two, three, or four miRNA binding sites.

41. The nucleic acid sequence of claim 40, wherein the at least one, two, three, or four miRNA binding sites are selected from substantially similar miRNA binding sites.

42. The nucleic acid sequence of claim 40 or 41, wherein the at least one, two, three, or four miRNA binding sites are selected from substantially different miRNA binding sites.

43. The nucleic acid sequence of claims 24 to 42, wherein the at least one second miRNA binding site sequence comprises or consists of at least two or three miRNA-122 binding sites, preferably miRNA-122-5p.

44. The nucleic acid sequence of claims 24 to 43, wherein the at least one second miRNA binding site sequence comprises or consists of at least two or three miRNA-192 binding sites, preferably miRNA-192-5p.

45. The nucleic acid sequence of claims 24 to 44, wherein the at least one second miRNA binding site sequence comprises or consists of at least two or three miRNA-194 binding sites, preferably miRNA-194-5p.

46. The nucleic acid sequence of claims 24 to 45, wherein the at least one second miRNA binding site sequence comprises or consists of a nucleic acid sequence selected or derived from SEQ ID No 249, SEQ ID No 250, SEQ ID No 251, SEQ ID No 252, SEQ ID No 253, SEQ ID No 254, SEQ ID No 255, SEQ ID No 256, SEQ ID No 257 or SEQ ID No 258, SEQ ID No: 300, SEQ ID No: 301, SEQ ID No: 302, SEQ ID No: 303 or a fragment or variant of any of these.

47. The nucleic acid sequence of claim 46, wherein the at least one second miRNA binding site sequence comprises or consists of a nucleic acid sequence selected or derived from SEQ ID NO: 249, SEQ ID NO: 255, SEQ ID NO 257, or a fragment or variant of any of these.

48. The nucleic acid sequence of any of the preceding claims, wherein the at least one coding region is a codon modified coding sequence, wherein the amino acid sequence encoded by the at least one codon modified coding sequence is preferably not being modified compared to the amino acid sequence encoded by the corresponding reference or original coding sequence.

49. The nucleic acid sequence of claim 48, wherein the at least one codon modified coding sequence is selected from a C increased coding sequence, a CAI increased coding sequence, a human codon usage adapted coding sequence, a G/C content modified coding sequence, or a G/C optimized coding sequence, or any combination thereof, preferably a G/C optimized coding sequence.

50. The nucleic acid sequence of any of the preceding claims, wherein the nucleic acid sequence comprises at least one modified nucleotide and/or at least one nucleotide analogue or nucleotide derivative.

51. The nucleic acid sequence of claim 50, wherein the at least one modified nucleotide and/or at least one nucleotide analogues is selected from a backbone modified nucleotide, a sugar modified nucleotide and/or a base modified nucleotide, or any combination thereof.

52. The nucleic acid sequence of claim 50 or 51, wherein the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, N-1-methylpseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, 2-aminoadenosine or 5-(isopentenylaminomethyl)-2′-O-methyluridine or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or O(6)-methylguanine.

53. The nucleic acid sequence of claims 50 to 52, wherein the least one modified nucleotide and/or the at least one nucleotide analog is selected from pseudouridine or N-1-methylpseudouridine.

54. The nucleic acid sequence of claims 1 to 49, wherein the nucleic acid sequence consists of non-modified nucleotides and optionally comprises a 5′ terminal cap structure.

55. The nucleic acid sequence of claims 3 to 54, wherein the at least one 5′-UTR and/or the at least one 3′-UTR are heterologous UTRs.

56. The nucleic acid sequence of claims 3 to 55, wherein the at least one 5′-UTR comprises a nucleic acid sequence selected or derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

57. The nucleic acid sequence of claim 56, wherein the at least one 5′-UTR comprises a nucleic acid sequence selected or derived from a 5′-UTR of a gene selected from HSD17B4, or from a homolog, a fragment or variant of that gene.

58. The nucleic acid of any of the preceding claims, wherein the nucleic acid sequence comprises a HSD17B4 5′-UTR and at least one miRNA binding site sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 304-342, or fragments or variants of any of these.

59. The nucleic acid sequence of claims 3 to 58, wherein the at least one 3′-UTR comprises a nucleic acid sequence selected or derived from a 3′-UTR of a gene selected from PSMB3, ALB7, alpha-globin, CASP1, COX6B1, GNAS, NDUFA1 and RPS9 or from a homolog, a fragment or a variant of any one of these genes.

60. The nucleic acid sequence of claim 59, wherein nucleic acid sequence the at least one 3′-UTR comprises a nucleic acid sequence selected or derived from a 3′-UTR of a gene selected from PSMB3 or from a homolog, a fragment or a variant of that gene.

61. The nucleic acid sequence of any of the preceding claims, wherein the nucleic acid sequence comprises a PSMB3 3′-UTR and at least one miRNA binding site sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 343-347, SEQ ID NO: 352-377, SEQ ID NO: 379-381, or fragments or variants of any of these.

62. The nucleic acid sequence of any of the preceding claims, wherein the nucleic acid sequence is selected from DNA or RNA, preferably from plasmid DNA, viral DNA, template DNA, viral RNA, self-replicating RNA, circular RNA, replicon RNA, or an mRNA.

63. The nucleic acid sequence of any of the preceding claims, wherein the nucleic acid sequence is a linear nucleic acid, preferably a single-stranded linear nucleic acid.

64. The nucleic acid sequence of any of the preceding claims, wherein the nucleic acid is not selected or derived from an adenoviral vector or wherein the nucleic acid is not isolated from a cell, tissue, or organism.

65. The nucleic acid sequence of any of the preceding claims, wherein the nucleic acid is an in vitro transcribed RNA.

66. The nucleic acid sequence of any of the preceding claims, wherein the nucleic acid is an mRNA, preferably an in vitro transcribed mRNA.

67. The nucleic acid sequence of any of the preceding claims, wherein the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, a viral antigen, a bacterial antigen, a protozoan antigen, an allergen, an autoimmune antigen, a tumor antigen, cytostatic or cytotoxic peptide or protein, or fragments, variants, or combinations of any of these.

68. The nucleic acid sequence of any of the preceding claims, wherein the miRNA binding site sequence allows a cell type specific expression from the nucleic acid sequence within a target organ or organs.

69. The nucleic acid sequence of any of the preceding claims, wherein the protein expression of the nucleic acid sequence is reduced in the liver.

70. The nucleic acid sequence of any of the preceding claims, wherein the at least one first miRNA binding site sequence comprises at least one miRNA binding site for reducing or preventing protein expression in the liver.

71. The nucleic acid sequence of claim 69 or 70, wherein the at least one first miRNA binding site sequence comprises at least on miRNA binding site for reducing or preventing protein expression in the liver is selected or derived from one or more of the group consisting of binding sites for miRNA-122, miRNA-148a, miRNA-101, miRNA-192, miRNA-194.

72. The nucleic acid sequence of claim 71, wherein the at least one first miRNA binding site sequence for reducing or preventing expression in liver comprises or consists of at least one binding site for miRNA-122, preferably miRNA-122-5p.

73. The nucleic acid sequence of claim 71 or 72, wherein the at least one first miRNA binding site sequence for reducing or preventing expression in liver comprises or consists of at least one binding site for miRNA-148a, preferably miRNA-148a-3p.

74. The nucleic acid sequence of claims 69 to 73, wherein the at least one first miRNA binding site sequence is located in 5′ direction relative to the coding sequence, preferably relative to a 5′ UTR, wherein the miRNA binding site sequence comprises one or more miRNA-122 and/or miRNA-148a binding sites.

75. The nucleic acid sequence of claims 69 to 74, comprising
I) at least one 5′ UTR preferably selected or derived from a gene;
II) at least one first miRNA binding site sequence located in 5′ direction relative to the 5′ UTR, wherein the miRNA binding site sequence comprises or consists of at least two or three miRNA-122 binding sites.

76. The nucleic acid sequence of claims 69 to 74, comprising
I) at least one 5′ UTR preferably selected or derived from a gene;
II) at least one first miRNA binding site sequence located in 5′ direction relative to the 5′ UTR comprising or consisting of at least one miRNA-122 binding site and at least one first miRNA binding site sequence located within the 5′ UTR comprising or consisting of at least one miRNA-122 binding site.

77. The nucleic acid sequence of claims 69 to 76, additionally comprising at least one second miRNA binding site sequence that comprises at least one miRNA binding site for reducing or preventing protein expression in the liver.

78. The nucleic acid sequence of claim 77, wherein the at least one second miRNA binding site sequence comprises at least on miRNA binding site for reducing or preventing protein expression in the liver is selected or derived from one or more of the group consisting of binding sites for miRNA-122, miRNA-148a, miRNA-101, miRNA-192, miRNA-194.

79. The nucleic acid sequence of claim 78, wherein the at least one second miRNA binding site sequence for reducing or preventing expression in liver comprises or consists of at least one binding site for miRNA-122, preferably miRNA-122-5p.

80. The nucleic acid sequence of claim 78 or 79, wherein the at least one second miRNA binding site sequence for reducing or preventing expression in liver comprises or consists of at least one binding site for miRNA-192, preferably miRNA-192-5p.

81. The nucleic acid sequence of claims 78 to 80, wherein the at least one second miRNA binding site sequence for reducing or preventing expression in liver comprises or consists of at least one binding site for miRNA-194, preferably miRNA-194-5p.

82. The nucleic acid sequence of claims 77 to 81, wherein the at least one second miRNA binding site sequence is located in 3′ direction relative to the coding sequence, preferably relative to a 3′ UTR, wherein the at least one second miRNA binding site sequence comprises one or more miRNA-122 binding sites and/or miRNA-192 binding sites and/or miRNA-194 binding sites.

83. The nucleic acid sequence of claims 77 to 82, comprising
I) at least one 3′ UTR preferably selected or derived from a gene;
II) at least one second miRNA binding site sequence located in 3′ direction relative to the 3′ UTR, wherein the at least one second miRNA binding site sequence comprises at least one miRNA-192 binding sites and/or at least one miRNA-194 binding sites.

84. The nucleic acid sequence of claims 69 to 83, wherein the nucleic acid comprises at least one first miRNA binding site sequence as defined in any one of the claims 69 to 76 and at least one second miRNA binding site sequence as defined in any one of the claims 77 to 83.

85. The nucleic acid sequence of claims 69 to 84, comprising,
I) at least one 5′ UTR preferably selected or derived from a gene;
II) at least one first miRNA binding site sequence located in 5′ direction relative to the 5′ UTR, wherein the at least one first miRNA binding site sequence comprises one or more miRNA-122 and/or miRNA-148a binding sites.
III) at least one 3′ UTR preferably selected or derived from a gene;
IV) at least one second miRNA binding site sequence located in 3′ direction relative to the 3′ UTR, wherein the at least one second miRNA binding site sequence comprises one or more miRNA-122 binding sites and/or miRNA-192 binding sites and/or miRNA-194 binding sites.

86. The nucleic acid sequence of claims 69 to 85, comprising
I) at least one 5′ UTR preferably selected or derived from a gene;
II) at least one first miRNA binding site sequence located in 5′ direction relative to the 5′ UTR, wherein the at least one first miRNA binding site sequence comprises at least two or three miRNA-122 binding sites;
III) at least one 3′ UTR preferably selected or derived from a gene;
IV) at least one second miRNA binding site sequence located in 3′ direction relative to the 3′ UTR, wherein the at least one second miRNA binding site sequence comprises one or more miRNA-122 binding sites and/or miRNA-192 binding sites and/or miRNA-194 binding sites.

87. The nucleic acid sequence of claims 69 to 85, comprising
I) at least one 5′ UTR preferably selected or derived from a gene;
II) at least one first miRNA binding site sequence located in 5′ direction relative to the 5′ UTR, wherein the at least one first miRNA binding site sequence comprises at least two or three miRNA-122 binding sites;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-192 binding site.

88. The nucleic acid sequence of claims 69 to 85, comprising
I) at least one 5′ UTR preferably selected or derived from a gene;
II) at least one first miRNA binding site sequence located in 5′ direction relative to the 5′ UTR, wherein the at least one first miRNA binding site sequence comprises at least two or three miRNA-122 binding sites;
III) at least one 3′ UTR preferably selected or derived from a gene;
IV) a second miRNA binding site sequence located in 3′ direction relative to the 3′ UTR, wherein the second miRNA binding site sequence comprises at least one miRNA-194 binding site.

89. The nucleic acid sequence of claims 69 to 85, comprising
I) at least one 5′ UTR preferably selected or derived from a gene;
II) at least one first miRNA binding site sequence located in 5′ direction relative to the 5′ UTR comprising or consisting of at least one miRNA-122 binding site and at least one first miRNA binding site sequence located within the 5′ UTR comprising or consisting of at least one miRNA-122 binding site;
III) at least one 3′ UTR preferably selected or derived from a gene;
IV) at least one second miRNA binding site sequence located in 3′ direction relative to the 3′ UTR, wherein the at least one second miRNA binding site sequence comprises one or more miRNA-122 binding sites and/or miRNA-192 binding sites and/or miRNA-194 binding sites.

90. The nucleic acid sequence of claims 69 to 85, comprising
I) at least one 5′ UTR preferably selected or derived from a gene;
II) at least one first miRNA binding site sequence located in 5′ direction relative to the 5′ UTR comprising or consisting of at least one miRNA-122 binding site and at least one first miRNA binding site sequence located within the 5′ UTR comprising or consisting of at least one miRNA-122 binding site;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-192 binding site.

91. The nucleic acid sequence of claims 69 to 85, comprising
I) at least one 5′ UTR preferably selected or derived from a gene;
II) at least one first miRNA binding site sequence located in 5′ direction relative to the 5′ UTR comprising or consisting of at least one miRNA-122 binding site and at least one first miRNA binding site sequence located within the 5′ UTR comprising or consisting of at least one miRNA-122 binding site;
III) at least one 3′ UTR preferably selected or derived from a gene;
IV) at least one second miRNA binding site sequence located in 3′ direction relative to the 3′ UTR, wherein the at least one second miRNA binding site sequence comprises at least one miRNA-194 binding site.

92. The nucleic acid sequence of claims 69 to 91, wherein the encoded peptide or protein is selected or derived from an antigen or epitope of an antigen.

93. The nucleic acid of claim 92, wherein the antigen or epitope of an antigen is selected from a pathogen antigen, preferably a viral antigen, a bacterial antigen

94. The nucleic acid of claim 92, wherein the antigen or epitope of an antigen is selected from a tumor antigen.

95. The nucleic acid sequence of claims 69 to 94, wherein the 5′ UTR is selected or derived from HSD17B4 and wherein the 3′ UTR is selected or derived from PSMB3 and wherein the nucleic acid additionally comprises at least one 5′ Cap structure, preferably a Cap1, and at least one 3′ terminal Poly(A) Sequence

96. The nucleic acid sequence claims 69 to 95, wherein upon administration of the nucleic acid to a cell or subject the expression of the encoded peptide or protein is reduced in the liver by at least 10%, 20%, 30%, 40%, 50%, 60%, 70%, 80% or 90% compared to a reference nucleic acid lacking the respective miRNA binding site sequence.

97. The nucleic acid sequence claims 69 to 96, wherein upon administration of the nucleic acid to a cell or subject, the encoded peptide or protein is expressed in non-liver cells, preferably in immune cells or muscle cells.

98. The nucleic acid sequence claims 69 to 97, wherein upon administration of the nucleic acid to a cell or subject, 80%, 85%, 90%, 95%, 96%, 97%, 98%, 99% of the expressed peptide or protein is produced in non-liver cells, preferably in immune cells or muscle cells.

99. The nucleic acid sequence claims 69 to 98, wherein the administration is an intramuscular administration.

100. The nucleic acid sequence of claims 1 to 69, wherein the protein expression of the nucleic acid sequence is reduced in immune cells.

101. The nucleic acid sequence of any of the preceding claims, wherein the at least one first miRNA binding site sequence comprises at least one miRNA binding site for reducing or preventing protein expression in immune cells.

102. The nucleic acid sequence of claim 100 or 101, wherein the at least one first miRNA binding site sequence comprises at least on miRNA binding site for reducing or preventing protein expression in immune cells selected or derived from miRNA-142 and miRNA-223.

103. The nucleic acid sequence of claims 100 to 102, wherein the at least one first miRNA binding site sequence for reducing or preventing expression in immune cells comprises or consists of at least one binding site for miRNA-223, preferably miRNA-223-3p.

104. The nucleic acid sequence of claims 100 to 103, wherein the at least one first miRNA binding site sequence for reducing or preventing expression in immune cells comprises or consists of at least one binding site for miRNA-142, preferably miRNA-142-3p.

105. The nucleic acid sequence of claim 100 to 104, wherein the at least one first miRNA binding site sequence is located in 5′ direction relative to the coding sequence, preferably relative to a 5′ UTR, wherein the miRNA binding site sequence comprises miRNA-142 and/or miRNA-223 binding sites.

106. The nucleic acid sequence of claim 100 to 105, comprising
I) at least one 5′ UTR preferably selected or derived from a gene;
II) at least one first miRNA binding site sequence located in 5′ direction relative to the 5′ UTR, wherein the at least one first miRNA binding site sequence comprises at least one miRNA-142 binding sites and/or at least one miRNA-223 binding sites.

107. The nucleic acid sequence of claim 100 to 106, additionally comprising at least one second miRNA binding site sequence that comprises at least one miRNA binding site for reducing or preventing protein expression in immune cells.

108. The nucleic acid sequence of claim 107, wherein the at least one second miRNA binding site sequence comprises at least on miRNA binding site for reducing or preventing protein expression in immune cells is selected or derived from miRNA-142 and miRNA-223.

109. The nucleic acid sequence of claim 107 or 108, wherein the at least one second miRNA binding site sequence for reducing or preventing expression in immune cells comprises or consists of at least one binding site for miRNA-223, preferably miRNA-223-3p.

110. The nucleic acid sequence of claims 107 to 109, wherein the at least one second miRNA binding site sequence for reducing or preventing expression in immune cells comprises or consists of at least one binding site for miRNA-142, preferably miRNA-142-3p.

111. The nucleic acid sequence of claim 107 to 110, wherein the at least one second miRNA binding site sequence is located in 5′ direction relative to the coding sequence, preferably relative to a 5′ UTR, wherein the miRNA binding site sequence comprises miRNA-142 and/or miRNA-223 binding sites.

112. The nucleic acid sequence of claim 100 to 105, comprising
I) at least one 5′ UTR preferably selected or derived from a gene;
II) at least one first miRNA binding site sequence located in 5′ direction relative to the 5′ UTR, wherein the at least one first miRNA binding site sequence comprises at least one miRNA-142 binding sites and/or at least one miRNA-223 binding sites.

113. The nucleic acid sequence of claim 100 to 112, wherein the nucleic acid comprises at least one first miRNA binding site sequence as defined in any one of the claims 100 to 106 and at least one second miRNA binding site sequence as defined in any one of the claims 107 to 112.

114. The nucleic acid sequence of claim 100 to 113, wherein the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, cytostatic or cytotoxic peptide or protein, or fragments, variants, or combinations of any of these.

115. The nucleic acid sequence of claim 100 to 114, wherein the 5′ UTR is selected or derived from HSD17B4 and wherein the 3′ UTR is selected or derived from PSMB3 and wherein the nucleic acid additionally comprises at least one 5′ Cap structure, preferably a Cap1, and at least one 3′ terminal Poly(A) Sequence

116. The nucleic acid sequence claim 100 to 115, wherein upon administration of the nucleic acid to a cell or subject the expression of the encoded peptide or protein is reduced in immune cells by at least 10%, 20%, 30%, 40%, 50%, 60%, 70%, 80% or 90% compared to a reference nucleic acid lacking the respective miRNA binding site sequence.

117. The nucleic acid sequence claim 100 to 115, wherein upon administration of the nucleic acid to a cell or subject, the encoded peptide or protein is expressed in non-immune cells, preferably in liver cells.

118. The nucleic acid sequence claim 100 to 116, wherein upon administration of the nucleic acid to a cell or subject, 80%, 85%, 90%, 95%, 96%, 97%, 98%, 99% of the expressed peptide or protein is produced in non-immune cells, preferably in the liver.

119. The nucleic acid sequence claim 100 to 117, wherein the administration is an intravenous administration.

120. The nucleic acid sequence of any of the preceding claims, wherein the protein expression of the nucleic acid sequence is reduced in liver cells and in immune cells.

121. The nucleic acid sequence of any of the preceding claims, wherein the at least one first miRNA binding site sequence comprises at least one miRNA binding site for reducing or preventing protein expression in liver cells and/or immune cells, preferably wherein the at least one first miRNA binding site sequence is located in 5′ direction relative to the coding sequence.

122. The nucleic acid sequence of claim 120 or 121, wherein the at least one first miRNA binding site sequence comprises at least on miRNA binding site for reducing or preventing protein expression in the liver is selected or derived from one or more of the group consisting of binding sites for miRNA-122, miRNA-148a, miRNA-101, miRNA-192, miRNA-194, preferably miRNA-122 and/or miRNA-148a binding sites.

123. The nucleic acid sequence of claims 120 to 122, wherein the at least one first miRNA binding site sequence comprises at least on miRNA binding site for reducing or preventing protein expression in immune cells selected or derived from miRNA-142 and miRNA-223.

124. The nucleic acid sequence of claim 120 to 123, additionally comprising at least one second miRNA binding site sequence that comprises at least one miRNA binding site for reducing or preventing protein expression in liver cells and/or immune cells, preferably wherein the at least one second miRNA binding site sequence is located in 3′ direction relative to the coding sequence.

125. The nucleic acid sequence of claim 124, wherein the at least one second miRNA binding site sequence comprises at least on miRNA binding site for reducing or preventing protein expression in the liver is selected or derived from one or more of the group consisting of binding sites for miRNA-122, miRNA-148a, miRNA-101, miRNA-192, miRNA-194, preferably miRNA-122 binding sites and/or miRNA-192 binding sites and/or miRNA-194 binding sites.

126. The nucleic acid sequence of claim 124 or 125, wherein the at least one second miRNA binding site sequence comprises at least on miRNA binding site for reducing or preventing protein expression in immune cells selected or derived from miRNA-142 and miRNA-223.

127. The nucleic acid sequence of claim 120 to 126, wherein the nucleic acid comprises at least one first miRNA binding site sequence as defined in any one of the claims 120 to 123 and at least one second miRNA binding site sequence as defined in any one of the claims 124 to 126.

128. The nucleic acid sequence of claim 120 to 127, wherein the nucleic acid comprises at least one first miRNA binding site sequence for reducing or preventing protein expression in liver as defined in any one of claims 70 to 83 and at least one second miRNA binding site sequence for reducing or preventing protein expression in immune cells as defined in any one of the claims 107 to 112.

129. The nucleic acid sequence of claim 120 to 127, wherein the nucleic acid comprises at least one first miRNA binding site sequence for reducing or preventing protein expression in immune cells as defined in any one of claims 100 to 106 and at least one second miRNA binding site sequence for reducing or preventing protein expression in liver as defined in any one of the claims 77 to 83.

130. The nucleic acid sequence of claim 120 to 129, wherein the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, a transcription factor inhibitor, an enzyme, a peptide or protein hormone, a growth factor, a cytokine, a structural protein, a cytoplasmic protein, a cytoskeletal protein, an allergen, cytostatic or cytotoxic peptide or protein, or fragments, variants, or combinations of any of these.

131. The nucleic acid sequence of claim 120 to 130, wherein the 5′ UTR is selected or derived from HSD17B4 and wherein the 3′ UTR is selected or derived from PSMB3 and wherein the nucleic acid additionally comprises at least one 5′ Cap structure, preferably a Cap1, and at least one 3′ terminal Poly(A) Sequence

132. The nucleic acid sequence of claim 120 to 131, wherein upon administration of the nucleic acid to a cell or subject the expression of the encoded peptide or protein is reduced in immune cells and in liver cells by at least 10%, 20%, 30%, 40%, 50%, 60%, 70%, 80% or 90% compared to a reference nucleic acid lacking the respective miRNA binding site sequence.

133. The nucleic acid sequence of claim 120 to 132, wherein upon administration of the nucleic acid to a cell or subject, the encoded peptide or protein is expressed in non-immune cells and in non-liver cells.

134. The nucleic acid sequence of claim 120 to 133, wherein upon administration of the nucleic acid to a cell or subject, 80%, 85%, 90%, 95%, 96%, 97%, 98%, 99% of the expressed peptide or protein is produced in non-immune cells and non liver cells.

135. The nucleic acid sequence of claim 120 to 134, wherein the administration is an intravenous, intrapulmonal, intratumoral, or intraocular administration.

136. A pharmaceutical composition comprising at least one nucleic acid sequence as defined in any one of the claims 1 to 135, additionally comprising one or more pharmaceutically acceptable excipients, carriers, diluents and/or vehicles.

137. The pharmaceutical composition of claim 136, wherein the nucleic acid sequence is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.

138. The pharmaceutical composition of claim 136 or 137, wherein the nucleic acid sequence is complexed or associated with one or more lipids, thereby forming lipid-based carriers.

139. The pharmaceutical composition of claim 138, wherein the lipid-based carriers are selected from liposomes, lipid nanoparticles (LNP), lipoplexes, and/or nanoliposomes.

140. The pharmaceutical composition of claim 139, wherein the nucleic acid sequence is complexed or associated with one or more lipids thereby forming lipid nanoparticles (LNP).

141. The pharmaceutical composition of claims 138 to 140, wherein the lipid-based carriers, preferably the lipid nanoparticles, encapsulate the artificial nucleic acid.

142. The pharmaceutical composition of claims 138 to 141, wherein the lipid-based carriers, preferably the lipid nanoparticles, comprise at least one aggregation-reducing lipid, at least one cationic lipid or ionizable lipid, at least one neutral lipid or phospholipid, at least one steroid or steroid analog, or any combinations thereof.

143. The pharmaceutical composition of claims 138 to 142, wherein the lipid-based carriers, preferably the lipid nanoparticles, comprise an aggregation reducing lipid selected from a polymer conjugated lipid.

144. The pharmaceutical composition of claim 142, wherein aggregation reducing lipid is a polymer conjugated lipid, preferably a PEG-lipid, selected or derived from formula (IVa): 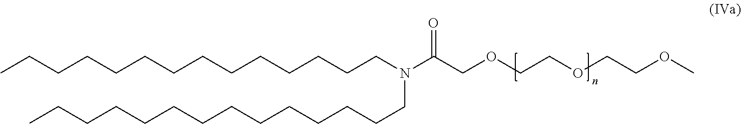
wherein n has a mean value ranging from 30 to 60, preferably wherein n has a mean value of about 45, 46, 47, 48, 49, 50, 51, 52, 53, 54, more preferably wherein n has a mean value of 49 or 45.

145. The pharmaceutical composition of claim 143 to 144, wherein the polymer conjugated lipid is a PEG-conjugated lipid preferably selected or derived from DMG-PEG 2000, C10-PEG2K, Cer8-PEG2K, or ALC-0159 (lipid of formula IVa), preferably wherein the polymer conjugated lipid is ALC-0159.

146. The pharmaceutical composition of claim 143, wherein the polymer conjugated lipid is not a PEG-conjugated lipid.

147. The pharmaceutical composition of claims 142 to 146, wherein the at least one cationic lipid is a lipid according to formula (III) or a lipid derived from formula (III), preferably a lipid according to formula (III-3) or a lipid derived from formula (III-3)

148. The pharmaceutical composition of claims 142 to 147, wherein the at least one cationic lipid is a lipid selected or derived from ALC-0315 (lipid of formula III), SM-102, SS-33/4PE-15, HEXA-C5DE-PipSS, or compound C26, preferably wherein the at least one cationic lipid is ALC-0315.

149. The pharmaceutical composition of claims 142 to 148, wherein the at least one neutral lipid is selected or derived from DSPC, DHPC, or DphyPE, preferably wherein the at least one neutral lipid is DSPC.

150. The pharmaceutical composition of claims 142 to 149, wherein the at least one steroid or steroid analog selected or derived from cholesterol, cholesteryl hemisuccinate (CHEMS), preferably cholesterol.

151. The pharmaceutical composition of claims 138 to 150, wherein the lipid-based carriers, preferably the lipid nanoparticles, comprise
(i) at least one cationic lipid, preferably as defined in claim 147 or 148;
(ii) at least one neutral lipid, preferably as defined in claim 149;
(iii) at least one steroid or steroid analogue, preferably as defined in claim 150; and
(iv) at least one aggregation-reducing lipid, preferably as defined in claims 143 to 146.

152. The pharmaceutical composition of claims 138 to 151, wherein the lipid-based carriers comprise (i) the cationic lipid ALC-0315 (lipid of formula III), (ii) the neutral lipid DSPC, (iii) cholesterol, and (iv) the aggregation reducing lipid ALC-0159 (lipid of formula IVa).

153. The pharmaceutical composition of claim 151 or 152, wherein (i) to (iv) are in a molar ratio of about 20-60% cationic lipid, about 5-25% neutral lipid, about 25-55% steroid or steroid analogue, and about 0.5-15% aggregation reducing lipid.

154. The pharmaceutical composition of claims 136 to 153, wherein the pharmaceutical composition is suitable for administration to a cell or subject, preferably a human subject.

155. The pharmaceutical composition of claim 154, wherein the administration is systemically or locally.

156. The pharmaceutical composition of claims 154 to 155, wherein the administration is transdermally, intradermally, intravenously, intramuscularly, intraaterially, intranasally, intrapulmonally, intracranially, intralesionally, intratumorally, intraocularily, intravitreally, subcutaneously or via sublingual, preferably intramuscularly, intranodally, intradermally, intratumorally or intravenously,

157. The pharmaceutical composition of claims 154 to 156, wherein the administration is intramuscularly or intravenously.

158. The pharmaceutical composition of claims 136 to 157, wherein the pharmaceutical composition comprises at least one nucleic acid sequence comprising at least one miRNA binding site sequence for reducing or preventing protein expression in the liver, preferably wherein the nucleic acid sequence is characterized by any one of the features as defined in claims 69 to 99.

159. The pharmaceutical composition of claim 158, wherein the nucleic acid sequence is formulated in lipid-based carriers, preferably a lipid based carrier as defined in any one of the claims 138 to 152.

160. The pharmaceutical composition of claim 158 or 159, wherein upon administration of the composition to a cell or subject the expression of the encoded peptide or protein is reduced in the liver by at least 10%, 20%, 30%, 40%, 50%, 60%, 70%, 80% or 90% compared to a reference composition comprising a nucleic acid sequence lacking the respective miRNA binding site sequence.

161. The pharmaceutical composition of claims 158 to 160, wherein upon administration of the composition to a cell or subject, the encoded peptide or protein is expressed in non-liver cells, preferably in immune cells or muscle cells.

162. The pharmaceutical composition of claims 158 to 161, wherein upon administration of the composition to a cell or subject, 80%, 85%, 90%, 95%, 96%, 97%, 98%, 99% of the expressed peptide or protein is produced in non-liver cells, preferably in immune cells or muscle cells.

163. The pharmaceutical composition of claims 136 to 157, wherein the pharmaceutical composition comprises at least one nucleic acid sequence comprising at least one miRNA binding site sequence for reducing or preventing protein expression in immune cells, preferably wherein the nucleic acid sequence is characterized by any one of the features as defined in claims 100 to 119.

164. The pharmaceutical composition of claim 163, wherein the nucleic acid sequence is formulated in lipid-based carriers, preferably a lipid based carrier as defined in any one of the claims 138 to 152.

165. The pharmaceutical composition of claim 163 or 164, wherein upon administration of the composition to a cell or subject the expression of the encoded peptide or protein is reduced in immune cells by at least 10%, 20%, 30%, 40%, 50%, 60%, 70%, 80% or 90% compared to a reference composition comprising a nucleic acid sequence lacking the respective miRNA binding site sequence.

166. The pharmaceutical composition of claims 163 to 165, wherein upon administration of the composition to a cell or subject, the encoded peptide or protein is expressed in non-immune cells, preferably in liver cells.

167. The pharmaceutical composition of claims 163 to 166, wherein upon administration of the composition to a cell or subject, 80%, 85%, 90%, 95%, 96%, 97%, 98%, 99% of the expressed peptide or protein is produced in non-immune cells, preferably in liver cells.

168. The pharmaceutical composition of claims 136 to 157, wherein the pharmaceutical composition comprises at least one nucleic acid sequence comprising at least one miRNA binding site sequence for reducing or preventing protein expression in immune cells and in liver cells, preferably wherein the nucleic acid sequence is characterized by any one of the features as defined in claims 120 to 135.

169. The pharmaceutical composition of claim 169, wherein the nucleic acid sequence is formulated in lipid-based carriers, preferably a lipid based carrier as defined in any one of the claims 138 to 152.

170. The pharmaceutical composition of claim 168 or 169, wherein upon administration of the composition to a cell or subject the expression of the encoded peptide or protein is reduced in immune cells and liver cells by at least 10%, 20%, 30%, 40%, 50%, 60%, 70%, 80% or 90% compared to a reference composition comprising a nucleic acid sequence lacking the respective miRNA binding site sequence.

171. The pharmaceutical composition of claims 168 to 170, wherein upon administration of the composition to a cell or subject, the encoded peptide or protein is expressed in non-immune cells and non-liver cells.

172. The pharmaceutical composition of claims 168 to 171, wherein upon administration of the composition to a cell or subject, 80%, 85%, 90%, 95%, 96%, 97%, 98%, 99% of the expressed peptide or protein is produced in non-immune cells and non-liver cells.

173. A Vaccine comprising at least one nucleic acid sequence as defined in any one of the claims 1 to 99 or comprising the pharmaceutical composition as defined in any one of the claims 136 to 162.

174. The vaccine of claim 173, wherein the nucleic acid, preferably the RNA of the vaccine, is formulated in lipid-based carriers, preferably LNPs as defined in any one of the claims 138 to 152.

175. The vaccine of claim 173 or 174, wherein the nucleic acid sequence is characterized by any one of the features of claims 69 to 99.

176. The vaccine of claims 173 to 175, wherein the vaccine is against a pathogen, preferably against a virus.

177. The vaccine of claims 173 to 176, wherein upon administration of the vaccine to a cell or subject, 80%, 85%, 90%, 95%, 96%, 97%, 98%, 99% of the expressed peptide or protein is produced in muscle cells or immune cells and expression of the encoded peptide or protein is reduced in liver cells by at least 10%, 20%, 30%, 40%, 50%, 60%, 70%, 80% or 90% compared to a reference composition comprising a nucleic acid sequence lacking the respective miRNA binding site sequence.

178. The vaccine of claims 173 to 177, wherein the vaccine is a tumor vaccine.

179. Kit or kit of parts comprising the nucleic acid sequence as defined in claims 1 to 135, or the pharmaceutical composition as defined in claims 136 to 172, or the vaccine as defined in claims 173 to 178, optionally comprising a liquid vehicle for solubilizing, and, optionally, technical instructions providing information on administration and/or dosage of the components.

180. A nucleic acid sequence as defined in claims 1 to 135, or a pharmaceutical composition as defined in claims 136 to 172, the vaccine as defined in claims 173 to 178, or a kit or kit of parts as defined in claim 179, for use as medicament.

181. A nucleic acid sequence as defined in claims 1 to 135, or a pharmaceutical composition as defined in claims 136 to 172, the vaccine as defined in claims 173 to 178, or the kit or kit of parts as defined in claim 179, for use in the prevention or treatment of cancer, autoimmune diseases, infectious diseases, allergies or protein deficiency disorders.

182. A nucleic acid sequence as defined in claims 1 to 99, or a pharmaceutical composition as defined in claims 136 to 162, the vaccine as defined in claims 173 to 178, or the kit or kit of parts as defined in claim 179, for use in treating or preventing a non-liver disease and/or a disease where a production of the target peptide or protein in the liver causes side effects.

183. A nucleic acid sequence as defined in claims 1 to 68 and 100 to 119, or a pharmaceutical composition as defined in claims 136 to 157 and 163 to 167, or the kit or kit of parts as defined in claim 179, for use in treating or preventing a non-immune cell disease and/or a disease where a production of the target peptide or protein in immune cells causes side effects.

184. A nucleic acid sequence as defined in claims 1 to 68 and 120 to 135, or a pharmaceutical composition as defined in claims 136 to 157 and 168 to 172, or the kit or kit of parts as defined in claim 179, for use in treating or preventing a non-immune cell and non-liver disease and/or a disease where a production of the target peptide or protein in immune cells and the liver causes side effects.

185. A method of treatment or preventing a disorder, wherein the method comprises applying or administering to a subject in need thereof the nucleic acid sequence as defined in claims 1 to 135, or the pharmaceutical composition as defined in claims 136 to 172, the vaccine as defined in claims 173 to 178, or the kit or kit of parts as defined in claim 179.

186. Method of treatment or preventing a disorder of claim 185, wherein the administration or applying is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, intranodal, or intratumoral, preferably intramuscular, intradermal, intravenous, or intratumoral, most preferably intramuscular.

187. Method of treatment according to claim 185 or 186, wherein the subject in need is a mammalian subject, preferably a human subject.

188. A method to promote a cell-type specific expression of a peptide or protein within a target organ or organs by using a nucleic acid sequence as defined in claims 1 to 135, the pharmaceutical composition as defined in claims 136 to 172, the vaccine as defined in claims 173 to 178, or the kit according to claims as defined in claim 179.


SEQUENCE LISTING (382 sequences)

SEQ ID NO: 1 - DNA, 62 bp, Artificial Sequence
  5UTR of human peroxisomal multifunctional enzyme type 2 lacking the 5′-terminal oligopyrimidine tract (HSD17B4)
gtcccgcagtcggcgtccagcggctctgcttgttcgtgtgtgtgtcgttgcaggccttattc

SEQ ID NO: 2 - RNA, 62 nt, Artificial Sequence
  5UTR of human peroxisomal multifunctional enzyme type 2 lacking the 5′-terminal oligopyrimidine tract (HSD17B4) mRNA
gucccgcagucggcguccagcggcucugcuuguucgugugugugucguugcaggccuuauuc

SEQ ID NO: 3 - DNA, 42 bp, Artificial Sequence
  5UTR of human ribosomal protein Large 32 lacking the 5′-terminal oligopyrimidine tract (RPL32, 32L4, 32L3)
ggcgctgcctacggaggtggcagccatctccttctcggcatc

SEQ ID NO: 4 - RNA, 42 nt, Artificial Sequence
  5UTR of human ribosomal protein Large 32 lacking the 5′-terminal oligopyrimidine tract (RPL32, 32L4, 32L3) mRNA
ggcgcugccuacggagguggcagccaucuccuucucggcauc

SEQ ID NO: 5 - DNA, 31 bp, Artificial Sequence
  5UTR of human androgen-induced gene 1 protein lacking the 5′-terminal oligopyrimidine tract (AIG1)
gccgcccagccggtccaggcctctggcgaac

SEQ ID NO: 6 - RNA, 31 nt, Artificial Sequence
  5UTR of human androgen-induced gene 1 protein lacking the 5′-terminal oligopyrimidine tract (AIG1) mRNA
gccgcccagccgguccaggccucuggcgaac

SEQ ID NO: 7 - DNA, 42 bp, Artificial Sequence
  5UTR of human acid ceramidase lacking the 5′-terminal oligopyrimidine tract (ASAH1)
gcctctgctggagtccggggagtggcgttggctgctagagcg

SEQ ID NO: 8 - RNA, 42 nt, Artificial Sequence
  5UTR of human acid ceramidase lacking the 5′-terminal oligopyrimidine tract (ASAH1) mRNA
gccucugcuggaguccggggaguggcguuggcugcuagagcg

SEQ ID NO: 9 - DNA, 75 bp, Artificial Sequence
  5UTR of human mitochondrial ATP synthase subunit alpha lacking the 5′-terminal oligopyrimidine tract (ATP5A1)
gcggctcggccattttgtcccagtcagtccggaggctgcggctgcagaagtaccgcctgcggagtaactgcaaag

SEQ ID NO: 10 - RNA, 75 nt, Artificial Sequence
  5UTR of human mitochondrial ATP synthase subunit alpha lacking the 5′-terminal oligopyrimidine tract (ATP5A1) mRNA
gcggcucggccauuuugucccagucaguccggaggcugcggcugcagaaguaccgccugcggaguaacugcaaag

SEQ ID NO: 11 - DNA, 56 bp, Artificial Sequence
  5UTR of human cytochrome c oxidase subunit 6C lacking the 5′-terminal oligopyrimidine tract (COX6C)
agtcaggaaggacgttggtgttgaggttagcatacgtatcaaggacagtaactacc

SEQ ID NO: 12 - RNA, 56 nt, Artificial Sequence
  5UTR of human cytochrome c oxidase subunit 6C lacking the 5′-terminal oligopyrimidine tract (COX6C) mRNA
agucaggaaggacguugguguugagguuagcauacguaucaaggacaguaacuacc

SEQ ID NO: 13 - DNA, 232 bp, Artificial Sequence
  5UTR of mouse dihydropyrimidinase-like 2 (Dpysl2)
ctctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagag

SEQ ID NO: 14 - RNA, 232 nt, Artificial Sequence
  5UTR of mouse dihydropyrimidinase-like 2 (Dpysl2) mRNA
cucucucccucuuucuuuuuuccgcccuagcuggaucuguguuggaggagaggaaaagagacagaggauugcaucuguuuugaaaucuccuaauagcaagaccaguuaagggauuguaccuuuuuccuacaaauauaaauauauauauauuuuaaaccaagucuuuuuuuuuccggcuaucuuugcuuuaaagcuguccucuugagauuacuuccucccgccccccggagag

SEQ ID NO: 15 - DNA, 22 bp, Artificial Sequence
  5UTR of human ATP binding cassette subfamily B member 7 lacking the 5′-terminal oligopyrimidine tract (MDR, ABCB7)
ggttcctctttcctcgctcaag

SEQ ID NO: 16 - RNA, 22 nt, Artificial Sequence
  5UTR of human ATP binding cassette subfamily B member 7 lacking the 5′-terminal oligopyrimidine tract (MDR, ABCB7) mRNA
gguuccucuuuccucgcucaag

SEQ ID NO: 17 - DNA, 54 bp, Artificial Sequence
  5UTR of mouse RIKEN cDNA 2010107E04 gene (Mp68, 2010107E04Rik)
ctttcccattctgtagcagaatttggtgttgcctgtggtcttggtcccgcggag

SEQ ID NO: 18 - RNA, 54 nt, Artificial Sequence
  5UTR of mouse RIKEN cDNA 2010107E04 gene (Mp68, 2010107E04Rik) mRNA
cuuucccauucuguagcagaauuugguguugccuguggucuuggucccgcggag

SEQ ID NO: 19 - DNA, 81 bp, Artificial Sequence
  5UTR of mouse NADH dehydrogenase (ubiquinone) 1 alpha subcomplex, 4 (Ndufa4)
gtccgctcagccaggttgcagaagcggcttagcgtgtgtcctaatcttctctctgcgtgtaggtaggcctgtgccgcaaac

SEQ ID NO: 20 - RNA, 81 nt, Artificial Sequence
  5UTR of mouse NADH dehydrogenase (ubiquinone) 1 alpha subcomplex, 4 (Ndufa4) mRNA
guccgcucagccagguugcagaagcggcuuagcguguguccuaaucuucucucugcguguagguaggccugugccgcaaac

SEQ ID NO: 21 - DNA, 108 bp, Artificial Sequence
  5UTR of mouse nitric oxide synthase interacting protein (Nosip)
ctcctgtcgggcggaagtaggaggagtagagtttaaaaacagtactctttttccggttcgggacgtagttgaagcaacgacaagccggataaccgctcttgagacagg

SEQ ID NO: 22 - RNA, 108 nt, Artificial Sequence
  5UTR of mouse nitric oxide synthase interacting protein (Nosip) mRNA
cuccugucgggcggaaguaggaggaguagaguuuaaaaacaguacucuuuuuccgguucgggacguaguugaagcaacgacaagccggauaaccgcucuugagacagg

SEQ ID NO: 23 - DNA, 84 bp, Artificial Sequence
  5UTR of mouse ribosomal protein L31 (Rpl31)
cccgtgacccggaagttgtacggctacgcgactttccctcccacaaaccctcgcgcccttcctttcctacttgggcccggcaga

SEQ ID NO: 24 - RNA, 84 nt, Artificial Sequence
  5UTR of mouse ribosomal protein L31 (Rpl31) mRNA
cccgugacccggaaguuguacggcuacgcgacuuucccucccacaaacccucgcgcccuuccuuuccuacuugggcccggcaga

SEQ ID NO: 25 - DNA, 56 bp, Artificial Sequence
  5UTR of human 60S ribosomal protein L35a lacking the 5′-terminal oligopyrimidine tract (RPL35A)
accgccatcttggctcctgtggaggcctgctgggaacgggacttctaaaaggaact

SEQ ID NO: 26 - RNA, 56 nt, Artificial Sequence
  5UTR of human 60S ribosomal protein L35a lacking the 5′-terminal oligopyrimidine tract (RPL35A) mRNA
accgccaucuuggcuccuguggaggccugcugggaacgggacuucuaaaaggaacu

SEQ ID NO: 27 - DNA, 159 bp, Artificial Sequence
  5UTR of mouse solute carrier family 7 (cationic amino acid transporter, y+ system), member 3 (Slc7a3)
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 28 - RNA, 159 nt, Artificial Sequence
  5UTR of mouse solute carrier family 7 (cationic amino acid transporter, y+ system), member 3 (Slc7a3) mRNA
gggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuaga

SEQ ID NO: 29 - DNA, 102 bp, Artificial Sequence
  5UTR of human tubulin beta 4B class Ivb (TUBB4B)
atataagcgttggcggagcgtcggttgtagcactctgcgcgcccgctcttctgctgctgtttgtctacttcctcctgcttccccgccgccgccgccgccatc

SEQ ID NO: 30 - RNA, 102 nt, Artificial Sequence
  5UTR of human tubulin beta 4B class Ivb (TUBB4B) mRNA
auauaagcguuggcggagcgucgguuguagcacucugcgcgcccgcucuucugcugcuguuugucuacuuccuccugcuuccccgccgccgccgccgccauc

SEQ ID NO: 31 - DNA, 222 bp, Artificial Sequence
  5UTR of mouse ubiquilin 2 (Ubqln2)
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 32 - RNA, 222 nt, Artificial Sequence
  5UTR of mouse ubiquilin 2 (Ubqln2) mRNA
cggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgcc

SEQ ID NO: 33 - DNA, 57 bp, Artificial Sequence
  synthetic-v1 5UTR
gggaaataagagagaaaagaagagtaagaagaaatataagaccccggcgccgccacc

SEQ ID NO: 34 - RNA, 57 nt, Artificial Sequence
  synthetic-v1 5UTR mRNA
gggaaauaagagagaaaagaagaguaagaagaaauauaagaccccggcgccgccacc

SEQ ID NO: 35 - DNA, 57 bp, Artificial Sequence
  synthetic-v2 5UTR
aggagataagagagaaaagaagagtaagaagaaatataagaccccggcgccgccacc

SEQ ID NO: 36 - RNA, 57 nt, Artificial Sequence
  synthetic-v2 5UTR mRNA
aggagauaagagagaaaagaagaguaagaagaaauauaagaccccggcgccgccacc

SEQ ID NO: 37 - DNA, 47 bp, Artificial Sequence
  synthetic-v3 5UTR
gggaaataagagagaaaagaagagtaagaagaaatataagagccacc

SEQ ID NO: 38 - RNA, 47 nt, Artificial Sequence
  synthetic-v3 5UTR mRNA
gggaaauaagagagaaaagaagaguaagaagaaauauaagagccacc

SEQ ID NO: 39 - DNA, 47 bp, Artificial Sequence
  synthetic-v4 5UTR
aggagataagagagaaaagaagagtaagaagaaatataagagccacc

SEQ ID NO: 40 - RNA, 47 nt, Artificial Sequence
  synthetic-v4 5UTR mRNA
aggagauaagagagaaaagaagaguaagaagaaauauaagagccacc

SEQ ID NO: 41 - DNA, 53 bp, Artificial Sequence
  HBA1-v1 5UTR
agaataaactagtattcttctggtccccacagactcagagagaacccgccacc

SEQ ID NO: 42 - RNA, 53 nt, Artificial Sequence
  HBA1-v1 5UTR mRNA
agaauaaacuaguauucuucugguccccacagacucagagagaacccgccacc

SEQ ID NO: 43 - DNA, 56 bp, Artificial Sequence
  HBA1-v2 5UTR
aggagaataaactagtattcttctggtccccacagactcagagagaacccgccacc

SEQ ID NO: 44 - RNA, 56 nt, Artificial Sequence
  HBA1-v2 5UTR mRNA
aggagaauaaacuaguauucuucugguccccacagacucagagagaacccgccacc

SEQ ID NO: 45 - DNA, 53 bp, Artificial Sequence
  HBA1-v3 5UTR
aggagaaactagtattcttctggtccccacagactcagagagaacccgccacc

SEQ ID NO: 46 - RNA, 53 nt, Artificial Sequence
  HBA1-v3 5UTR mRNA
aggagaaacuaguauucuucugguccccacagacucagagagaacccgccacc

SEQ ID NO: 47 - DNA, 53 bp, Artificial Sequence
  HBA1-v4 5UTR
agattaaactagtattcttctggtccccacagactcagagagaacccgccacc

SEQ ID NO: 48 - RNA, 53 nt, Artificial Sequence
  HBA1-v4 5UTRv
agauuaaacuaguauucuucugguccccacagacucagagagaacccgccacc

SEQ ID NO: 49 - DNA, 52 bp, Artificial Sequence
  HBA1-v5 5UTR
agacgaactagtattcttctggtccccacagactcagagagaacccgccacc

SEQ ID NO: 50 - RNA, 52 nt, Artificial Sequence
  HBA1-v5 5UTRv
agacgaacuaguauucuucugguccccacagacucagagagaacccgccacc

SEQ ID NO: 51 - DNA, 52 bp, Artificial Sequence
  HBA1-v6 5UTR
gggcgaactagtattcttctggtccccacagactcagagagaacccgccacc

SEQ ID NO: 52 - RNA, 52 nt, Artificial Sequence
  HBA1-v6 5UTR mRNA
gggcgaacuaguauucuucugguccccacagacucagagagaacccgccacc

SEQ ID NO: 53 - DNA, 15 bp, Artificial Sequence
  minimal 5UTR
aggagaaagcttacc

SEQ ID NO: 54 - RNA, 15 nt, Artificial Sequence
  minimal 5UTR mRNA
aggagaaagcuuacc

SEQ ID NO: 55 - DNA, 77 bp, Artificial Sequence
  HSD17B4-v2 5UTR
aggagagtcccgcagtcggcgtccagcggctctgcttgttcgtgtgtgtgtcgttgcaggccttattcaagcttacc

SEQ ID NO: 56 - RNA, 77 nt, Artificial Sequence
  HSD17B4-v2 5UTR mRNA
aggagagucccgcagucggcguccagcggcucugcuuguucgugugugugucguugcaggccuuauucaagcuuacc

SEQ ID NO: 57 - DNA, 57 bp, Artificial Sequence
  RPL32_(RPL32var,32L4, 32L3)-v2 5UTR
aggagaggcgctgcctacggaggtggcagccatctccttctcggcatcaagcttacc

SEQ ID NO: 58 - RNA, 57 nt, Artificial Sequence
  RPL32_(RPL32var,32L4, 32L3)-v2 5UTR mRNA
aggagaggcgcugccuacggagguggcagccaucuccuucucggcaucaagcuuacc

SEQ ID NO: 59 - DNA, 46 bp, Artificial Sequence
  AIG1-v2 5UTR
aggagagccgcccagccggtccaggcctctggcgaacaagcttacc

SEQ ID NO: 60 - RNA, 46 nt, Artificial Sequence
  AIG1-v2 5UTR mRNA
aggagagccgcccagccgguccaggccucuggcgaacaagcuuacc

SEQ ID NO: 61 - DNA, 57 bp, Artificial Sequence
  ASAH1-v2 5UTR
aggagagcctctgctggagtccggggagtggcgttggctgctagagcgaagcttacc

SEQ ID NO: 62 - RNA, 57 nt, Artificial Sequence
  ASAH1-v2 5UTR mRNA
aggagagccucugcuggaguccggggaguggcguuggcugcuagagcgaagcuuacc

SEQ ID NO: 63 - DNA, 90 bp, Artificial Sequence
  ATP5A1-v2 5UTR
aggagagcggctcggccattttgtcccagtcagtccggaggctgcggctgcagaagtaccgcctgcggagtaactgcaaagaagcttacc

SEQ ID NO: 64 - RNA, 90 nt, Artificial Sequence
  ATP5A1-v2 5UTR mRNA
aggagagcggcucggccauuuugucccagucaguccggaggcugcggcugcagaaguaccgccugcggaguaacugcaaagaagcuuacc

SEQ ID NO: 65 - DNA, 71 bp, Artificial Sequence
  COX6C-v2 5UTR
aggagaagtcaggaaggacgttggtgttgaggttagcatacgtatcaaggacagtaactaccaagcttacc

SEQ ID NO: 66 - RNA, 71 nt, Artificial Sequence
  COX6C-v2 5UTR mRNA
aggagaagucaggaaggacguugguguugagguuagcauacguaucaaggacaguaacuaccaagcuuacc

SEQ ID NO: 67 - DNA, 247 bp, Artificial Sequence
  Dpysl2-v2 5UTR
aggagactctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagagaagcttacc

SEQ ID NO: 68 - RNA, 247 nt, Artificial Sequence
  Dpysl2-v2 5UTR mRNA
aggagacucucucccucuuucuuuuuuccgcccuagcuggaucuguguuggaggagaggaaaagagacagaggauugcaucuguuuugaaaucuccuaauagcaagaccaguuaagggauuguaccuuuuuccuacaaauauaaauauauauauauuuuaaaccaagucuuuuuuuuuccggcuaucuuugcuuuaaagcuguccucuugagauuacuuccucccgccccccggagagaagcuuacc

SEQ ID NO: 69 - DNA, 37 bp, Artificial Sequence
  MDR (ABCB7)-v2 5UTR
aggagaggttcctctttcctcgctcaagaagcttacc

SEQ ID NO: 70 - RNA, 37 nt, Artificial Sequence
  MDR (ABCB7)-v2 5UTR mRNA
aggagagguuccucuuuccucgcucaagaagcuuacc

SEQ ID NO: 71 - DNA, 69 bp, Artificial Sequence
  Mp68_(2010107E04Rik)-v2 5UTR
aggagactttcccattctgtagcagaatttggtgttgcctgtggtcttggtcccgcggagaagcttacc

SEQ ID NO: 72 - RNA, 69 nt, Artificial Sequence
  Mp68_(2010107E04Rik)-v2 5UTR mRNA
aggagacuuucccauucuguagcagaauuugguguugccuguggucuuggucccgcggagaagcuuacc

SEQ ID NO: 73 - DNA, 96 bp, Artificial Sequence
  Ndufa4-v2 5UTR
aggagagtccgctcagccaggttgcagaagcggcttagcgtgtgtcctaatcttctctctgcgtgtaggtaggcctgtgccgcaaacaagcttacc

SEQ ID NO: 74 - RNA, 96 nt, Artificial Sequence
  Ndufa4-v2 5UTR mRNA
aggagaguccgcucagccagguugcagaagcggcuuagcguguguccuaaucuucucucugcguguagguaggccugugccgcaaacaagcuuacc

SEQ ID NO: 75 - DNA, 123 bp, Artificial Sequence
  Nosip-v2 5UTR
aggagactcctgtcgggcggaagtaggaggagtagagtttaaaaacagtactctttttccggttcgggacgtagttgaagcaacgacaagccggataaccgctcttgagacaggaagcttacc

SEQ ID NO: 76 - RNA, 123 nt, Artificial Sequence
  Nosip-v2 5UTR mRNA
aggagacuccugucgggcggaaguaggaggaguagaguuuaaaaacaguacucuuuuuccgguucgggacguaguugaagcaacgacaagccggauaaccgcucuugagacaggaagcuuacc

SEQ ID NO: 77 - DNA, 99 bp, Artificial Sequence
  Rpl31-v2 5UTR
aggagacccgtgacccggaagttgtacggctacgcgactttccctcccacaaaccctcgcgcccttcctttcctacttgggcccggcagaaagcttacc

SEQ ID NO: 78 - RNA, 99 nt, Artificial Sequence
  Rpl31-v2 5UTR mRNA
aggagacccgugacccggaaguuguacggcuacgcgacuuucccucccacaaacccucgcgcccuuccuuuccuacuugggcccggcagaaagcuuacc

SEQ ID NO: 79 - DNA, 71 bp, Artificial Sequence
  RPL35A-v2 5UTR
aggagaaccgccatcttggctcctgtggaggcctgctgggaacgggacttctaaaaggaactaagcttacc

SEQ ID NO: 80 - RNA, 71 nt, Artificial Sequence
  RPL35A-v2 5UTR mRNA
aggagaaccgccaucuuggcuccuguggaggccugcugggaacgggacuucuaaaaggaacuaagcuuacc

SEQ ID NO: 81 - DNA, 174 bp, Artificial Sequence
  Slc7a3-v2 5UTR
aggagagggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactagaaagcttacc

SEQ ID NO: 82 - RNA, 174 nt, Artificial Sequence
  Slc7a3-v2 5UTR mRNA
aggagagggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuagaaagcuuacc

SEQ ID NO: 83 - DNA, 117 bp, Artificial Sequence
  TUBB4B-v2 5UTR
aggagaatataagcgttggcggagcgtcggttgtagcactctgcgcgcccgctcttctgctgctgtttgtctacttcctcctgcttccccgccgccgccgccgccatcaagcttacc

SEQ ID NO: 84 - RNA, 117 nt, Artificial Sequence
  TUBB4B-v2 5UTR mRNA
aggagaauauaagcguuggcggagcgucgguuguagcacucugcgcgcccgcucuucugcugcuguuugucuacuuccuccugcuuccccgccgccgccgccgccaucaagcuuacc

SEQ ID NO: 85 - DNA, 237 bp, Artificial Sequence
  Ubqln2-v2 5UTR
aggagacggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgccaagcttacc

SEQ ID NO: 86 - RNA, 237 nt, Artificial Sequence
  Ubqln2-v2 5UTR mRNA
aggagacggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgccaagcuuacc

SEQ ID NO: 87 - DNA, 57 bp, Artificial Sequence
  3UTR of human proteasome subunit beta 3 (PSMB3)
ccctgttcccagagcccacttttttttctttttttgaaataaaatagcctgtctttc

SEQ ID NO: 88 - RNA, 57 nt, Artificial Sequence
  3UTR of human proteasome subunit beta 3 (PSMB3) mRNA
cccuguucccagagcccacuuuuuuuucuuuuuuugaaauaaaauagccugucuuuc

SEQ ID NO: 89 - DNA, 44 bp, Artificial Sequence
  Center, alpha-complex-binding portion of the 3UTR of an alpha-globin gene (muag)
gcccgatgggcctcccaacgggccctcctcccctccttgcaccg

SEQ ID NO: 90 - RNA, 44 nt, Artificial Sequence
  Center, alpha-complex-binding portion of the 3UTR of an alpha-globin gene (muag) mRNA
gcccgaugggccucccaacgggcccuccuccccuccuugcaccg

SEQ ID NO: 91 - DNA, 187 bp, Artificial Sequence
  3UTR ALB7
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 92 - RNA, 187 nt, Artificial Sequence
  3UTR ALB7 mRNA
gcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccu

SEQ ID NO: 93 - DNA, 266 bp, Artificial Sequence
  3UTR of human annexin A4 (ANXA4)
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 94 - RNA, 266 nt, Artificial Sequence
  3UTR of human annexin A4 (ANXA4) mRNA
aauaaaaaucccagaaggacaggaggauucucaacacuuugaauuuuuuuaacuucauuuuucuacacugcuauuaucauuaucucagaaugcuuauuuccaauuaaaacgccuacagcugccuccuagaauauagacugucuguauuauuauucaccuauaauuagucauuaugaugcuuuaaagcuguacuugcauuucaaagcuuauaagauauaaauggagauuuuaaaguagaaauaaauauguauuccauguuuuuaaaa

SEQ ID NO: 95 - DNA, 121 bp, Artificial Sequence
  3UTR of human caspase 1 (CASP1)
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 96 - RNA, 121 nt, Artificial Sequence
  3UTR of human caspase 1 (CASP1) mRNA
aauaaggaaacuguaugaaugucugugggcaggaagugaagagauccuucuguaaagguuuuuggaauuaugucugcugaauaauaaacuuuuuugaaauaauaaaucugguagaaaaaug

SEQ ID NO: 97 - DNA, 137 bp, Artificial Sequence
  3UTR of human cytochrome c oxidase subunit 6B1 (COX6B1)
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 98 - RNA, 137 nt, Artificial Sequence
  3UTR of human cytochrome c oxidase subunit 6B1 (COX6B1) mRNA
acuggcugcaucucccuuuccucuguccuccauccuucucccaggauggugaagggggaccugguacccagugauccccaccccaggauccuaaaucaugacuuaccugcuaauaaaaacucauuggaaaagugaga

SEQ ID NO: 99 - DNA, 166 bp, Artificial Sequence
  3UTR of human FIG4 phosphoinositide 5-phosphatase (FIG4)
aaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgc

SEQ ID NO: 100 - RNA, 166 nt, Artificial Sequence
  3UTR of human FIG4 phosphoinositide 5-phosphatase (FIG4) mRNA
aaagagcgcagguccaccugguggacacgucugauuagcuuagaaccugucuugucucaucuucaaaagguaacuuauuaaaaguccuuugcgucugaagccuuucuccuuuucugucacuugcaaauuccaaauuauagcuaauaaagaugacuagauaauuugc

SEQ ID NO: 101 - DNA, 353 bp, Artificial Sequence
  3UTR of mouse GNAS (guanine nucleotide binding protein, alpha stimulating) complex locus (Gnas)
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 102 - RNA, 353 nt, Artificial Sequence
  3UTR of mouse GNAS (guanine nucleotide binding protein, alpha stimulating) complex locus (Gnas) mRNA
gaagggaacacccaaauuuaauucagccuuaagcacaauuaauuaagagugaaacguaauuguacaagcaguuggucacccaccauagggcaugaucaacaccgcaaccuuuccuuuuucccccagugauucugaaaaaccccucuucccuucagcuugcuuagauguuccaaauuuaguaagcuuaaggcggccuacagaagaaaaagaaaaaaaaggccacaaaaguucccucucacuuucaguaaauaaaauaaaagcagcaacagaaauaaagaaauaaaugaaauucaaaaugaaauaaauauuguguugugcagcauuaaaaaaucaauaaaaauuaaaaaugagca

SEQ ID NO: 103 - DNA, 133 bp, Artificial Sequence
  3UTR of mouse NADH dehydrogenase (ubiquinone) 1 alpha subcomplex, 1 (Ndufa1)
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 104 - RNA, 133 nt, Artificial Sequence
  3UTR of mouse NADH dehydrogenase (ubiquinone) 1 alpha subcomplex, 1 (Ndufa1) mRNA
ggaagcauuuuccuggcugauuaaaagaaauuacucagcuauggucaucuguuccuguuagaaggcuaugcagcauauuauauacuaugcgcauguuaugaaaugcauaauaaaaaauuuuaaaaaaucuaaa

SEQ ID NO: 105 - DNA, 70 bp, Artificial Sequence
  3UTR of human ribosomal protein S9 (RPS9)
gtccacctgtccctcctgggctgctggattgtctcgttttcctgccaaataaacaggatcagcgctttac

SEQ ID NO: 106 - RNA, 70 nt, Artificial Sequence
  3UTR of human ribosomal protein S9 (RPS9) mRNA
guccaccugucccuccugggcugcuggauugucucguuuuccugccaaauaaacaggaucagcgcuuuac

SEQ ID NO: 107 - DNA, 240 bp, Artificial Sequence
  3UTR of mouse solute carrier family 7 (cationic amino acid transporter, y+ system), member 3 (Slc7a3)
cctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgt

SEQ ID NO: 108 - RNA, 240 nt, Artificial Sequence
  3UTR of mouse solute carrier family 7 (cationic amino acid transporter, y+ system), member 3 (Slc7a3) mRNA
ccuaaccauaccuaauuguugucuuguucuccuauacaauaauggagaguacucuugaccccacgaagagcugggccucucaugugguguuagugacggauaugaaauaaugcucuguauuucuugugaaaugauggcuaugucuuugcuguuucucauuuguuuuuaacuuguauacuuugaaauagucucuguaguuguuuccuggcuuugaaaaaaauguuauuaaaggaaaauugu

SEQ ID NO: 109 - DNA, 151 bp, Artificial Sequence
  3UTR of human tubulin beta 4B class Ivb (TUBB4B)
agccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtg

SEQ ID NO: 110 - RNA, 151 nt, Artificial Sequence
  3UTR of human tubulin beta 4B class Ivb (TUBB4B) mRNA
agccuucagucacuggggaaagcagggaagcagugugaacucuuuauucacucccagccuguccuguggccugucccacugugugcacuugcuguuuucccuguccacauccaugcuguacagacaccaccauuaaagcauuuucauagug

SEQ ID NO: 111 - DNA, 110 bp, Artificial Sequence
  HBA1-v1 3UTR
gctggagcctcggtggccatgcttcttgccccttgggcctccccccagcccctcctccccttcctgcacccgtacccccgtggtctttgaataaagtctgagtgggcggc

SEQ ID NO: 112 - RNA, 110 nt, Artificial Sequence
  HBA1-v1 3UTR mRNA
gcuggagccucgguggccaugcuucuugccccuugggccuccccccagccccuccuccccuuccugcacccguacccccguggucuuugaauaaagucugagugggcggc

SEQ ID NO: 113 - DNA, 119 bp, Artificial Sequence
  HBA1-v2 3UTR
tgataataggctggagcctcggtggcctagcttcttgccccttgggcctccccccagcccctcctccccttcctgcacccgtacccccgtggtctttgaataaagtctgagtgggcggc

SEQ ID NO: 114 - RNA, 119 nt, Artificial Sequence
  HBA1-v2 3UTR mRNA
ugauaauaggcuggagccucgguggccuagcuucuugccccuugggccuccccccagccccuccuccccuuccugcacccguacccccguggucuuugaauaaagucugagugggcggc

SEQ ID NO: 115 - DNA, 119 bp, Artificial Sequence
  HBA1-v3 3UTR
tgataataggctggagcctcggtggccatgcttcttgccccttgggcctccccccagcccctcctccccttcctgcacccgtacccccgtggtctttgaataaagtctgagtgggcggc

SEQ ID NO: 116 - RNA, 119 nt, Artificial Sequence
  HBA1-v33UTR mRNA
ugauaauaggcuggagccucgguggccaugcuucuugccccuugggccuccccccagccccuccuccccuuccugcacccguacccccguggucuuugaauaaagucugagugggcggc

SEQ ID NO: 117 - DNA, 108 bp, Artificial Sequence
  HBA2 3UTR
gctggagcctcggtagccgttcctcctgcccgctgggcctcccaacgggccctcctcccctccttgcaccggcccttcctggtctttgaataaagtctgagtgggcag

SEQ ID NO: 118 - RNA, 108 nt, Artificial Sequence
  HBA2 3UTR mRNA
gcuggagccucgguagccguuccuccugcccgcugggccucccaacgggcccuccuccccuccuugcaccggcccuuccuggucuuugaauaaagucugagugggcag

SEQ ID NO: 119 - DNA, 132 bp, Artificial Sequence
  HBB-v1 3UTR
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 120 - RNA, 132 nt, Artificial Sequence
  HBB-v1 3UTR mRNA
gcucgcuuucuugcuguccaauuucuauuaaagguuccuuuguucccuaaguccaacuacuaaacugggggauauuaugaagggccuugagcaucuggauucugccuaauaaaaaacauuuauuuucauugc

SEQ ID NO: 121 - DNA, 311 bp, Artificial Sequence
  HBB-v2 3UTR
ctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagc

SEQ ID NO: 122 - RNA, 311 nt, Artificial Sequence
  HBB-v2 3UTR mRNA
cucgagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgucgagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgucgagaccugguccagagucgcuagc

SEQ ID NO: 123 - DNA, 295 bp, Artificial Sequence
  AES-12S-v1 3UTR
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 124 - RNA, 295 nt, Artificial Sequence
  AES-12S-v1 3UTR mRNA
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagc

SEQ ID NO: 125 - DNA, 317 bp, Artificial Sequence
  AES-12S-v2 3UTR
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 126 - RNA, 317 nt, Artificial Sequence
  AES-12S-v2 3UTR mRNA
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 127 - DNA, 95 bp, Artificial Sequence
  PSMB3-v2 3UTR
ggactagtccctgttcccagagcccacttttttttctttttttgaaataaaatagcctgtctttcagatctcaaaggctcttttcagagccacca

SEQ ID NO: 128 - RNA, 95 nt, Artificial Sequence
  PSMB3-v2 3UTR mRNA
ggacuagucccuguucccagagcccacuuuuuuuucuuuuuuugaaauaaaauagccugucuuucagaucucaaaggcucuuuucagagccacca

SEQ ID NO: 129 - DNA, 98 bp, Artificial Sequence
  muag-v2 3UTR
ggactagttataagactgactagcccgatgggcctcccaacgggccctcctcccctccttgcaccgagattaatcaaaggctcttttcagagccacca

SEQ ID NO: 130 - RNA, 98 nt, Artificial Sequence
  muag-v2 3UTR mRNA
ggacuaguuauaagacugacuagcccgaugggccucccaacgggcccuccuccccuccuugcaccgagauuaaucaaaggcucuuuucagagccacca

SEQ ID NO: 131 - DNA, 104 bp, Artificial Sequence
  muag-v3 3UTR
ggactagttataagactgactagcccgatgggcctcccaacgggccctcctcccctccttgcaccgagattaatagatctcaaaggctcttttcagagccacca

SEQ ID NO: 132 - RNA, 104 nt, Artificial Sequence
  muag-v3 3UTR mRNA
ggacuaguuauaagacugacuagcccgaugggccucccaacgggcccuccuccccuccuugcaccgagauuaauagaucucaaaggcucuuuucagagccacca

SEQ ID NO: 133 - DNA, 98 bp, Artificial Sequence
  muag-v4 3UTR
ggactagttataagactgactagcccgatgggcctcccaacgggccctcctcccctccttgcaccgagagatctcaaaggctcttttcagagccacca

SEQ ID NO: 134 - RNA, 98 nt, Artificial Sequence
  muag-v4 3UTR mRNA
ggacuaguuauaagacugacuagcccgaugggccucccaacgggcccuccuccccuccuugcaccgagagaucucaaaggcucuuuucagagccacca

SEQ ID NO: 135 - DNA, 225 bp, Artificial Sequence
  ALB7-v2 3UTR
ggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctcaaaggctcttttcagagccacca

SEQ ID NO: 136 - RNA, 225 nt, Artificial Sequence
  ALB7-v2 3UTR mRNA
ggacuagugcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccuagaucucaaaggcucuuuucagagccacca

SEQ ID NO: 137 - DNA, 304 bp, Artificial Sequence
  ANXA4-v2 3UTR
ggactagtaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaaagatctcaaaggctcttttcagagccacca

SEQ ID NO: 138 - RNA, 304 nt, Artificial Sequence
  ANXA4-v2 3UTR mRNA
ggacuaguaauaaaaaucccagaaggacaggaggauucucaacacuuugaauuuuuuuaacuucauuuuucuacacugcuauuaucauuaucucagaaugcuuauuuccaauuaaaacgccuacagcugccuccuagaauauagacugucuguauuauuauucaccuauaauuagucauuaugaugcuuuaaagcuguacuugcauuucaaagcuuauaagauauaaauggagauuuuaaaguagaaauaaauauguauuccauguuuuuaaaaagaucucaaaggcucuuuucagagccacca

SEQ ID NO: 139 - DNA, 159 bp, Artificial Sequence
  CASP1-v2 3UTR
ggactagtaataaggaaactgtatgaatgtctgtgggcaggaagtgaagagatccttctgtaaaggtttttggaattatgtctgctgaataataaacttttttgaaataataaatctggtagaaaaatgagatctcaaaggctcttttcagagccacca

SEQ ID NO: 140 - RNA, 159 nt, Artificial Sequence
  CASP1-v2 3UTR mRNA
ggacuaguaauaaggaaacuguaugaaugucugugggcaggaagugaagagauccuucuguaaagguuuuuggaauuaugucugcugaauaauaaacuuuuuugaaauaauaaaucugguagaaaaaugagaucucaaaggcucuuuucagagccacca

SEQ ID NO: 141 - DNA, 175 bp, Artificial Sequence
  COX6B1-v2 3UTR
ggactagtactggctgcatctccctttcctctgtcctccatccttctcccaggatggtgaagggggacctggtacccagtgatccccaccccaggatcctaaatcatgacttacctgctaataaaaactcattggaaaagtgagaagatctcaaaggctcttttcagagccacca

SEQ ID NO: 142 - RNA, 175 nt, Artificial Sequence
  COX6B1-v2 3UTR mRNA
ggacuaguacuggcugcaucucccuuuccucuguccuccauccuucucccaggauggugaagggggaccugguacccagugauccccaccccaggauccuaaaucaugacuuaccugcuaauaaaaacucauuggaaaagugagaagaucucaaaggcucuuuucagagccacca

SEQ ID NO: 143 - DNA, 204 bp, Artificial Sequence
  FIG4-v2 3UTR
ggactagtaaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgcagatctcaaaggctcttttcagagccacca

SEQ ID NO: 144 - RNA, 204 nt, Artificial Sequence
  FIG4-v2 3UTR mRNA
ggacuaguaaagagcgcagguccaccugguggacacgucugauuagcuuagaaccugucuugucucaucuucaaaagguaacuuauuaaaaguccuuugcgucugaagccuuucuccuuuucugucacuugcaaauuccaaauuauagcuaauaaagaugacuagauaauuugcagaucucaaaggcucuuuucagagccacca

SEQ ID NO: 145 - DNA, 391 bp, Artificial Sequence
  Gnas-v2 3UTR
ggactagtgaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaagatctcaaaggctcttttcagagccacca

SEQ ID NO: 146 - RNA, 391 nt, Artificial Sequence
  Gnas-v2 3UTRv mRNA
ggacuagugaagggaacacccaaauuuaauucagccuuaagcacaauuaauuaagagugaaacguaauuguacaagcaguuggucacccaccauagggcaugaucaacaccgcaaccuuuccuuuuucccccagugauucugaaaaaccccucuucccuucagcuugcuuagauguuccaaauuuaguaagcuuaaggcggccuacagaagaaaaagaaaaaaaaggccacaaaaguucccucucacuuucaguaaauaaaauaaaagcagcaacagaaauaaagaaauaaaugaaauucaaaaugaaauaaauauuguguugugcagcauuaaaaaaucaauaaaaauuaaaaaugagcaagaucucaaaggcucuuuucagagccacca

SEQ ID NO: 147 - DNA, 171 bp, Artificial Sequence
  Ndufa1-v2 3UTR
ggactagtggaagcattttcctggctgattaaaagaaattactcagctatggtcatctgttcctgttagaaggctatgcagcatattatatactatgcgcatgttatgaaatgcataataaaaaattttaaaaaatctaaaagatctcaaaggctcttttcagagccacca

SEQ ID NO: 148 - RNA, 171 nt, Artificial Sequence
  Ndufa1-v2 3UTR mRNA
ggacuaguggaagcauuuuccuggcugauuaaaagaaauuacucagcuauggucaucuguuccuguuagaaggcuaugcagcauauuauauacuaugcgcauguuaugaaaugcauaauaaaaaauuuuaaaaaaucuaaaagaucucaaaggcucuuuucagagccacca

SEQ ID NO: 149 - DNA, 108 bp, Artificial Sequence
  RPS9-v2 3UTR
ggactagtgtccacctgtccctcctgggctgctggattgtctcgttttcctgccaaataaacaggatcagcgctttacagatctcaaaggctcttttcagagccacca

SEQ ID NO: 150 - RNA, 108 nt, Artificial Sequence
  RPS9-v2 3UTR mRNA
ggacuaguguccaccugucccuccugggcugcuggauugucucguuuuccugccaaauaaacaggaucagcgcuuuacagaucucaaaggcucuuuucagagccacca

SEQ ID NO: 151 - DNA, 278 bp, Artificial Sequence
  Slc7a3-v2 3UTR
ggactagtcctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgtagatctcaaaggctcttttcagagccacca

SEQ ID NO: 152 - RNA, 278 nt, Artificial Sequence
  Slc7a3-v2 3UTR mRNA
ggacuaguccuaaccauaccuaauuguugucuuguucuccuauacaauaauggagaguacucuugaccccacgaagagcugggccucucaugugguguuagugacggauaugaaauaaugcucuguauuucuugugaaaugauggcuaugucuuugcuguuucucauuuguuuuuaacuuguauacuuugaaauagucucuguaguuguuuccuggcuuugaaaaaaauguuauuaaaggaaaauuguagaucucaaaggcucuuuucagagccacca

SEQ ID NO: 153 - DNA, 189 bp, Artificial Sequence
  TUBB4B-v2 3UTR
ggactagtagccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtgagatctcaaaggctcttttcagagccacca

SEQ ID NO: 154 - RNA, 189 nt, Artificial Sequence
  TUBB4B-v2 3UTR mRNA
ggacuaguagccuucagucacuggggaaagcagggaagcagugugaacucuuuauucacucccagccuguccuguggccugucccacugugugcacuugcuguuuucccuguccacauccaugcuguacagacaccaccauuaaagcauuuucauagugagaucucaaaggcucuuuucagagccacca

SEQ ID NO: 155 - DNA, 148 bp, Artificial Sequence
  HBA1-v2 3UTR
ggactagtgctggagcctcggtggccatgcttcttgccccttgggcctccccccagcccctcctccccttcctgcacccgtacccccgtggtctttgaataaagtctgagtgggcggcagatctcaaaggctcttttcagagccacca

SEQ ID NO: 156 - RNA, 148 nt, Artificial Sequence
  HBA1-v2 3UTR mRNA
ggacuagugcuggagccucgguggccaugcuucuugccccuugggccuccccccagccccuccuccccuuccugcacccguacccccguggucuuugaauaaagucugagugggcggcagaucucaaaggcucuuuucagagccacca

SEQ ID NO: 157 - DNA, 146 bp, Artificial Sequence
  HBA2-v2 3UTR
ggactagtgctggagcctcggtagccgttcctcctgcccgctgggcctcccaacgggccctcctcccctccttgcaccggcccttcctggtctttgaataaagtctgagtgggcagagatctcaaaggctcttttcagagccacca

SEQ ID NO: 158 - RNA, 146 nt, Artificial Sequence
  HBA2-v2 3UTR mRNA
ggacuagugcuggagccucgguagccguuccuccugcccgcugggccucccaacgggcccuccuccccuccuugcaccggcccuuccuggucuuugaauaaagucugagugggcagagaucucaaaggcucuuuucagagccacca

SEQ ID NO: 159 - DNA, 170 bp, Artificial Sequence
  HBB-v2 3UTR
ggactagtgctcgctttcttgctgtccaatttctattaaaggttcctttgttccctaagtccaactactaaactgggggatattatgaagggccttgagcatctggattctgcctaataaaaaacatttattttcattgcagatctcaaaggctcttttcagagccacca

SEQ ID NO: 160 - RNA, 170 nt, Artificial Sequence
  HBB-v2 3UTR mRNA
ggacuagugcucgcuuucuugcuguccaauuucuauuaaagguuccuuuguucccuaaguccaacuacuaaacugggggauauuaugaagggccuugagcaucuggauucugccuaauaaaaaacauuuauuuucauugcagaucucaaaggcucuuuucagagccacca

SEQ ID NO: 161 - DNA, 71 bp, Artificial Sequence
  PSMB3-v3 3UTR
ggactagtccctgttcccagagcccacttttttttctttttttgaaataaaatagcctgtctttcagatct

SEQ ID NO: 162 - RNA, 71 nt, Artificial Sequence
  PSMB3-v3 3UTR mRNA
ggacuagucccuguucccagagcccacuuuuuuuucuuuuuuugaaauaaaauagccugucuuucagaucu

SEQ ID NO: 163 - DNA, 74 bp, Artificial Sequence
  muag-v5 3UTR
ggactagttataagactgactagcccgatgggcctcccaacgggccctcctcccctccttgcaccgagattaat

SEQ ID NO: 164 - RNA, 74 nt, Artificial Sequence
  muag-v5 3UTR mRNA
ggacuaguuauaagacugacuagcccgaugggccucccaacgggcccuccuccccuccuugcaccgagauuaau

SEQ ID NO: 165 - DNA, 80 bp, Artificial Sequence
  muag-v6 3UTR
ggactagttataagactgactagcccgatgggcctcccaacgggccctcctcccctccttgcaccgagattaatagatct

SEQ ID NO: 166 - RNA, 80 nt, Artificial Sequence
  muag-v6 3UTR mRNA
ggacuaguuauaagacugacuagcccgaugggccucccaacgggcccuccuccccuccuugcaccgagauuaauagaucu

SEQ ID NO: 167 - DNA, 74 bp, Artificial Sequence
  muag-v7 3UTR
ggactagttataagactgactagcccgatgggcctcccaacgggccctcctcccctccttgcaccgagagatct

SEQ ID NO: 168 - RNA, 74 nt, Artificial Sequence
  muag-v7 3UTR mRNA
ggacuaguuauaagacugacuagcccgaugggccucccaacgggcccuccuccccuccuugcaccgagagaucu

SEQ ID NO: 169 - DNA, 201 bp, Artificial Sequence
  ALB7-v3 3UTR
ggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatct

SEQ ID NO: 170 - RNA, 201 nt, Artificial Sequence
  ALB7-v3 3UTR mRNA
ggacuagugcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccuagaucu

SEQ ID NO: 171 - DNA, 280 bp, Artificial Sequence
  ANXA4-v3 3UTR
ggactagtaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaaagatct

SEQ ID NO: 172 - RNA, 280 nt, Artificial Sequence
  ANXA4-v3 3UTR mRNA
ggacuaguaauaaaaaucccagaaggacaggaggauucucaacacuuugaauuuuuuuaacuucauuuuucuacacugcuauuaucauuaucucagaaugcuuauuuccaauuaaaacgccuacagcugccuccuagaauauagacugucuguauuauuauucaccuauaauuagucauuaugaugcuuuaaagcuguacuugcauuucaaagcuuauaagauauaaauggagauuuuaaaguagaaauaaauauguauuccauguuuuuaaaaagaucu

SEQ ID NO: 173 - DNA, 135 bp, Artificial Sequence
  CASP1-v3 3UTR
ggactagtaataaggaaactgtatgaatgtctgtgggcaggaagtgaagagatccttctgtaaaggtttttggaattatgtctgctgaataataaacttttttgaaataataaatctggtagaaaaatgagatct

SEQ ID NO: 174 - RNA, 135 nt, Artificial Sequence
  CASP1-v3 3UTR mRNA
ggacuaguaauaaggaaacuguaugaaugucugugggcaggaagugaagagauccuucuguaaagguuuuuggaauuaugucugcugaauaauaaacuuuuuugaaauaauaaaucugguagaaaaaugagaucu

SEQ ID NO: 175 - DNA, 151 bp, Artificial Sequence
  COX6B1-v3 3UTR
ggactagtactggctgcatctccctttcctctgtcctccatccttctcccaggatggtgaagggggacctggtacccagtgatccccaccccaggatcctaaatcatgacttacctgctaataaaaactcattggaaaagtgagaagatct

SEQ ID NO: 176 - RNA, 151 nt, Artificial Sequence
  COX6B1-v3 3UTR mRNA
ggacuaguacuggcugcaucucccuuuccucuguccuccauccuucucccaggauggugaagggggaccugguacccagugauccccaccccaggauccuaaaucaugacuuaccugcuaauaaaaacucauuggaaaagugagaagaucu

SEQ ID NO: 177 - DNA, 180 bp, Artificial Sequence
  FIG4-v3 3UTR
ggactagtaaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgcagatct

SEQ ID NO: 178 - RNA, 180 nt, Artificial Sequence
  FIG4-v3 3UTR mRNA
ggacuaguaaagagcgcagguccaccugguggacacgucugauuagcuuagaaccugucuugucucaucuucaaaagguaacuuauuaaaaguccuuugcgucugaagccuuucuccuuuucugucacuugcaaauuccaaauuauagcuaauaaagaugacuagauaauuugcagaucu

SEQ ID NO: 179 - DNA, 367 bp, Artificial Sequence
  Gnas-v3 3UTR
ggactagtgaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaagatct

SEQ ID NO: 180 - RNA, 367 nt, Artificial Sequence
  Gnas-v3 3UTR mRNA
ggacuagugaagggaacacccaaauuuaauucagccuuaagcacaauuaauuaagagugaaacguaauuguacaagcaguuggucacccaccauagggcaugaucaacaccgcaaccuuuccuuuuucccccagugauucugaaaaaccccucuucccuucagcuugcuuagauguuccaaauuuaguaagcuuaaggcggccuacagaagaaaaagaaaaaaaaggccacaaaaguucccucucacuuucaguaaauaaaauaaaagcagcaacagaaauaaagaaauaaaugaaauucaaaaugaaauaaauauuguguugugcagcauuaaaaaaucaauaaaaauuaaaaaugagcaagaucu

SEQ ID NO: 181 - DNA, 147 bp, Artificial Sequence
  Ndufa1-v3 3UTR
ggactagtggaagcattttcctggctgattaaaagaaattactcagctatggtcatctgttcctgttagaaggctatgcagcatattatatactatgcgcatgttatgaaatgcataataaaaaattttaaaaaatctaaaagatct

SEQ ID NO: 182 - RNA, 147 nt, Artificial Sequence
  Ndufa1-v3 3UTR mRNA
ggacuaguggaagcauuuuccuggcugauuaaaagaaauuacucagcuauggucaucuguuccuguuagaaggcuaugcagcauauuauauacuaugcgcauguuaugaaaugcauaauaaaaaauuuuaaaaaaucuaaaagaucu

SEQ ID NO: 183 - DNA, 84 bp, Artificial Sequence
  RPS9-v3 3UTR
ggactagtgtccacctgtccctcctgggctgctggattgtctcgttttcctgccaaataaacaggatcagcgctttacagatct

SEQ ID NO: 184 - RNA, 84 nt, Artificial Sequence
  RPS9-v3 3UTR mRNA
ggacuaguguccaccugucccuccugggcugcuggauugucucguuuuccugccaaauaaacaggaucagcgcuuuacagaucu

SEQ ID NO: 185 - DNA, 254 bp, Artificial Sequence
  Slc7a3-v3 3UTR
ggactagtcctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgtagatct

SEQ ID NO: 186 - RNA, 254 nt, Artificial Sequence
  Slc7a3-v3 3UTR mRNA
ggacuaguccuaaccauaccuaauuguugucuuguucuccuauacaauaauggagaguacucuugaccccacgaagagcugggccucucaugugguguuagugacggauaugaaauaaugcucuguauuucuugugaaaugauggcuaugucuuugcuguuucucauuuguuuuuaacuuguauacuuugaaauagucucuguaguuguuuccuggcuuugaaaaaaauguuauuaaaggaaaauuguagaucu

SEQ ID NO: 187 - DNA, 165 bp, Artificial Sequence
  TUBB4B-v3 3UTR
ggactagtagccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtgagatct

SEQ ID NO: 188 - RNA, 165 nt, Artificial Sequence
  TUBB4B-v3 3UTR mRNA
ggacuaguagccuucagucacuggggaaagcagggaagcagugugaacucuuuauucacucccagccuguccuguggccugucccacugugugcacuugcuguuuucccuguccacauccaugcuguacagacaccaccauuaaagcauuuucauagugagaucu

SEQ ID NO: 189 - DNA, 124 bp, Artificial Sequence
  HBA1-v3 3UTR
ggactagtgctggagcctcggtggccatgcttcttgccccttgggcctccccccagcccctcctccccttcctgcacccgtacccccgtggtctttgaataaagtctgagtgggcggcagatct

SEQ ID NO: 190 - RNA, 124 nt, Artificial Sequence
  HBA1-v3 3UTR mRNA
ggacuagugcuggagccucgguggccaugcuucuugccccuugggccuccccccagccccuccuccccuuccugcacccguacccccguggucuuugaauaaagucugagugggcggcagaucu

SEQ ID NO: 191 - DNA, 122 bp, Artificial Sequence
  HBA2-v3 3UTR
ggactagtgctggagcctcggtagccgttcctcctgcccgctgggcctcccaacgggccctcctcccctccttgcaccggcccttcctggtctttgaataaagtctgagtgggcagagatct

SEQ ID NO: 192 - RNA, 122 nt, Artificial Sequence
  HBA2-v3 3UTR mRNA
ggacuagugcuggagccucgguagccguuccuccugcccgcugggccucccaacgggcccuccuccccuccuugcaccggcccuuccuggucuuugaauaaagucugagugggcagagaucu

SEQ ID NO: 193 - DNA, 146 bp, Artificial Sequence
  HBB-v3 3UTR
ggactagtgctcgctttcttgctgtccaatttctattaaaggttcctttgttccctaagtccaactactaaactgggggatattatgaagggccttgagcatctggattctgcctaataaaaaacatttattttcattgcagatct

SEQ ID NO: 194 - RNA, 146 nt, Artificial Sequence
  HBB-v3 3UTR mRNA
ggacuagugcucgcuuucuugcuguccaauuucuauuaaagguuccuuuguucccuaaguccaacuacuaaacugggggauauuaugaagggccuugagcaucuggauucugccuaauaaaaaacauuuauuuucauugcagaucu

SEQ ID NO: 195 - RNA, 6 nt, Artificial Sequence
  mRNA 5-end
gggaga

SEQ ID NO: 196 - RNA, 6 nt, Artificial Sequence
  mRNA 5-end
aggaga

SEQ ID NO: 197 - RNA, 6 nt, Artificial Sequence
  mRNA 5-end
gggaaa

SEQ ID NO: 198 - RNA, 6 nt, Artificial Sequence
  mRNA 5-end
agaaua

SEQ ID NO: 199 - RNA, 6 nt, Artificial Sequence
  mRNA 5-end
agauua

SEQ ID NO: 200 - RNA, 6 nt, Artificial Sequence
  mRNA 5-end
gauggg

SEQ ID NO: 201 - RNA, 5 nt, Artificial Sequence
  mRNA 5-end
gggcg

SEQ ID NO: 202 - DNA, 13 bp, Artificial Sequence
  Kozak sequence
gccgccaccatgg

SEQ ID NO: 203 - RNA, 13 nt, Artificial Sequence
  Kozak sequence mRNA
gccgccaccaugg

SEQ ID NO: 204 - DNA, 9 bp, Artificial Sequence
  Kozak sequence
gccgccacc

SEQ ID NO: 205 - RNA, 9 nt, Artificial Sequence
  Kozak sequence mRNA
gccgccacc

SEQ ID NO: 206 - DNA, 6 bp, Artificial Sequence
  Kozak sequence
gccacc

SEQ ID NO: 207 - RNA, 6 nt, Artificial Sequence
  Kozak sequence mRNA
gccacc

SEQ ID NO: 208 - DNA, 3 bp, Artificial Sequence
  Kozak sequence
acc

SEQ ID NO: 209 - RNA, 3 nt, Artificial Sequence
  Kozak sequence mRNA
acc

SEQ ID NO: 210 - DNA, 24 bp, Artificial Sequence
  histone stem-loop sequence (histoneSL, hSL)
caaaggctcttttcagagccacca

SEQ ID NO: 211 - RNA, 24 nt, Artificial Sequence
  histone stem-loop sequence (histoneSL, hSL) mRNA
caaaggcucuuuucagagccacca

SEQ ID NO: 212 - RNA, 128 nt, Artificial Sequence
  mRNA 3-end A64-N5-C30-histoneSL-N5
aaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 213 - RNA, 129 nt, Artificial Sequence
  mRNA 3-end histoneSL-A100-N5
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 214 - RNA, 93 nt, Artificial Sequence
  mRNA 3-end histoneSL-A64-N5
caaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 215 - RNA, 105 nt, Artificial Sequence
  mRNA 3-end A100-N5
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 216 - RNA, 69 nt, Artificial Sequence
  mRNA 3-end A64-N5
aaaaaaaaaaaaaaaaaaaaaaaaaaaaaaaaaaaaaaaaaaaaaaaaaaaaaaaaaaaaaaaagaauu

SEQ ID NO: 217 - RNA, 124 nt, Artificial Sequence
  mRNA 3-end histoneSL-A100
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 218 - RNA, 88 nt, Artificial Sequence
  mRNA 3-end histoneSL-A64
caaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 219 - RNA, 100 nt, Artificial Sequence
  mRNA 3-end A100
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 220 - RNA, 64 nt, Artificial Sequence
  mRNA 3-end A64
aaaaaaaaaaaaaaaaaaaaaaaaaaaaaaaaaaaaaaaaaaaaaaaaaaaaaaaaaaaaaaaa

SEQ ID NO: 221 - RNA, 93 nt, Artificial Sequence
  mRNA 3-end A64-histoneSL-N5
aaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 222 - RNA, 134 nt, Artificial Sequence
  mRNA 3-end N6-A64-N5-C30-histoneSL-N5
auuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 223 - RNA, 134 nt, Artificial Sequence
  mRNA 3-end N6-A64-N5-C30-histoneSL-N5
agaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 224 - RNA, 135 nt, Artificial Sequence
  mRNA 3-end N6-histoneSL-A100-N5
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 225 - RNA, 135 nt, Artificial Sequence
  mRNA 3-end N6-histoneSL-A100-N5
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 226 - RNA, 99 nt, Artificial Sequence
  mRNA 3-end N6-histoneSL-A64-N5
auuaaucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 227 - RNA, 99 nt, Artificial Sequence
  mRNA 3-end N6-histoneSL-A64-N5
agaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 228 - RNA, 111 nt, Artificial Sequence
  mRNA 3-end N6-A100-N5
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 229 - RNA, 111 nt, Artificial Sequence
  mRNA 3-end N6-A100-N5
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 230 - RNA, 75 nt, Artificial Sequence
  mRNA 3-end N6-A64-N5
auuaauaaaaaaaaaaaaaaaaaaaaaaaaaaaaaaaaaaaaaaaaaaaaaaaaaaaaaaaaaaaaaaaagaauu

SEQ ID NO: 231 - RNA, 75 nt, Artificial Sequence
  mRNA 3-end N6-A64-N5
agaucuaaaaaaaaaaaaaaaaaaaaaaaaaaaaaaaaaaaaaaaaaaaaaaaaaaaaaaaaaaaaaaaagaauu

SEQ ID NO: 232 - RNA, 130 nt, Artificial Sequence
  mRNA 3-end N6-histoneSL-A100
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 233 - RNA, 130 nt, Artificial Sequence
  mRNA 3-end N6-histoneSL-A100
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 234 - RNA, 94 nt, Artificial Sequence
  mRNA 3-end N6-histoneSL-A64
auuaaucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 235 - RNA, 94 nt, Artificial Sequence
  mRNA 3-end N6-histoneSL-A64
agaucucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 236 - RNA, 106 nt, Artificial Sequence
  mRNA 3-end N6-A100
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 237 - RNA, 106 nt, Artificial Sequence
  mRNA 3-end N6-A100
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 238 - RNA, 70 nt, Artificial Sequence
  mRNA 3-end N6-A64
auuaauaaaaaaaaaaaaaaaaaaaaaaaaaaaaaaaaaaaaaaaaaaaaaaaaaaaaaaaaaaaaaaaa

SEQ ID NO: 239 - RNA, 70 nt, Artificial Sequence
  mRNA 3-end N6-A64
agaucuaaaaaaaaaaaaaaaaaaaaaaaaaaaaaaaaaaaaaaaaaaaaaaaaaaaaaaaaaaaaaaaa

SEQ ID NO: 240 - RNA, 99 nt, Artificial Sequence
  mRNA 3-end N6-A64-histoneSL-N5
auuaauaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 241 - RNA, 99 nt, Artificial Sequence
  mRNA 3-end N6-A64-histoneSL-N5
agaucuaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 242 - RNA, 105 nt, Artificial Sequence
  mRNA 3-end A100-N5
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 243 - RNA, 110 nt, Artificial Sequence
  mRNA 3-end A30-N10-A70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 244 - protein, 14 aa, Artificial Sequence
  CR12C carrier peptide
CRRRRRRRRRRRRC

SEQ ID NO: 245 - protein, 13 aa, Artificial Sequence
  CR12 carrier peptide
CRRRRRRRRRRRR

SEQ ID NO: 246 - protein, 14 aa, Artificial Sequence
  WR12C carrier peptide
WRRRRRRRRRRRRC

SEQ ID NO: 247 - protein, 18 aa, Artificial Sequence
  CH6R4H6C carrier peptide
CHHHHHHRRRRHHHHHHC

SEQ ID NO: 248 - protein, 18 aa, Artificial Sequence
  CGH5R4H5GC carrier peptide
CGHHHHHRRRRHHHHHGC

SEQ ID NO: 249 - RNA, 22 nt, Artificial sequence
  miRNA binding site 122-5p
caaacaccauugucacacucca

SEQ ID NO: 250 - RNA, 22 nt, Artificial sequence
  miRNA binding site 122-3p
uauuuagugugauaauggcguu

SEQ ID NO: 251 - RNA, 22 nt, Artificial sequence
  miRNA binding site 148a-5p
agucggagugucucagaacuuu

SEQ ID NO: 252 - RNA, 22 nt, Artificial sequence
  miRNA binding site 148a-3p
acaaaguucuguagugcacuga

SEQ ID NO: 253 - RNA, 22 nt, Artificial sequence
  miRNA binding site 101-5p
agcaucagcacugugauaacug

SEQ ID NO: 254 - RNA, 21 nt, Artificial sequence
  miRNA binding site 101-3p
uucaguuaucacaguacugua

SEQ ID NO: 255 - RNA, 21 nt, Artificial sequence
  miRNA binding site 192-5p
ggcugucaauucauaggucag

SEQ ID NO: 256 - RNA, 22 nt, Artificial sequence
  miRNA binding site 192-3p
cugugaccuauggaauuggcag

SEQ ID NO: 257 - RNA, 22 nt, Artificial sequence
  miRNA binding site 194-5p
cagauaacagcagccccacugg

SEQ ID NO: 258 - RNA, 22 nt, Artificial sequence
  miRNA binding site 194-3p
uccacauggaguugcuguuaca

SEQ ID NO: 259 - RNA, 1947 nt, Artificial sequence
  1x miRNA-148a-3p binding site in front of 5UTR in PpLuc encoding mRNA
aggagaacaaaguucuguagugcacugagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 260 - RNA, 1947 nt, Artificial sequence
  1x miRNA-148a-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucacaaaguucuguagugcacugaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 261 - RNA, 1947 nt, Artificial sequence
  1x miRNA-122-5p binding site in front of 5UTR in PpLuc encoding mRNA
aggagacaaacaccauugucacacuccagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 262 - RNA, 1947 nt, Artificial sequence
  1x miRNA-122-5p downstream of 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuuccaaacaccauugucacacuccaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 263 - RNA, 1946 nt, Artificial sequence
  1x miRNA-101-3p binding site in front of 5UTR in PpLuc encoding mRNA
aggagauucaguuaucacaguacuguagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 264 - RNA, 1946 nt, Artificial sequence
  1x miRNA-101-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucuucaguuaucacaguacuguaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 265 - RNA, 1946 nt, Artificial sequence
  1x miRNA-192-5p binding site in front of 5UTR in PpLuc encoding mRNA
aggagaggcugucaauucauaggucaggucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 266 - RNA, 1946 nt, Artificial sequence
  1x miRNA-192-5p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucggcugucaauucauaggucagagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 267 - RNA, 1925 nt, Artificial sequence
  control mRNA encoding PpLuc
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 268 - RNA, 1969 nt, Artificial sequence
  2x miRNA-148a-3p binding site in front of 5UTR in PpLuc encoding mRNA
aggagaacaaaguucuguagugcacugaacaaaguucuguagugcacugagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 269 - RNA, 1969 nt, Artificial sequence
  2x miRNA-148a-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucacaaaguucuguagugcacugaacaaaguucuguagugcacugaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 270 - RNA, 1969 nt, Artificial sequence
  2x miRNA-122-5p binding site in front and within 5UTR in PpLuc encoding mRNA
aggagacaaacaccauugucacacuccagucccgcagucggcguccagcaaacaccauugucacacuccacggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 271 - RNA, 1969 nt, Artificial sequence
  2x miRNA-122-5p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuuccaaacaccauugucacacuccacaaacaccauugucacacuccaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 272 - RNA, 1967 nt, Artificial sequence
  2x miRNA-101-3p binding site in front and within 5UTR in PpLuc encoding mRNA
aggagauucaguuaucacaguacuguagucccgcagucggcguccaguucaguuaucacaguacuguacggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 273 - RNA, 1967 nt, Artificial sequence
  2x miRNA-101-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucuucaguuaucacaguacuguauucaguuaucacaguacuguaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 274 - RNA, 1968 nt, Artificial sequence
  miRNAs 122-5p/192-5p binding sites in PpLuc encoding mRNA
aggagacaaacaccauugucacacuccagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucggcugucaauucauaggucagagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 275 - RNA, 1968 nt, Artificial sequence
  miRNAs 101-3p/148a-3p binding sites in PpLuc encoding mRNA
aggagauucaguuaucacaguacuguagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucacaaaguucuguagugcacugaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 276 - RNA, 1969 nt, Artificial sequence
  miRNAs 148a-3p/194-5p binding sites in PpLuc encoding mRNA
aggagaacaaaguucuguagugcacugagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucuccacauggaguugcuguuacaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 277 - RNA, 1968 nt, Artificial sequence
  miRNAs 101-3p/194-5p binding sites in PpLuc encoding mRNA
aggagagucccgcagucggcguccaguucaguuaucacaguacuguacggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucuccacauggaguugcuguuacaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 278 - RNA, 1969 nt, Artificial sequence
  miRNAs 148a-3p/miR-122-5p binding sites in PpLuc encoding mRNA
aggagaacaaaguucuguagugcacugagucccgcagucggcguccagcaaacaccauugucacacuccacggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 279 - RNA, 1968 nt, Artificial sequence
  miRNAs 192-5p/194-5p binding site in PpLuc encoding mRNA
aggagaggcugucaauucauaggucaggucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauuccacauggaguugcuguuacaaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 280 - RNA, 1991 nt, Artificial sequence
  3x miRNA-148a-3p binding site in front of 5UTR in PpLuc encoding mRNA
aggagaacaaaguucuguagugcacugaacaaaguucuguagugcacugaacaaaguucuguagugcacugagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 281 - RNA, 1991 nt, Artificial sequence
  3x miRNA-148a-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucacaaaguucuguagugcacugaacaaaguucuguagugcacugaacaaaguucuguagugcacugaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 282 - RNA, 1991 nt, Artificial sequence
  3x miRNA-122-5p binding site in front of 5UTR in PpLuc encoding mRNA
aggagacaaacaccauugucacacuccacaaacaccauugucacacuccacaaacaccauugucacacuccagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 283 - RNA, 1991 nt, Artificial sequence
  3x miRNA-122-5p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuuccaaacaccauugucacacuccacaaacaccauugucacacuccacaaacaccauugucacacuccaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 284 - RNA, 1988 nt, Artificial sequence
  3x miRNA-101-3p in front of the 5UTR in PpLuc encoding mRNA
aggagauucaguuaucacaguacuguauucaguuaucacaguacuguauucaguuaucacaguacuguagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 285 - RNA, 1988 nt, Artificial sequence
  3x miRNA-101-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucuucaguuaucacaguacuguauucaguuaucacaguacuguauucaguuaucacaguacuguaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 286 - RNA, 1947 nt, Artificial sequence; identical to SEQ ID NO: 259
  1x miRNA-148-3p binding site in front of 5UTR in PpLuc encoding mRNA

SEQ ID NO: 287 - RNA, 1946 nt, Artificial sequence; identical to SEQ ID NO: 265
  1x miRNA-192-5p binding site in front of 5UTR in PpLuc encoding mRNA

SEQ ID NO: 288 - RNA, 1947 nt, Artificial sequence; identical to SEQ ID NO: 261
  1x miRNA-122-5p binding site in front of 5UTR in PpLuc encoding mRNA

SEQ ID NO: 289 - RNA, 1947 nt, Artificial sequence; identical to SEQ ID NO: 260
  1x miRNA-148-3p binding site after 3UTR in PpLuc encoding mRNA

SEQ ID NO: 290 - RNA, 1946 nt, Artificial sequence; identical to SEQ ID NO: 266
  1x miRNA-192-5p after 3UTR in PpLuc encoding mRNA

SEQ ID NO: 291 - RNA, 1947 nt, Artificial sequence; identical to SEQ ID NO: 262
  1x miRNA-148-3p binding site after 3UTR in PpLuc encoding mRNA

SEQ ID NO: 292 - RNA, 1925 nt, Artificial sequence; identical to SEQ ID NO: 267
  control mRNA encoding PpLuc

SEQ ID NO: 293 - RNA, 1845 nt, Artificial sequence
  control mRNA encoding IL-12
aggagagucccgcagucggcguccagcggcucugcuuguucgugugugugucguugcaggccuuauucaagcuuaccaugugccaccagcagcuggugaucagcugguucucccucgucuuccuggccagcccccugguggccaucugggagcucaagaaggacguguacgucguggagcuggacugguacccggacgcgcccggcgagauggugguccugaccugcgacacccccgaggaggacgggaucacguggacccucgaccaguccagcgaggugcugggcuccgggaagacccugaccauccaggugaaggaguucggcgacgccgggcaguacacgugccacaagggcggcgagguccucagccacucccugcugcuccugcacaagaaggaggacgggaucuggagcaccgacauccugaaggaccagaaggagcccaagaacaagaccuuccuccgcugcgaggccaagaacuacuccggccgguucacgugcugguggcugaccaccaucagcaccgaccugaccuucuccgucaagagcucccgcgggagcuccgacccgcagggcgugacgugcggggccgccacccucagcgcggagcgggugcgcggcgacaacaaggaguacgaguacuccguggagugccaggaggacagcgccugccccgccgccgaggagucccugcccaucgaggugauggucgacgcggugcacaagcugaaguacgagaacuacaccagcagcuucuucauccgggacaucaucaagcccgacccgcccaagaaccuccagcugaagccccugaagaacucccgccagguggaggucagcugggaguaccccgacaccugguccacgccgcacagcuacuucucccucaccuucugcguccaggugcagggcaagagcaagcgggagaagaaggaccgcguguucaccgacaagaccuccgccacggugaucugccggaagaacgccagcaucuccgugcgcgcccaggaccgguacuacagcuccagcugguccgagugggccagcguccccugcuccggcaguacuucgggcagugguaagccugguaguggugaggguaguacuaagggucggaaccugccgguggccacccccgaccccggcauguuccccugccugcaccacagccagaaccuccugcgcgccguguccaacaugcugcagaaggcccggcagacgcucgaguucuacccgugcaccagcgaggagaucgaccacgaggacaucaccaaggacaagaccuccacggucgaggccugccugccccuggagcucaccaagaacgagagcugccugaacucccgcgagaccagcuucaucaccaacggguccugccuggcgagccggaagacguccuucaugauggcccucugccugagcuccaucuacgaggaccugaagauguaccagguggaguucaagaccaugaacgccaagcuccugauggaccccaagcgccagaucuuccuggaccagaacaugcucgccgugaucgacgagcugaugcaggcgcugaacuucaacagcgagaccgucccccagaaguccagccucgaggagccggacuucuacaagaccaagaucaagcugugcauccugcuccacgccuuccggauccgcgccgugacgaucgaccgggugauguccuaccugaacgccagcugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaa

SEQ ID NO: 294 - RNA, 1867 nt, Artificial sequence
  1x miRNA-122-5p in front of 5UTR in IL12 encoding encoding mRNA
aggagacaaacaccauugucacacuccagucccgcagucggcguccagcggcucugcuuguucgugugugugucguugcaggccuuauucaagcuuaccaugugccaccagcagcuggugaucagcugguucucccucgucuuccuggccagcccccugguggccaucugggagcucaagaaggacguguacgucguggagcuggacugguacccggacgcgcccggcgagauggugguccugaccugcgacacccccgaggaggacgggaucacguggacccucgaccaguccagcgaggugcugggcuccgggaagacccugaccauccaggugaaggaguucggcgacgccgggcaguacacgugccacaagggcggcgagguccucagccacucccugcugcuccugcacaagaaggaggacgggaucuggagcaccgacauccugaaggaccagaaggagcccaagaacaagaccuuccuccgcugcgaggccaagaacuacuccggccgguucacgugcugguggcugaccaccaucagcaccgaccugaccuucuccgucaagagcucccgcgggagcuccgacccgcagggcgugacgugcggggccgccacccucagcgcggagcgggugcgcggcgacaacaaggaguacgaguacuccguggagugccaggaggacagcgccugccccgccgccgaggagucccugcccaucgaggugauggucgacgcggugcacaagcugaaguacgagaacuacaccagcagcuucuucauccgggacaucaucaagcccgacccgcccaagaaccuccagcugaagccccugaagaacucccgccagguggaggucagcugggaguaccccgacaccugguccacgccgcacagcuacuucucccucaccuucugcguccaggugcagggcaagagcaagcgggagaagaaggaccgcguguucaccgacaagaccuccgccacggugaucugccggaagaacgccagcaucuccgugcgcgcccaggaccgguacuacagcuccagcugguccgagugggccagcguccccugcuccggcaguacuucgggcagugguaagccugguaguggugaggguaguacuaagggucggaaccugccgguggccacccccgaccccggcauguuccccugccugcaccacagccagaaccuccugcgcgccguguccaacaugcugcagaaggcccggcagacgcucgaguucuacccgugcaccagcgaggagaucgaccacgaggacaucaccaaggacaagaccuccacggucgaggccugccugccccuggagcucaccaagaacgagagcugccugaacucccgcgagaccagcuucaucaccaacggguccugccuggcgagccggaagacguccuucaugauggcccucugccugagcuccaucuacgaggaccugaagauguaccagguggaguucaagaccaugaacgccaagcuccugauggaccccaagcgccagaucuuccuggaccagaacaugcucgccgugaucgacgagcugaugcaggcgcugaacuucaacagcgagaccgucccccagaaguccagccucgaggagccggacuucuacaagaccaagaucaagcugugcauccugcuccacgccuuccggauccgcgccgugacgaucgaccgggugauguccuaccugaacgccagcugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaa

SEQ ID NO: 295 - RNA, 1867 nt, Artificial sequence
  1x miRNA-122-5p after 3UTR in IL-12 encoding mRNA
aggagagucccgcagucggcguccagcggcucugcuuguucgugugugugucguugcaggccuuauucaagcuuaccaugugccaccagcagcuggugaucagcugguucucccucgucuuccuggccagcccccugguggccaucugggagcucaagaaggacguguacgucguggagcuggacugguacccggacgcgcccggcgagauggugguccugaccugcgacacccccgaggaggacgggaucacguggacccucgaccaguccagcgaggugcugggcuccgggaagacccugaccauccaggugaaggaguucggcgacgccgggcaguacacgugccacaagggcggcgagguccucagccacucccugcugcuccugcacaagaaggaggacgggaucuggagcaccgacauccugaaggaccagaaggagcccaagaacaagaccuuccuccgcugcgaggccaagaacuacuccggccgguucacgugcugguggcugaccaccaucagcaccgaccugaccuucuccgucaagagcucccgcgggagcuccgacccgcagggcgugacgugcggggccgccacccucagcgcggagcgggugcgcggcgacaacaaggaguacgaguacuccguggagugccaggaggacagcgccugccccgccgccgaggagucccugcccaucgaggugauggucgacgcggugcacaagcugaaguacgagaacuacaccagcagcuucuucauccgggacaucaucaagcccgacccgcccaagaaccuccagcugaagccccugaagaacucccgccagguggaggucagcugggaguaccccgacaccugguccacgccgcacagcuacuucucccucaccuucugcguccaggugcagggcaagagcaagcgggagaagaaggaccgcguguucaccgacaagaccuccgccacggugaucugccggaagaacgccagcaucuccgugcgcgcccaggaccgguacuacagcuccagcugguccgagugggccagcguccccugcuccggcaguacuucgggcagugguaagccugguaguggugaggguaguacuaagggucggaaccugccgguggccacccccgaccccggcauguuccccugccugcaccacagccagaaccuccugcgcgccguguccaacaugcugcagaaggcccggcagacgcucgaguucuacccgugcaccagcgaggagaucgaccacgaggacaucaccaaggacaagaccuccacggucgaggccugccugccccuggagcucaccaagaacgagagcugccugaacucccgcgagaccagcuucaucaccaacggguccugccuggcgagccggaagacguccuucaugauggcccucugccugagcuccaucuacgaggaccugaagauguaccagguggaguucaagaccaugaacgccaagcuccugauggaccccaagcgccagaucuuccuggaccagaacaugcucgccgugaucgacgagcugaugcaggcgcugaacuucaacagcgagaccgucccccagaaguccagccucgaggagccggacuucuacaagaccaagaucaagcugugcauccugcuccacgccuuccggauccgcgccgugacgaucgaccgggugauguccuaccugaacgccagcugaggacuagucccuguucccagagcccacuuuuuuuucuuuuuuugaaauaaaauagccugucuuuccaaacaccauugucacacuccaagaucuaaaaaaaaaaaaaaaaaaaaaaaaaaaaaaaaaaaaaaaaaaaaaaaaaaaaaaaaaaaaaaaaa

SEQ ID NO: 296 - RNA, 1948 nt, Artificial sequence
  1 x miRNA-142-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggccccgccccguucuacccccuggaggacgggaccgcgggcgagcagcuccacaaggccaugaagcgguacgcccuggugcccgggaccaucgccuucacggacgcccacaucgaggucgacaucaccuacgcggaguacuucgagaugagcgugcgccuggccgaggccaugaagcgguacggccucaacaccaaccaccgcaucguggucugcuccgagaacagccugcaguucuucaugcccgugcugggggcccucuucaucggcguggcggucgccccggccaacgacaucuacaacgagcgggagcugcugaacuccaugggcaucagccagcccaccgugguguucgucuccaagaaggggcuccagaagauccugaacgugcagaagaagcugccgaucauccagaagaucaucaucauggacagcaagacggacuaccagggcuuccaguccauguauaccuucgugaccagccaccuccccccgggguucaacgaguacgacuucguccccgaguccuucgaccgcgacaagaccaucgcccugaucaugaacagcuccggcagcacggggcugcccaagggcguggcccucccccaccggaccgcgugcgugcgcuucucccacgcccgggacccgaucuucggcaaccagaucauccccgacaccgccauccugagcgucgugcccuuccaccacggguucggcauguucaccacgcugggguaccucaucugcggcuuccgcgugguccugauguaccgguucgaggaggagcuguuccuccgcucccugcaggacuacaagauccagagcgcccugcucgugcccacccuguucuccuucuucgccaagagcacccugaucgacaaguacgaccucuccaaccugcacgagaucgcgagcggcggggccccgcugagcaaggaggugggcgaggccgucgccaagcgguuccaccuccccgggauccgccagggcuacgggcugaccgagacgaccuccgccauccugaucacccccgagggcgacgacaagcccggcgcgguggggaagguggucccguucuucgaggccaagguggucgaccucgacaccggcaagacgcugggggugaaccagcggggcgagcugugcgugcgcgggcccaugaucaugagcggcuacgucaacaaccccgaggccaccaacgcccucaucgacaaggacggcuggcugcacuccggggacaucgccuacugggacgaggacgagcacuucuucaucguggaccggcucaagagccucaucaaguacaagggcuaccagguggcgcccgccgagcuggaguccauccugcuccagcacccgaacaucuucgacgccggggucgccggccugcccgacgacgacgcgggggagcugcccgccgccguggugguccucgagcacggcaagaccaugaccgagaaggagaucguggacuacguggccagccaggucacgaccgccaagaagcugcgcggcggggugguguucgucgacgaggugcccaagggccugaccgggaagcuggacgcgcggaagauccgcgagauccucaucaaggccaagaagggcggcaagaucgccgucugaggacuagucccuguucccagagcccacuuuuuuuucuuuuuuugaaauaaaauagccugucuuucuccauaaaguaggaaacacuacaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 297 - RNA, 1948 nt, Artificial sequence
  1 x miRNA-142-3p binding site before 5′UTR in PpLuc encoding mRNA
aggagauccauaaaguaggaaacacuacagucccgcagucggcguccagcggcucugcuuguucgugugugugucguugcaggccuuauucaagcuuaccauggaggacgccaagaacaucaagaagggccccgccccguucuacccccuggaggacgggaccgcgggcgagcagcuccacaaggccaugaagcgguacgcccuggugcccgggaccaucgccuucacggacgcccacaucgaggucgacaucaccuacgcggaguacuucgagaugagcgugcgccuggccgaggccaugaagcgguacggccucaacaccaaccaccgcaucguggucugcuccgagaacagccugcaguucuucaugcccgugcugggggcccucuucaucggcguggcggucgccccggccaacgacaucuacaacgagcgggagcugcugaacuccaugggcaucagccagcccaccgugguguucgucuccaagaaggggcuccagaagauccugaacgugcagaagaagcugccgaucauccagaagaucaucaucauggacagcaagacggacuaccagggcuuccaguccauguauaccuucgugaccagccaccuccccccgggguucaacgaguacgacuucguccccgaguccuucgaccgcgacaagaccaucgcccugaucaugaacagcuccggcagcacggggcugcccaagggcguggcccucccccaccggaccgcgugcgugcgcuucucccacgcccgggacccgaucuucggcaaccagaucauccccgacaccgccauccugagcgucgugcccuuccaccacggguucggcauguucaccacgcugggguaccucaucugcggcuuccgcgugguccugauguaccgguucgaggaggagcuguuccuccgcucccugcaggacuacaagauccagagcgcccugcucgugcccacccuguucuccuucuucgccaagagcacccugaucgacaaguacgaccucuccaaccugcacgagaucgcgagcggcggggccccgcugagcaaggaggugggcgaggccgucgccaagcgguuccaccuccccgggauccgccagggcuacgggcugaccgagacgaccuccgccauccugaucacccccgagggcgacgacaagcccggcgcgguggggaagguggucccguucuucgaggccaagguggucgaccucgacaccggcaagacgcugggggugaaccagcggggcgagcugugcgugcgcgggcccaugaucaugagcggcuacgucaacaaccccgaggccaccaacgcccucaucgacaaggacggcuggcugcacuccggggacaucgccuacugggacgaggacgagcacuucuucaucguggaccggcucaagagccucaucaaguacaagggcuaccagguggcgcccgccgagcuggaguccauccugcuccagcacccgaacaucuucgacgccggggucgccggccugcccgacgacgacgcgggggagcugcccgccgccguggugguccucgagcacggcaagaccaugaccgagaaggagaucguggacuacguggccagccaggucacgaccgccaagaagcugcgcggcggggugguguucgucgacgaggugcccaagggccugaccgggaagcuggacgcgcggaagauccgcgagauccucaucaaggccaagaagggcggcaagaucgccgucugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 298 - RNA, 1947 nt, Artificial sequence
  1 x miRNA-223-3p binding site before 5UTR in PpLuc encoding mRNA
aggagaugggguauuugacaaacugacagucccgcagucggcguccagcggcucugcuuguucgugugugugucguugcaggccuuauucaagcuuaccauggaggacgccaagaacaucaagaagggccccgccccguucuacccccuggaggacgggaccgcgggcgagcagcuccacaaggccaugaagcgguacgcccuggugcccgggaccaucgccuucacggacgcccacaucgaggucgacaucaccuacgcggaguacuucgagaugagcgugcgccuggccgaggccaugaagcgguacggccucaacaccaaccaccgcaucguggucugcuccgagaacagccugcaguucuucaugcccgugcugggggcccucuucaucggcguggcggucgccccggccaacgacaucuacaacgagcgggagcugcugaacuccaugggcaucagccagcccaccgugguguucgucuccaagaaggggcuccagaagauccugaacgugcagaagaagcugccgaucauccagaagaucaucaucauggacagcaagacggacuaccagggcuuccaguccauguauaccuucgugaccagccaccuccccccgggguucaacgaguacgacuucguccccgaguccuucgaccgcgacaagaccaucgcccugaucaugaacagcuccggcagcacggggcugcccaagggcguggcccucccccaccggaccgcgugcgugcgcuucucccacgcccgggacccgaucuucggcaaccagaucauccccgacaccgccauccugagcgucgugcccuuccaccacggguucggcauguucaccacgcugggguaccucaucugcggcuuccgcgugguccugauguaccgguucgaggaggagcuguuccuccgcucccugcaggacuacaagauccagagcgcccugcucgugcccacccuguucuccuucuucgccaagagcacccugaucgacaaguacgaccucuccaaccugcacgagaucgcgagcggcggggccccgcugagcaaggaggugggcgaggccgucgccaagcgguuccaccuccccgggauccgccagggcuacgggcugaccgagacgaccuccgccauccugaucacccccgagggcgacgacaagcccggcgcgguggggaagguggucccguucuucgaggccaagguggucgaccucgacaccggcaagacgcugggggugaaccagcggggcgagcugugcgugcgcgggcccaugaucaugagcggcuacgucaacaaccccgaggccaccaacgcccucaucgacaaggacggcuggcugcacuccggggacaucgccuacugggacgaggacgagcacuucuucaucguggaccggcucaagagccucaucaaguacaagggcuaccagguggcgcccgccgagcuggaguccauccugcuccagcacccgaacaucuucgacgccggggucgccggccugcccgacgacgacgcgggggagcugcccgccgccguggugguccucgagcacggcaagaccaugaccgagaaggagaucguggacuacguggccagccaggucacgaccgccaagaagcugcgcggcggggugguguucgucgacgaggugcccaagggccugaccgggaagcuggacgcgcggaagauccgcgagauccucaucaaggccaagaagggcggcaagaucgccgucugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 299 - RNA, 1947 nt, Artificial sequence
  1 x miRNA-223-3p binding site after 3UTR in PpLuc encoding mRNA
aggagagucccgcagucggcguccagcggcucugcuuguucgugugugugucguugcaggccuuauucaagcuuaccauggaggacgccaagaacaucaagaagggccccgccccguucuacccccuggaggacgggaccgcgggcgagcagcuccacaaggccaugaagcgguacgcccuggugcccgggaccaucgccuucacggacgcccacaucgaggucgacaucaccuacgcggaguacuucgagaugagcgugcgccuggccgaggccaugaagcgguacggccucaacaccaaccaccgcaucguggucugcuccgagaacagccugcaguucuucaugcccgugcugggggcccucuucaucggcguggcggucgccccggccaacgacaucuacaacgagcgggagcugcugaacuccaugggcaucagccagcccaccgugguguucgucuccaagaaggggcuccagaagauccugaacgugcagaagaagcugccgaucauccagaagaucaucaucauggacagcaagacggacuaccagggcuuccaguccauguauaccuucgugaccagccaccuccccccgggguucaacgaguacgacuucguccccgaguccuucgaccgcgacaagaccaucgcccugaucaugaacagcuccggcagcacggggcugcccaagggcguggcccucccccaccggaccgcgugcgugcgcuucucccacgcccgggacccgaucuucggcaaccagaucauccccgacaccgccauccugagcgucgugcccuuccaccacggguucggcauguucaccacgcugggguaccucaucugcggcuuccgcgugguccugauguaccgguucgaggaggagcuguuccuccgcucccugcaggacuacaagauccagagcgcccugcucgugcccacccuguucuccuucuucgccaagagcacccugaucgacaaguacgaccucuccaaccugcacgagaucgcgagcggcggggccccgcugagcaaggaggugggcgaggccgucgccaagcgguuccaccuccccgggauccgccagggcuacgggcugaccgagacgaccuccgccauccugaucacccccgagggcgacgacaagcccggcgcgguggggaagguggucccguucuucgaggccaagguggucgaccucgacaccggcaagacgcugggggugaaccagcggggcgagcugugcgugcgcgggcccaugaucaugagcggcuacgucaacaaccccgaggccaccaacgcccucaucgacaaggacggcuggcugcacuccggggacaucgccuacugggacgaggacgagcacuucuucaucguggaccggcucaagagccucaucaaguacaagggcuaccagguggcgcccgccgagcuggaguccauccugcuccagcacccgaacaucuucgacgccggggucgccggccugcccgacgacgacgcgggggagcugcccgccgccguggugguccucgagcacggcaagaccaugaccgagaaggagaucguggacuacguggccagccaggucacgaccgccaagaagcugcgcggcggggugguguucgucgacgaggugcccaagggccugaccgggaagcuggacgcgcggaagauccgcgagauccucaucaaggccaagaagggcggcaagaucgccgucugaggacuagucccuguucccagagcccacuuuuuuuucuuuuuuugaaauaaaauagccugucuuucugggguauuugacaaacugacaagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 300 - RNA, 21 nt, Artificial sequence
  miRNA binding site 142-5p
aguagugcuuucuacuuuaug

SEQ ID NO: 301 - RNA, 23 nt, Artificial sequence
  miRNA binding site 142-3p
uccauaaaguaggaaacacuaca

SEQ ID NO: 302 - RNA, 22 nt, Artificial sequence
  miRNA binding site 223-5p
aacucagcuugucaaauacacg

SEQ ID NO: 303 - RNA, 22 nt, Artificial sequence
  miRNA binding site 223-3p
ugggguauuugacaaacugaca

SEQ ID NO: 304 - RNA, 84 nt, Artificial sequence
  Hs_hsa-miR-194-5p_binding_site.Hs_5UTR_HSD17B4_var.1
uccacauggaguugcuguuacagucccgcagucggcguccagcggcucugcuuguucgugugugugucguugcaggccuuauuc

SEQ ID NO: 305 - RNA, 84 nt, Artificial sequence
  Hs_hsa-miR-148a-3p_binding_site.Hs_5UTR_HSD17B4_var.1
acaaaguucuguagugcacugagucccgcagucggcguccagcggcucugcuuguucgugugugugucguugcaggccuuauuc

SEQ ID NO: 306 - RNA, 83 nt, Artificial sequence
  Hs_hsa-miR-101-3p_binding_site.Hs_5UTR_HSD17B4_var.1
uucaguuaucacaguacuguagucccgcagucggcguccagcggcucugcuuguucgugugugugucguugcaggccuuauuc

SEQ ID NO: 307 - RNA, 83 nt, Artificial sequence
  Hs_hsa-miR-192-5p_binding_site.Hs_5UTR_HSD17B4_var.1
ggcugucaauucauaggucaggucccgcagucggcguccagcggcucugcuuguucgugugugugucguugcaggccuuauuc

SEQ ID NO: 308 - RNA, 84 nt, Artificial sequence
  Hs_hsa-miR-122-5p_binding_site.Hs_5UTR_HSD17B4_var.1
caaacaccauugucacacuccagucccgcagucggcguccagcggcucugcuuguucgugugugugucguugcaggccuuauuc

SEQ ID NO: 309 - RNA, 106 nt, Artificial sequence
  Hs_hsa-miR-148a-3p_binding_site.Hs_hsa-miR-148a-3p_binding_site.H s_5UTR_HSD17B4_var.1
acaaaguucuguagugcacugaacaaaguucuguagugcacugagucccgcagucggcguccagcggcucugcuuguucgugugugugucguugcaggccuuauuc

SEQ ID NO: 310 - RNA, 104 nt, Artificial sequence
  Hs_hsa-miR-101-3p_binding_site.Hs_hsa-miR-101-3p_binding_site.Hs_ 5UTR_HSD17B4_var.1
uucaguuaucacaguacuguauucaguuaucacaguacuguagucccgcagucggcguccagcggcucugcuuguucgugugugugucguugcaggccuuauuc

SEQ ID NO: 311 - RNA, 104 nt, Artificial sequence
  Hs_hsa-miR-192-5p_binding_site.Hs_hsa-miR-192-5p_binding_site.Hs_ 5UTR_HSD17B4_var.1
ggcugucaauucauaggucagggcugucaauucauaggucaggucccgcagucggcguccagcggcucugcuuguucgugugugugucguugcaggccuuauuc

SEQ ID NO: 312 - RNA, 106 nt, Artificial sequence
  Hs_hsa-miR-122-5p_binding_site.Hs_hsa-miR-122-5p_binding_site.Hs_ 5UTR_HSD17B4_var.1
caaacaccauugucacacuccacaaacaccauugucacacuccagucccgcagucggcguccagcggcucugcuuguucgugugugugucguugcaggccuuauuc

SEQ ID NO: 313 - RNA, 128 nt, Artificial sequence
  Hs_hsa-miR-148a-3p_binding_site.Hs_hsa-miR-148a-3p_binding_site.H s_hsa-miR-148a-3p_binding_site.Hs_5UTR_HSD17B4_var.1
acaaaguucuguagugcacugaacaaaguucuguagugcacugaacaaaguucuguagugcacugagucccgcagucggcguccagcggcucugcuuguucgugugugugucguugcaggccuuauuc

SEQ ID NO: 314 - RNA, 125 nt, Artificial sequence
  Hs_hsa-miR-101-3p_binding_site.Hs_hsa-miR-101-3p_binding_site.Hs_ hsa-miR-101-3p_binding_site.Hs_5UTR_HSD17B4_var.1
uucaguuaucacaguacuguauucaguuaucacaguacuguauucaguuaucacaguacuguagucccgcagucggcguccagcggcucugcuuguucgugugugugucguugcaggccuuauuc

SEQ ID NO: 315 - RNA, 125 nt, Artificial sequence
  Hs_hsa-miR-192-5p_binding_site.Hs_hsa-miR-192-5p_binding_site.Hs_ hsa-miR-192-5p_binding_site.Hs_5UTR_HSD17B4_var.1
ggcugucaauucauaggucagggcugucaauucauaggucagggcugucaauucauaggucaggucccgcagucggcguccagcggcucugcuuguucgugugugugucguugcaggccuuauuc

SEQ ID NO: 316 - RNA, 128 nt, Artificial sequence
  Hs_hsa-miR-122-5p_binding_site.Hs_hsa-miR-122-5p_binding_site.Hs_ hsa-miR-122-5p_binding_site.Hs_5UTR_HSD17B4_var.1
caaacaccauugucacacuccacaaacaccauugucacacuccacaaacaccauugucacacuccagucccgcagucggcguccagcggcucugcuuguucgugugugugucguugcaggccuuauuc

SEQ ID NO: 317 - RNA, 106 nt, Artificial sequence
  Hs_hsa-miR-194-5p_binding_site.Hs_hsa-miR-194-5p_binding_site.Hs_ 5UTR_HSD17B4_var.1
uccacauggaguugcuguuacauccacauggaguugcuguuacagucccgcagucggcguccagcggcucugcuuguucgugugugugucguugcaggccuuauuc

SEQ ID NO: 318 - RNA, 128 nt, Artificial sequence
  Hs_hsa-miR-194-5p_binding_site.Hs_hsa-miR-194-5p_binding_site.Hs_ hsa-miR-194-5p_binding_site.Hs_5UTR_HSD17B4_var.1
uccacauggaguugcuguuacauccacauggaguugcuguuacauccacauggaguugcuguuacagucccgcagucggcguccagcggcucugcuuguucgugugugugucguugcaggccuuauuc

SEQ ID NO: 319 - RNA, 105 nt, Artificial sequence
  Hs_hsa-miR-148a-3p_binding_site.Hs_5UTR_HSD17B4_var_1-20.Hs_hsa- miR-101-3p_binding_site.Hs_5UTR_HSD17B4_var_21-62.1
acaaaguucuguagugcacugagucccgcagucggcguccaguucaguuaucacaguacuguacggcucugcuuguucgugugugugucguugcaggccuuauuc

SEQ ID NO: 320 - RNA, 106 nt, Artificial sequence
  Hs_hsa-miR-148a-3p_binding_site.Hs_5UTR_HSD17B4_var_1-20.Hs_hsa- miR-122-5p_binding_site.Hs_5UTR_HSD17B4_var_21-62.1
acaaaguucuguagugcacugagucccgcagucggcguccagcaaacaccauugucacacuccacggcucugcuuguucgugugugugucguugcaggccuuauuc

SEQ ID NO: 321 - RNA, 105 nt, Artificial sequence
  Hs_hsa-miR-101-3p_binding_site.Hs_5UTR_HSD17B4_var_1-20.Hs_hsa- miR-122-5p_binding_site.Hs_5UTR_HSD17B4_var_21-62.1
uucaguuaucacaguacuguagucccgcagucggcguccagcaaacaccauugucacacuccacggcucugcuuguucgugugugugucguugcaggccuuauuc

SEQ ID NO: 322 - RNA, 104 nt, Artificial sequence
  Hs_hsa-miR-192-5p_binding_site.Hs_5UTR_HSD17B4_var_1-20.Hs_hsa- miR-101-3p_binding_site.Hs_5UTR_HSD17B4_var_21-62.1
ggcugucaauucauaggucaggucccgcagucggcguccaguucaguuaucacaguacuguacggcucugcuuguucgugugugugucguugcaggccuuauuc

SEQ ID NO: 323 - RNA, 105 nt, Artificial sequence
  Hs_hsa-miR-192-5p_binding_site.Hs_5UTR_HSD17B4_var_1-20.Hs_hsa- miR-122-5p_binding_site.Hs_5UTR_HSD17B4_var_21-62.1
ggcugucaauucauaggucaggucccgcagucggcguccagcaaacaccauugucacacuccacggcucugcuuguucgugugugugucguugcaggccuuauuc

SEQ ID NO: 324 - RNA, 105 nt, Artificial sequence
  Hs_hsa-miR-122-5p_binding_site.Hs_5UTR_HSD17B4_var_1-20.Hs_hsa- miR-101-3p_binding_site.Hs_5UTR_HSD17B4_var_21-62.1
caaacaccauugucacacuccagucccgcagucggcguccaguucaguuaucacaguacuguacggcucugcuuguucgugugugugucguugcaggccuuauuc

SEQ ID NO: 325 - RNA, 104 nt, Artificial sequence
  Hs_hsa-miR-101-3p_binding_site.Hs_5UTR_HSD17B4_var_1-20.Hs_hsa- miR-101-3p_binding_site.Hs_5UTR_HSD17B4_var_21-62.1
uucaguuaucacaguacuguagucccgcagucggcguccaguucaguuaucacaguacuguacggcucugcuuguucgugugugugucguugcaggccuuauuc

SEQ ID NO: 326 - RNA, 106 nt, Artificial sequence
  Hs_hsa-miR-122-5p_binding_site.Hs_5UTR_HSD17B4_var_1-20.Hs_hsa- miR-122-5p_binding_site.Hs_5UTR_HSD17B4_var_21-62.1
caaacaccauugucacacuccagucccgcagucggcguccagcaaacaccauugucacacuccacggcucugcuuguucgugugugugucguugcaggccuuauuc

SEQ ID NO: 327 - RNA, 106 nt, Artificial sequence
  Hs_hsa-miR-148a-3p_binding_site.Hs_5UTR_HSD17B4_var_1-59.Hs_hsa- miR-148a-3p_binding_site.Hs_5UTR_HSD17B4_var_60-62.1
acaaaguucuguagugcacugagucccgcagucggcguccagcggcucugcuuguucgugugugugucguugcaggccuuaacaaaguucuguagugcacugauuc

SEQ ID NO: 328 - RNA, 105 nt, Artificial sequence
  Hs_hsa-miR-101-3p_binding_site.Hs_5UTR_HSD17B4_var_1-59.Hs_hsa- miR-148a-3p_binding_site.Hs_5UTR_HSD17B4_var_60-62.1
uucaguuaucacaguacuguagucccgcagucggcguccagcggcucugcuuguucgugugugugucguugcaggccuuaacaaaguucuguagugcacugauuc

SEQ ID NO: 329 - RNA, 106 nt, Artificial sequence
  Hs_hsa-miR-122-5p_binding_site.Hs_5UTR_HSD17B4_var_1-59.Hs_hsa- miR-148a-3p_binding_site.Hs_5UTR_HSD17B4_var_60-62.1
caaacaccauugucacacuccagucccgcagucggcguccagcggcucugcuuguucgugugugugucguugcaggccuuaacaaaguucuguagugcacugauuc

SEQ ID NO: 330 - RNA, 125 nt, Artificial sequence
  Hs_hsa-miR-101-3p_binding_site.Hs_5UTR_HSD17B4_var_1-20.Hs_hsa- miR-101-3p_binding_site.Hs_5UTR_HSD17B4_var_21-52.Hs_hsa-miR-101- 3p_binding_site.Hs_5UTR_HSD17B4_var_53-62
uucaguuaucacaguacuguagucccgcagucggcguccaguucaguuaucacaguacuguacggcucugcuuguucgugugugugucguugcauucaguuaucacaguacuguaggccuuauuc

SEQ ID NO: 331 - RNA, 128 nt, Artificial sequence
  Hs_hsa-miR-122-5p_binding_site.Hs_5UTR_HSD17B4_var_1-20.Hs_hsa- miR-122-5p_binding_site.Hs_5UTR_HSD17B4_var_21-52.Hs_hsa-miR-122- 5p_binding_site.Hs_5UTR_HSD17B4_var_53-62
caaacaccauugucacacuccagucccgcagucggcguccagcaaacaccauugucacacuccacggcucugcuuguucgugugugugucguugcacaaacaccauugucacacuccaggccuuauuc

SEQ ID NO: 332 - RNA, 84 nt, Artificial sequence
  Hs_5UTR_HSD17B4_var_1-59.Hs_hsa-miR-148a-3p_binding_site.Hs_5UTR_ HSD17B4_var_60-62.1
gucccgcagucggcguccagcggcucugcuuguucgugugugugucguugcaggccuuaacaaaguucuguagugcacugauuc

SEQ ID NO: 333 - RNA, 83 nt, Artificial sequence
  Hs_5UTR_HSD17B4_var_1-20.Hs_hsa-miR-101-3p_binding_site.Hs_5UTR_H SD17B4_var_21-62.1
gucccgcagucggcguccaguucaguuaucacaguacuguacggcucugcuuguucgugugugugucguugcaggccuuauuc

SEQ ID NO: 334 - RNA, 84 nt, Artificial sequence
  Hs_5UTR_HSD17B4_var_1-20.Hs_hsa-miR-122-5p_binding_site.Hs_5UTR_H SD17B4_var_21-62.1
gucccgcagucggcguccagcaaacaccauugucacacuccacggcucugcuuguucgugugugugucguugcaggccuuauuc

SEQ ID NO: 335 - RNA, 84 nt, Artificial sequence
  Hs_5UTR_HSD17B4_var_1-11.Hs_hsa-miR-194-5p_binding_site.Hs_5UTR_H SD17B4_var_12-62.1
gucccgcagucuccacauggaguugcuguuacaggcguccagcggcucugcuuguucgugugugugucguugcaggccuuauuc

SEQ ID NO: 336 - RNA, 84 nt, Artificial sequence
  Hs_5UTR_HSD17B4_var_1-22.Hs_hsa-miR-194-5p_binding_site.Hs_5UTR_H SD17B4_var_23-62.1
gucccgcagucggcguccagcguccacauggaguugcuguuacagcucugcuuguucgugugugugucguugcaggccuuauuc

SEQ ID NO: 337 - RNA, 84 nt, Artificial sequence
  Hs_5UTR_HSD17B4_var_1-43.Hs_hsa-miR-122-5p_binding_site.Hs_5UTR_H SD17B4_var_44-62.1
gucccgcagucggcguccagcggcucugcuuguucgugugugucaaacaccauugucacacuccagucguugcaggccuuauuc

SEQ ID NO: 338 - RNA, 84 nt, Artificial sequence
  Hs_5UTR_HSD17B4_var_1-52.Hs_hsa-miR-122-5p_binding_site.Hs_5UTR_H SD17B4_var_53-62.1
gucccgcagucggcguccagcggcucugcuuguucgugugugugucguugcacaaacaccauugucacacuccaggccuuauuc

SEQ ID NO: 339 - RNA, 85 nt, Artificial sequence
  Hs_hsa-miR-142-3p_binding_site.Hs_5UTR_HSD17B4_var.1
uccauaaaguaggaaacacuacagucccgcagucggcguccagcggcucugcuuguucgugugugugucguugcaggccuuauuc

SEQ ID NO: 340 - RNA, 85 nt, Artificial sequence
  Hs_5UTR_HSD17B4_var_1-5.Hs_hsa-miR-142-3p_binding_site.Hs_5UTR_HS D17B4_var_6-62.1
gucccuccauaaaguaggaaacacuacagcagucggcguccagcggcucugcuuguucgugugugugucguugcaggccuuauuc

SEQ ID NO: 341 - RNA, 85 nt, Artificial sequence
  Hs_5UTR_HSD17B4_var_1-40.Hs_hsa-miR-142-3p_binding_site.Hs_5UTR_H SD17B4_var_41-62.1
gucccgcagucggcguccagcggcucugcuuguucguguguccauaaaguaggaaacacuacaugugucguugcaggccuuauuc

SEQ ID NO: 342 - RNA, 84 nt, Artificial sequence
  Hs_hsa-miR-223-3p_binding_site.Hs_5UTR_HSD17B4_var.1
ugggguauuugacaaacugacagucccgcagucggcguccagcggcucugcuuguucgugugugugucguugcaggccuuauuc

SEQ ID NO: 343 - RNA, 79 nt, Artificial sequence
  Hs_3UTR_PSMB3.Hs_hsa-miR-194-5p_binding_site.1
cccuguucccagagcccacuuuuuuuucuuuuuuugaaauaaaauagccugucuuucuccacauggaguugcuguuaca

SEQ ID NO: 344 - RNA, 79 nt, Artificial sequence
  Hs_3UTR_PSMB3.Hs_hsa-miR-148a-3p_binding_site.1
cccuguucccagagcccacuuuuuuuucuuuuuuugaaauaaaauagccugucuuucacaaaguucuguagugcacuga

SEQ ID NO: 345 - RNA, 78 nt, Artificial sequence
  Hs_3UTR_PSMB3.Hs_hsa-miR-101-3p_binding_site.1
cccuguucccagagcccacuuuuuuuucuuuuuuugaaauaaaauagccugucuuucuucaguuaucacaguacugua

SEQ ID NO: 346 - RNA, 78 nt, Artificial sequence
  Hs_3UTR_PSMB3.Hs_hsa-miR-192-5p_binding_site.1
cccuguucccagagcccacuuuuuuuucuuuuuuugaaauaaaauagccugucuuucggcugucaauucauaggucag

SEQ ID NO: 347 - RNA, 79 nt, Artificial sequence
  Hs_3UTR_PSMB3.Hs_hsa-miR-122-5p_binding_site.1
cccuguucccagagcccacuuuuuuuucuuuuuuugaaauaaaauagccugucuuuccaaacaccauugucacacucca

SEQ ID NO: 348 - RNA, 88 nt, Artificial sequence
  Hs_3UTR_muag.Hs_hsa-miR-194-5p_binding_site.1
uauaagacugacuagcccgaugggccucccaacgggcccuccuccccuccuugcaccgagauuaauuccacauggaguugcuguuaca

SEQ ID NO: 349 - RNA, 88 nt, Artificial sequence
  Hs_3UTR_muag.Hs_hsa-miR-148a-3p_binding_site.1
uauaagacugacuagcccgaugggccucccaacgggcccuccuccccuccuugcaccgagauuaauacaaaguucuguagugcacuga

SEQ ID NO: 350 - RNA, 87 nt, Artificial sequence
  Hs_3UTR_muag.Hs_hsa-miR-101-3p_binding_site.1
uauaagacugacuagcccgaugggccucccaacgggcccuccuccccuccuugcaccgagauuaauuucaguuaucacaguacugua

SEQ ID NO: 351 - RNA, 87 nt, Artificial sequence
  Hs_3UTR_muag.Hs_hsa-miR-192-5p_binding_site.1
uauaagacugacuagcccgaugggccucccaacgggcccuccuccccuccuugcaccgagauuaauggcugucaauucauaggucag

SEQ ID NO: 352 - RNA, 101 nt, Artificial sequence
  Hs_3UTR_PSMB3.Hs_hsa-miR-194-5p_binding_site.Hs_hsa-miR-194-5p_ binding_site.2
cccuguucccagagcccacuuuuuuuucuuuuuuugaaauaaaauagccugucuuucuccacauggaguugcuguuacauccacauggaguugcuguuaca

SEQ ID NO: 353 - RNA, 101 nt, Artificial sequence
  Hs_3UTR_PSMB3.Hs_hsa-miR-148a-3p_binding_site.Hs_hsa-miR-148a-3p_ binding_site.2
cccuguucccagagcccacuuuuuuuucuuuuuuugaaauaaaauagccugucuuucacaaaguucuguagugcacugaacaaaguucuguagugcacuga

SEQ ID NO: 354 - RNA, 99 nt, Artificial sequence
  Hs_3UTR_PSMB3.Hs_hsa-miR-101-3p_binding_site.Hs_hsa-miR-101-3p_ binding_site.2
cccuguucccagagcccacuuuuuuuucuuuuuuugaaauaaaauagccugucuuucuucaguuaucacaguacuguauucaguuaucacaguacugua

SEQ ID NO: 355 - RNA, 99 nt, Artificial sequence
  Hs_3UTR_PSMB3.Hs_hsa-miR-192-5p_binding_site.Hs_hsa-miR-192-5p_ binding_site.2
cccuguucccagagcccacuuuuuuuucuuuuuuugaaauaaaauagccugucuuucggcugucaauucauaggucagggcugucaauucauaggucag

SEQ ID NO: 356 - RNA, 101 nt, Artificial sequence
  Hs_3UTR_PSMB3.Hs_hsa-miR-122-5p_binding_site.Hs_hsa-miR-122-5p_ binding_site.2
cccuguucccagagcccacuuuuuuuucuuuuuuugaaauaaaauagccugucuuuccaaacaccauugucacacuccacaaacaccauugucacacucca

SEQ ID NO: 357 - RNA, 123 nt, Artificial sequence
  Hs_3UTR_PSMB3.Hs_hsa-miR-194-5p_binding_site.Hs_hsa-miR-194-5p_ binding_site.Hs_hsa-miR-194-5p_binding_site.1
cccuguucccagagcccacuuuuuuuucuuuuuuugaaauaaaauagccugucuuucuccacauggaguugcuguuacauccacauggaguugcuguuacauccacauggaguugcuguuaca

SEQ ID NO: 358 - RNA, 123 nt, Artificial sequence
  Hs_3UTR_PSMB3.Hs_hsa-miR-148a-3p_binding_site.Hs_hsa-miR-148a-3p_ binding_site.Hs_hsa-miR-148a-3p_binding_site.1
cccuguucccagagcccacuuuuuuuucuuuuuuugaaauaaaauagccugucuuucacaaaguucuguagugcacugaacaaaguucuguagugcacugaacaaaguucuguagugcacuga

SEQ ID NO: 359 - RNA, 120 nt, Artificial sequence
  Hs_3UTR_PSMB3.Hs_hsa-miR-101-3p_binding_site.Hs_hsa-miR-101-3p_ binding_site.Hs_hsa-miR-101-3p_binding_site.1
cccuguucccagagcccacuuuuuuuucuuuuuuugaaauaaaauagccugucuuucuucaguuaucacaguacuguauucaguuaucacaguacuguauucaguuaucacaguacugua

SEQ ID NO: 360 - RNA, 120 nt, Artificial sequence
  Hs_3UTR_PSMB3.Hs_hsa-miR-192-5p_binding_site.Hs_hsa-miR-192-5p_ binding_site.Hs_hsa-miR-192-5p_binding_site.1
cccuguucccagagcccacuuuuuuuucuuuuuuugaaauaaaauagccugucuuucggcugucaauucauaggucagggcugucaauucauaggucagggcugucaauucauaggucag

SEQ ID NO: 361 - RNA, 123 nt, Artificial sequence
  Hs_3UTR_PSMB3.Hs_hsa-miR-122-5p_binding_site.Hs_hsa-miR-122-5p_ binding_site.Hs_hsa-miR-122-5p_binding_site.1
cccuguucccagagcccacuuuuuuuucuuuuuuugaaauaaaauagccugucuuuccaaacaccauugucacacuccacaaacaccauugucacacuccacaaacaccauugucacacucca

SEQ ID NO: 362 - RNA, 99 nt, Artificial sequence
  Hs_3UTR_PSMB3_1-8.Hs_hsa-miR-192-5p_binding_site.Hs_3UTR_PSMB3_9- 57.Hs_hsa-miR-192-5p_binding_site.1
cccuguucggcugucaauucauaggucagccagagcccacuuuuuuuucuuuuuuugaaauaaaauagccugucuuucggcugucaauucauaggucag

SEQ ID NO: 363 - RNA, 78 nt, Artificial sequence
  Hs_3UTR_PSMB3_1-9.Hs_hsa-miR-192-5p_binding_site.Hs_3UTR_PSMB3_10 -57.1
cccuguuccggcugucaauucauaggucagcagagcccacuuuuuuuucuuuuuuugaaauaaaauagccugucuuuc

SEQ ID NO: 364 - RNA, 78 nt, Artificial sequence
  Hs_3UTR_PSMB3_1-8.Hs_hsa-miR-192-5p_binding_site.Hs_3UTR_PSMB3_9- 57.1
cccuguucggcugucaauucauaggucagccagagcccacuuuuuuuucuuuuuuugaaauaaaauagccugucuuuc

SEQ ID NO: 365 - RNA, 79 nt, Artificial sequence
  Hs_3UTR_PSMB3_1-40.Hs_hsa-miR-194-5p_binding_site.Hs_3UTR_PSMB3_4 1-57.1
cccuguucccagagcccacuuuuuuuucuuuuuuugaaauuccacauggaguugcuguuacaaaaauagccugucuuuc

SEQ ID NO: 366 - RNA, 79 nt, Artificial sequence
  Hs_3UTR_PSMB3_1-53.Hs_hsa-miR-122-5p_binding_site.Hs_3UTR_PSMB3_5 4-57.1
cccuguucccagagcccacuuuuuuuucuuuuuuugaaauaaaauagccuguccaaacaccauugucacacuccauuuc

SEQ ID NO: 367 - RNA, 79 nt, Artificial sequence
  Hs_3UTR_PSMB3_1-52.Hs_hsa-miR-122-5p_binding_site.Hs_3UTR_PSMB3_5 3-57.1
cccuguucccagagcccacuuuuuuuucuuuuuuugaaauaaaauagccugucaaacaccauugucacacuccacuuuc

SEQ ID NO: 368 - RNA, 100 nt, Artificial sequence
  Hs_3UTR_PSMB3_1-40.Hs_hsa-miR-194-5p_binding_site.Hs_3UTR_PSMB3_4 1-57.Hs_hsa-miR-194-5p_binding_site.1
cccuguucccagagcccacuuuuuuuucuuuuuuugaaauuccacauggaguugcuguuacaaaaauagccugucuuucggcugucaauucauaggucag

SEQ ID NO: 369 - RNA, 100 nt, Artificial sequence
  Hs_3UTR_PSMB3_1-8.Hs_hsa-miR-192-5p_binding_site.Hs_3UTR_PSMB3_9- 57.Hs_hsa-miR-194-5p_binding_site.1
cccuguucggcugucaauucauaggucagccagagcccacuuuuuuuucuuuuuuugaaauaaaauagccugucuuucuccacauggaguugcuguuaca

SEQ ID NO: 370 - RNA, 100 nt, Artificial sequence
  Hs_3UTR_PSMB3_1-8.Hs_hsa-miR-192-5p_binding_site.Hs_3UTR_PSMB3_9- 57.Hs_hsa-miR-148a-3p_binding_site.1
cccuguucggcugucaauucauaggucagccagagcccacuuuuuuuucuuuuuuugaaauaaaauagccugucuuucacaaaguucuguagugcacuga

SEQ ID NO: 371 - RNA, 101 nt, Artificial sequence
  Hs_3UTR_PSMB3_1-40.Hs_hsa-miR-194-5p_binding_site.Hs_3UTR_PSMB3_4 1-57.Hs_hsa-miR-148a-3p_binding_site.1
cccuguucccagagcccacuuuuuuuucuuuuuuugaaauuccacauggaguugcuguuacaaaaauagccugucuuucacaaaguucuguagugcacuga

SEQ ID NO: 372 - RNA, 100 nt, Artificial sequence; identical to SEQ ID NO: 368
  Hs_3UTR_PSMB3_1-40.Hs_hsa-miR-194-5p_binding_site.Hs_3UTR_PSMB3_4 1-57.Hs_hsa-miR-192-5p_binding_site.1

SEQ ID NO: 373 - RNA, 101 nt, Artificial sequence
  Hs_3UTR_PSMB3_1-40.Hs_hsa-miR-194-5p_binding_site.Hs_3UTR_PSMB3_4 1-57.Hs_hsa-miR-122-5p_binding_site.1
cccuguucccagagcccacuuuuuuuucuuuuuuugaaauuccacauggaguugcuguuacaaaaauagccugucuuuccaaacaccauugucacacucca

SEQ ID NO: 374 - RNA, 101 nt, Artificial sequence; identical to SEQ ID NO: 373
  Hs_3UTR_PSMB3_1-40.Hs_hsa-miR-194-5p_binding_site.Hs_3UTR_PSMB3_4 1-57.Hs_hsa-miR-122-5p_binding_site.1

SEQ ID NO: 375 - RNA, 123 nt, Artificial sequence
  Hs_3UTR_PSMB3_1-19.Hs_hsa-miR-194-5p_binding_site.Hs_3UTR_PSMB3_2 0-40.Hs_hsa-miR-194-5p_binding_site.Hs_3UTR_PSMB3_41-57.Hs_hsa-mi R-194-5p_binding_site.1
cccuguucccagagcccacuccacauggaguugcuguuacauuuuuuuucuuuuuuugaaauuccacauggaguugcuguuacaaaaauagccugucuuucuccacauggaguugcuguuaca

SEQ ID NO: 376 - RNA, 80 nt, Artificial sequence
  Hs_3UTR_PSMB3.Hs_hsa-miR-142-3p_binding_site.1
cccuguucccagagcccacuuuuuuuucuuuuuuugaaauaaaauagccugucuuucuccauaaaguaggaaacacuaca

SEQ ID NO: 377 - RNA, 80 nt, Artificial sequence
  Hs_3UTR_PSMB3_1-19.Hs_hsa-miR-142-3p_binding_site.Hs_3UTR_PSMB3_2 0-57.1
cccuguucccagagcccacuccauaaaguaggaaacacuacauuuuuuuucuuuuuuugaaauaaaauagccugucuuuc

SEQ ID NO: 378 - RNA, 89 nt, Artificial sequence
  Hs_3UTR_muag.Hs_hsa-miR-142-3p_binding_site.1
uauaagacugacuagcccgaugggccucccaacgggcccuccuccccuccuugcaccgagauuaauuccauaaaguaggaaacacuaca

SEQ ID NO: 379 - RNA, 79 nt, Artificial sequence
  Hs_3UTR_PSMB3.Hs_hsa-miR-223-3p_binding_site.1
cccuguucccagagcccacuuuuuuuucuuuuuuugaaauaaaauagccugucuuucugggguauuugacaaacugaca

SEQ ID NO: 380 - RNA, 79 nt, Artificial sequence
  Hs_3UTR_PSMB3_1-54.Hs_hsa-miR-223-3p_binding_site.Hs_3UTR_PSMB3_5 5-57.1
cccuguucccagagcccacuuuuuuuucuuuuuuugaaauaaaauagccugucuugggguauuugacaaacugacauuc

SEQ ID NO: 381 - RNA, 79 nt, Artificial sequence; identical to SEQ ID NO: 380
  Hs_3UTR_PSMB3_1-53.Hs_hsa-miR-223-3p_binding_site.Hs_3UTR_PSMB3_5 4-57.1

SEQ ID NO: 382 - RNA, 88 nt, Artificial sequence
  Hs_3UTR_muag.Hs_hsa-miR-223-3p_binding_site.1
uauaagacugacuagcccgaugggccucccaacgggcccuccuccccuccuugcaccgagauuaauugggguauuugacaaacugaca